(19) United States
(12) Patent Application Publication
(10) Pub. No.: US 2018/0243427 A1
(43) Pub. Date: Aug. 30, 2018

(54) TUBULYSIN COMPOUNDS AND CONJUGATES THEREOF

(71) Applicant: Mersana Therapeutics, Inc., Cambridge, MA (US)

(72) Inventors: Aleksandr V. YURKOVETSKIY, Littleton, MA (US); Timothy B. LOWINGER, Carlisle, MA (US)

(21) Appl. No.: 15/705,828

(22) Filed: Sep. 15, 2017

Related U.S. Application Data
(63) Continuation of application No. 14/776,449, filed on Sep. 14, 2015, now abandoned.
(60) Provisional application No. 61/785,820, filed on Mar. 14, 2013.

(51) Int. Cl. A61K 47/59; C08G 4/00; A61K 39/395; A61K 47/68; A61K 9/00; A61K 38/05

(52) U.S. Cl. CPC A61K 47/59; C08G 4/00; A61K 39/3955; A61K 38/05; A61K 9/0085; A61K 9/0019; A61K 47/6851

(57) ABSTRACT

A tubulysin compound conjugate is provided herein. The conjugate comprises a protein based recognition-molecule (PBRM) and a polymeric carrier substituted with one or more -L^{D}-D, the protein based recognition-molecule being connected to the polymeric carrier by L^{P}. Each occurrence of D is independently a tubulysin compound having a molecular weight ≤5 kDa. L^{D} and L^{P} are distinct linkers connecting the tubulysin compound and PBRM to the polymeric carrier respectively. Also disclosed are polymeric scaffolds useful for conjugating with a PBRM to form a polymer-tubulysin compound-PBRM conjugate described herein, compositions comprising the conjugates, methods of their preparation, and methods of treating various disorders with the conjugates or their compositions.

### RELATED APPLICATIONS

This application is a continuation application, filed under 35 U.S.C. § 120, of U.S. patent application Ser. No. 14/776,449, filed Sep. 14, 2015, which is a U.S. National Phase application, filed under 35 U.S.C. § 371, of International Application No. PCT/US14/26387, filed Mar. 13, 2014, which claims the benefit of and priority to U.S. Provisional Patent Application No. 61/785,820, filed Mar. 14, 2013, the contents of each of which are hereby incorporated by reference in their entireties.

### BACKGROUND OF THE INVENTION

Traditionally, pharmaceuticals have primarily consisted of small molecules that are dispensed orally (as solid pills and liquids) or as injectables. Over the past three decades, formulations (i.e., compositions that control the route and/or rate of drug delivery and allow delivery of the therapeutic agent at the site where it is needed) have become increasingly common and complex. Nevertheless, many questions and challenges regarding the development of new treatments as well as the mechanisms with which to administer them remain to be addressed. For example, many drugs exhibit limited or otherwise reduced potencies and therapeutic effects because they are either generally subject to partial degradation before they reach a desired target in the body, or accumulate in tissues other than the target, or both.

One objective in the field of drug delivery systems, therefore, is to deliver medications intact to specifically targeted areas of the body through a system that can stabilize the drug and control the in vivo transfer of the therapeutic agent utilizing either physiological or chemical mechanisms, or both.

Antibody-drug conjugates have been developed as target-specific therapeutic agents. Antibodies against various cancer cell-surface antigens have been conjugated with different cytotoxic agents that inhibit various essential cellular targets such as microtubules (maytansinoids, auristatins, taxanes: U.S. Pat. Nos. 5,208,020; 5,416,064; 6,333,410; 6,441,163; 6,340,701; 6,372,738; 6,436,931; 6,596,757; and 7,276,497); DNA (calicheamicin, doxorubicin, CC-1065 analogs; U.S. Pat. Nos. 5,475,092; 5,585,499; 5,846,545; 6,534,660; 6,756,397; and 6,630,579). Antibody conjugates with some of these cytotoxic drugs are actively being investigated in the clinic for cancer therapy (Ricart, A. D., and Tolcher, A. W., 2007, Nature Clinical Practice, 4, 245-255; Krop et al., 2010, J. Clin. Oncol., 28, 2698-2704). However, existing antibody-drug conjugates have exhibited a few limitations. A major limitation is their inability to deliver a sufficient concentration of drug to the target site because of the limited number of targeted antigens and the relatively moderate cytotoxicity of cancer drugs like methotrexate, daunorubicin, maytansinoids, taxanes, and vincristine. One approach to achieving significant cytotoxicity is by linkage of a large number of drug molecules either directly or indirectly to the antibody. However such heavily modified antibodies often display impaired binding to the target antigen and fast in vivo clearance from the blood stream. Therefore, there is a need to improve the ability to deliver a sufficient concentration of a drug to the target such that maximum cytotoxicity for the drug is achieved.

### SUMMARY OF THE INVENTION

The present invention relates to a protein-polymer-drug conjugate (i.e., a protein-polymer-tubulysin compound conjugate) that is biodegradable, biocompatible and exhibits high drug load as well as strong binding to target antigen. The present invention also relates to a polymeric scaffold useful to conjugate with a protein based recognition-molecule (PBRM) so as to obtain the protein-polymer-drug conjugate.

In one aspect, the invention relates to a polymeric scaffold of Formula (Ia) useful to conjugate with a protein based recognition-molecule (PBRM):

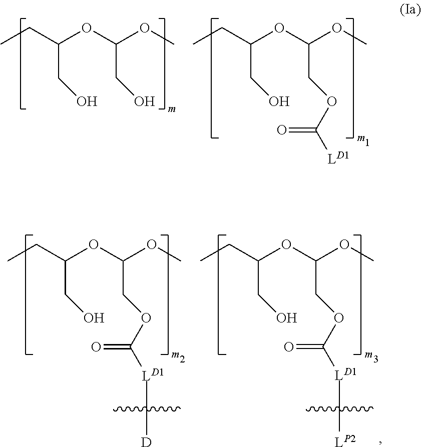

wherein:

the scaffold comprises poly(1-hydroxymethylethylene hydroxymethyl-formal) (PHF) having a molecular weight (i.e., MW of the unmodified PHF) ranging from 2 kDa to 40 kDa when the PBRM to be conjugated has a molecular weight greater than 40 kDa, or the scaffold comprises PHF having a molecular weight ranging from 20 kDa to 300 kDa when the PBRM to be conjugated has a molecular weight of less than 200 kDa (e.g., less than 80 kDa);

each occurrence of D is independently a tubulysin compound (e.g., a naturally occurring tubulysin or an analog or derivative thereof) having a molecular weight of ≤5 kDa;

L^{D1} is a carbonyl-containing moiety;

each occurrence of

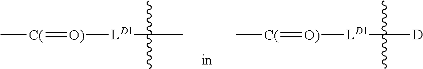

in is independently a first linker that contains a biodegradable bond so that when the bond is broken, D is released in an active form for its intended therapeutic effect; in which the

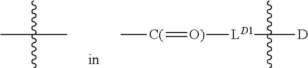

between L^{D1} and D denotes direct or indirect attachment of D to L^{D1};

each occurrence of

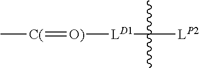

is independently a second linker not yet connected to the PBRM, in which L^{P2} is a moiety containing a functional group that is capable of forming and not yet formed a covalent bond with a functional group of a PBRM, and the

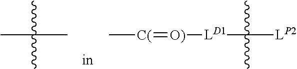

between L^{D1} and L^{P2} denotes direct or indirect attachment of L^{P2} to L^{D1}, and each occurrence of the second linker is distinct from each occurrence of the first linker;

m is an integer from 1 to 2200

m₁ is an integer from 1 to 660,

m₂ is an integer from 1 to 300,

m₃ is an integer from 1 to 110, and

the sum of m, m₁, m₂ and m₃ ranges from 15 to about 2200.

The scaffold of (Ia) can include one or more of the following features:

When the PHF in Formula (Ia) has a molecular weight ranging from about 2 kDa to about 40 kDa (i.e., the sum of m, m₁, m₂, and m₃ ranging from about 15 to about 300), m₂ is an integer from 1 to about 40, m₃ is an integer from 1 to about 18, and/or m₁ is an integer from 1 to about 140 (e.g., m₁ being about 1-90).

When the PHF in Formula (Ia) has a molecular weight ranging from about 6 kDa to about 20 kDa (i.e., the sum of m, m₁, m₂, and m₃ ranging from about 45 to about 150), m₂ is an integer from 2 to about 20, m₃ is an integer from 1 to about 9, and/or m₁ is an integer from 1 to about 75 (e.g., m₁ being about 4-45).

When the PHF in Formula (Ia) has a molecular weight ranging from about 8 kDa to about 15 kDa (i.e., the sum of m, m₁, m₂, and m₃ ranging from about 60 to about 110), m₂ is an integer from 2 to about 15, m is an integer from 1 to about 7, and/or m₁ is an integer from 1 to about 55 (e.g., m₁ being about 4-30).

When the PHF in Formula (Ia) has a molecular weight ranging from 20 kDa to 300 kDa (i.e., the sum of m, m₁, m₂, and m₃ ranging from about 150 to about 2200), m₂ is an integer from 3 to about 300, m₃ is an integer from 1 to about 110, and/or m₁ is an integer from 1 to about 660 (e.g., m₁ being about 10-250).

When the PHF in Formula (Ia) has a molecular weight ranging from 20 kDa to 150 kDa (i.e., the sum of m, m₁, m₂ and m₃ ranging from about 150 to about 1100), m₂ is an integer from 3 to about 150, m₃ is an integer from 1 to about 55, and/or m₁ is an integer from 1 to about 330 (e.g., m₁ being about 10-330 or about 15-100). This scaffold can be used, for example, for conjugating a PBRM having a molecular weight of about 4 kDa to about 80 kDa.

When the PHF in Formula (Ia) has a molecular weight ranging from 30 kDa to 100 kDa (i.e., the sum of m, m₁, m₂, and m₃ ranging from about 220 to about 740), m₂ is an integer from 3 to 100 (e.g., 5-100), m₃ is an integer from 1 to about 40, and/or m₁ is an integer from 1 to about 220 (e.g., m₁ being about 15-80).

For conjugating a PBRM having a molecular weight of 40 kDa or greater (e.g., 80 kDa or greater), the polymeric carrier of the scaffold of the invention is a polyacetal, e.g., a PHF having a molecular weight (i.e., MW of the unmodified PHF) ranging from about 2 kDa to about 40 kDa (e.g., about 6-20 kDa or about 8-15 kDa).

For conjugating a PBRM having a molecular weight of 200 kDa or less (e.g., 80 kDa or less), the polymeric carrier of the scaffold of the invention is a polyacetal, e.g., a PHF having a molecular weight (i.e., MW of the unmodified PHF) ranging from about 20 kDa to about 300 kDa (e.g., about 20-150 kDa or about 30-100 kDa).

The functional group of L^{P2} is selected from —SR^{p}, —S—S-LG, maleimido, and halo, in which LG is a leaving group and R^{p} is H or a sulfur protecting group.

L^{D1} comprises —X—(CH₂)ᵥ—C(═O)— with X directly connected to the carbonyl group of

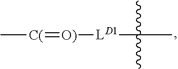

in which X is CH₂, O, or NH, and v is an integer from 1 to 6.

L^{P2} contains a biodegradable bond.

The tubulysin compound D, before conjugating with PHF or directly conjugating with a PBRM, is of Formula (IIA) or a pharmaceutically acceptable salt thereof:

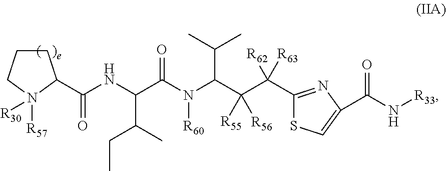

wherein:

e is 2;

R₅₅ is hydrogen;

R₅₆ is hydrogen or OH; or R₅₅ and R₅₆ together form an oxo group (═O);

R₅₇ is methyl or ethyl, or —C(O)R₅₈ and R₃₀ is absent or R₅₇ is methyl and R₃₀ is O;

R₅₈ is C₁₋₆ alkyl, CF₃ or C₆₋₁₀ aryl;

R₆₀ is hydrogen, methyl, —CH₂OR₆₅, or —CH₂NHR₆₅;

R₆₂ is hydrogen or alkyl;

R₆₃ is hydrogen, halo, OH, —O—C₁₋₄ alkyl or O—C(O)—R₃₄, in which R₃₄ is C₁₋₄ alkyl, C₂-7 alkenyl, or C₆₋₁₀ aryl; or R₆₂ and R₆₃ together form an oxo group (═O);

R₆₅ is hydrogen, C₁₋₆ alkyl optionally substituted with OH or SH, C₂₋₇ alkenyl, or C(O)R₆₇; and

R₆₇ is C₁₋₆ alkyl, C₂₋₇ alkenyl, C₆₋₁₀ aryl or heteroaryl;

R₃₃ is

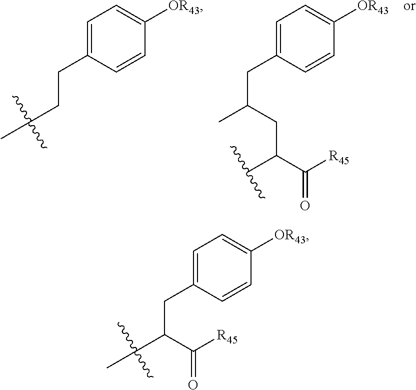

in which R₄₅ is mono- or di-alkylamino, —OR₄₂ or —NHR₄₀, and provided that at least one of R₄₃, R₄₂ and R₄₀ cannot be hydrogen;

R₄₀ is hydrogen, —OH, or —NH₂; R₄₂ is hydrogen; or each of R₄₀ and R₄₂, independently is selected from the following structures:

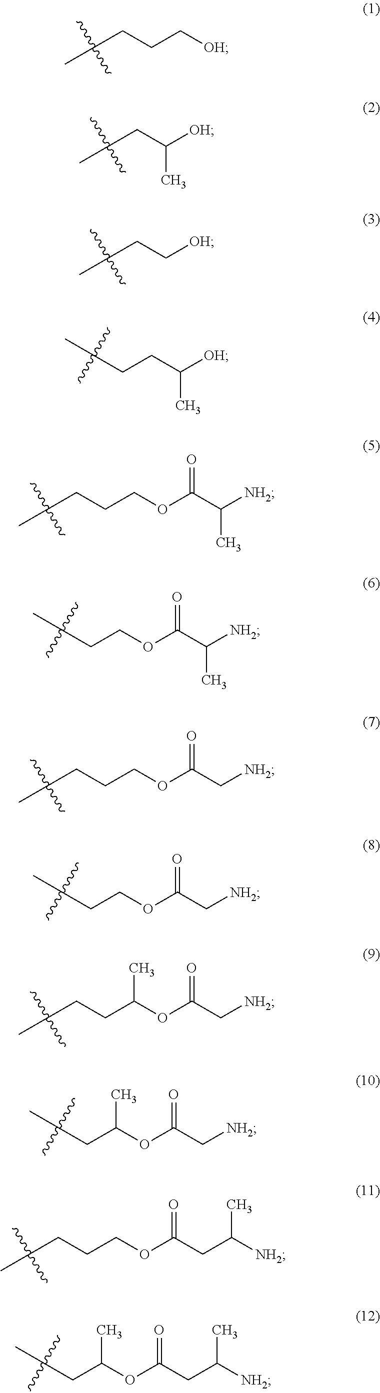

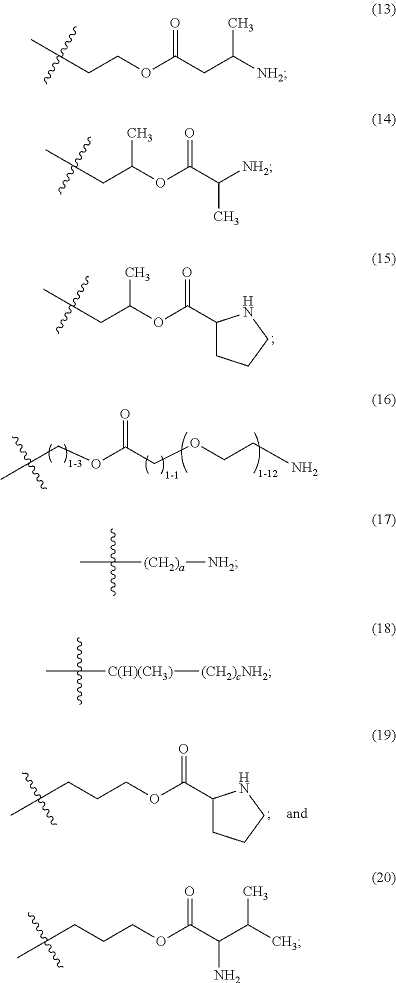

in which a is an integer from 1 to 6; and c is an integer from 0 to 3;

R₄₃ is H or —R₄₆—R₄₇;

R₄₆ is —C(O)—; —C(O)—O—, —C(O)—NH— or absent;

R₄₇ is an amino group, —R₉—[C(R₂₀R₂₁)]ₐ—R₁₀, —R₉—C₅₋₁₂ heterocycloalkyl-C₁₋₆ alkyl-R₁₀, 5 to 12-membered heterocycloalkyl, or —R₉—C₆₋₁₀ aryl;

R₉ is absent, N—(R₈₃) or oxygen;

R₁₀ is —OH, —NHR₈₃, —N—(R₈₃)R₁₁, —COOH, —R₈₂—C(O)(CH₂)_{c}—C(H)(R₂₃)—N(H)(R₂₃), —R₈₂—C(O)(CH₂)_{d}—(OCH₂—CH₂)_{f}—N(H)(R₂₃), —R₈₂—(C(O)—CH(X²)—NH)_{d}—R₇₇ or —R₈₂—C(O)—[C(R₂₀R₂₁)]ₐ—R₈₂—R₈₃ or

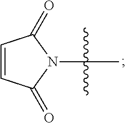 [SMILES: *N1C(=O)C=CC1=O]

X² is a side chain of a natural or unnatural amino acid;

R₇₇ is hydrogen or X² and NR₇₇ form a nitrogen containing cyclic compound;

R₈₂ is —NH or oxygen;

R₈₃ is hydrogen or CH₃;

each of R₂₀ and R₂₁ independently is hydrogen, C₁₋₆ alkyl, C₆₋₁₀ aryl, hydroxylated C₆-10 aryl, polyhydroxylated C₆₋₁₀ aryl, 5 to 12-membered heterocycle, C₃₋₈ cycloalkyl, hydroxylated C₃₋₈ cycloalkyl, polyhydroxylated C₃₋₈ cycloalkyl or a side chain of a natural or unnatural amino acid;

each R₂₃ independently is hydrogen, C₁₋₆ alkyl, C₆₋₁₀ aryl, C₃₋₈ cycloalkyl, —COOH, or —COO—C₁₋₆ alkyl;

a is an integer from 1 to 6;

c is an integer from 0 to 3;

d is an integer from 1 to 3;

f is an integer from 1 to 12;

R₁₁ is:

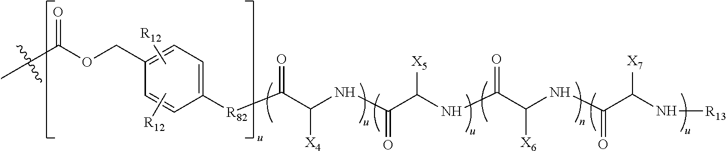

each R₁₂ independently is hydrogen, chloride, —CH₃ or —OCH₃;

R₁₃ is hydrogen or —C(O)—(CH₂)_{d}—(O—CH₂—CH₂)_{f}—NH₂;

R₈₂ is —NH or oxygen

X₄ is the side chain of lysine, arginine, citrulline, alanine or glycine;

X₅ is the side chain of phenylalanine, valine, leucine, isoleucine or tryptophan;

each of X₆ and X₇ is independently the side chain of glycine, alanine, serine, valine or proline;

each u independently is an integer 0 or 1;

or R₁₁ is —Yᵤ—W_{q}—R₈₈,

wherein:

Y is any one of the following structures:

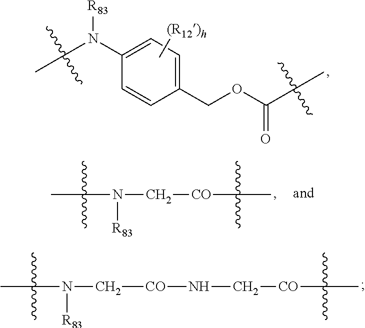

in each of which the terminal NR₈₃ group of Y is proximal to R₈₈;

R₈₃ is hydrogen or CH₃;

each W is an amino acid unit;

each R₁₂′ independently is halogen, —C₁₋₈ alkyl, —O—C₁₋₈ alkyl, nitro or cyano;

R₈₈ is hydrogen or —C(O)—(CH₂)_{ff}—(NH—C(O))ₐₐ-Eⱼ-(CH₂)_{bb}—R₈₅

R₈₅ is NH₂, OH or

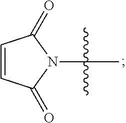 [SMILES: *N1C(=O)C=CC1=O]

E is —CH₂— or —CH₂CH₂O—;

q is an integer from 0 to 12;

aa is an integer 0 or 1;

bb is an integer 0 or 2;

ff is an integer from 0 to 10;

h is an integer from 0 to 4;

j is an integer from 0 to 12; and

when E is —CH₂—, bb is 0 and j is an integer from 0 to 10; and when E is —CH₂CH₂—O—, bb is 2 and j is an integer from 1 to 12;

or R₁₁ is

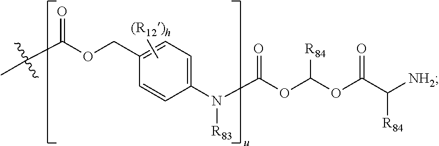

wherein:

R₈₃ is hydrogen or CH₃;

R₈₄ is C₁₋₆ alkyl or C₆₋₁₀ aryl;

each R₁₂′ independently is halogen, —C₁₋₈ alkyl, —O—C₁₋₈ alkyl, nitro or cyano; and

h is an integer from 0 to 4.

The scaffold of Formula (Ia) further comprises a PBRM connected to the polymeric carrier via L^{P}.

The scaffold of Formula (Ia) is of Formula (Ib):

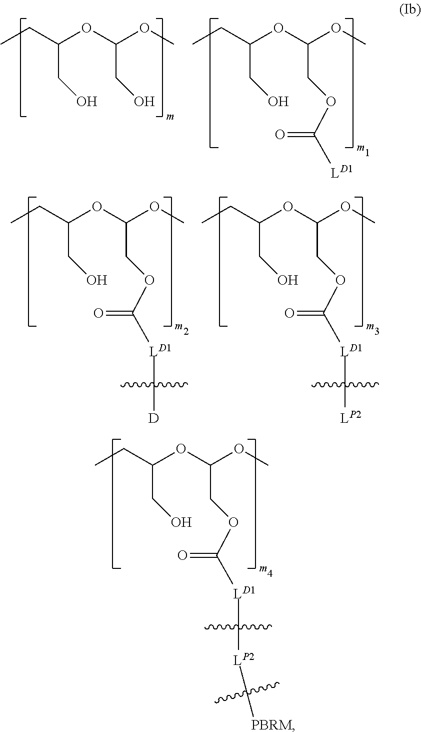

wherein:

the

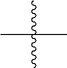

between L^{P2} and PBRM in

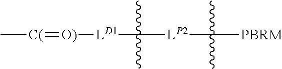

denotes direct or indirect attachment of PBRM to L^{P2}, such that the D-carrying polymeric carrier is connected to the PBRM,

each occurrence of PBRM independently has a molecular weight of less than 200 kDa (e.g., less than 80 kDa),

m is an integer from 1 to 2200,

m₁ is an integer from 1 to 660,

m₂ is an integer from 3 to 300,

m₃ is an integer from 0 to 110,

m₄ is an integer from 1 to 60; and

the sum of m, m₁, m₂, m₃ and m₄ ranges from 150 to 2200.

In Formula (Ib), m₁ is an integer from about 10 to about 660 (e.g., about 10-250).

When the PHF in Formula (Ib) has a molecular weight ranging from 20 kDa to 150 kDa (i.e., the sum of m, m₁, m₂, m₃, and m₄ ranging from about 150 to about 1100), m₂ is an integer from 3 to about 150, m₃ is an integer from 0 to about 55, m₄ is an integer from 1 to about 30, and/or m₁ is an integer from 1 to about 330 (e.g., m₁ being about 10-330 or about 15-100). For example, the sum of m₁ and m₂ is an integer from 14 to 330, and the sum of m₃ and m₄ is an integer from 1 to 55.

When the PHF in Formula (Ib) has a molecular weight ranging from 30 kDa to 100 kDa, the m₁ an integer from 1 to 220 (e.g., m₁ being about 10-220 or about 15-120), m₂ is an integer from 3 to 100, m₃ is an integer from 0 to 40, and/or m₄ is an integer from 1 to 20. For example, the sum of m₁ and m₂ is an integer from 18 to 220, and the sum of m₃ and m₄ is an integer from 1 to 40.

In Formula (Ib), the ratio of D to PBRM is between 5:1 and 40:1.

Other features of scaffold of Formula (Ia) or (Ib) include those described herein where applicable.

In another aspect, the invention features a polymeric scaffold useful to conjugate with a PBRM. The scaffold comprises a polymeric carrier, one or more -L^{D}-D connected to the polymeric carrier, and one or more L^{P} connected to the polymeric carrier which is suitable for connecting a PBRM to the polymeric carrier, wherein:

each occurrence of D is independently a tubulysin compound (e.g., a naturally occurring tubulysin or an analog or derivative thereof) having a molecular weight ≤5 kDa;

the polymeric carrier is a polyacetal or a polyketal,

L^{D} is a first linker having the structure:

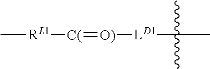

with R^{L1} connected to an oxygen atom of the polymeric carrier and L^{D1} connected to D, and

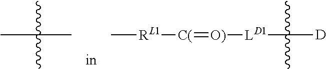

denotes direct or indirect attachment of D to L^{D1}, and L^{D} contains a biodegradable bond so that when the bond is broken, D is released in an active form for its intended therapeutic effect;

L^{D1} is a carbonyl-containing moiety;

L^{P} is a second linker having the structure: —R^{L2}—C(═O)-L^{P1} with R^{L2} connected to an oxygen atom of the polymeric carrier and L^{P1} suitable for connecting directly or indirectly to a PBRM which is not yet connected, and each occurrence of the second linker is distinct from each occurrence of the first linker

each of R^{L1} and R^{L2} independently is absent, alkyl, heteroalkyl, cycloalkyl, or heterocycloalkyl; and

L^{P1} is a moiety containing a functional group that is capable of forming a covalent bond with a functional group of a PBRM but has not yet formed a covalent bond.

The polymeric scaffold can include one or more of the following features.

L^{P} is a second linker having the structure:

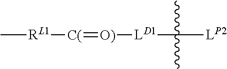

in which L^{P2} is a moiety containing a functional group that is capable of forming and not yet formed a covalent bond with a functional group of a PBRM, and

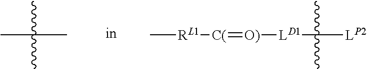

denotes direct or indirect attachment of L^{P2} to L^{D1}.

The functional group of L^{P1} or L^{P2} is selected from —SR^{p}, —S—S-LG, maleimido, and halo, in which LG is a leaving group and R^{p} is H or a sulfur protecting group.

L^{D1} comprises —X—(CH₂)ᵥ—C(═O)— with X directly connected to the carbonyl group of

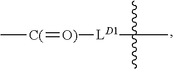

in which X is CH₂, O, or NH, and v is an integer from 1 to 6.

L^{P1} or L^{P2} contains a biodegradable bond.

Each of R^{L1} and R^{L2} is absent.

The polymeric carrier of the scaffold of the invention is a polyacetal, e.g., a PHF having a molecular weight (i.e., MW of the unmodified PHF) ranging from about 2 kDa to about 300 kDa.

For conjugating a PBRM having a molecular weight of 40 kDa or greater (e.g., 80 kDa or greater), the polymeric carrier of the scaffold of the invention is a polyacetal, e.g., a PHF having a molecular weight (i.e., MW of the unmodified PHF) ranging from about 2 kDa to about 40 kDa (e.g., about 6-20 kDa or about 8-15 kDa).

For conjugating a PBRM having a molecular weight of 200 kDa or less (e.g., 80 kDa or less), the polymeric carrier of the scaffold of the invention is a polyacetal, e.g., a PHF having a molecular weight (i.e., MW of the unmodified PHF) ranging from about 20 kDa to about 300 kDa (e.g., about 40-150 kDa or about 50-100 kDa).

Alternatively or additionally, one or more drug-carrying polymeric carriers are connected to one PBRM. The scaffold (e.g., a PBRM-polymer-drug conjugate) comprises a PBRM with a molecular weight of greater than 40 kDa and one or more D-carrying polymeric carriers connected to the PBRM, in which each of the D-carrying polymeric carrier independently is of Formula (Ic):

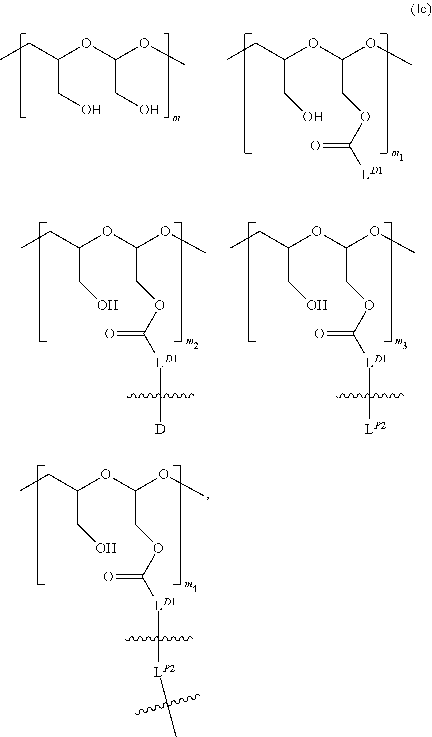

wherein:

the terminal

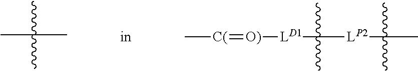

in denotes direct or indirect attachment of L^{P2} to PBRM such that the D-carrying polymeric carrier is connected to the PBRM,

m is an integer from 1 to 300,

m₁ is an integer from 1 to 140,

m₂ is an integer from 1 to 40,

m₃ is an integer from 0 to 18,

m₄ is an integer from 1 to 10; and

the sum of m, m₁, m₂, m₃, and m₄ ranges from 15 to 300; provided that the total number of L^{P2} attached to the PBRM is 10 or less.

In Formula (Ic), m₁ is an integer from 1 to about 120 (e.g., about 1-90) and/or m₃ is an integer from 1 to about 10 (e.g., about 1-8).

When the PHF in Formula (Ic) has a molecular weight ranging from about 6 kDa to about 20 kDa (i.e., the sum of m, m₁, m₂, m₃, and m₄ ranging from about 45 to about 150), m₂ is an integer from 2 to about 20, m₃ is an integer from 1 to about 9, and/or m₁ is an integer from 1 to about 75 (e.g., m₁ being about 4-45).

When the PHF in Formula (Ic) has a molecular weight ranging from about 8 kDa to about 15 kDa (i.e., the sum of m, m₁, m₂, m₃, and m₄ ranging from about 60 to about 110), m₂ is an integer from 2 to about 15, m₃ is an integer from 1 to about 7, and/or m₁ is an integer from 1 to about 55 (e.g., m₁ being about 4-30).

In Formula (Ic), the ratio of D to PBRM is between 5:1 and 40:1 (e.g., 25:1, 20:1, 15:1, 10:1, 9:1, 8:1, 7:1, 6:1, 5:1, 4:1, 3:1, or 2:1).

Each occurrence of D independently is selected from tubulysin compounds and analogs thereof (e.g., a naturally occurring tubulysin or an analog or derivative thereof).

L^{D} is —R^{L1}—C(═O)—X^{D}-M^{D1}-Y^{D}-M^{D2}-Z^{D}-M^{D3}-Q^{D}-M^{D4}—with M^{D4} directly connected to D, in which

X^{D} is —O—, —S—, —N(R¹)—, or absent, in which R¹ is hydrogen, an aliphatic, heteroaliphatic, carbocyclic, or heterocycloalkyl moiety, —C(═O)R^{1B}, —C(═O)OR^{1B}, or —SO₂R^{1B}, or —N(R¹)— is a heterocycloalkyl moiety, wherein R^{1B} is hydrogen, an aliphatic, heteroaliphatic, carbocyclic, or heterocycloalkyl moiety;

each of Y^{D}, Z^{D}, and Q^{D}, independently, is absent or a biodegradable linker moiety selected from the group consisting of —S—S—, —C(═O)O—, —C(═O)NR²—, —OC(═O)—, —NR²C(═O)—, —OC(═O)O—, —OC(═O)NR²—, —NR²C(═O)O—, —NR²C(═O)NR³—, —C(OR²)O—, —C(OR²)S—, —C(OR²)NR³—, —C(SR²)O—, —C(SR²)S—, —C(SR²)NR³—, —C(NR²R³)O—, —C(NR²R³)S—, —C(NR²R³)NR⁴—, —C(═O)S—, —SC(═O)—, —SC(═O)S—, —OC(═O)S—, —SC(═O)O—, —C(═S)S—, —SC(═S)—, —OC(═S)—, —C(═S)O—, —SC(═S)O—, —OC(═S)S—, —OC(═S)O—, —SC(═S)S—, —C(═NR²)O—, —C(═NR²)S—, —C(═NR²)NR³—, —OC(═NR²)—, —SC(═NR²)—, —NR³C(═NR²)—, —NR²SO₂—, —NR²NR³—, —C(═O)NR²NR³—, —NR²NR³C(═O)—, —OC(═O)NR²NR³—, —NR²NR³C(═O)O—, —C(═S)NR²NR³—, —NR²NR³C(═S)—, —C(═NR⁴)NR²NR³—, —NR²NR³C(═NR⁴)—, —O(N═CR³)—, —(CR³═N)O—, —C(═O)NR²—(N═CR³)—, —(CR³═N)—NR²C(═O)—, —SO₃—, —NR²SO₂NR³—, —SO₂NR²—, and polyamide, wherein each occurrence of R², R³, and R⁴ independently is hydrogen or an aliphatic, heteroaliphatic, carbocyclic, or heterocyclic moiety, or each occurrence of —NR²— or —NR²NR³— is a heterocycloalkyl moiety; and

each of M^{D1}, M^{D2}, M^{D3}, and M^{D4} independently, is absent or a non-biodegradable linker moiety selected from the group consisting of alkyl, alkenyl, alkynyl, heteroalkyl, heteroalkenyl, heteroalkynyl, a carbocyclic moiety, a heterocyclic moiety, and a combination thereof, and each of M^{D1}, M^{D2}, and M^{D3} optionally contains one or more —(C═O)— but does not contain any said biodegradable linker moiety;

provided that for each L^{D1}, at least one of X^{D}, Y^{D}, Z^{D}, and Q^{D} is not absent.

Each

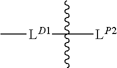

when not connected to PBRM, independently comprises a terminal group W^{P}, in which each W^{P} independently is:

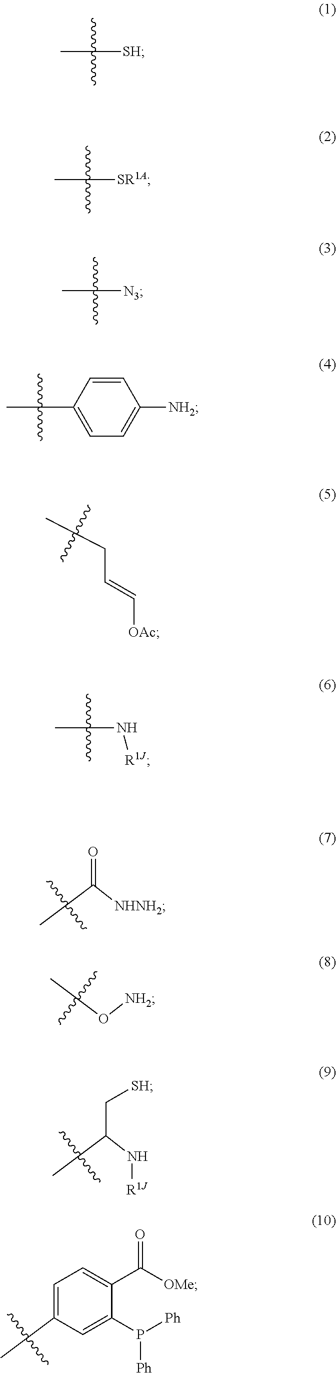

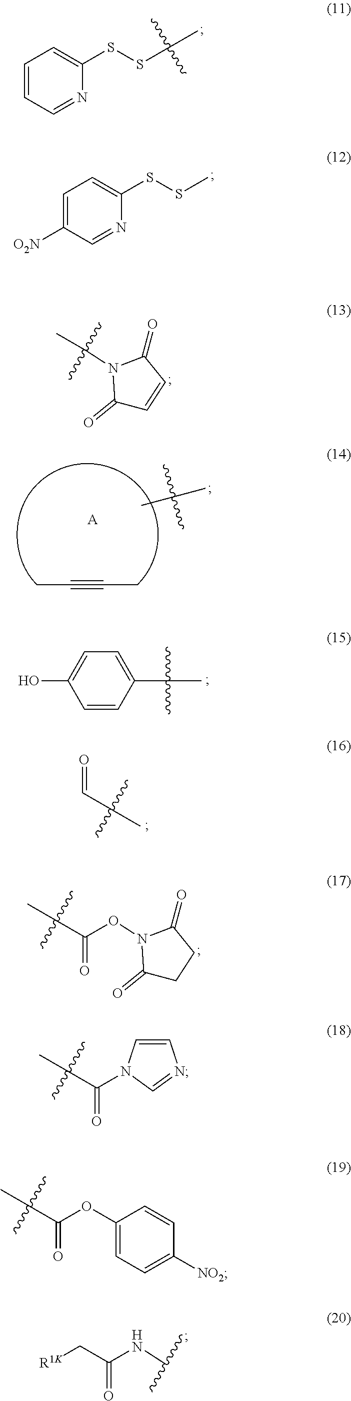

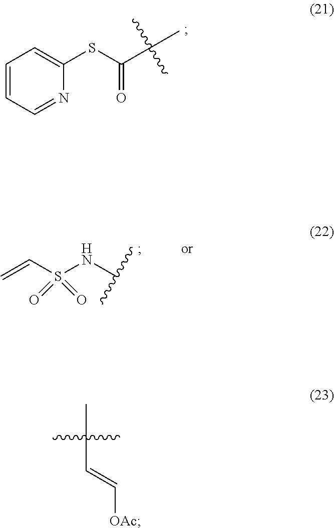

in which R^{1K} is a leaving group (e.g., halide or RC(O)O— in which R is hydrogen, an aliphatic, heteroaliphatic, carbocyclic, or heterocycloalkyl moiety), R^{1A} is a sulfur protecting group, and ring A is cycloalkyl or heterocycloalkyl, and R^{1J} is hydrogen, an aliphatic, heteroaliphatic, carbocyclic, or heterocycloalkyl moiety.

Each R^{1A} independently is

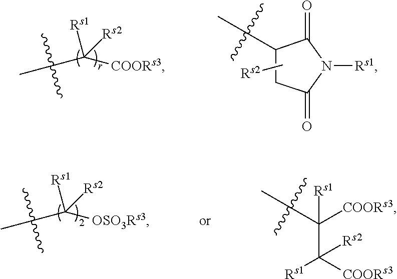

in which r is 1 or 2 and each of R^{s1}, R^{s2}, and R^{s3} is hydrogen, an aliphatic, heteroaliphatic, carbocyclic, or heterocycloalkyl moiety.

Each

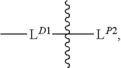

when connected to PBRM, independently is —X^{P}-M^{P1}-Y^{P}-M^{P2}-Z^{P}-M^{P3}-Q^{P}-M^{P4}-, with X^{P} directly connected to the carbonyl group of

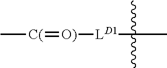

and M^{P4} directly connected to PBRM, in which

X^{P} is —O—, —S—, —N(R¹)—, or absent, in which R¹ is hydrogen, an aliphatic, heteroaliphatic, carbocyclic, or heterocycloalkyl moiety, —C(═O)R^{1B}, —C(═O)OR^{1B}, or —SO₂R^{1B}, or —N(R¹)— is a heterocycloalkyl moiety, wherein R^{1B} is hydrogen, an aliphatic, heteroaliphatic, carbocyclic, or heterocycloalkyl moiety;

each of Y^{P}, Z^{P}, and Q^{P}, independently, is absent or a biodegradable linker moiety selected from the group consisting of —S—S—, —C(═O)O—, —C(═O)NR²—, —OC(═O)—, —NR²C(═O)—, —OC(═O)O—, —OC(═O)NR²—, —NR²C(═O)O—, —NR²C(═O)NR³—, —C(OR²)O—, —C(OR²)S—, —C(OR²)NR³—, —C(SR²)O—, —C(SR²)S—, —C(SR²)NR³—, —C(NR²R³)O—, —C(NR²R³)S—, —C(NR²R³)NR⁴—, —C(═O)S—, —SC(═O)—, —SC(═O)S—, —OC(═O)S—, —SC(═O)O—, —C(═S)S—, —SC(═S)—, —OC(═S)—, —C(═S)O—, —SC(═S)O—, —OC(═S)S—, —OC(═S)O—, —SC(═S)S—, —C(═NR²)O—, —C(═NR²)S—, —C(═NR²)NR³—, —OC(═NR²)—, —SC(═NR²)—, —NR³C(═NR²)—, —NR²SO₂—, —NR²NR³—, —C(═O)NR²NR³—, —NR²NR³C(═O)—, —OC(═O)NR²NR³—, —NR²NR³C(═O)O—, —C(═S)NR²NR³—, —NR²NR³C(═S)—, —C(═NR⁴)NR²NR³—, —NR²NR³C(═NR⁴)—, —O(N═CR³)—, —(CR³═N)O—, —C(═O)NR²—(N═CR³)—, —(CR³═N)—NR²C(═O)—, —SO₃—, —NR²SO₂NR³—, —SO₂NR²—, and polyamide, wherein each occurrence of R², R³, and R⁴ independently is hydrogen or an aliphatic, heteroaliphatic, carbocyclic, or heterocyclic moiety, or each occurrence of —NR²— or —NR²NR³— is a heterocycloalkyl moiety; and

each of M^{P1}, M^{P2}, M^{P3}, and M^{P4} independently, is absent or a non-biodegradable linker moiety selected from the group consisting of alkyl, alkenyl, alkynyl, heteroalkyl, heteroalkenyl, heteroalkynyl, a carbocyclic moiety, a heterocyclic moiety, and a combination thereof, and each of MP1, M^{P2}, and M^{P3} optionally contains one or more —(C═O)— but does not contain any said biodegradable linker moiety;

provided that for each

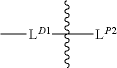

connected to PBRM, at least one of X^{P}, Y^{P}, Z^{P}, and Q^{P} is not absent.

Each of M^{D1} and M^{P1} independently is C₁₋₆ alkyl or C₁₋₆ heteroalkyl.

Each of M^{D2}, M^{D3}, M^{D4}, M^{P2}, M^{P3}, and M^{P4}, independently is absent, C₁₋₆ alkyl, cycloalkyl, heteroalkyl, heterocycloalkyl, or a combination thereof.

In each

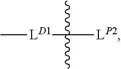

at most one of M^{P2} and M^{P3} has one of the following structures:

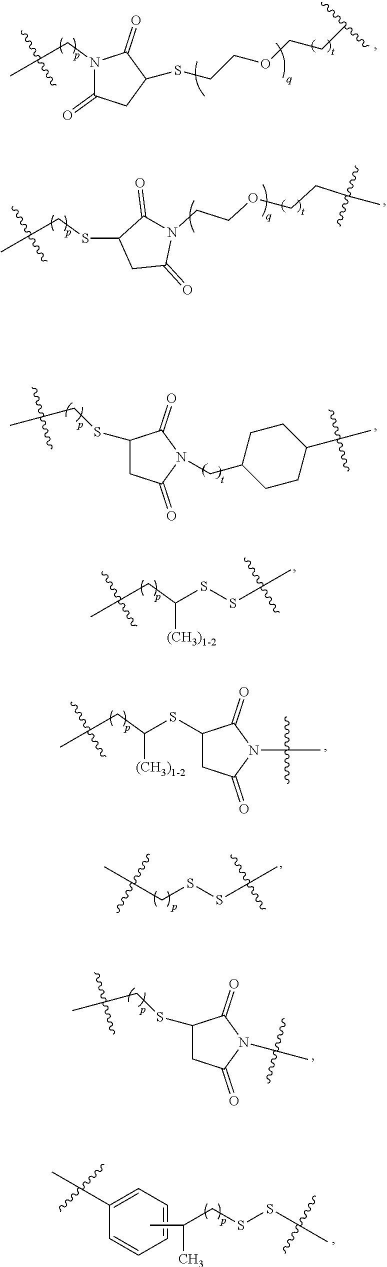

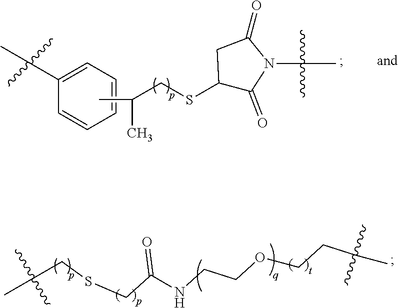

in which q is an integer from 0 to 12 and each of p and t independently is an integer from 0 to 3.

In yet another aspect, the invention encompasses a conjugate comprising a polymeric carrier, one or more -L^{D}-D connected to the polymeric carrier, and a protein based recognition-molecule (PBRM) connected to the polymeric carrier via L^{P}, wherein:

each occurrence of D is independently a tubulysin compound (e.g., a naturally occurring tubulysin or an analog or derivative thereof) having a molecular weight ≤5 kDa;

the polymeric carrier is a polyacetal or polyketal,

L^{D} is a linker having the structure: —R^{L1}—C(═O)—X^{D}-M^{D1}-Y^{D}-M^{D2}-Z^{D}-M^{D3}-Q^{D}-M^{D4}-, with R^{L1} connected to an oxygen atom of the polymeric carrier and M^{D4} connected to D;

L^{P} is a linker having the structure: —R^{L2}—C(═O)—X^{P}-M^{P1}-Y^{P}-M^{P2}-Z^{P}-M^{P3}-Q^{P}-M^{P4}-, with R^{L2} connected to an oxygen atom of the polymeric carrier and M^{P4} connected to the protein based recognition-molecule;

each of R^{L1} and R^{L2} independently is absent, alkyl, cycloalkyl, heteroalkyl, or heterocycloalkyl;

each of X^{D} and X^{P}, independently is —O—, —S—, —N(R¹)—, or absent, in which R¹ is hydrogen, an aliphatic, heteroaliphatic, carbocyclic, or heterocycloalkyl moiety, —C(═O)R^{1B}, —C(═O)OR^{1B}, —SO₂R^{1B} or —N(R¹)— is a heterocycloalkyl moiety, wherein R^{1B} is hydrogen, an aliphatic, heteroaliphatic, carbocyclic, or heterocycloalkyl moiety;

each of Y^{D}, Y^{P}, Z^{D}, Z^{P}, Q^{D}, and Q^{P}, independently, is absent or a biodegradable linker moiety selected from the group consisting of —S—S—, —C(═O)O—, —C(═O)NR²—, —OC(═O)—, —NR²C(═O)—, —OC(═O)O—, —OC(═O)NR²—, —NR²C(═O)O—, —NR²C(═O)NR³—, —C(OR²)O—, —C(OR²)S—, —C(OR²)NR³—, —C(SR²)O—, —C(SR²)S—, —C(SR²)NR³—, —C(NR²R³)O—, —C(NR²R³)S—, —C(NR²R³)NR⁴—, —C(═O)S—, —SC(═O)—, —SC(═O)S—, —OC(═O)S—, —SC(═O)O—, —C(═S)S—, —SC(═S)—, —OC(═S)—, —C(═S)O—, —SC(═S)O—, —OC(═S)S—, —OC(═S)O—, —SC(═S)S—, —C(═NR²)O—, —C(═NR²)S—, —C(═NR²)NR³—, —OC(═NR²)—, —SC(═NR²)—, —NR³C(═NR²)—, —NR²SO₂—, —NR²NR³—, —C(═O)NR²NR³—, —NR²NR³C(═O)—, —OC(═O)NR₂NR³—, —NR²NR³C(═O)O—, —C(═S)NR²NR³—, —NR²NR³C(═S)—, —C(═NR⁴)NR²NR³—, —NR²NR³C(═NR⁴)—, —O(N═CR³)—, —(CR³═N)O—, —C(═O)NR²—(N═CR³)—, —(CR³═N)—NR²C(═O)—, —SO₃—, —NR²SO₂NR³—, —SO₂NR²—, and polyamide, wherein each occurrence of R², R³, and R⁴ independently is hydrogen or an aliphatic, heteroaliphatic, carbocyclic, or heterocyclic moiety, or each occurrence of —NR²— or —NR²NR³— is a heterocycloalkyl moiety; and

each of M^{D1}, M^{D2}, M^{D3}, M^{D4}, M^{P1}, M^{P2}, M^{P3} and M^{P4}, independently, is absent or a non-biodegradable linker moiety selected from the group consisting of alkyl, alkenyl, alkynyl, heteroalkyl, heteroalkenyl, heteroalkynyl, a carbocyclic moiety, a heterocyclic moiety, and a combination thereof, and each of M^{D1}, M^{D2}, M^{D3}, MP1, M^{P2}, and M^{P3} optionally contains one or more —(C═O)— but does not contain any said biodegradable linker moiety;

provided that for each L^{D}, at least one of X^{D}, Y^{D}, Z^{D}, and Q^{D} is not absent, and for each L^{P}, at least one of X^{P}, Y^{P}, Z^{P}, and Q^{P} is not absent.

The conjugate can include one or more of the following features.

The polymeric carrier can be a polyacetal, e.g., PHF.

For each L^{D}, M^{D1} is not absent when X^{D} is absent.

For each L^{P}, M^{P1} is not absent when X^{P} is absent.

The polymeric carrier can be further substituted with one or more —R^{L1}—C(═O)—X^{D}-M^{D1}-Y^{D}-M^{D2}-W^{D}, in which each W^{D} independently is:

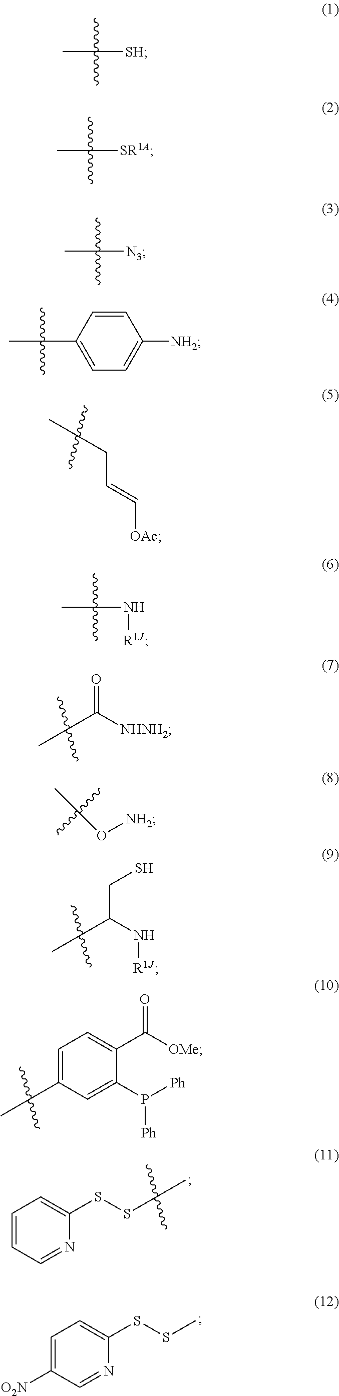

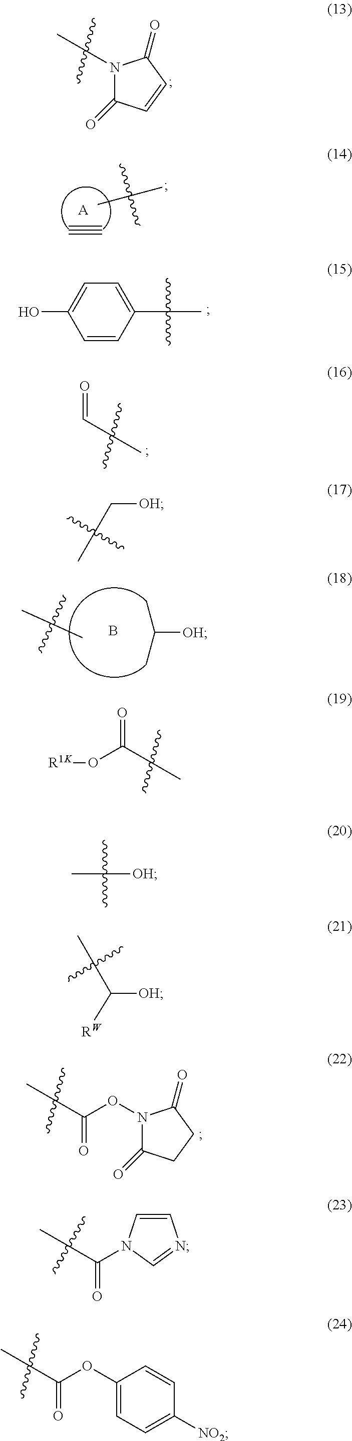

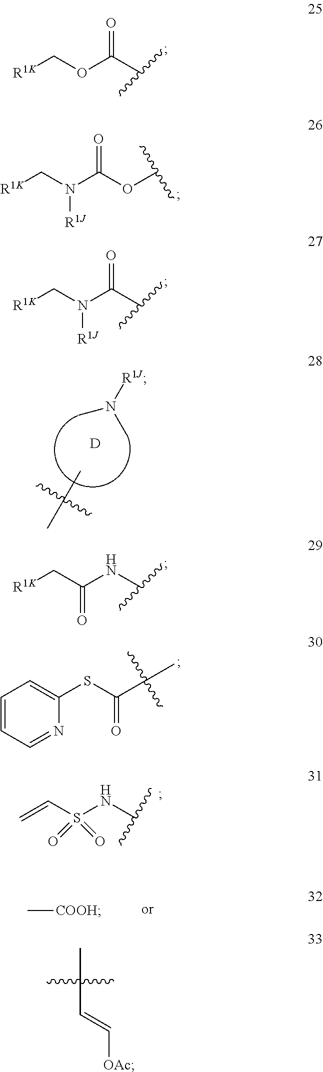

in which R^{1A} is a sulfur protecting group, each of ring A and B, independently, is cycloalkyl or heterocycloalkyl, R^{W} is an aliphatic, heteroaliphatic, carbocyclic or heterocycloalkyl moiety; ring D is heterocycloalkyl; R^{1J} is hydrogen, an aliphatic, heteroaliphatic, carbocyclic, or heterocycloalkyl moiety; and R^{1K} is a leaving group (e.g., halide or RC(O)O— in which R is hydrogen, an aliphatic, heteroaliphatic, carbocyclic, or heterocycloalkyl moiety).

The polymeric carrier can be further substituted with one or more —R^{L2}—C(═O)—X^{P}-M^{P1}-Y^{P}-M^{P2}-W^{P} in which each W^{P} independently is:

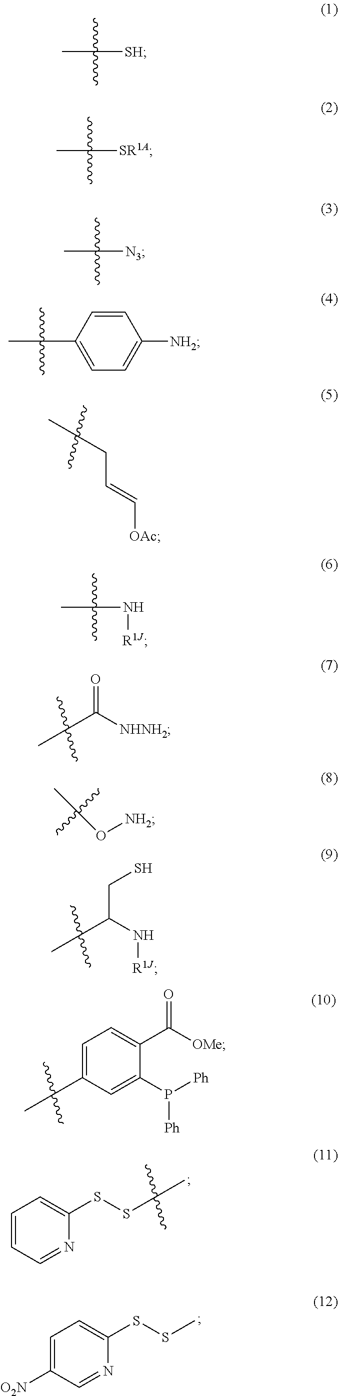

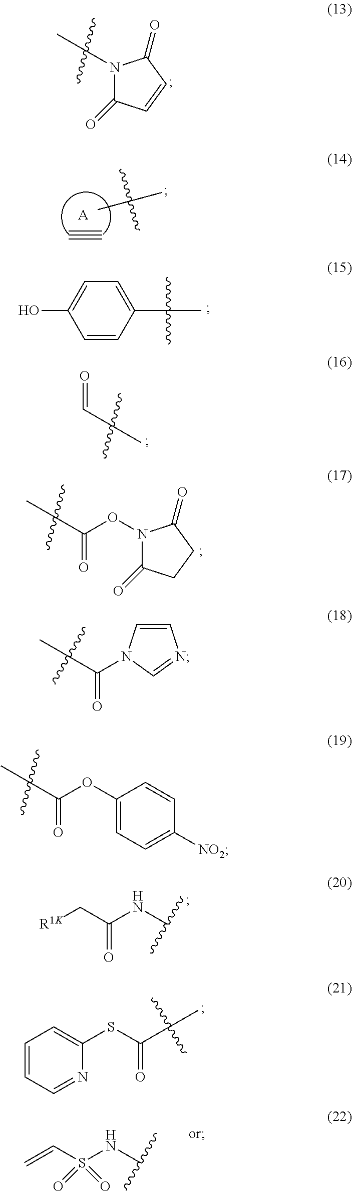

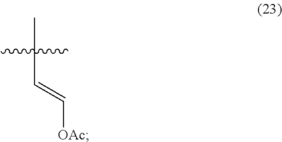

in which R^{1K} is a leaving group (e.g., halide or RC(O)O— in which R is hydrogen, an aliphatic, heteroaliphatic, carbocyclic, or heterocycloalkyl moiety), R^{1A} is a sulfur protecting group, and ring A is cycloalkyl or heterocycloalkyl, and R^{1J} is hydrogen, an aliphatic, heteroaliphatic, carbocyclic, or heterocycloalkyl moiety. For example, R^{1A} is

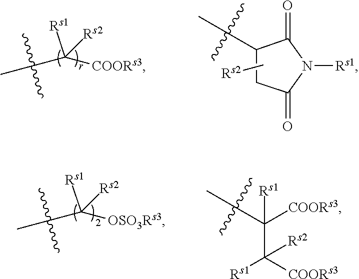

in which r is 1 or 2 and each of Rₛ₁, R^{s2}, and R^{s3} is hydrogen, an aliphatic, heteroaliphatic, carbocyclic, or heterocycloalkyl moiety.

Ring A can be C₃₋₈ cycloalkyl or 5-19 membered heterocycloalkyl.

Ring A can be

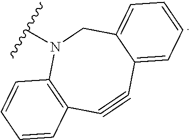 [SMILES: *N1Cc2ccccc2C#Cc2ccccc21]

Ring B can be C₃₋₈ cycloalkyl or 3-12 membered heterocycloalkyl.

Ring D can be piperazinyl or piperidinyl.

Each of R^{s1}, R^{s2}, and R^{s3} can be hydrogen or C₁₋₆ alkyl.

Each PBRM independently can be a peptide, a peptide mimetic, an antibody, or an antibody fragment.

Each of M^{D1} and M^{P1} independently can be C₁₋₆ alkyl or C₁₋₆ heteroalkyl.

Each of M^{D2}, M^{D3}, M^{D4}, M^{P2}, M^{P3}, and M^{P4}, independently can be absent, C₁₋₆ alkyl, cycloalkyl, heteroalkyl, heterocycloalkyl, or a combination thereof.

For each L^{D}, at most two of M^{D2}, M^{D3}, and M^{D4} can be absent.

For each L^{P}, at most two of M^{P2}, M^{P3}, and M^{P4} can be absent.

For each L^{D}, at most one of M^{D2} and M^{D3} can have one of the following structures:

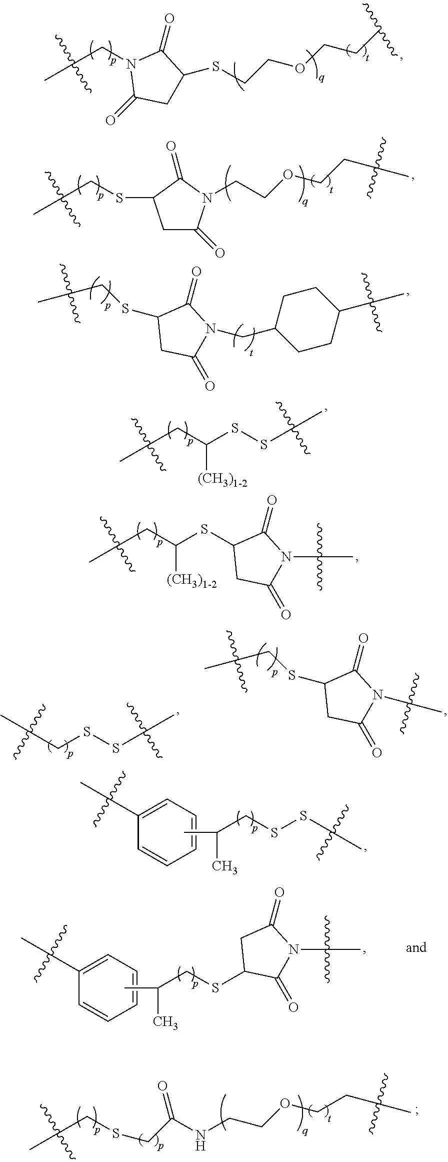

in which q is an integer from 0 to 12 and each of p and t independently is an integer from 0 to 3.

For each L^{P}, at most one of M^{P2} and M^{P3} can have one of the following structures:

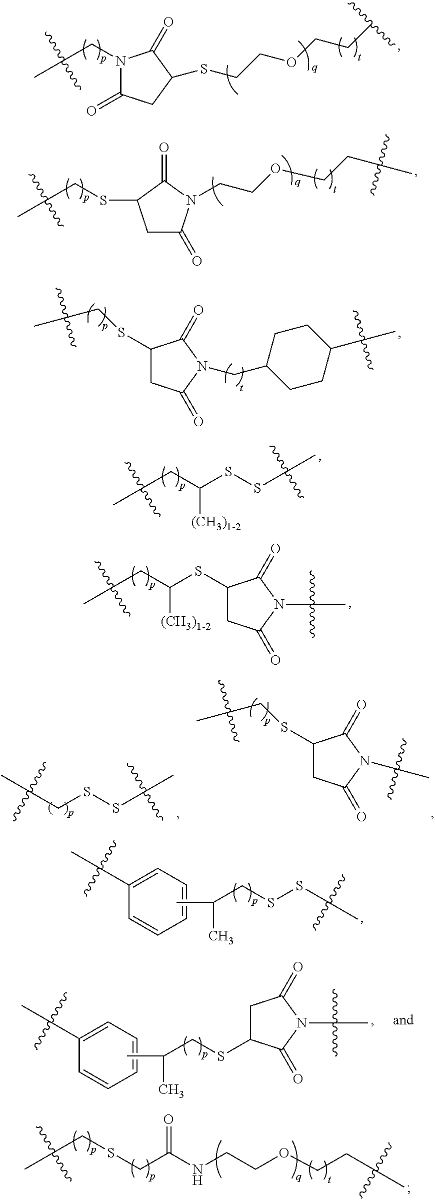

in which q is an integer from 0 to 12 and each of p and t independently is an integer from 0 to 3.

For each L^{D}, each of -M^{D2}-Z^{D}—Z^{D}-M^{D3}-, —Z^{D}-M^{D2}-, and -M^{D3}-Z^{D}-, independently can have one of the following structures:

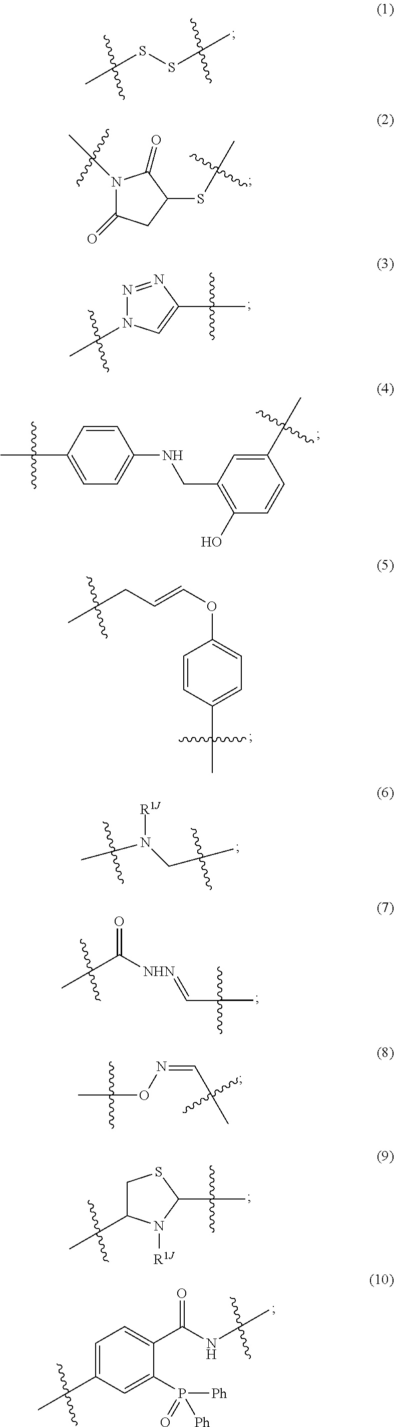

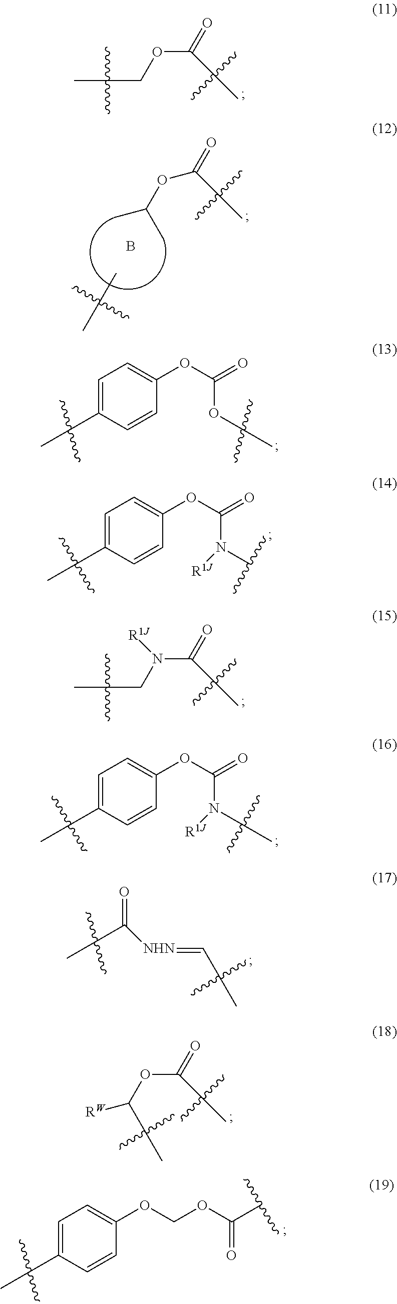

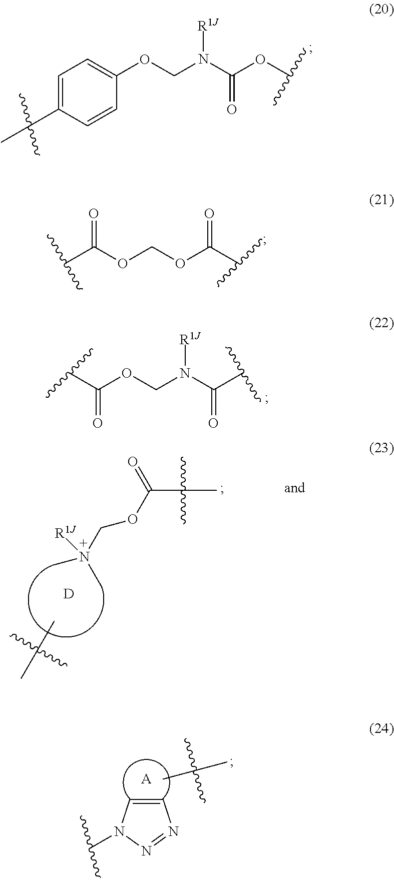

in which ring A or B independently is cycloalkyl or heterocycloalkyl; R^{W} is an aliphatic, heteroaliphatic, carbocyclic, or heterocycloalkyl moiety; R^{1J} is hydrogen, an aliphatic, heteroaliphatic, carbocyclic, or heterocycloalkyl moiety; and ring D is heterocycloalkyl.

For each L^{P}, each of -M^{P2}-Z^{P}—, —Z^{P}-M^{P3}-, —Z^{P}-M^{P2}-, and -M^{P3}-Z^{P}—, independently, can have one of the following structures:

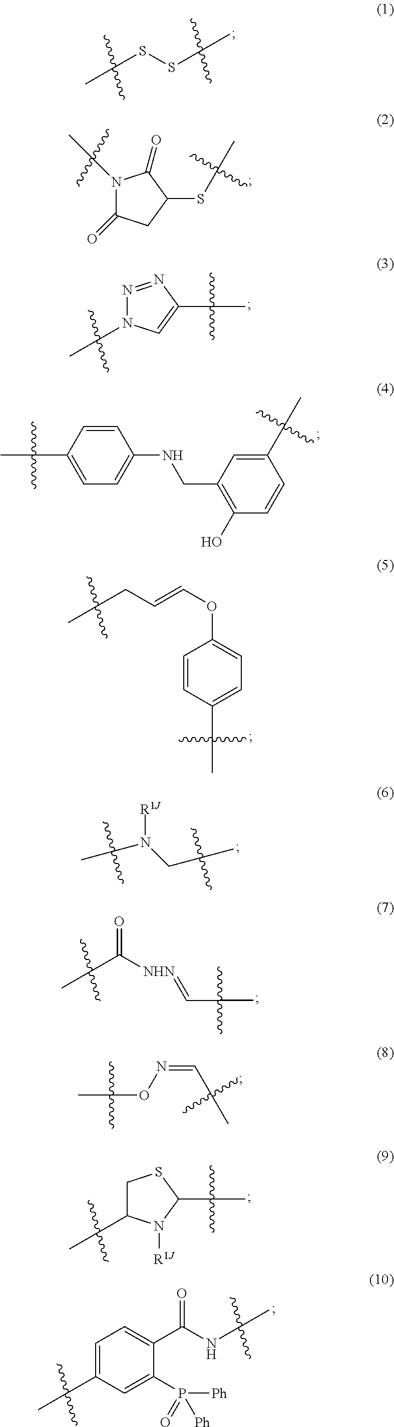

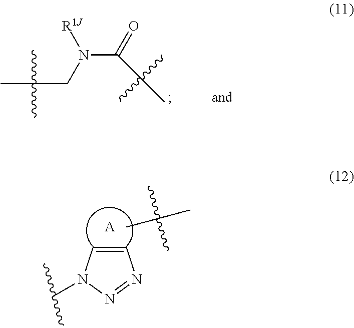

in which ring A is cycloalkyl or heterocycloalkyl and R^{1J} is hydrogen, an aliphatic, heteroaliphatic, carbocyclic, or heterocycloalkyl moiety.

Each of X^{D} and X^{P}, independently can be absent.

Each of X^{D} and X^{P}, independently can be O or NH.

Each of X^{D} and X^{P}, independently can be

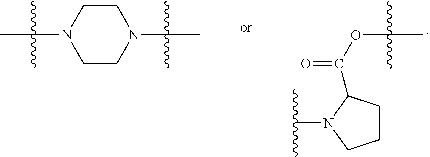 [SMILES: *N1CCN(*)CC1 ; *OC(=O)C1CCCN1*]

Each of Y^{D} and Y^{P} independently can be —S—S—, —OCO—, —COO—, —CONH—, or —NHCO—.

Each of Q^{D} and Q^{P} independently can be absent, —S—S—, —OCO—, —COO—, —CONH—, —NHCO—, —OCONHNH— or —NHNHCOO—.

In particular, this invention features a conjugate of Formula (I):

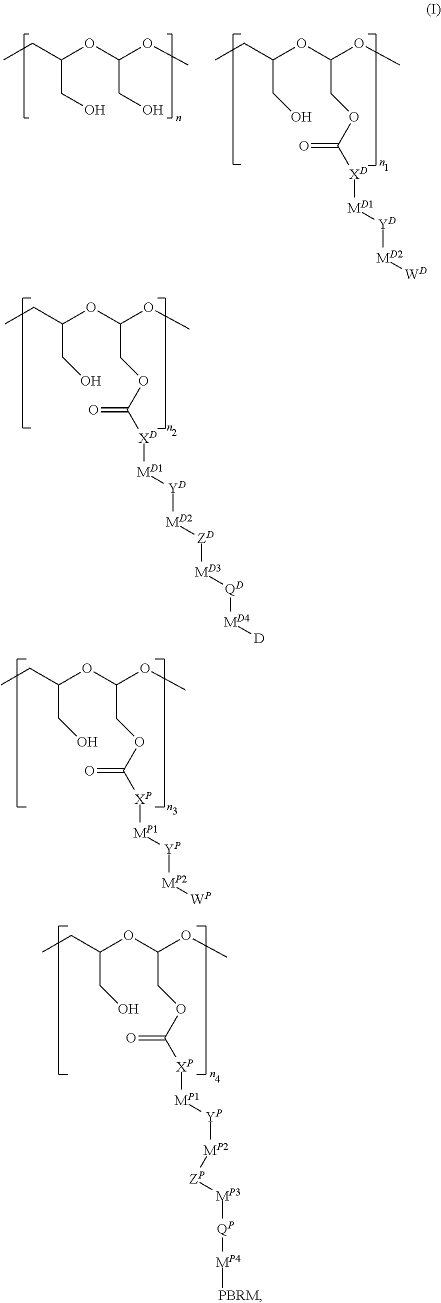

wherein each of n, n₁, n₂, n₃, and n₄, is the molar fraction of the corresponding polymer unit ranging between 0 and 1; n+n₁+n₂+n₃+n₄=1; provided that none of n, n₂, and n₄ is 0.

In all formulae disclosed herein the disconnection or gap between the polyacetal units indicates that the units can be connected to each other in any order. In other words, e.g., in Formula (I) above, the appending groups that contain D, PBRM, W^{D}, and W^{P}, can be randomly distributed along the polymer backbone.

In the protein-polymer-drug conjugate of Formula (I), each D can be the same or different tubulysin compound and each PBRM can be the same or a different moiety.

The ratio between n₂ and n₄ can be greater than 1:1, and up to 200:1 (e.g., up to 100:1), e.g., between 2:1 and 40:1; between 5:1 and 20:1; between 10:1 and 50:1, between 25:1 and 50:1, or between 30:1 and 50:1.

The ratio between n₂ and n₄ can be about 50:1, 40:1, 25:1, 20:1, 10:1, 5:1 or 2:1.

For example the ratio between D and PBRM can be greater than 1:1, and up to 200:1 (e.g., up to 100:1), e.g., between 2:1 and 40:1; between 5:1 and 20:1; between 10:1 and 50:1, between 25:1 and 50:1, or between 30:1 and 50:1. Examples of PBRM include but are not limited to, full length antibodies such as IgG and IgM, antibody fragments such as Fabs, scFv, camelids, Fab2, and the like, small proteins, and peptides.

In one embodiment the ratio between D and PBRM can be about 50:1, 40:1, 25:1, 20:1, 15:1, 10:1, 9:1, 8:1, 7:1, 6:1, 5:1, 4:1, 3:1, or 2:1.

In another embodiment the ratio between D and PBRM can be about 25:1, 20:1, 15:1, 10:1, 5:1 or 2:1.

The polymeric scaffold, e.g., that of Formula (I), can also include one or more features of Formula (Ia), (Ib), or (Ic) described herein where applicable.

In another aspect, the invention provides compositions comprising the conjugates, methods for their preparation, and methods of use thereof in the treatment of various disorders, including, but not limited to cancer.

The invention also features a drug-polymer conjugate (e.g., tubulysin compound-polymer conjugate) that is similar to the protein-polymer-drug conjugate described above except that drug-polymer conjugate does not contain a PBRM. In this embodiment the polymer-drug conjugate may comprise a plurality of drug moieties in which each D can be the same or different. In this embodiment, n₄ is 0 in the conjugate of Formula (I). The methods of producing the drug-polymer conjugates and methods of treating various disorders (e.g., cancer) are also contemplated and described herein.

The invention also features a protein-polymer conjugate (e.g., PBRM-polymer conjugate) that is similar to the protein-polymer-drug conjugate described above except that protein-polymer conjugate does not contain a drug. In this embodiment the protein-polymer conjugate may comprise a plurality of protein moieties in which each PBRM can be the same or different. In this embodiment, n₂ is 0 in the conjugate of Formula (I). The methods of producing the drug-polymer conjugates or polymeric scaffolds and methods of treating various disorders (e.g., cancer) are also contemplated and described herein. The target cancer can be anal, astrocytoma, leukemia, lymphoma, head and neck, liver, testicular, cervical, sarcoma, hemangioma, esophageal, eye, laryngeal, mouth, mesothelioma, skin, myeloma, oral, rectal, throat, bladder, breast, uterus, ovary, prostate, lung, colon, pancreas, renal, or gastric cancer.

The invention further relates to a pharmaceutical composition comprising a polymeric scaffold or conjugate described herein and a pharmaceutically acceptable carrier.

In yet another aspect, the invention relates to a method of diagnosing a disorder in a subject suspected of having the disorder. The method comprises administering an effective amount of the conjugate described herein to the subject suspected of having the disorder or performing an assay to detect a target antigen/receptor in a sample from the subject so as to determine whether the subject expresses target antigen or receptor.

Also within the scope of the invention is a method of preparing a scaffold described above. The method comprises providing a polymeric carrier that is substituted with one or more D and one or more —R^{L1}—C(═O)-L^{D1}, and reacting the polymeric carrier with a compound containing an L^{P2} moiety to produce the scaffold comprising a polymeric carrier substituted both with one or more D and with one or more

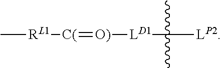

Alternatively, the method comprises providing a polymeric carrier that is substituted with one or more

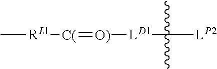

and one or more —R^{L1}—C(═O)-L^{D1}, and reacting the polymeric carrier with D containing a functional group that is capable of forming and not yet formed a covalent bond with —R^{L1}—C(═O)-L^{D1} to produce the scaffold comprising a polymeric carrier substituted both with one or more D and with one or more

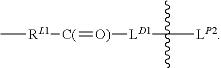

As used herein, the terms “polymeric scaffold” or simply “scaffold” and “conjugate” are used interchangeably when the scaffold comprises one or more PBRM and one or more D molecules (i.e., tubulysin compounds).

As used herein the terms “polymer,” and “polymeric carrier” are used interchangeably.

As used herein, the expression “capable of” or “suitable for” connecting to, conjugating with, or forming, in one embodiment, refers to an association (e.g., a bond such as a covalent bond) but such association (e.g., bond) is not yet present.

Unless otherwise defined, all technical and scientific terms used herein have the same meaning as commonly understood by one of ordinary skill in the art to which this invention belongs. In the specification, the singular forms also include the plural unless the context clearly dictates otherwise. Although methods and materials similar or equivalent to those described herein can be used in the practice or testing of the present invention, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting.

One of the advantages of the present invention is that the protein-polymer-drug conjugates or the polymeric scaffolds described herein greatly enhances the bioavailability of the drugs to be delivered and/or enhances the bioavailability of the protein attached to the polymeric carrier. Another advantage of the present invention is that the efficacy of the protein-polymer-drug conjugates described herein increases or at least remains substantially the same with increases in the drug load of the conjugates. Yet another advantage of the present invention is that the protein-polymer conjugates via thiol conjugation to the cysteine moiety of the protein exhibits substantially improved stability. Other features and advantages of the invention will be apparent from the following detailed description and claims.

### BRIEF DESCRIPTION OF FIGURES

FIG. 1 is a group of tables listing “m” values per PHF scaffold and polymer/PBRM ratios of embodiments of the invention. Table 1 relates to PBRM-drug polymer conjugates in which the PBRMs have a molecular weight of 40 kDa or greater (e.g., 60 kDa or greater, 80 kDa or greater, 100 kDa or greater, 120 kDa or greater, 140 kDa or greater, 160 kDa or greater or 180 kDa or greater) and one or more PHF-Drug scaffolds are attached to one PBRM, Table 2 relates to PBRM-drug polymer conjugates in which the PBRMs have a molecular weight of 200 kDa or less (e.g., 120 kDa or less, 80 kDa or less, 60 kDa or less, 40 kDa or less, 20 kDa or less or 10 kDa or less) and one or more PBRMs are attached to one PHF-Drug scaffold.

### DETAILED DESCRIPTION OF CERTAIN PREFERRED EMBODIMENTS OF THE INVENTION

The present invention provides novel protein-polymer-tubulysin compound conjugates, polymeric scaffolds for making the conjugates, synthetic methods for making the conjugates or polymeric scaffolds, pharmaceutical compositions containing them and various uses of the conjugates.

The present invention also provides novel polymer-tubulysin compound conjugates, synthetic methods for making the conjugates, pharmaceutical compositions containing them and various uses of the conjugates.

The present invention further provides novel tubulysin compound derivatives, synthetic methods for making the derivatives, pharmaceutical compositions containing them and various uses of the drug derivatives.

### Definitions/Terminology

Certain compounds of the present invention, and definitions of specific functional groups are also described in more detail herein. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in “Organic Chemistry”, Thomas Sorrell, University Science Books, Sausalito: 1999, the entire contents of which are incorporated herein by reference. Furthermore, it will be appreciated by one of ordinary skill in the art that the synthetic methods, as described herein, utilize a variety of protecting groups.

The use of the articles “a”, “an”, and “the” in both the following description and claims are to be construed to cover both the singular and the plural, unless otherwise indicated herein or clearly contradicted by context. The terms “comprising”, “having”, “including”, and “containing” are to be construed as open terms (i.e., meaning “including but not limited to”) unless otherwise noted. Additionally whenever “comprising” or another open-ended term is used in an embodiment, it is to be understood that the same embodiment can be more narrowly claimed using the intermediate term “consisting essentially of” or the closed term “consisting of.”

The term “about”, “approximately”, or “approximate”, when used in connection with a numerical value, means that a collection or range of values is included. For example, “about X” includes a range of values that are ±20%, ±10%, ±5%, ±2%, ±1%, ±0.5%, ±0.2%, or ±0.1% of X, where X is a numerical value. In one embodiment, the term “about” refers to a range of values which are 5% more or less than the specified value. In another embodiment, the term “about” refers to a range of values which are 2% more or less than the specified value. In another embodiment, the term “about” refers to a range of values which are 1% more or less than the specified value.

Recitation of ranges of values are merely intended to serve as a shorthand method of referring individually to each separate value falling within the range, unless otherwise indicated herein, and each separate value is incorporated into the specification as if it were individually recited herein. A range used herein, unless otherwise specified, includes the two limits of the range. For example, the expressions “x being an integer between 1 and 6” and “x being an integer of 1 to 6” both mean “x being 1, 2, 3, 4, 5, or 6”.

“Protecting group”: as used herein, the term protecting group means that a particular functional moiety, e.g., O, S, or N, is temporarily blocked so that a reaction can be carried out selectively at another reactive site in a multifunctional compound. In preferred embodiments, a protecting group reacts selectively in good yield to give a protected substrate that is stable to the projected reactions; the protecting group must be selectively removed in good yield by readily available, preferably nontoxic reagents that do not attack the other functional groups; the protecting group forms an easily separable derivative (more preferably without the generation of new stereogenic centers); and the protecting group has a minimum of additional functionality to avoid further sites of reaction. As detailed herein, oxygen, sulfur, nitrogen and carbon protecting groups may be utilized. For example, in certain embodiments, certain exemplary oxygen protecting groups may be utilized. These oxygen protecting groups include, but are not limited to methyl ethers, substituted methyl ethers (e.g., MOM (methoxymethyl ether), MTM (methylthiomethyl ether), BOM (benzyloxymethyl ether), and PMBM (p-methoxybenzyloxymethyl ether)), substituted ethyl ethers, substituted benzyl ethers, silyl ethers (e.g., TMS (trimethylsilyl ether), TES (triethylsilyl ether), TIPS (triisopropylsilyl ether), TBDMS (t-butyldimethylsilyl ether), tribenzyl silyl ether, and TBDPS (t-butyldiphenyl silyl ether), esters (e.g., formate, acetate, benzoate (Bz), trifluoroacetate, and dichloroacetate), carbonates, cyclic acetals and ketals. In certain other exemplary embodiments, nitrogen protecting groups are utilized. Nitrogen protecting groups, as well as protection and deprotection methods are known in the art. Nitrogen protecting groups include, but are not limited to, carbamates (including methyl, ethyl and substituted ethyl carbamates (e.g., Troc), amides, cyclic imide derivatives, N-Alkyl and N-Aryl amines, imine derivatives, and enamine derivatives. In yet other embodiments, certain exemplary sulfur protecting groups may be utilized. The sulfur protecting groups include, but are not limited to those oxygen protecting group describe above as well as aliphatic carboxylic acid (e.g., acrylic acid), maleimide, vinyl sulfonyl, and optionally substituted maleic acid. Certain other exemplary protecting groups are detailed herein, however, it will be appreciated that the present invention is not intended to be limited to these protecting groups; rather, a variety of additional equivalent protecting groups can be readily identified using the above criteria and utilized in the present invention. Additionally, a variety of protecting groups are described in “Protective Groups in Organic Synthesis” Third Ed. Greene, T. W. and Wuts, P. G., Eds., John Wiley & Sons, New York: 1999, the entire contents of which are hereby incorporated by reference.

“Leaving group” refers to a molecular fragment that departs with a pair of electrons in heterolytic bond cleavage. Leaving groups can be anions or neutral molecules. Leaving groups include, but are not limited to halides such as Cl⁻, Br⁻, and I⁻, sulfonate esters, such as para-toluenesulfonate (“tosylate”, TsO⁻), methanesulfonyl (mesyl), p-toluenesulfonyl (tosyl), trifluoromethylsulfonyl (triflate), trifluoromethylsulfonate and RC(O)O— in which R is hydrogen, an aliphatic, heteroaliphatic, carbocyclic, or heterocycloalkyl moiety.

All methods described herein can be performed in any suitable order unless otherwise indicated herein or otherwise clearly contradicted by context. The use of any and all examples, or exemplary language (e.g., “such as”) provided herein, is intended merely to better illustrate the invention and is not to be construed as a limitation on the scope of the claims unless explicitly otherwise claimed. No language in the specification is to be construed as indicating that any non-claimed element is essential to what is claimed.

“Antibody” refers to a full-length antibody or functional fragment of an antibody comprising an immunoglobulin. By a “functional fragment” it is meant a sufficient portion of the immunoglobulin or antibody is provided that the moiety effectively binds or complexes with the cell surface molecule for its target cell population.

An immunoglobulin may be purified, generated recombinantly, generated synthetically, or combinations thereof, using techniques known to those of skill in the art. While immunoglobulins within or derived from IgG antibodies are particularly well-suited for use in this invention, immunoglobulins from any of the classes or subclasses may be selected, e.g., IgG, IgA, IgM, IgD and IgE. Suitably, the immunoglobulin is of the class IgG including but not limited to IgG subclasses (IgG1, 2, 3 and 4) or class IgM which is able to specifically bind to a specific epitope on an antigen. Antibodies can be intact immunoglobulins derived from natural sources or from recombinant sources and can be immunoreactive portions of intact immunoglobulins. Antibodies may exist in a variety of forms including, for example, polyclonal antibodies, monoclonal antibodies, camelized single domain antibodies, intracellular antibodies (“intrabodies”), recombinant antibodies, anti-idiotypic antibodies, domain antibodies, linear antibody, multispecific antibody, antibody fragments, such as, Fv, Fab, F(ab)₂, F(ab)₃, Fab′, Fab′-SH, F(ab′)2, single chain variable fragment antibodies (scFv), tandem/bis-scFv, Fc, pFc′, scFvFc, (or scFv-Fc), disulfide Fv (dsfv), bispecific antibodies (bc-scFv) such as BiTE antibodies; camelid antibodies, resurfaced antibodies, murine antibodies, humanized antibodies, fully human antibodies, single-domain antibody (sdAb, also known as NANOBODY®), chimeric antibodies, chimeric antibodies comprising at least one human constant region, dual-affinity antibodies such as, dual-affinity retargeting proteins (DART™), divalent (or bivalent) single-chain variable fragments (di-scFvs, bi-scFvs) including but not limited to minibodies, diabodies, triabodies or tribodies, tetrabodies, and the like, and multivalent antibodies. “Antibody fragment” refers to at least a portion of the variable region of the immunoglobulin molecule that binds to its target, i.e., the antigen-binding region. As used herein, the term “antibody” refers to both the full-length antibody and antibody fragments unless otherwise specified.

“Protein based recognition-molecule” or “PBRM” refers to a molecule that recognizes and binds to a cell surface marker or receptor such as, a transmembrane protein, surface immobilized protein, or proteoglycan. Examples of PBRMs include but are not limited to, antibodies (e.g., Trastuzumab, Cetuximab, Rituximab, Bevacizumab, Epratuzumab, Veltuzumab, Labetuzumab, B7-H4, B7-H3, CA125, CD33, CXCR2, EGFR, FGFR1, FGFR2, FGFR3, FGFR4, HER2, PD-L1 and anti-5T4) or peptides (LHRH receptor targeting peptides, EC-1 peptide), lipocalins, such as, for example, anticalins, proteins such as, for example, interferons, lymphokines, growth factors, colony stimulating factors, and the like, peptides or peptide mimics, and the like. The protein based recognition molecule, in addition to targeting the modified polymer conjugate to a specific cell, tissue or location, may also have certain therapeutic effect such as antiproliferative (cytostatic and/or cytotoxic) activity against a target cell or pathway. The protein based recognition molecule comprises or may be engineered to comprise at least one chemically reactive group such as, —COOH, primary amine, secondary amine —NHR, —SH, or a chemically reactive amino acid moiety or side chains such as, for example, tyrosine, histidine, cysteine, or lysine. In one embodiment, a PBRM may be a ligand (LG) or targeting moiety which specifically binds or complexes with a cell surface molecule, such as a cell surface receptor or antigen, for a given target cell population. Following specific binding or complexing of the ligand with its receptor, the cell is permissive for uptake of the ligand or ligand-drug-conjugate, which is then internalized into the cell. As used herein, a ligand that “specifically binds or complexes with” or “targets” a cell surface molecule preferentially associates with a cell surface molecule via intermolecular forces. For example, the ligand can preferentially associate with the cell surface molecule with a K_{d} of less than about 50 nM, less than about 5 nM, or less than 500 pM. Techniques for measuring binding affinity of a ligand to a cell surface molecule are well-known; for example, one suitable technique is surface plasmon resonance (SPR). In one embodiment, the ligand is used for targeting, e.g., a cell surface molecule, and has no detectable therapeutic effect, as compared to the drug which it delivers. In another embodiment, the ligand functions both as a targeting moiety and as a therapeutic or immunomodulatory agent (e.g., it modulates or enhances the activity of the active drug or prodrug).

“Biocompatible” as used herein is intended to describe compounds that exert minimal destructive or host response effects while in contact with body fluids or living cells or tissues. Thus a biocompatible group, as used herein, refers to an aliphatic, cycloalkyl, heteroaliphatic, heterocycloalkyl, aryl, or heteroaryl moiety, which falls within the definition of the term biocompatible, as defined above and herein. The term “Biocompatibility” as used herein, is also taken to mean that the compounds exhibit minimal interactions with recognition proteins, e.g., naturally occurring antibodies, cell proteins, cells and other components of biological systems, unless such interactions are specifically desirable. Thus, substances and functional groups specifically intended to cause the above minimal interactions, e.g., drugs and prodrugs, are considered to be biocompatible. Preferably (with exception of compounds intended to be cytotoxic, such as, e.g., antineoplastic agents), compounds are “biocompatible” if their addition to normal cells in vitro, at concentrations similar to the intended systemic in vivo concentrations, results in less than or equal to 1% cell death during the time equivalent to the half-life of the compound in vivo (e.g., the period of time required for 50% of the compound administered in vivo to be eliminated/cleared), and their administration in vivo induces minimal and medically acceptable inflammation, foreign body reaction, immunotoxicity, chemical toxicity and/or other such adverse effects. In the above sentence, the term “normal cells” refers to cells that are not intended to be destroyed or otherwise significantly affected by the compound being tested.

“Biodegradable”: As used herein, “biodegradable” polymers are polymers that are susceptible to biological processing in vivo. As used herein, “biodegradable” compounds or moieties are those that, when taken up by cells, can be broken down by the lysosomal or other chemical machinery or by hydrolysis into components that the cells can either reuse or dispose of without significant toxic effect on the cells. The term “biocleavable” as used herein has the same meaning of “biodegradable”. The degradation fragments preferably induce little or no organ or cell overload or pathological processes caused by such overload or other adverse effects in vivo. Examples of biodegradation processes include enzymatic and non-enzymatic hydrolysis, oxidation and reduction. Suitable conditions for non-enzymatic hydrolysis of the biodegradable protein-polymer-drug conjugates (or their components, e.g., the biodegradable polymeric carrier and the linkers between the carrier and the antibody or the drug molecule) described herein, for example, include exposure of the biodegradable conjugates to water at a temperature and a pH of lysosomal intracellular compartment. Biodegradation of some protein-polymer-drug conjugates (or their components, e.g., the biodegradable polymeric carrier and the linkers between the carrier and the antibody or the drug molecule), can also be enhanced extracellularly, e.g., in low pH regions of the animal body, e.g., an inflamed area, in the close vicinity of activated macrophages or other cells releasing degradation facilitating factors. In certain preferred embodiments, the effective size of the polymer carrier at pH-7.5 does not detectably change over 1 to 7 days, and remains within 50% of the original polymer size for at least several weeks. At pH-5, on the other hand, the polymer carrier preferably detectably degrades over 1 to 5 days, and is completely transformed into low molecular weight fragments within a two-week to several-month time frame. Polymer integrity in such tests can be measured, for example, by size exclusion HPLC. Although faster degradation may be in some cases preferable, in general it may be more desirable that the polymer degrades in cells with the rate that does not exceed the rate of metabolization or excretion of polymer fragments by the cells. In preferred embodiments, the polymers and polymer biodegradation byproducts are biocompatible.

“Bioavailability”: The term “bioavailability” refers to the systemic availability (i.e., blood/plasma levels) of a given amount of drug or compound administered to a subject. Bioavailability is an absolute term that indicates measurement of both the time (rate) and total amount (extent) of drug or compound that reaches the general circulation from an administered dosage form.

“Hydrophilic”: The term “hydrophilic” as it relates to substituents, e.g., on the polymer monomeric units does not essentially differ from the common meaning of this term in the art, and denotes chemical moieties which contain ionizable, polar, or polarizable atoms, or which otherwise may be solvated by water molecules. Thus a hydrophilic group, as used herein, refers to an aliphatic, cycloalkyl, heteroaliphatic, heterocycloalkyl, aryl or heteroaryl moiety, which falls within the definition of the term hydrophilic, as defined above. Examples of particular hydrophilic organic moieties which are suitable include, without limitation, aliphatic or heteroaliphatic groups comprising a chain of atoms in a range of between about one and twelve atoms, hydroxyl, hydroxyalkyl, amine, carboxyl, amide, carboxylic ester, thioester, aldehyde, nitryl, isonitryl, nitroso, hydroxylamine, mercaptoalkyl, heterocycle, carbamates, carboxylic acids and their salts, sulfonic acids and their salts, sulfonic acid esters, phosphoric acids and their salts, phosphate esters, polyglycol ethers, polyamines, polycarboxylates, polyesters and polythioesters. In certain embodiments of the present invention, at least one of the polymer monomeric units include a carboxyl group (COOH), an aldehyde group (CHO), a methylol (CH₂OH), a glycol (for example, CHOH—CH₂OH or CH—(CH₂OH)₂), a ketone group (COC₁₋₄ alkyl), NH₂, F, cyano, SO₃H, PO₃H, and the like.

The term “hydrophilic” as it relates to the polymers of the invention generally does not differ from usage of this term in the art, and denotes polymers comprising hydrophilic functional groups as defined above. In a preferred embodiment, hydrophilic polymer is a water-soluble polymer. Hydrophilicity of the polymer can be directly measured through determination of hydration energy, or determined through investigation between two liquid phases, or by chromatography on solid phases with known hydrophobicity, such as, for example, C4 or C18.

“Polymeric Carrier”: The term polymeric carrier, as used herein, refers to a polymer or a modified polymer, which is suitable for covalently attaching to or can be covalently attached to one or more drug molecules with a designated linker and/or one or more PBRMs with a designated linker.

“Physiological conditions”: The phrase “physiological conditions”, as used herein, relates to the range of chemical (e.g., pH, ionic strength) and biochemical (e.g., enzyme concentrations) conditions likely to be encountered in the extracellular fluids of living tissues. For most normal tissues, the physiological pH ranges from about 7.0 to 7.4. Circulating blood plasma and normal interstitial liquid represent typical examples of normal physiological conditions.

“Polysaccharide”, “carbohydrate” or “oligosaccharide”: The terms “polysaccharide”, “carbohydrate”, or “oligosaccharide” are known in the art and refer, generally, to substances having chemical formula (CH₂O)ₙ, where generally n>2, and their derivatives. Carbohydrates are polyhydroxyaldehydes or polyhydroxyketones, or change to such substances on simple chemical transformations, such as hydrolysis, oxidation or reduction. Typically, carbohydrates are present in the form of cyclic acetals or ketals (such as, glucose or fructose). These cyclic units (monosaccharides) may be connected to each other to form molecules with few (oligosaccharides) or several (polysaccharides) monosaccharide units. Often, carbohydrates with well-defined number, types and positioning of monosaccharide units are called oligosaccharides, whereas carbohydrates consisting of mixtures of molecules of variable numbers and/or positioning of monosaccharide units are called polysaccharides. The terms “polysaccharide”, “carbohydrate”, and “oligosaccharide”, are used herein interchangeably. A polysaccharide may include natural sugars (e.g., glucose, fructose, galactose, mannose, arabinose, ribose, and xylose) and/or derivatives of naturally occurring sugars (e.g., 2′-fluororibose, 2′-deoxyribose, and hexose).

“Prodrug”: As used herein the term “prodrug” refers to a precursor of an active drug, that is, a compound that can be transformed to an active drug. Typically such a prodrug is subject to processing in vivo, which converts the drug into a physiologically active form. In some instances, a prodrug may itself have a desired physiologic effect. A desired physiologic effect may be, e.g., therapeutic, cytotoxic, immunomodulatory, or the like.

“Cytotoxic”: As used herein the term “cytotoxic” means toxic to cells or a selected cell population (e.g., cancer cells). The toxic effect may result in cell death and/or lysis. In certain instances, the toxic effect may be a sub lethal destructive effect on the cell, e.g., slowing or arresting cell growth. In order to achieve a cytotoxic effect, the drug or prodrug may be a DNA damaging agent, a microtubule disrupting agent, or a cell cycle arresting agent, etc., and can be a cytotoxic protein or polypeptide, amongst others.

“Cytostatic”: As used herein the term “cytostatic” refers to a drug or other compound which inhibits or stops cell growth and/or multiplication.

“Drug”: As used herein, the term “drug” refers to a compound which is biologically active and provides a desired physiological effect following administration to a subject in need thereof (e.g., an active pharmaceutical ingredient).

“Drug derivative” or “modified drug” or the like as used herein, refers to a compound that comprises the drug molecule intended to be delivered by the conjugate of the invention and a functional group capable of attaching the drug molecule to the polymeric carrier.

“Active form” as used herein refers to a form of a compound that exhibits intended pharmaceutical efficacy in vivo or in vitro. In particular, when a drug molecule intended to be delivered by the conjugate of the invention is released from the conjugate, the active form can be the drug itself or its derivatives, which exhibit the intended therapeutic properties. The release of the drug from the conjugate can be achieved by cleavage of a biodegradable bond of the linker which attaches the drug to the polymeric carrier. The active drug derivatives accordingly can comprise a portion of the linker.

“Diagnostic label”: As used herein, the term diagnostic label refers to an atom, group of atoms, moiety or functional group, a nanocrystal, or other discrete element of a composition of matter, that can be detected in vivo or ex vivo using analytical methods known in the art. When associated with a conjugate of the present invention, such diagnostic labels permit the monitoring of the conjugate in vivo. Alternatively or additionally, constructs and compositions that include diagnostic labels can be used to monitor biological functions or structures. Examples of diagnostic labels include, without limitation, labels that can be used in medical diagnostic procedures, such as, radioactive isotopes (radionuclides) for gamma scintigraphy and Positron Emission Tomography (PET), contrast agents for Magnetic Resonance Imaging (MRI) (for example paramagnetic atoms and superparamagnetic nanocrystals), contrast agents for computed tomography and other X-ray-based imaging methods, agents for ultrasound-based diagnostic methods (sonography), agents for neutron activation (e.g., boron, gadolinium), fluorophores for various optical procedures, and, in general moieties which can emit, reflect, absorb, scatter or otherwise affect electromagnetic fields or waves (e.g., gamma-rays, X-rays, radiowaves, microwaves, light), particles (e.g., alpha particles, electrons, positrons, neutrons, protons) or other forms of radiation, e.g., ultrasound.

“Aliphatic”: In general, the term aliphatic, as used herein, includes both saturated and unsaturated, straight chain (i.e., unbranched) or branched aliphatic hydrocarbons, which are optionally substituted with one or more functional groups. As will be appreciated by one of ordinary skill in the art, “aliphatic” is intended herein to include, but is not limited to, alkyl, alkenyl, alkynyl moieties. Thus, as used herein, the term “alkyl” includes straight and branched alkyl groups. An analogous convention applies to other generic terms such as “alkenyl”, “alkynyl” and the like. In certain embodiments, as used herein, “lower alkyl” is used to indicate those alkyl groups (substituted, unsubstituted, branched or unbranched) having about 1-6 carbon atoms. “Substituted alkyl” refers to alkyl groups that are substituted with one or more functional groups. Substituents include, but are not limited to, any of the substituents mentioned below, i.e., the substituents recited below resulting in the formation of a stable compound.

“Alkenyl”: the term alkenyl denotes a monovalent group derived from a hydrocarbon moiety having at least one carbon-carbon double bond by the removal of a single hydrogen atom. “Substituted alkenyl” groups are substituted with one or more functional groups. Substituents include, but are not limited to, any of the substituents mentioned below, i.e., the substituents recited below resulting in the formation of a stable compound. Alkenyl groups include, for example, ethenyl, propenyl, butenyl, 1-methyl-2-buten-1-yl, and the like.

“Alkynyl”: the term alkynyl as used herein refers to a monovalent group derived from a hydrocarbon having at least one carbon-carbon triple bond by the removal of a single hydrogen atom. “Substituted alkenyl” groups are substituted with one or more functional groups. Substituents include, but are not limited to, any of the substituents mentioned below, i.e., the substituents recited below resulting in the formation of a stable compound. Representative alkynyl groups include ethynyl, 2-propynyl (propargyl), 1-propynyl, and the like.

In certain embodiments, the alkyl, alkenyl and alkynyl groups employed in the invention contain about 1-20 aliphatic carbon atoms. In certain other embodiments, the alkyl, alkenyl, and alkynyl groups employed in the invention contain about 1-10 aliphatic carbon atoms. In yet other embodiments, the alkyl, alkenyl, and alkynyl groups employed in the invention contain about 1-8 aliphatic carbon atoms. In still other embodiments, the alkyl, alkenyl, and alkynyl groups employed in the invention contain about 1-6 aliphatic carbon atoms. In yet other embodiments, the alkyl, alkenyl, and alkynyl groups employed in the invention contain about 1-4 carbon atoms. Illustrative aliphatic groups thus include, but are not limited to, for example, methyl, ethyl, n-propyl, isopropyl, allyl, n-butyl, sec-butyl, isobutyl, tert-butyl, n-pentyl, sec-pentyl, isopentyl, tert-pentyl, n-hexyl, sec-hexyl, moieties and the like, which again, may bear one or more substituents. Alkenyl groups include, but are not limited to, for example, ethenyl, propenyl, butenyl, 1-methyl-2-buten-1-yl, and the like. Representative alkynyl groups include, but are not limited to, ethynyl, 2-propynyl (propargyl), 1-propynyl and the like.

“Alkylene” as used herein, the term alkylene by itself or part of another term refers to a saturated, branched or straight chain having two monovalent radical centers derived by the removal of two hydrogen atoms from the same or two different carbon atoms of a parent alkane. Alkylene radicals include, but are not limited to, methylene, 1,2, ethylene, 1,3-propyl, and the like. Suitable alkylenes include, but are not limited to methylene, ethylene, propylene, butylene, pentylene, hexylene, heptylene, ocytylene, nonylene, decalene, and the like. The term “cycloalkylene” similarly refers to bivalent cycloalkyl. Cycloalkylene radicals include, but are not limited to, 1,1-cyclopentylene, 1,2-cyclopentylene, 1,1-cyclobutylene, 1,3-cyclobutylene, etc.

“Heteroaliphatic”: as used herein, the term heteroaliphatic refers to aliphatic moieties in which one or more carbon atoms in the main chain have been substituted with a heteroatom. Thus, a heteroaliphatic group refers to an aliphatic chain which contains one or more oxygen, sulfur, nitrogen, phosphorus or silicon atoms, e.g., in place of carbon atoms. Heteroaliphatic moieties may be branched or linear unbranched. In certain embodiments, heteroaliphatic moieties are substituted (“substituted heteroaliphatic”) by independent replacement of one or more of the hydrogen atoms thereon with one or more moieties including, but not limited to aliphatic; heteroaliphatic; cycloalkyl; heterocycloalkyl; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —N_{O2}; —CN; —C_{F3}; —C_{H2}C_{F3}; —CHCₗ₂; —C_{H2}OH; —C_{H2}C_{H2}OH; —C_{H2}N_{H2}; —C_{H2}S_{O2}C_{H3}; - or -G^{RG1} wherein G is —O—, —S—, —N^{RG2}—, —C(═O)—, —S(═O)—, —S_{O2}—, —C(═O)O—, —C(═O)N^{RG2}—, —OC(═O)—, —N^{RG2}C(═O)—, —OC(═O)O—, —OC(═O)N^{RG2}—, —N^{RG2}C(═O)O—, —N^{RG2}C(═O)N^{RG2}—, —C(═S)—, —C(═S)S—, —SC(═S)—, —SC(═S)S—, —C(═N^{RG2})—, —C(═N^{RG2})O—, —C(═N^{RG2})N^{RG3}—, —OC(═N^{RG2})—, —N^{RG2}C(═N^{RG3})—, —N^{RG2}S_{O2}—, —N^{RG2}S_{O2}N^{RG3}—, or —S_{O2}N^{RG2}—, wherein each occurrence of ^{RG1}, ^{RG2} and ^{RG3} independently includes, but is not limited to, hydrogen, halogen, or an aliphatic, heteroaliphatic, cycloalkyl, heterocycloalkyl, aryl, heteroaryl, alkylaryl, or alkylheteroaryl moiety, each of which is optionally substituted. Additional examples of generally applicable substituents are illustrated by the specific embodiments shown in the Examples that are described herein.

“Cycloalkyl”: as used herein, the term cycloalkyl refers to a saturated or unsaturated nonaromatic hydrocarbon mono- or multi-ring system having 3 to 30 carbon atoms (e.g., C₃-C₁₀). Suitable cycloalkyls include, but are not limited to cyclopropyl, cyclobutyl, cyclopentyl, cyclohexyl, cycloheptyl, cyclooctyl, cyclopentenyl, cyclohexenyl, cycloheptenyl, cycloheptynyl, adamantyl, and the like.

“Heterocycloalkyl” as used herein refers to a saturated or unsaturated nonaromatic 3-8 membered monocyclic, 8-12 membered bicyclic, or 11-19 membered tricyclic ring system having one or more heteroatoms (such as O, N, S, or Se), unless specified otherwise. In certain embodiments, the term “heterocycloalkyl” refers to a non-aromatic 5-, 6-, 7- or 8-membered ring or a polycyclic group, including, but not limited to a bi- or tri-cyclic group comprising fused six-membered rings having between one and three heteroatoms independently selected from oxygen, sulfur and nitrogen, wherein (i) each 5-membered ring has 0 to 2 double bonds and each 6-membered ring has 0 to 2 double bonds, (ii) the nitrogen and sulfur heteroatoms may optionally be oxidized, (iii) the nitrogen heteroatom may optionally be quaternized, and (iv) any of the above heterocycloalkyl; rings may be fused to an aryl or heteroaryl ring. Examples of heterocycloalkyl groups include, but are not limited to, piperidinyl, piperazinyl, pyrrolidinyl, dioxanyl, tetrahydrofuranyl, tetrahydrothienyl, isoindolinyl, indolinyl, imidazolidinyl, pyrazolidinyl, oxazolidinyl, isoxazolidinyl, triazolidinyl, tetrahyrofuranyl, oxiranyl, azetidinyl, oxetanyl, thietanyl, 1,2,3,6-tetrahydropyridinyl, tetrahydro-2H-pyranyl, 3,6-dihydro-2H-pyranyl, morpholinyl, and the like.

“Aryl”: as used herein, refers to groups with aromaticity, including “conjugated,” or multicyclic systems with at least one aromatic ring and do not contain any heteroatom in the ring structure. Examples include phenyl, benzyl, 1,2,3,4-tetrahydronaphthalenyl, etc.

“Heteroaryl”: as used herein, refers to aryl groups, as defined above, except having from one to four heteroatoms in the ring structure, and may also be referred to as “aryl heterocycles” or “heteroaromatics.” As used herein, the term “heteroaryl” is intended to include a stable 5-, 6-, or 7-membered monocyclic or 7-, 8-, 9-, 10-, 11- or 12-membered bicyclic aromatic heterocyclic ring which consists of carbon atoms and one or more heteroatoms, e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulfur. The nitrogen atom may be substituted or unsubstituted (i.e., N or NR wherein R is H or other substituents, as defined). The nitrogen and sulfur heteroatoms may optionally be oxidized (i.e., N→O and S(O)ₚ, where p=1 or 2). It is to be noted that total number of S and O atoms in the aromatic heterocycle is not more than 1. Examples of heteroaryl include pyridyl, pyrazinyl, pyrimidinyl, pyrrolyl, pyrazolyl, imidazolyl, thiazolyl, isothiazolyl, tetrazolyl, oxazolyl, isooxazolyl, thiadiazolyl, oxadiazolyl, thiophenyl, furanyl, quinolinyl, isoquinolinyl, tetrazolyl, pyridazinyl, quinazolinyl, dihydroquinazolyl, and tetrahydroquinazolyl and the like.

Furthermore, the terms “aryl” and “heteroaryl” include multicyclic aryl and heteroaryl groups, e.g., tricyclic, bicyclic, e.g., naphthalene, benzoxazole, benzodioxazole, benzothiazole, benzoimidazole, benzothiophene, methylenedioxyphenyl, quinoline, isoquinoline, naphthrydine, indole, benzofuran, purine, benzofuran, deazapurine, indolizine.

In the case of multicyclic aromatic rings, only one of the rings needs to be aromatic (e.g., 2,3-dihydroindole), although all of the rings may be aromatic (e.g., quinoline). The second ring can also be fused or bridged.

“Carbocycle” or “carbocyclic moiety” as used herein, is intended to include any stable monocyclic, bicyclic or tricyclic ring having the specified number of carbons, any of which may be saturated, unsaturated, or aromatic. Carbocycle includes cycloalkyl and aryl. For example, a C₃-C₁₄ carbocycle is intended to include a monocyclic, bicyclic or tricyclic ring having 3, 4, 5, 6, 7, 8, 9, 10, 11, 12, 13 or 14 carbon atoms. Examples of carbocycles include, but are not limited to, cyclopropyl, cyclobutyl, cyclobutenyl, cyclopentyl, cyclopentenyl, cyclohexyl, cycloheptenyl, cycloheptyl, cycloheptenyl, cyclooctyl, cyclooctenyl, cyclooctadienyl, fluorenyl, phenyl, naphthyl, indanyl, adamantyl and tetrahydronaphthyl. Bridged rings are also included in the definition of carbocycle, including, for example, [3.3.0]bicyclooctane, [4.3.0]bicyclononane, [4.4.0]bicyclodecane and

bicyclooctane. A bridged ring occurs when one or more carbon atoms link two non-adjacent carbon atoms. In one embodiment, bridge rings are one or two carbon atoms. It is noted that a bridge always converts a monocyclic ring into a tricyclic ring. When a ring is bridged, the substituents recited for the ring may also be present on the bridge. Fused (e.g., naphthyl, tetrahydronaphthyl) and spiro rings are also included.

“Heterocycle” or “heterocyclic moiety” as used herein, includes any ring structure (saturated, unsaturated, or aromatic) which contains at least one ring heteroatom (e.g., N, O or S). Heterocycle includes heterocycloalkyl and heteroaryl. Examples of heterocycles include, but are not limited to, morpholine, pyrrolidine, tetrahydrothiophene, piperidine, piperazine and tetrahydrofuran.

Examples of heterocyclic groups include, but are not limited to, acridinyl, azocinyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, 1,2,4-oxadiazol5(4H)-one, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl and xanthenyl. Multiple-ring heterocycle can include fused, bridged or spiro rings.

The cycloalkyl, heterocycloalkyl, aryl, or heteroaryl ring (or the carbocyclic or heterocyclic group) can be substituted at one or more ring positions (e.g., the ring-forming carbon or heteroatom such as N) with such substituents as described above, for example, aliphatic; heteroaliphatic; cycloalkyl; heterocycloalkyl; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —NO₂; —CN; —CF₃; —CH₂CF₃; —CHCl₂; —CH₂OH; —CH₂CH₂OH; —CH₂NH₂; —CH₂SO₂CH₃; - or -GR^{G1} wherein G is —O—, —S—, —NR^{G2}—, —C(═O)—, —S(═O)—, —SO₂—, —C(═O)O—, —C(═O)NR^{G2}—, —OC(═O)—, —NR^{G2}C(═O)—, —OC(═O)O—, —OC(═O)NR^{G2}—, —NR^{G2}C(═O)O—, —NR^{G2}C(═O)NR^{G2}—, —C(═S)—, —C(═S)S—, —SC(═S)—, —SC(═S)S—, —C(═NR^{G2})—, —C(═NR^{G2})O—, —C(═NR^{G2})NR^{G3}—, —OC(═NR^{G2})—, —NR^{G2}C(═NR^{G3})—, —NR^{G2}SO₂—, —NR^{G2}SO₂NR^{G3}—, or —SO₂NR^{G2}—, wherein each occurrence of R^{G1}, R^{G2} and R^{G3} independently includes, but is not limited to, hydrogen, halogen, or an aliphatic, heteroaliphatic, cycloalkyl, heterocycloalkyl; aryl, heteroaryl, alkylaryl, or alkylheteroaryl moiety, each of which is optionally substituted. Aryl and heteroaryl groups can also be fused or bridged with cycloalkyl or heterocyclic rings, which are not aromatic so as to form a multicyclic system (e.g., tetralin, methylenedioxyphenyl).

“Alkoxy” (or “alkyloxy”): as used herein, the term alkoxy (or alkyloxy) refers to an alkyl group, as previously defined, attached to the parent molecular moiety through an oxygen atom (“alkoxy”). In certain embodiments, the alkyl group contains about 1-20 aliphatic carbon atoms. In certain other embodiments, the alkyl group contains about 1-10 aliphatic carbon atoms. In yet other embodiments, the alkyl group contains about 1-8 aliphatic carbon atoms. In still other embodiments, the alkyl group contains about 1-6 aliphatic carbon atoms. In yet other embodiments, the alkyl group contains about 1-4 aliphatic carbon atoms. Examples of alkoxy groups, include but are not limited to, methoxy, ethoxy, propoxy, isopropoxy, n-butoxy, tert-butoxy, neopentoxy and n-hexoxy.

“Aryloxy”: as used herein, the term aryloxy refers to an aryl group, as defined herein, attached to the parent molecular moiety through an oxygen atom. Examples of aryloxy groups include but are not limited to phenoxy and napthyloxy.

“Heteroaryloxy”: as used herein, the term heteroaryloxy refers to a heteroaryl group, as defined herein, attached to the parent molecular moiety through an oxygen atom. Examples of heteroaryloxy groups include but are not limited to, quinolyloxy and isoquinolizinyloxy.

“Amine”: the term amine refers to a group having the structure —N(R)₂ wherein each occurrence of R is independently hydrogen, or an aliphatic or heteroaliphatic moiety, or the R groups, taken together, may form a heterocyclic moiety. In certain instances, an amine group can be charged (protonized) or quarternized, e.g., —HN⁺(R)₂ or —N⁺(R)₃.

“Alkylamino”: as used herein, the term alkylamino refers to amino substituted with at least one alkyl group, such as a group having the structure —NRR′ wherein R′ is alkyl, as defined herein and R is H, alkyl, alkenyl, alkynyl, aryl, cycloalkyl, heteroaryl and the like. The term “alkylamino” includes mono-alkylamino and di-alkylamino. The term “aminoalkyl” refers to a group having the structure NH₂R′—, wherein R′ is alkyl, as defined herein. In certain embodiments, the alkyl group contains about 1-20 aliphatic carbon atoms. In certain other embodiments, the alkyl group contains about 1-10 aliphatic carbon atoms. In yet other embodiments, the alkyl, alkenyl, and alkynyl groups employed in the invention contain about 1-8 aliphatic carbon atoms. In still other embodiments, the alkyl group contains about 1-6 aliphatic carbon atoms. In yet other embodiments, the alkyl group contains about 1-4 aliphatic carbon atoms. Examples of alkylamino include, but are not limited to, methylamino, ethylamino, iso-propylamino and the like.

“Alkylthio” (or “thioalkyl”) means an alkyl group as defined herein with the indicated number of carbon atoms attached through a sulfur atom. C₁₋₆ alkylthio, is intended to include C₁, C₂, C₃, C₄, C₅, and C₆ alkylthio groups. C₁₋₈ alkylthio, is intended to include C₁, C₂, C₃, C₄, C₅, C₆, C₇, and C₈ alkylthio groups. The thioalkyl groups can be substituted with groups such as alkyl, alkenyl, alkynyl, halogen, hydroxyl, alkylcarbonyloxy, arylcarbonyloxy, alkoxycarbonyloxy, aryloxycarbonyloxy, carboxylate, carboxyacid, alkylcarbonyl, arylcarbonyl, alkoxycarbonyl, aminocarbonyl, alkylaminocarbonyl, dialkylaminocarbonyl, alkylthiocarbonyl, alkoxyl, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl alkylaryl, or an aryl or heteroaryl moieties.

“Thiocarbonyl” or “thiocarboxy” includes compounds and moieties which contain a carbon connected with a double bond to a sulfur atom.

“Arylthio” (or “thioaryl”) means an aryl group as defined herein with the indicated number of carbon atoms attached through a sulfur atom.

“Carboxylic acid” as used herein refers to a compound comprising a group of formula —CO₂H.

“Dicarboxylic acid” refers to a compound comprising two groups of formula —CO₂H.

“Halo, halide and halogen”: The terms halo, halide and halogen as used herein refer to an atom selected from fluorine, chlorine, bromine, and iodine.

“Methylol”: The term methylol as used herein refers to an alcohol group of the structure —CH₂OH.

“Hydroxyalkyl”: As used herein, the term hydroxyalkyl refers to an alkyl group, as defined above, bearing at least one OH group.

“Mercaptoalkyl”: The term mercaptoalkyl as used therein refers to an alkyl group, as defined above, bearing at least one SH group.

“Acyl” includes moieties that contain the acyl radical (—C(O)—) or a carbonyl group. “Substituted acyl” includes acyl groups where one or more of the hydrogen atoms are replaced by, for example, alkyl groups, alkynyl groups, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aryl or heteroaryl moiety.

“Hydrocarbon”: The term hydrocarbon, as used herein, refers to any chemical group comprising hydrogen and carbon. The hydrocarbon may be substituted or unsubstituted. The hydrocarbon may be unsaturated, saturated, branched, unbranched, cyclic, polycyclic, or heterocyclic. Illustrative hydrocarbons include, for example, methyl, ethyl, n-propyl, iso-propyl, cyclopropyl, allyl, vinyl, n-butyl, tert-butyl, ethynyl, cyclohexyl, methoxy, diethylamino, heterocycloalkyl, aryl, heteroaryl, thioalkyl, and the like. As would be known to one skilled in this art, all valencies must be satisfied in making any substitutions.

“Alkylaryl” as used herein refers to an aryl group substituted with one or more alkyl groups (e.g., methylphenyl).

“Alkylarylamino” as used herein refers to —NR^{G4}R^{G5}, wherein R^{G4} is alkyl, as defined herein, and R^{G5} is an aryl, as defined herein, or at least one of R^{G4} and R^{G5} is an alkylaryl as defined herein.

“Substituted”: The terms substituted, whether preceded by the term “optionally” or not, and substituent, as used herein, refers to the replacement of a hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Heteroatoms such as nitrogen may have hydrogen substituents and/or any permissible substituents of organic compounds described herein which satisfy the valencies of the heteroatoms. Examples of substituents include, but are not limited to aliphatic; heteroaliphatic; cycloalkyl; heterocycloalkyl; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —NO₂; —CN; —CF₃; —CH₂CF₃; —CHCl₂; —CH₂OH; —CH₂CH₂OH; —CH₂NH₂; —CH₂SO₂CH₃; - or -GR^{G1} wherein G is —O—, —S—, —NR^{G2}—, —C(═O)—, —S(═O)—, —SO₂—, —C(═O)O—, —C(═O)NR^{G2}—, —OC(═O)—, —NR^{G2}C(═O)—, —OC(═O)O—, —OC(═O)NR^{G2}—, —NR^{G2}C(═O)O—, —NR^{G2}C(═O)NR^{G2}—, —C(═S)—, —C(═S)S—, —SC(═S)—, —SC(═S)S—, —C(═NR^{G2})—, —C(═NR^{G2})O—, —C(═NR^{G2})NR^{G3}—, —OC(═NR^{G2})—, —NR^{G2}C(═NR^{G3})—, —NR^{G2}SO₂—, —NR^{G2}SO₂NR^{G3}—, or —SO₂NR^{G2}—, wherein each occurrence of R^{G1}, R^{G2} and R^{G3} independently includes, but is not limited to, hydrogen, halogen, or an aliphatic, heteroaliphatic, cycloalkyl, heterocycloalkyl, aryl, heteroaryl, alkylaryl, or alkylheteroaryl moiety, each of which is optionally substituted. Additional examples of generally applicable substituents are illustrated by the specific embodiments shown in the Examples that are described herein.

The following are more general terms used throughout the present application:

“Animal”: The term animal, as used herein, refers to humans as well as non-human animals, at any stage of development, including, for example, mammals, birds, reptiles, amphibians, fish, worms and single cells. Cell cultures and live tissue samples are considered to be pluralities of animals. Preferably, the non-human animal is a mammal (e.g., a rodent, a mouse, a rat, a rabbit, a monkey, a dog, a cat, a primate, or a pig). An animal may be a transgenic animal or a human clone. The term “subject” encompasses animals.

“Efficient amount”: In general, as it refers to an active agent or drug delivery device, the term “efficient amount” refers to the amount necessary to elicit the desired biological response. As will be appreciated by those of ordinary skill in this art, the efficient amount of an agent or device may vary depending on such factors as the desired biological endpoint, the agent to be delivered, the composition of the encapsulating matrix, the target tissue, etc. For example, the efficient amount of microparticles containing an antigen to be delivered to immunize an individual is the amount that results in an immune response sufficient to prevent infection with an organism having the administered antigen.

“Natural amino acid” as used herein refers to any one of the common, naturally occurring L-amino acids found in naturally occurring proteins: glycine (Gly), alanine (Ala), valine (Val), leucine (Leu), isoleucine (Ile), lysine (Lys), arginine (Arg), histidine (His), proline (Pro), serine (Ser), threonine (Thr), phenylalanine (Phe), tyrosine (Tyr), tryptophan (Trp), aspartic acid (Asp), glutamic acid (Glu), asparagine (Asn), glutamine (Gln), cysteine (Cys) and methionine (Met).

“Unnatural amino acid” as used herein refers to any amino acid which is not a natural amino acid. This includes, for example, amino acids that comprise α-, β-, ω-, D-, L-amino acyl residues. More generally, the unnatural amino acid comprises a residue of the general formula

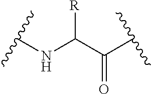

wherein the side chain R is other than the amino acid side chains occurring in nature. Exemplary unnatural amino acids, include, but are not limited to, sarcosine (N-methylglycine), citrulline (cit), homocitrulline, β-ureidoalanine, thiocitrulline, hydroxyproline, allothreonine, pipecolic acid (homoproline), α-aminoisobutyric acid, tert-butylglycine, tert-butylalanine, allo-isoleucine, norleucine, α-methylleucine, cyclohexylglycine, β-cyclohexylalanine, β-cyclopentylalanine, α-methylproline, phenylglycine, α-methylphenylalanine and homophenylalanine.

“Amino acyl”: More generally, the term amino acyl, as used herein, encompasses natural amino acid and unnatural amino acids.

“Polyamide”: refers to homo- or hetero-polymers of natural amino acid and unnatural amino acids. Illustrative homo-polymers include, but are not limited to, poly-lysine, poly-arginine, poly-γ-glutaric acid, and the like. Illustrative hetero-polymers include, but are not limited to, polymers comprising peptides fragments selected from peptidases, lysozymes, metalloproteinases, and the like.

“PHF” refers to poly(1-hydroxymethylethylene hydroxymethyl-formal).

As used herein, the terms “polymer unit”, “monomeric unit”, “monomer”, “monomer unit”, “unit” all refer to a repeatable structural unit in a polymer.

“Arylene” as used herein refers to an aryl group which has two covalent bonds and can be in the ortho, meta, or para configurations as shown in the following structures:

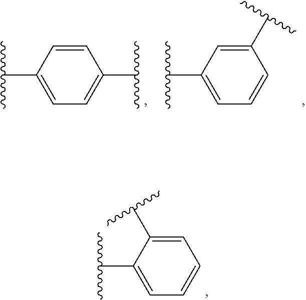

in which the phenyl group can be unsubstituted or substituted with up to four groups including, but not limited to, C₁₋₈ alkyl, —O—(C₁₋₈ alkyl), C₆₋₁₀ aryl, —C(O)R′, —OC(O)R′, —C(O)OR′, —C(O)NH₂, —C(O)NHR′, —C(O)N(R′)₂, NHC(O)R′, —S(O)₂R′, —S(O)R′, —OH, -halogen, —N₃, —NH₂, —NH(R′), —N(R′)₂ and —CN; wherein each R′ independently is H, —C₁₋₃ alkyl or C₆₋₁₀ aryl.

“Hydroxy- or oxo-substituted C₁₋₈ alkyl” as used herein refers to a lower alkyl group wherein a hydrogen on the lower alkyl group is replaced by —OH (for a hydroxy-substituted lower alkyl), or two hydrogens on a single carbon of the lower alkyl group are replaced by ═O (for an oxo-substituted lower alkyl).

“Tubulysin compounds” as used herein refers to a family of potent inhibitors of tubulin polymerization. Tubulysins are useful in treating diseases and disease states that include pathogenic cell populations, such as cancer. The term “tubulysin compounds” includes their derivatives or modified forms, such that they are suitable for conjugation with the polymers or polymeric scaffolds described herein and can convert into active forms when the compounds are released from the polymers. In some embodiments, tubulysin compounds described herein have cytotoxic activity against drug resistant tumors. In some embodiments, tubulysin compounds described herein are naturally occurring tubulysins (or natural tubulysins), analogs, and derivatives thereof. For example, naturally occurring tubulysins are selected from tubulysins A, B, C, G, and I, each of which is characterized by a including the tubutyrosine (Tut, an analog of tyrosine) residue, and tubulysins D, E, F, and H, each of which is characterized by a including the tubuphenylalanine (Tup, an analog of phenylalanine) residue.

As used herein, “molecular weight” or “MW” of a polymer or polymeric carrier/scaffold or polymer conjugates refers to the weight average molecular weight unless otherwise specified.

The present invention is intended to include all isotopes of atoms occurring in the present compounds. Isotopes include those atoms having the same atomic number but different mass numbers. By way of general example and without limitation, isotopes of hydrogen include tritium and deuterium. Isotopes of carbon include C-13 and C-14.

The present invention is intended to include all isomers of the compound, which refers to and includes, optical isomers, and tautomeric isomers, where optical isomers include enantiomers and diastereomers, chiral isomers and non-chiral isomers, and the optical isomers include isolated optical isomers as well as mixtures of optical isomers including racemic and non-racemic mixtures; where an isomer may be in isolated form or in a mixture with one or more other isomers.

### Polymeric Carriers

In certain exemplary embodiments, the conjugates of the invention find use in biomedical applications, such as drug delivery and tissue engineering, and the carrier is biocompatible and biodegradable. In certain embodiments, the carrier is a soluble polymer, nanoparticle, gel, liposome, micelle, suture, implant, etc. In certain embodiments, the term “soluble polymer” encompasses biodegradable biocompatible polymer such as a polyal (e.g., hydrophilic polyacetal or polyketal). In certain other embodiments, the carrier is a fully synthetic, semi-synthetic or naturally-occurring polymer. In certain other embodiments, the carrier is hydrophilic.

In certain exemplary embodiments, the carriers used in the present invention are biodegradable biocompatible polyals comprising at least one hydrolysable bond in each monomer unit positioned within the main chain. This ensures that the degradation process (via hydrolysis/cleavage of the monomer units) will result in fragmentation of the polymer conjugate to the monomeric components (i.e., degradation), and confers to the polymer conjugates of the invention their biodegradable properties. The properties (e.g., solubility, bioadhesivity and hydrophilicity) of biodegradable biocompatible polymer conjugates can be modified by subsequent substitution of additional hydrophilic or hydrophobic groups. Examples of biodegradable biocompatible polymers suitable for practicing the invention can be found inter alia in U.S. Pat. Nos. 5,811,510; 5,863,990; 5,958,398; 7,838,619 and 7,790,150; and U.S. Publication No. 2006/0058512; each of the above listed patent documents is incorporated herein by reference in its entirety. Guidance on the significance, preparation, and applications of this type of polymers may be found in the above-cited documents. In certain embodiments, it is anticipated that the present invention will be particularly useful in combination with the above-referenced patent documents, as well as U.S. Pat. Nos. 5,582,172 and 6,822,086, each of the above listed patent documents is incorporated herein by reference in its entirety.

The conjugates of this invention are hydrophilic, hydrolysable and comprise drug molecules (e.g., tubulysin s compounds, and analogs thereof) and antibodies (e.g., Trastuzumab, Cetuximab, Rituximab, Bevacizumab, Epratuzumab, Veltuzumab, Labetuzumab) or peptides (LHRH receptor targeting peptides, EC-1 peptide) covalently attached to the polymer carrier via linkages that contain one or more biodegradable bonds. Thus, in certain exemplary embodiments, carriers suitable for practicing the present invention are polyals having at least one acetal/ketal oxygen atom in each monomer unit positioned within the main chain. As discussed above, this ensures that the degradation process (via hydrolysis/cleavage of the polymer acetal/ketal groups) will result in fragmentation of the polyal conjugate to low molecular weight components (i.e., degradation).

In certain embodiments, biodegradable biocompatible polymer carriers, used for preparation of polymer conjugates of the invention, are naturally occurring polysaccharides, glycopolysaccharides, and synthetic polymers of polyglycoside, polyacetal, polyamide, polyether, and polyester origin and products of their oxidation, fictionalization, modification, cross-linking, and conjugation.

In certain other embodiments, the carrier is a hydrophilic biodegradable polymer selected from the group consisting of carbohydrates, glycopolysaccharides, glycolipids, glycoconjugates, polyacetals, polyketals, and derivatives thereof.

In certain exemplary embodiments, the carrier is a naturally occurring linear and/or branched biodegradable biocompatible homopolysaccharide selected from the group consisting of cellulose, amylose, dextran, levan, fucoidan, carraginan, inulin, pectin, amylopectin, glycogen and lixenan.

In certain other exemplary embodiments, the carrier is a naturally occurring linear and branched biodegradable biocompatible heteropolysaccharide selected from the group consisting of agarose, hyluronan, chondroitinsulfate, dermatansulfate, keratansulfate, alginic acid and heparin.

In yet other exemplary embodiments, the polymeric carrier comprises a copolymer of a polyacetal/polyketal and a hydrophilic polymer selected from the group consisting of polyacrylates, polyvinyl polymers, polyesters, polyorthoesters, polyamides, polypeptides, and derivatives thereof.

In yet another embodiment, the polymeric carrier is dextrin that is produced by the hydrolysis of a starch obtained from various natural products such as, for example, wheat, rice, maize and tapioca. Depending on the structure of the starch starting material each dextrin comprises a unique distribution of α-1,4 linkages and α-1,6 linkages. Since the rate of biodegradability of α-1,6 linkages is typically less than that for α-1,4 linkages, preferably the percentage of α-1,6 linkages is less than 10% and more preferably less than 5%. In one embodiment the molecular weight of the dextrin is in the range of about 1 kDa to about 200 kDa, more preferably from about 2 kDa to about 55 kDa.

In certain embodiments, the carrier comprises polysaccharides activated by selective oxidation of cyclic vicinal diols of 1,2-, 1,4-, 1,6-, and 2,6-pyranosides, and 1,2-, 1,5-, 1,6-furanosides, or by oxidation of lateral 6-hydroxy and 5,6-diol containing polysaccharides prior to conjugation with drug molecules or PBRMs.

In still other embodiments, the polymeric carrier comprises a biodegradable biocompatible polyacetal wherein at least a subset of the polyacetal repeat structural units have the following chemical structure:

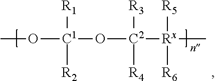

wherein for each occurrence of the n bracketed structure, one of R₁ and R₂ is hydrogen, and the other is a biocompatible group and includes a carbon atom covalently attached to C¹; R^{x} is a carbon atom covalently attached to C²; n″ is an integer; each occurrence of R₃, R₄, R₅ and R₆ is a biocompatible group and is independently hydrogen or an organic moiety; and for each occurrence of the bracketed structure n, at least one of R₁, R₂, R₃, R₄, R₅ and R₆ comprises a functional group suitable for coupling. In certain embodiments, the functional group is a hydroxyl moiety.

In one embodiment, the polymeric carrier comprises activated hydrophilic biodegradable biocompatible polymers comprising from 0.1% to 100% polyacetal moieties whose backbone is represented by the following chemical structure:

(—CH₂—CHR₇—O—CHR₈—O—)ₒ,

wherein:

R₇ and R₈ are independently hydrogen, hydroxyl, hydroxy alkyl (e.g., —CH₂OH, —CH(OH)—CH₂OH), —CHO, —CH(OH)—CHO or -carbonyl; and

o is an integer from 20 to 2000.

In yet other embodiments, the polymeric carrier comprises a biodegradable biocompatible polyketal wherein at least a subset of the polyketal repeatable structural units have the following chemical structure:

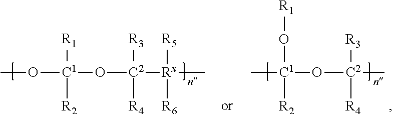

wherein each occurrence of R₁ and R₂ is a biocompatible group and R^{x}, R₃, R₄, R₅, R₆ and are as defined herein

In certain embodiments, the ketal units are monomers of Formula (IIa₁) or (IIb₁):

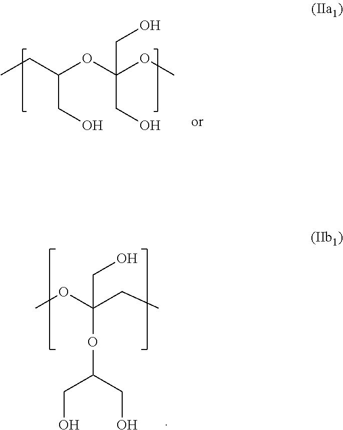

Biodegradable, biocompatible polyketal polymers and their methods of making have been described in U.S. Pat. Nos. 5,811,510, 7,790,150 and 7,838,619, which are hereby incorporated by reference in their entireties.

In one embodiment, the polymeric carrier can be obtained from partially oxidized dextran (β1→6)-D-glucose) followed by reduction. In this embodiment, the polymer comprises a random mixture of the unmodified dextran (A), partially oxidized dextran acetal units (B) and exhaustively dextran acetal units (C) of the following structures:

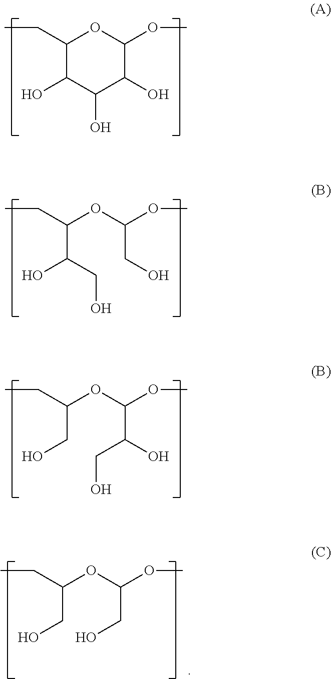

In another embodiment, the polymeric carrier comprises unmodified acetal units, i.e., polyacetal segments. In some embodiments, the polyacetals can be derived from exhaustively oxidized dextran followed by reduction. These polymers have been described in references, see, for example, U.S. Pat. No. 5,811,510, which is hereby incorporated by reference for its description of polyacetals at column 2, line 65 to column 8, line 55 and their synthesis at column 10, line 45 to column 11, line 14. In one embodiment, the unmodified polyacetal polymer is a poly(hydroxymethylethylene hydroxymethyl formal) polymer (PHF).

In addition to poly(hydroxymethylethylene hydroxymethyl formal) polymers, the backbone of the polymeric carrier can also comprise co-polymers of poly(hydroxymethylethylene hydroxymethyl formal) blocks and other acetal or non-acetal monomers or polymers. For example, polyethylene glycol polymers are useful as a stealth agent in the polymer backbone because they can decrease interactions between polymer side chains of the appended functional groups. Such groups can also be useful in limiting interactions such as between serum factors and the modified polymer. Other stealth agent monomers for inclusion in the polymer backbone include, for example, ethyleneimine, methacrylic acid, acrylamide, glutamic acid, and combinations thereof.

The acetal or ketal units are present in the modified polymer in an amount effective to promote biocompatibility. The unmodified acetal or ketal unit can be described as a “stealth agent” that provides biocompatibility and solubility to the modified polymers. In addition, conjugation to a polyacetal or a polyketal polymer can modify the susceptibility to metabolism and degradation of the moieties attached to it, and influence biodistribution, clearance and degradation.

The unmodified acetal units are monomers of Formula (III):

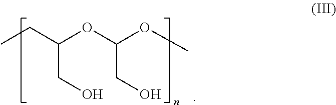

The molar fraction, n, of unmodified polyacetal units is the molar fraction available to promote biocompatibility, solubility and increase half-life, based on the total number of polymer units in the modified polymer. The molar fraction n may be the minimal fraction of unmodified monomer acetal units needed to provide biocompatibility, solubility, stability, or a particular half-life, or can be some larger fraction. The most desirable degree of cytotoxicity is substantially none, i.e., the modified polymer is substantially inert to the subject. However, as is understood by those of ordinary skill in the art, some degree of cytotoxicity can be tolerated depending on the severity of disease or symptom being treated, the efficacy of the treatment, the type and degree of immune response, and like considerations.

In one embodiment, the modified polymer backbone comprises units of Formula (IVa):

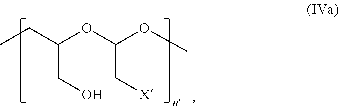

wherein X′ indicates the substituent for the hydroxyl group of the polymer backbone. As shown in Formula (IVa) and the other formulae described herein, each polyacetal unit has a single hydroxyl group attached to the glycerol moiety of the unit and an X′ group (or another substituent such as -L^{D}-D) attached to the glycolaldehyde moiety of the unit. This is for convenience only and it should be construed that the polymer having units of Formula (IVa) and other formulae described herein can contain a random distribution of units having a X′ group (or another substituent such as -L^{D}-D) attached to the glycolaldehyde moiety of the units and those having a single X′ group (or another substituent such as -L^{D}-D) attached to the glycerol moiety of the units as well as units having two X′ groups (or other substituents such as -L^{D}-D) with one attached to the glycolaldehyde moiety and the other attached to the glycerol moiety of the units.

In one embodiment, biodegradable biocompatible polyals suitable for practicing the present invention have a molecular weight of between about 0.5 and about 300 kDa. In a preferred embodiment of the present invention, the biodegradable biocompatible polyals have a molecular weight of between about 1 and about 300 kDa (e.g., between about 1 and about 200 kDa, between about 2 and about 300 kDa, between about 2 and about 200 kDa, between about 5 and about 100 kDa, between about 10 and about 70 kDa, between about 20 and about 50 kDa, between about 20 and about 300 kDa, between about 40 and about 150 kDa, between about 50 and about 100 kDa, between about 2 and about 40 kDa, between about 6 and about 20 kDa, or between about 8 and about 15 kDa).

In one embodiment, the biodegradable biocompatible polyals suitable for practicing the present invention are modified before conjugating with a drug or a PBRM. For example, the polyals contain the moiety —C(═O)—X—(CH₂)ᵥ—C(═O)— with X being CH₂, O, or NH, and v being an integer from 1 to 6. Table A below provides some examples of the modified polyals suitable for conjugating with a drug or PBRM or derivatives thereof.

TABLE A

| Ref # | Polymer Scaffold |
|---|---|
| X = NH Ex 1 X = CH₂ Ex 2 | 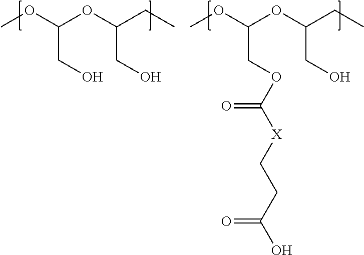 |
| X = CH₂ Ex 6 X = NH Ex 7 | 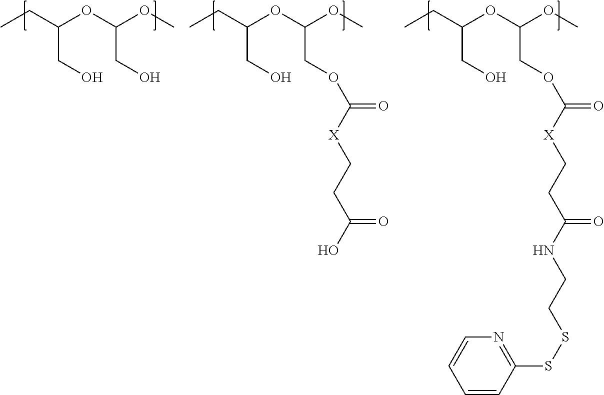 |
| | 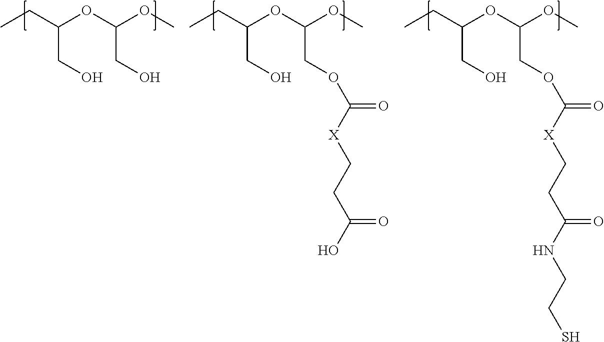 |
| X = CH₂ Ex 8 | 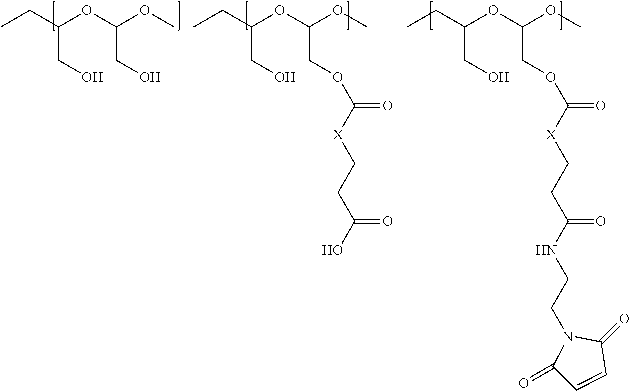 |
| X = CH₂ Ex 12 | 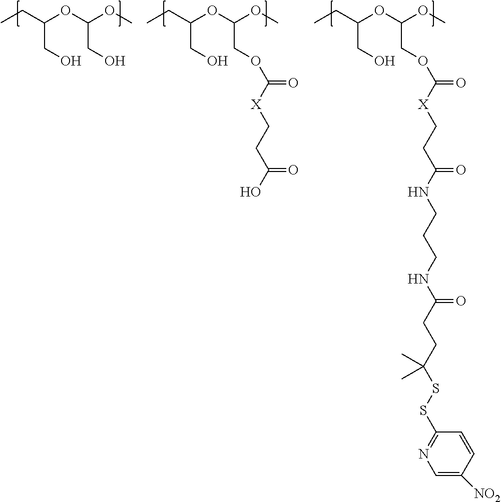 |
| | 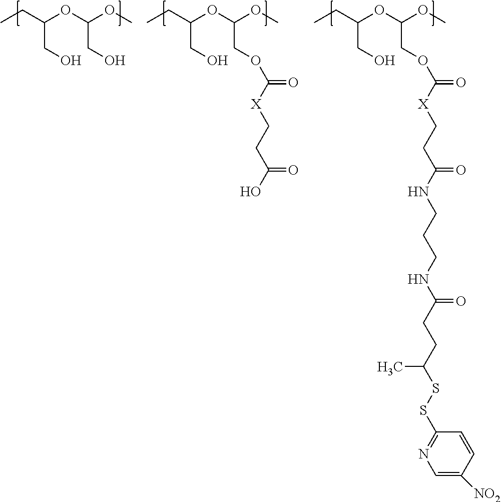 |
| | 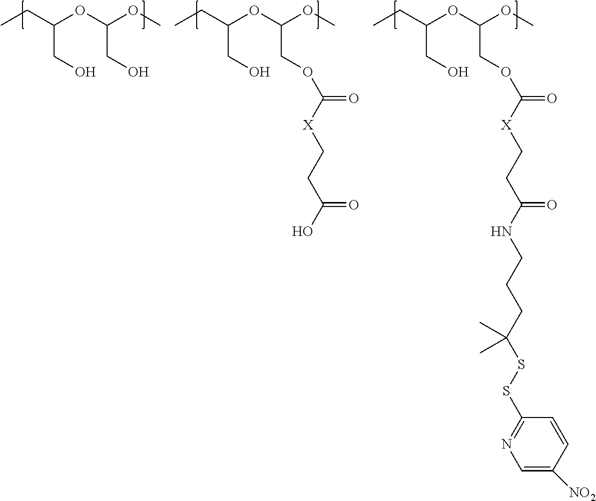 |
| | 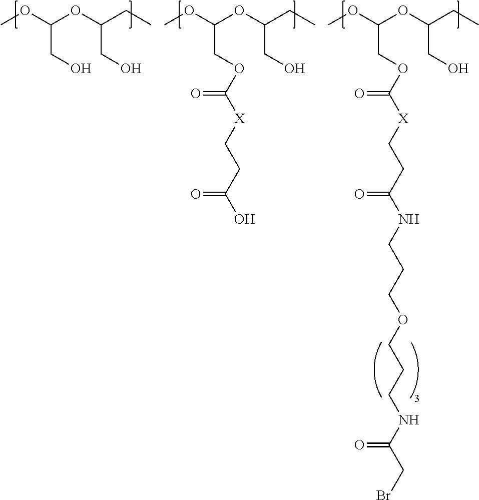 |
| | 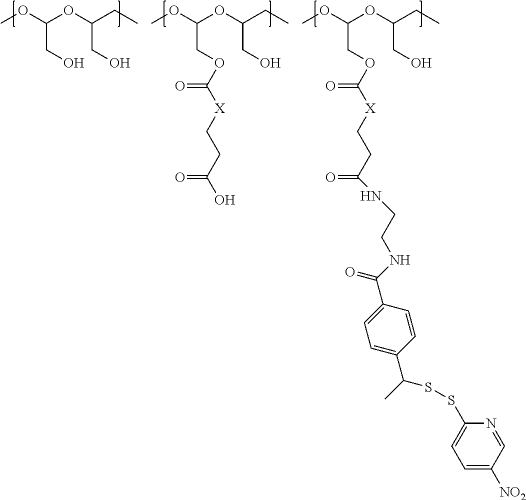 |
| | 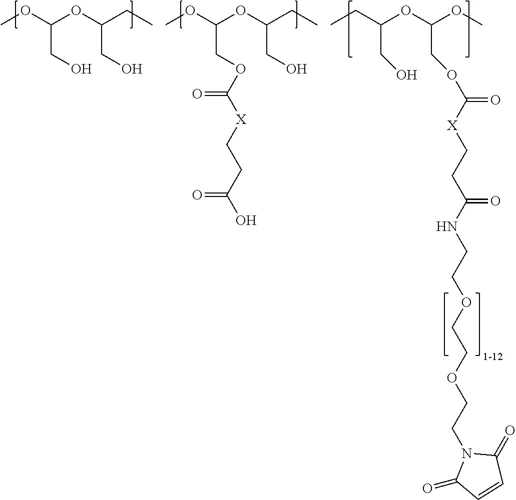 |
| | 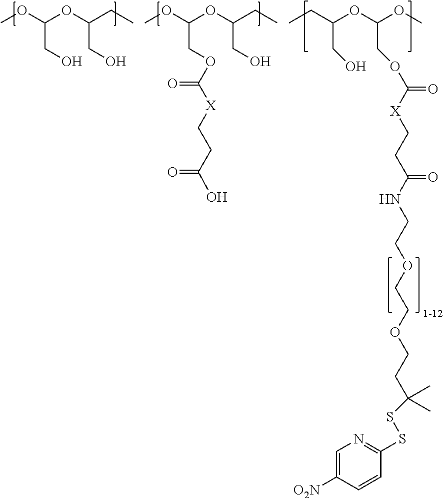 |
| | 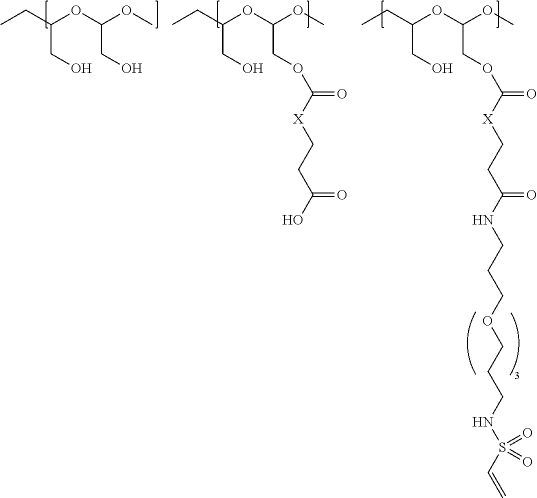 |
| | 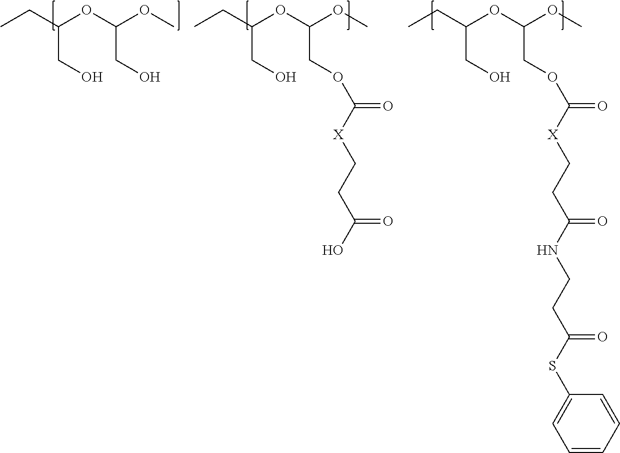 |

### Tubulysin Compounds

In one aspect, the invention relates to tubulysin compounds D (e.g., natural tubulysins or analogs thereof) that are modified such that they are suitable for conjugation with the PBRM, polymers or polymeric scaffolds described herein and can convert into active forms when the compounds are released from the PBRM, polymers or polymeric scaffolds.

In some embodiments, the tubulysin compound D has a molecular weight preferably ≤ about 5 kDa, more preferably ≤ about 4 kDa, more preferably ≤ about 3 kDa, most preferably ≤ about 1.5 kDa or ≤ about 1 kDa.

In certain embodiments, about 0.1 to about 25% monomers comprise a tubulysin compound, more preferably about 0.5 to about 20%, more preferably about 1 to about 15%, and even more preferably about 2 to about 10%.

In embodiments, tubulysin compound D, before conjugating with a polymer carrier (e.g., PHF) or directly conjugating with a PBRM, is a compound of the Formula (II) or a pharmaceutically acceptable salt thereof:

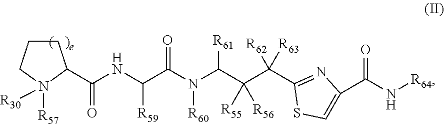

wherein:

each of R₅₅ and R₅₆ independently is hydrogen or OH; or R₅₅ and R₅₆ together form an oxo group (═O);

R₅₇ is C₁₋₄ alkyl and R₃₀ is O or R₅₇ is C₁₋₄ alkyl or —C(O)R₅₈ and R₃₀ is absent;

R₅₈ is C₁₋₆ alkyl, CF₃ or C₆₋₁₀ aryl;

R₅₉ is C₁₋₆ alkyl;

R₆₀ is hydrogen, C₁₋₆ alkyl, C₂₋₇ alkenyl, —CH₂-phenyl, CH₂OR₆₅, CH₂SR₆₅, CH₂NHR₆₅, CH₂OCOR₆₆ or CH₂—NHCO—C₁₋₆ alkyl;

R₆₁ is C₁₋₆ alkyl optionally substituted with C₃₋₁₀ cycloalkyl, or C₃₋₁₀ cycloalkyl optionally substituted with C₁₋₆ alkyl;

R₆₂ is hydrogen, OH, halogen, —O—C₁₋₄ alkyl or —O—C(O)—C₁₋₄ alkyl;

R₆₃ is hydrogen, OH, halogen, C₁₋₆ alkyl, —O—C₁₋₄ alkyl, —O—C(O)—C₁₋₄ alkyl, —O—C(O)—C₂₋₇ alkenyl, —O—C(O)—C₃₋₁₀ cycloalkyl, —O—C(O)—C₁₋₄ alkyl-C₆₋₁₀ aryl, or —O—C(O)—C₆₋₁₀ aryl; or R₆₂ and R₆₃ together form an oxo group (═O);

R₆₅ is hydrogen, C₁₋₆ alkyl optionally substituted with OH or SH, C₂₋₇ alkenyl, C₆₋₁₀ aryl, or C(O)R₆₇;

R₆₆ is C₁₋₆ alkyl, C₂₋₇ alkenyl optionally substituted with halo, C₃₋₁₀ cycloalkyl, —C₆H₅ or —CH₂-phenyl;

R₆₇ is C₁₋₆ alkyl, C₂₋₇ alkenyl, C₆₋₁₀ aryl or heteroaryl;

e is an integer from 1 to 3;

R₆₄ is:

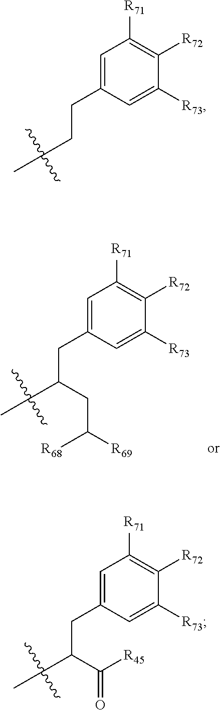

wherein:

R₆₈ is hydrogen or C₁-C₆ alkyl;

R₆₉ is CO₂R₇₀, C(O)—R₄₅, CONHNH₂, OH, NH₂, SH, or an optionally substituted alkyl, an optionally substituted cycloalkyl, an optionally substituted heteroalkyl or an optionally substituted heterocycloalkyl group;

R₇₀ is an optionally substituted alkyl (e.g., amino C₁₋₆ alkyl), an optionally substituted heteroalkyl or an optionally substituted heterocycloalkyl group;

each of R₇₁ and R₇₃ independently is hydrogen, OH, mono- or di-alkylamino, halo, —NO₂, —CN, —NHR₇₄, C₁₋₆ alkyl, haloalkyl, alkoxy or haloalkoxy;

R₇₂ is hydrogen, OR₄₃, alkoxy, halogen, —NHR₇₄, —O—C(O)—R₄₇, NO₂, —CN, C₆₋₁₀ aryl, C₁₋₆ alkyl, amino or dialkylamino;

R₇₄ is hydrogen, —CHO, —C(O)—C₁₋₄ alkyl, OH, amino group, alkyl amino or —[C(R₂₀R₂₁)]ₐ—R₂₂;

R₄₃ is H or —R₄₆—R₄₇;

R₄₆ is —C(O)—; —C(O)—O—, —C(O)—NH— or absent;

R₄₇ is an amino group, —R₉—[C(R₂₀R₂₁)]ₐ—R₁₀, —R₉—C₅₋₁₂ heterocycloalkyl-C₁₋₆ alkyl-R₁₀, 5 to 12-membered heterocycloalkyl, or —R₉—C₆₋₁₀ aryl;

R₉ is absent, N—(R₈₃) or oxygen;

R₁₀ is —OH, —NHR₈₃, —N—(R₈₃)R₁₁, —COOH, —R₈₂—C(O)(CH₂)_{c}—C(H)(R₂₃)—N(H)(R₂₃), —R₈₂—C(O)(CH₂)_{d}—(OCH₂—CH₂)_{f}—N(H)(R₂₃), —R₈₂—(C(O)—CH(X²)—NH)_{d}—R₇₇ or —R₈₂—C(O)—[C(R₂₀R₂₁)]ₐ—R₈₂—R₈₃ or

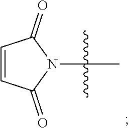 [SMILES: *N1C(=O)C=CC1=O]

X² is a side chain of a natural or unnatural amino acid;

R₇₇ is hydrogen or X² and NR₇₇ form a nitrogen containing cyclic compound;

R₈₂ is —NH or oxygen;

R₈₃ is hydrogen or CH₃;

R₄₅ is mono- or di-alkylamino, X³—R₇₅, or NH—R₁₉;

X³ is O or S;

R₁₉ is hydrogen, OH, amino group, alkyl amino or —[C(R₂₀R₂₁)]ₐ—R₂₂;

R₇₅ is a hydrogen, amino group, C₁₋₆ alkyl amino or —[C(R₂₀R₂₁)]ₐ—R₂₂;

each of R₂₀ and R₂₁ independently is hydrogen, C₁₋₆ alkyl, C₆₋₁₀ aryl, hydroxylated C₆-10 aryl, polyhydroxylated C₆₋₁₀ aryl, 5 to 12-membered heterocycle, C₃₋₈ cycloalkyl, hydroxylated C₃₋₈ cycloalkyl, polyhydroxylated C₃₋₈ cycloalkyl or a side chain of a natural or unnatural amino acid;

R₂₂ is —OH, —NH₂, —COOH, —R₈₂—C(O)(CH₂)_{c}—C(H)(R₂₃)—N(H)(R₂₃), —R₈₂—C(O)(CH₂)_{d}—(OCH₂—CH₂)_{f}—N(H)(R₂₃), or —R₈₂—(C(O)—CH(X²)—NH)_{d}—R₇₇;

each R₂₃ independently is hydrogen, C₁₋₆ alkyl, C₆₋₁₀ aryl, C₃₋₈ cycloalkyl, —COOH, or —COO—C₁₋₆ alkyl;

a is an integer from 1 to 6;

c is an integer from 0 to 3;

d is an integer from 1 to 3;

f is an integer from 1 to 12;

R₁₁ is 0

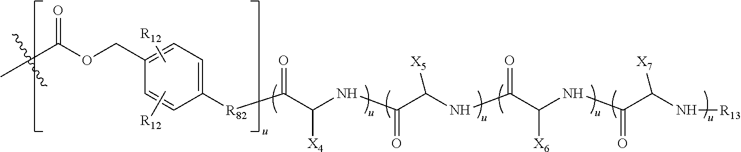

each R₁₂ independently is hydrogen, chloride, —CH₃ or —OCH₃;

R₁₃ is hydrogen or —C(O)—(CH₂)_{d}—(O—CH₂—CH₂)_{f}—NH₂;

R₈₂ is —NH or oxygen

X₄ is the side chain of lysine, arginine, citrulline, alanine or glycine;

X₅ is the side chain of phenylalanine, valine, leucine, isoleucine or tryptophan;

each of X₆ and X₇ is independently the side chain of glycine, alanine, serine, valine or proline;

each u independently is an integer 0 or 1;

or R₁₁ is —Yᵤ—W_{q}—R₈₈,

wherein:

Y is any one of the following structures:

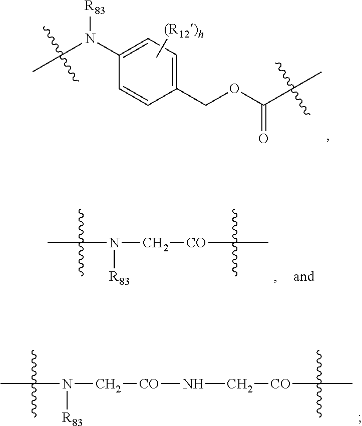

in each of which the terminal NR₈₃ group of Y is proximal to R₈₈;

R₈₃ is hydrogen or CH₃;

each W is an amino acid unit;

each R₁₂′ independently is halogen, —C₁₋₈ alkyl, —O—C₁₋₈ alkyl, nitro or cyano;

R₈₈ is hydrogen or —C(O)—(CH₂)_{ff}—(NH—C(O))ₐₐ-Eⱼ-(CH₂)_{bb}—R₈₅

R₈₅ is NH₂, OH or

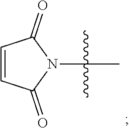 [SMILES: *N1C(=O)C=CC1=O]

E is —CH₂— or —CH₂CH₂O—;

q is an integer from 0 to 12;

aa is an integer 0 or 1;

bb is an integer 0 or 2;

ff is an integer from 0 to 10;

h is an integer from 0 to 4;

j is an integer from 0 to 12; and

when E is —CH₂—, bb is 0 and j is an integer from 0 to 10; and when E is —CH₂CH₂—O—, bb is 2 and j is an integer from 1 to 12;

or R₁₁ is

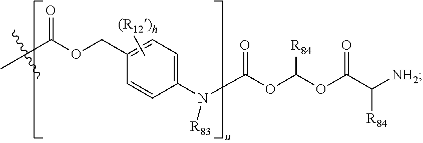

wherein:

R₈₃ is hydrogen or CH₃;

R₈₄ is C₁₋₆ alkyl or C₆₋₁₀ aryl;

each R₁₂′ independently is halogen, —C₁₋₈ alkyl, —O—C₁₋₈ alkyl, nitro or cyano; and

h is an integer from 0 to 4;

provided that (i) when R₆₄ is

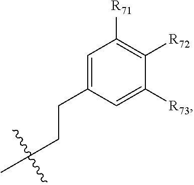

then at least one of R₇₁, R₇₂ and R₇₃ is —NHR₇₄, OR₄₃, or —O—C(O)—R₄₇, in which R₇₄ is —[C(R₂₀R₂₁)]ₐ—R₂₂, R₄₃ is —R₄₆—R₄₇, R₄₆ is —C(O)—; —C(O)—O—, or —C(O)—NH—, and R₄₇ is an amino group, —R₉—[C(R₂₀R₂₁)]ₐ—R₁₀, —R₉—C₅₋₁₂ heterocycloalkyl-C₁₋₆ alkyl-R₁₀, 5 to 12-membered heterocycloalkyl, or —R₉—C₆₋₁₀ aryl;

(ii) when R₆₄ is

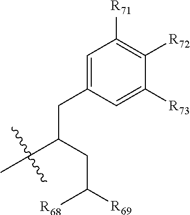

then at least one of R₇₁, R₇₂ and R₇₃ is —NHR₇₄, OR₄₃, or —O—C(O)—R₄₇, or R₆₉ is C(O)R₄₅ in which R₄₅ is X³—R₇₅ or NH—R₁₉; in which each of R₇₄, R₇₅, and R₁₉, independently, is —[C(R₂₀R₂₁)]ₐ—R₂₂, R₄₃ is —R₄₆—R₄₇, R₄₆ is —C(O)—; —C(O)—O—, or —C(O)—NH—, and R₄₇ is an amino group, —R₉—[C(R₂₀R₂₁)]ₐ—R₁₀, —R₉—C₅₋₁₂ heterocycloalkyl-C₁₋₆ alkyl-R₁₀, 5 to 12-membered heterocycloalkyl, or —R₉—C₆₋₁₀ aryl; or

(iii) when R₆₄ is

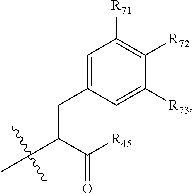

then at least one of R₇₁, R₇₂ and R₇₃ is —NHR₇₄, OR₄₃, or —O—C(O)—R₄₇, or R₄₅ is X³—R₇₅, or NH—R₁₉; in which each of R₇₄, R₇₅, and R₁₉, independently, is —[C(R₂₀R₂₁)]ₐ—R₂₂, R₄₃ is —R₄₆—R₄₇, R₄₆ is —C(O)—; —C(O)—O—, or —C(O)—NH—, and R₄₇ is an amino group, —R₉—[C(R₂₀R₂₁)]ₐ—R₁₀, —R₉—C₅₋₁₂ heterocycloalkyl-C₁₋₆ alkyl-R₁₀, 5 to 12-membered heterocycloalkyl, or —R₉—C₆₋₁₀ aryl.

In some embodiments, R₁₁ is:

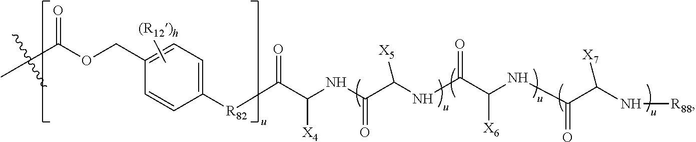

wherein:

each R₁₂′ independently is chloride, —CH₃ or —OCH₃;

R₈₈ is hydrogen or —C(O)—(CH₂)_{ff}—(CH₂—CH₂O)ⱼ—CH₂—CH₂—NH₂;

R₈₂ is —NH or oxygen

X₄ is the side chain of lysine, arginine, citrulline, alanine or glycine;

X₅ is the side chain of phenylalanine, valine, leucine, isoleucine or tryptophan;

each of X₆ and X₇ is independently the side chain of glycine, alanine, serine, valine or proline;

ff is an integer from 1 to 3;

j is an integer from 1 to 12

h is an integer from 0 to 4; and

each u independently is an integer 0 or 1.

In some embodiments,

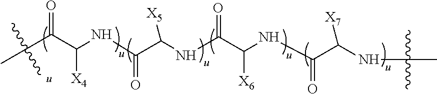

is citrulline-valine; lysine-phenylalanine; citrulline-phenylalanine; citrulline-leucine; citrulline-valine-glycine-glycine; glycine-phenylalanine-glycine-glycine; valine; proline; leucine or isoleucine.

In another embodiment, R₁₁ is any one of the following structures:

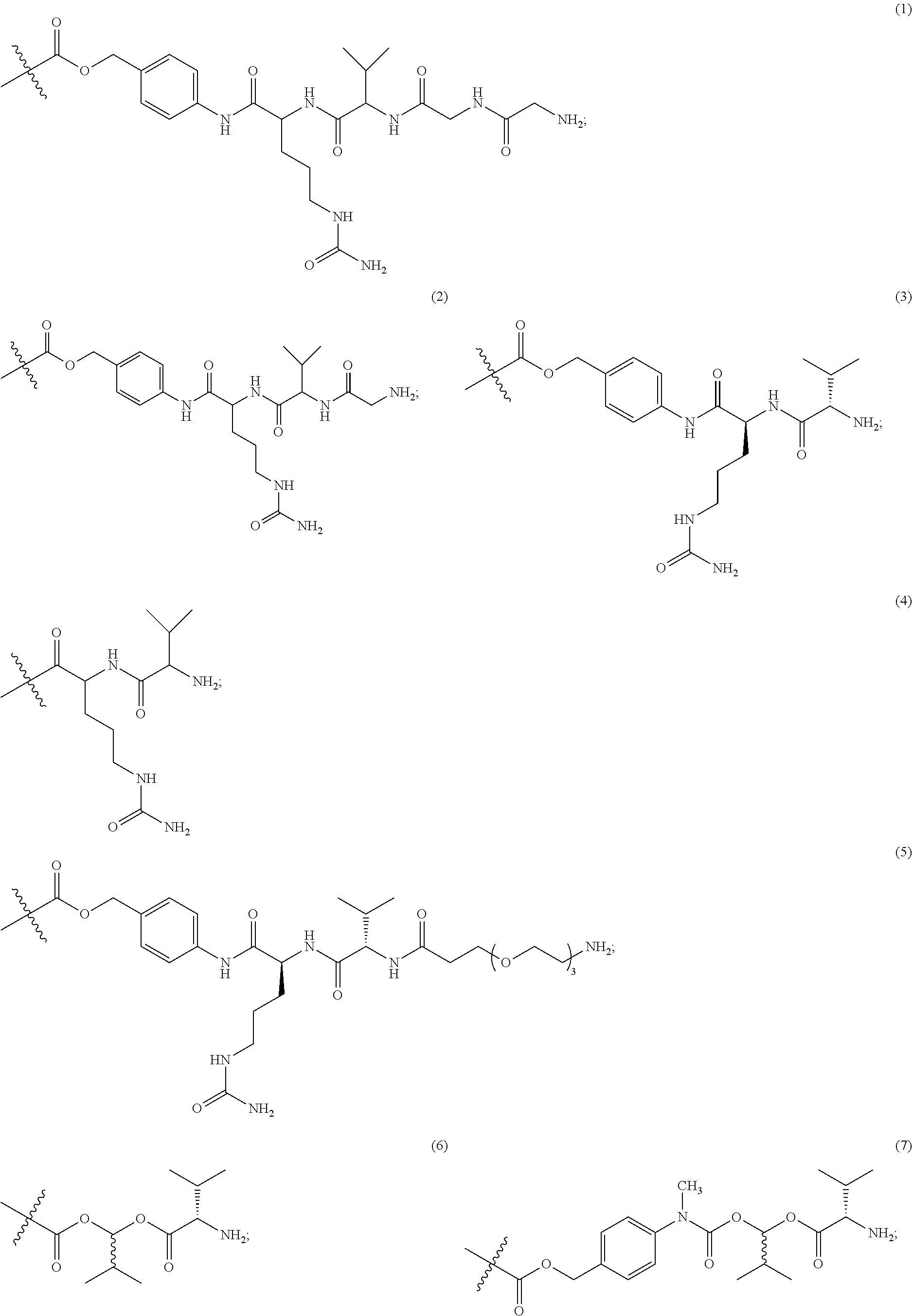

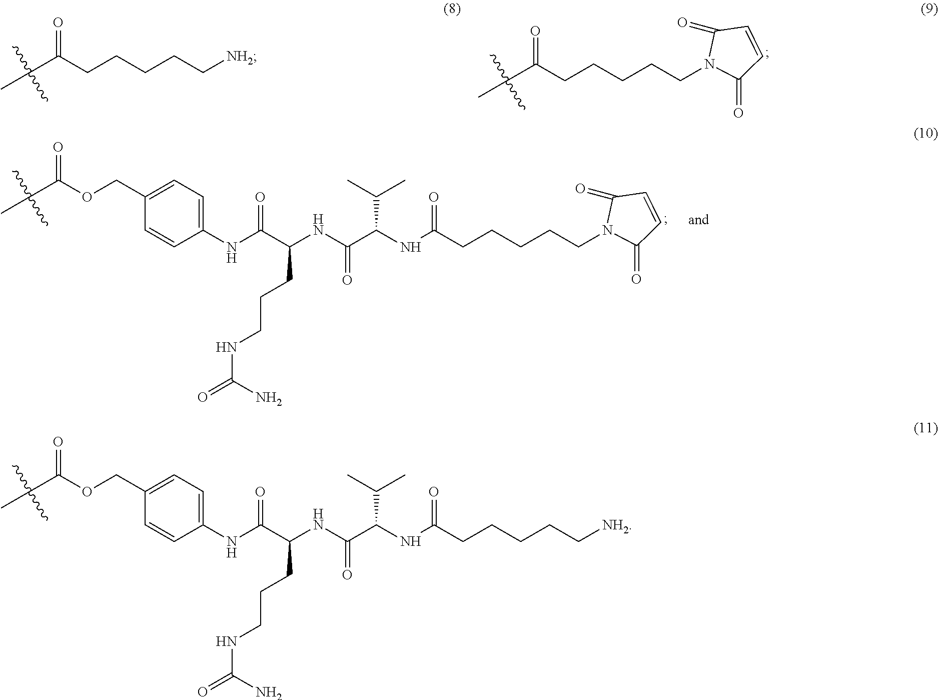 [SMILES: *C(=O)CCCCCN ; *C(=O)CCCCCN1C(=O)C=CC1=O ; *C(=O)OCc1ccc(NC(=O)[C@H](CCCNC(N)=O)NC(=O)[C@@H](NC(=O)CCCCCN2C(=O)C=CC2=O)C(C)C)cc1 ; *C(=O)OCc1ccc(NC(=O)[C@H](CCCNC(N)=O)NC(=O)[C@@H](NC(=O)CCCCCN)C(C)C)cc1]

The compounds of Formula (II) can include one or more of the following features:

For example, R₅₇ is methyl or ethyl and R₃₀ is absent.

For example, R₅₉ is ethyl, iso-propyl, iso-butyl, sec-butyl, cyclopropyl, or CH₂-cyclopropyl.

For example, R₆₀ is hydrogen, methyl, ethyl, propyl, isopropyl, —CH₂OR₆₅, CH₂OCOR₆₆, —CH₂SR₆₅, or —CH₂NHC(O)—CH₂CH(CH₃)₂.

For example, R₆₅ is methyl, ethyl, propyl, iso-propyl, butyl, iso-butyl, iso-pentyl, iso-butylene (—CH═C(CH₃)₂), —(CH₂)₂—CH═CH₂, —(CH₂)₂OH, or —(CH₂)₂SH.

For example, R₆₆ is methyl, ethyl, propyl, iso-propyl, butyl, iso-butyl, iso-pentyl, iso-butylene (—CH═C(CH₃)₂), —(CH₂)₂—CH═CH₂, —(CH═CH)—CH₂C₁, cyclopropyl, cyclobutyl, or cyclohexyl.

For example, R₆₁ is ethyl, iso-propyl, sec-butyl, iso-butyl, trifloromethyl, chloromethyl, cyclopropyl, CH₂-cyclopropyl, cyclopentyl or cyclohexyl.

For example, R₅₅ is hydrogen.

For example, R₅₆ is hydrogen or OH; or R₅₅ and R₅₆ together form an oxo group (═O).

For example, R₆₂ is hydrogen.

For example, R₆₃ is hydrogen, OH, O—C(O)—R₄₉.

For example, R₄₉ is methyl, ethyl, propyl, iso-propyl or phenyl.

For example, R₆₈ is-CH₃.

For example, R₆₉ is CO₂H or C(O)—R₄₅.

For example, R₄₅ is —OR₄₂ or —NHR₄₀, wherein R₄₀ is hydrogen, —OH, or —NH₂, R₄₂ is hydrogen, or each of R₄₀ and R₄₂, independently is selected from the following structures:

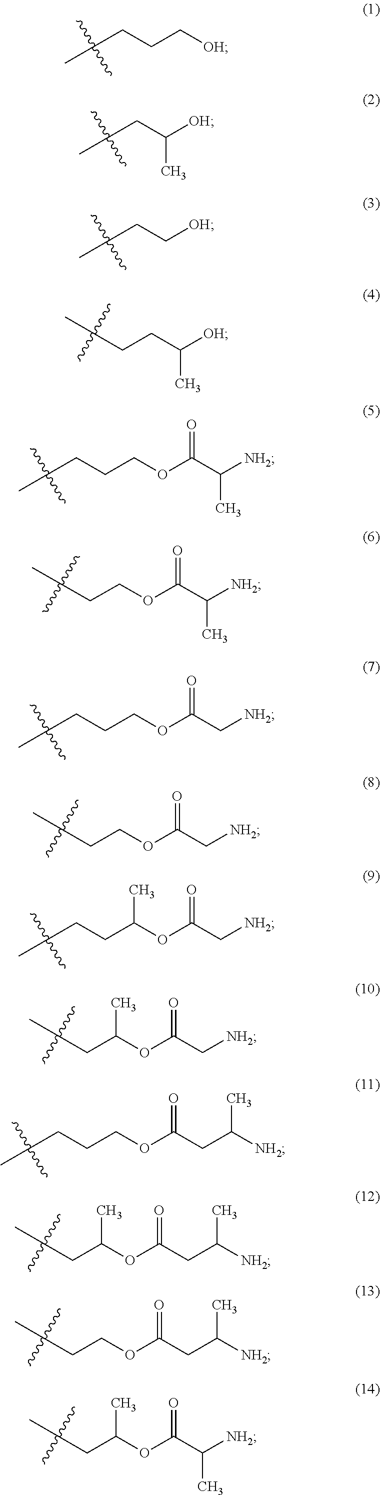

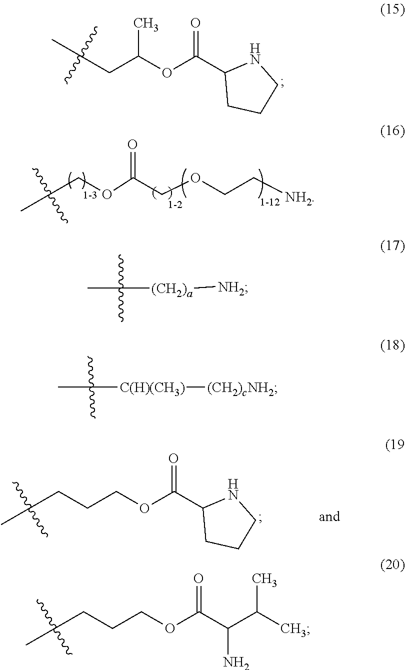

in which a is an integer from 1 to 6; and c is an integer from 0 to 3.

For example, R₆₄ is:

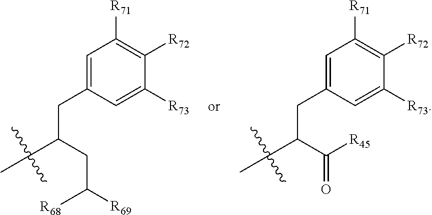

For example, each of R₇₁ and R₇₃ independently is hydrogen;

For example, R₇₂ is hydrogen, —OR₄₃ or OH, with the proviso that if R₇₂ is —OH, then R₄₂ or R₄₀ cannot be hydrogen; and if R₆₉ is COOH then R₇₂ must be —OR₄₃.

For example, R₄₃ is —R₄₆—R₄₇.

For example, R₄₆ is —C(O)—; —C(O)—O—, —C(O)—NH—, or absent.

For example, R₄₇ is —R₉—[C(R₂₀R₂₁)]ₐ—R₁₀, or —R₉—C₅₋₁₂ heterocycloalkyl-C₁₋₆ alkyl-R₁₀.

For example, R₁₀ is —OH, —NHR₈₃, —N—(R₈₃)R₁₁, or

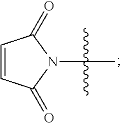

in which R₈₃ is hydrogen or CH₃.

For example, R₄₇ is any one of the following structures:

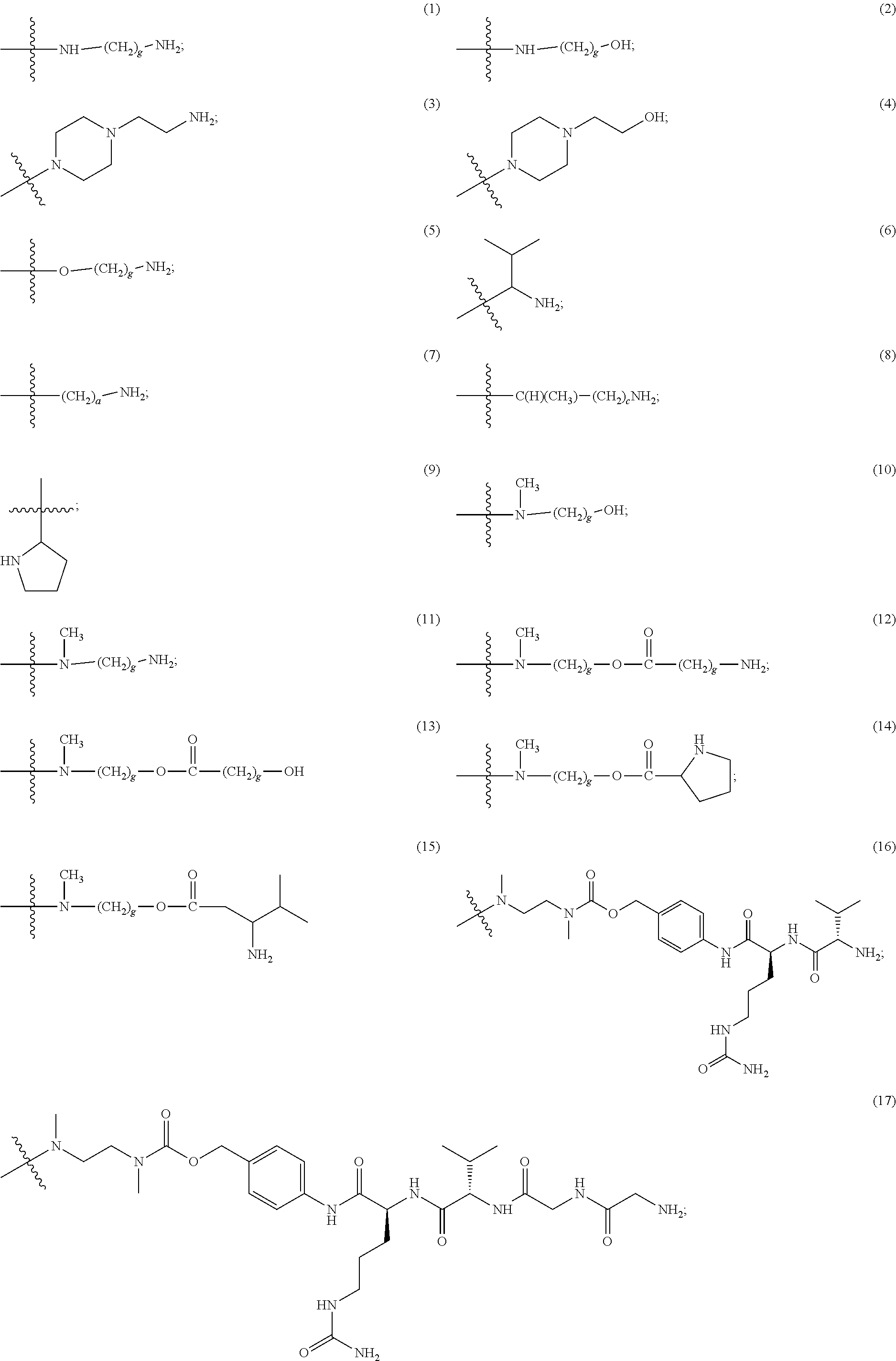

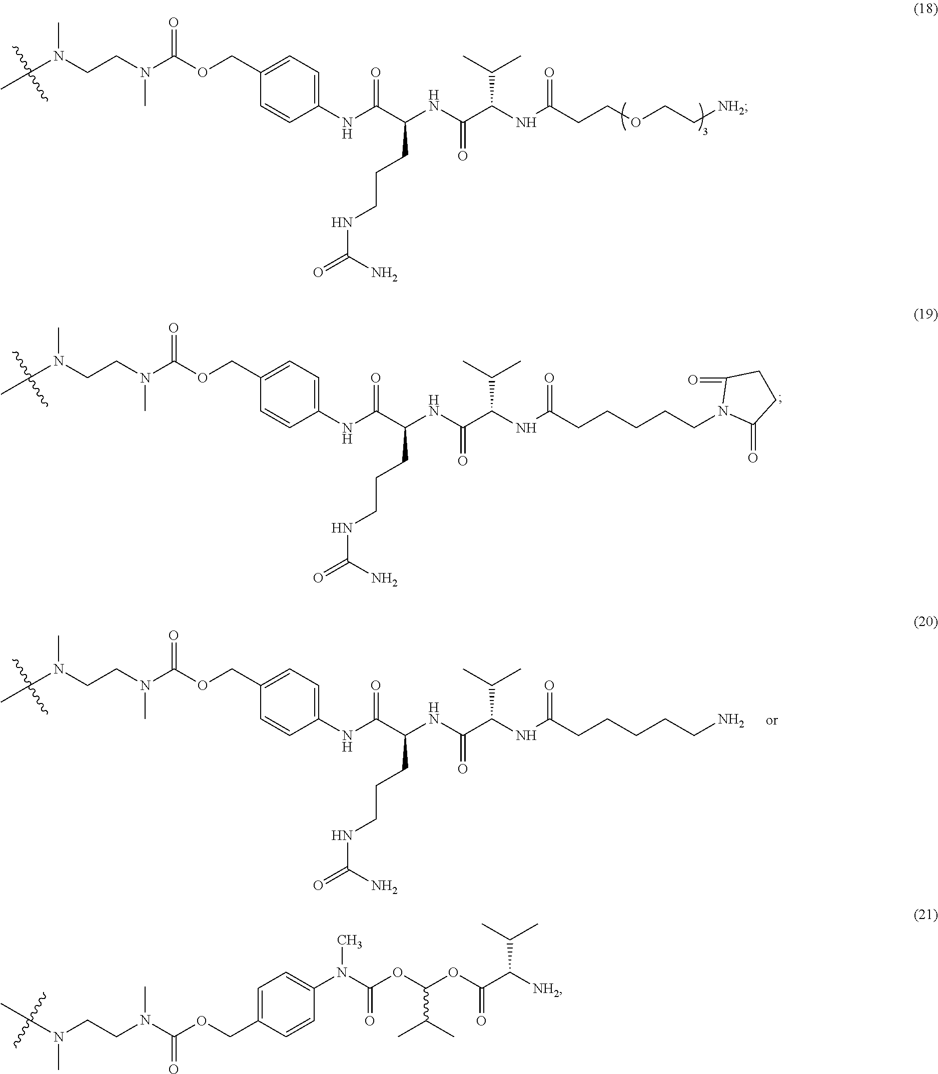

wherein a is an integer from 1 to 6; c is an integer from 0 to 3; and g is an integer from 2 to 6.

For example, e is the integer 2.

For example, tubulysin compound D, before conjugating with a polymer carrier (e.g., PHF) or directly conjugating with a PBRM, is a subset of the compounds of Formula (II) and is of Formula (IIA) or a pharmaceutically acceptable salt thereof:

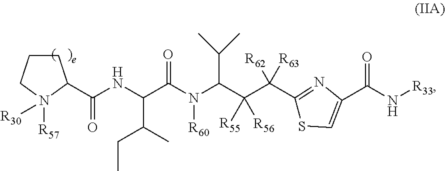

wherein

e is 2,

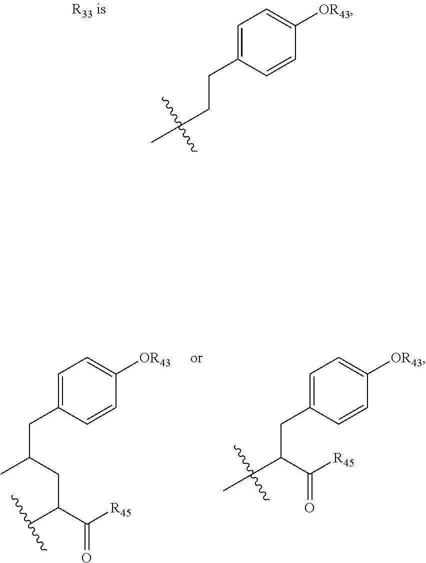

in which R₄₅ is mono- or di-alkylamino, —OR₄₂ or —NHR₄₀, and R₄₀, R₄₂ and R₄₃ are as defined herein for Formula (II); provided that at least one of R₄₃, R₄₂ and R₄₀ cannot be hydrogen;

R₅₅ is hydrogen;

R₅₆ is hydrogen or OH; or R₅₅ and R₅₆ together form an oxo group (═O);

R₅₇ is methyl or ethyl, or —C(O)R₅₈ and R₃₀ is absent or R₅₇ is methyl and R₃₀ is O;

R₅₈ is C₁₋₆ alkyl, CF₃ or C₆₋₁₀ aryl;

R₆₀ is hydrogen, methyl, —CH₂OR₆₅, or —CH₂NHR₆₅;

R₆₂ is hydrogen or alkyl;

R₆₃ is hydrogen, halo, OH, —O—C₁₋₄ alkyl or O—C(O)—R₃₄, in which R₃₄ is C₁₋₄ alkyl, C₂₋₇ alkenyl, or C₆₋₁₀ aryl; or R₆₂ and R₆₃ together form an oxo group (═O);

R₆₅ is hydrogen, C₁₋₆ alkyl optionally substituted with OH or SH, C₂₋₇ alkenyl, or C(O)R₆₇; and

R₆₇ is C₁₋₆ alkyl, C₂₋₇ alkenyl, C₆₋₁₀ aryl or heteroaryl.

For example, in compounds of Formula (IIA), R₄₃ is not H, e.g., R₄₃ is —R₄₆—R₄₇, wherein R₄₆ is —C(O)—; —C(O)—O—, —C(O)—NH—, or absent and R₄₇ is —R₉—[C(R₂₀R₂₁)]ₐ—R₁₀, or —R₉—C₅₋₁₂ heterocycloalkyl-C₁₋₆ alkyl-R₁₀.

For example, tubulysin compound D, before conjugating with a polymer carrier (e.g., PHF) or directly conjugating with a PBRM, is a subset of the compounds of Formula (II) and is of Formula (IIB) or a pharmaceutically acceptable salt thereof:

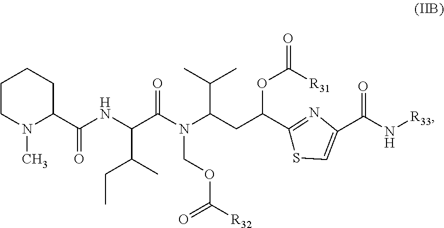

wherein

R₃₁ is C₁₋₄ alkyl,

R₃₂ is C₁₋₆ alkyl, C₂₋₇ alkenyl optionally substituted with halo, or C₃₋₆ cycloalkyl, and

R₃₃ is

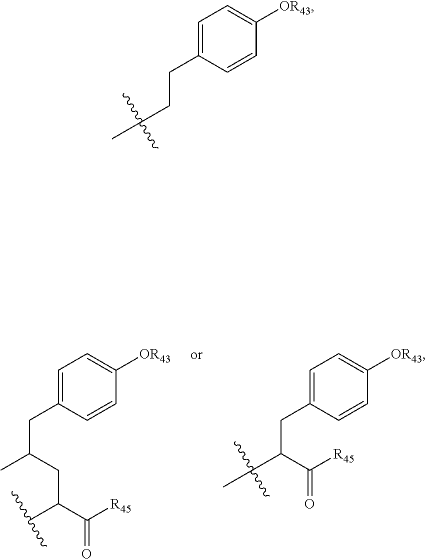

wherein R₄₅ is mono- or di-alkylamino, —OR₄₂ or —NHR₄₀, and R₄₀, R₄₂ and R₄₃ are as defined herein for Formula (II); provided that at least one of R₄₃, R₄₂ and R₄₀ cannot be hydrogen.

For example, in compounds of Formula (IIB), R₄₃ is not H, e.g., R₄₃ is —R₄₆—R₄₇, wherein R₄₆ is —C(O)—; —C(O)—O—, —C(O)—NH—, or absent and R₄₇ is —R₉—[C(R₂₀R₂₁)]ₐ—R₁₀, or —R₉—C₅₋₁₂ heterocycloalkyl-C₁₋₆ alkyl-R₁₀.

For example, tubulysin compound D, before conjugating with a polymer carrier (e.g., PHF) or directly conjugating with a PBRM, is any one of the compounds of Formula (V) or (Va), or a pharmaceutically acceptable salt thereof:

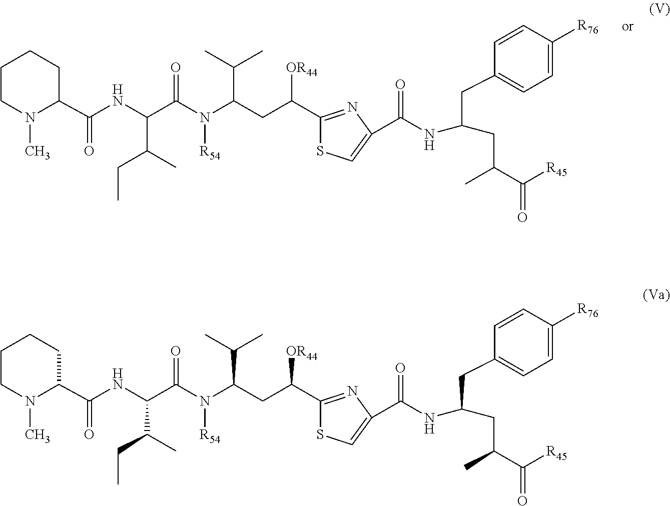

wherein

R₄₅ is mono- or di-alkylamino, —OR₄₂ or —NHR₄₀;

R₄₄, R₅₄ and R₇₆ are as defined in Table B below;

R₄₀, R₄₂ and R₄₃ are as defined herein for Formula (II);

with the proviso that at least one of R₄₃, R₄₂ and R₄₀ cannot be hydrogen.

TABLE B

| R₄₄ | R₅₄ | R₇₆ |
|---|---|---|
| —C(O)CH₃ | —CH₂OC(O)CH₂CH(CH₃)₂ | —OR₄₃ |
| —C(O)CH₃ | —CH₂OC(O)CH₂CH₂CH₃ | —OR₄₃ |
| —C(O)CH₃ | —CH₂OC(O)CH₂CH₃ | —OR₄₃ |
| —C(O)CH₃ | —CH₂OC(O)CH₂CH(CH₃)₂ | H |
| —C(O)CH₃ | —CH₂OC(O)CH₂CH₂CH₃ | H |
| —C(O)CH₃ | —CH₂OC(O)CH₂CH₃ | H |
| —C(O)CH₃ | —CH₂OC(O)CH═C(CH₃)₂ | —OR₄₃ |
| —C(O)CH₃ | —CH₂OC(O)CH₃ | H |
| —C(O)CH₃ | —CH₂OC(O)CH₃ | —OR₄₃ |
| —C(O)CH₃ | H | H |
| H | H | H |
| H | —CH₂OC(O)CH₂CH₂CH₃ | —OR₄₃ |
| —C(O)CH₃ | —CH₂OH | —OR₄₃ |
| —C(O)CH₃ | H | —OR₄₃ |
| H | H | —OR₄₃ |
| —C(O)CH₃ | H, CH₃, or CH₂CH₂CH₃ | Halogen |
| —C(O)CH₃ | CH₃ | —CH₃ |
| —C(O)CH₃ | CH₃ | —OCH₃ |
| —C(O)CH₃ | —CH₂OCH₃ | —OR₄₃ |
| —C(O)CH₃ | —CH₂O(CH₂)₂OH | —OR₄₃ |
| —C(O)CH₃ | —CH₂O(CH₂)₂CH(CH₃)₂ | —OR₄₃ |
| —C(O)CH₃ | —CH₂S(CH₂)₂SH | —OR₄₃ |
| —C(O)CH₃ | —(CH₂)₃—CH═CH₂ | —OR₄₃ |
| —C(O)CH₃ | —CH₂S(CH₂)₂OH | —OR₄₃ |
| —C(O)CH₃ | —CH₂OC(O)—CH═CH—CH₂Cl | —OR₄₃ |
| —C(O)CH₃ | —CH₂NHC(O)CH₂CH(CH₃)₂ | —OR₄₃ |
| —C(O)CH₃ | —CH₂O(CH₂)₂CH₃ | —OR₄₃ |
| —C(O)CH₃ | —CH₂S(CH₂)₂CH₃ | —OR₄₃ |

For example, tubulysin compound D, before conjugating with a polymer carrier (e.g., PHF) or directly conjugating with a PBRM, is any one of the Tubulysin-A compounds of Formula (VI) or (VIa), or Tubulysin-B compounds of Formula (VII) or (VIIa), or a pharmaceutically acceptable salt thereof:

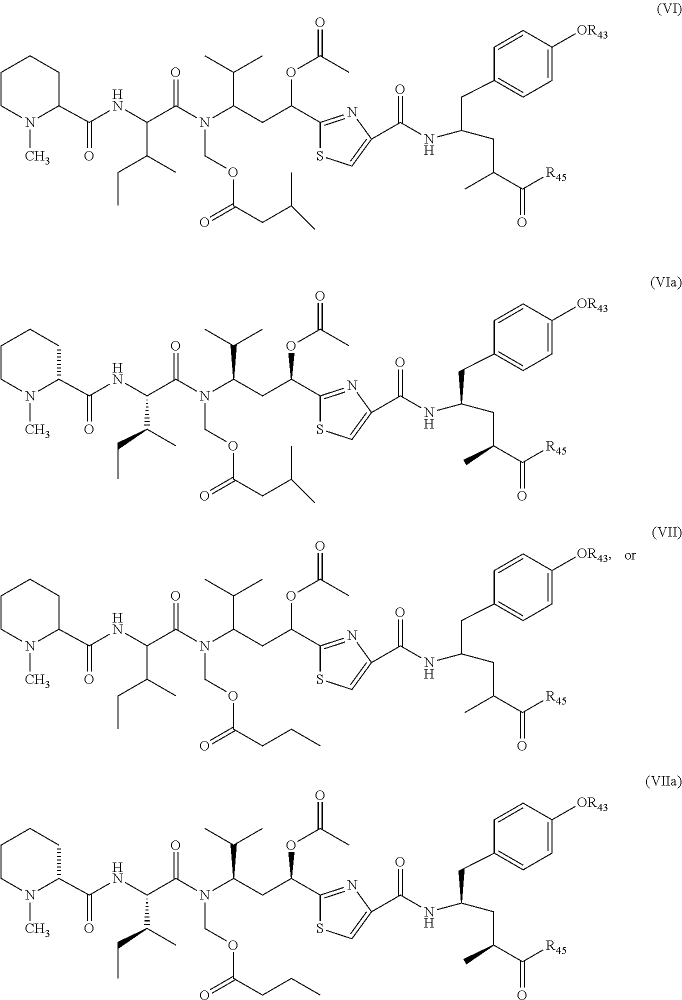

wherein:

R₄₅ is —OR₄₂ or —NHR₄₀ and

R₄₀, R₄₂ and R₄₃ are as defined herein for Formula (II);

with the proviso that at least one of R₄₀, R₄₂ and R₄₃ is not hydrogen.

For example, the tubulysin of Formula (II) is a compound of Formula (VIII), (VIIIa), (IX), or (IXa) or a pharmaceutically acceptable salt thereof:

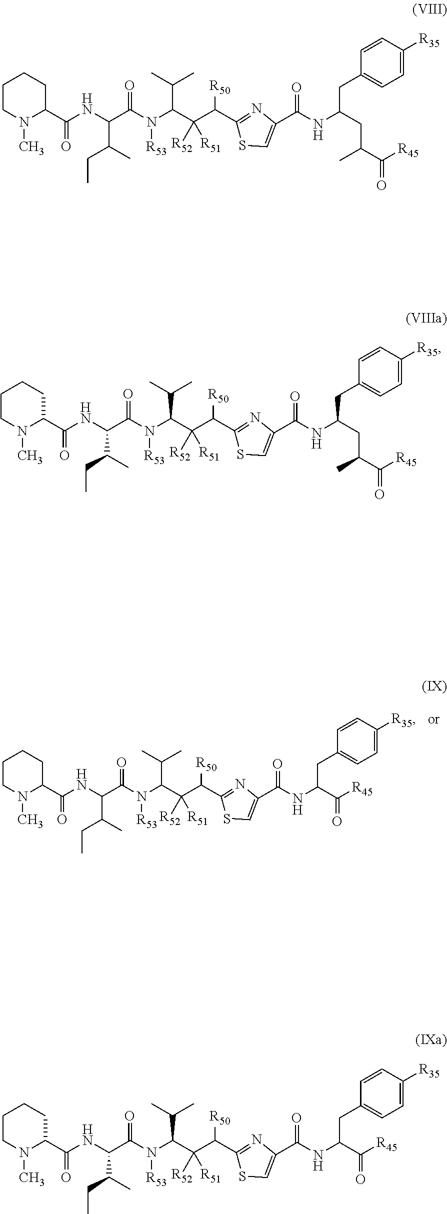

wherein

R₃₅ is H or —OR₄₃;

R₄₅ is mono- or di-alkylamino, —OR₄₂ or —NHR₄₀;

R₅₀, R₅₁, R₅₂ and R₅₃ are as defined in Table C below; and

R₄₀, R₄₂ and R₄₃ are as defined herein for Formula (II);

with the proviso that at least one of R₄₃, R₄₂ and R₄₀ cannot be hydrogen.

TABLE C

| R₅₀ | R₅₁, R₅₂ | R₅₃ |
|---|---|---|
| OH | H, H | —CH₂—OC(O)CH₂CH₂CH₃ |
| OH | H, H | —CH₂—OCOCH₂CH(CH₃)₂ |
| H | ═O | H |
| H | H, OH | H |
| H | H, H | H |
| OH | ═O | H |
| —OCOCH₃ | H, H | H |
| H | H, H | —CH₂OH |
| —OCOCH₃ | H, H | —CH₂OH |
| H | ═O | —CH₂OH |
| H | H, H | CH₃ |

For example, tubulysin compound D, before conjugating with a polymer carrier (e.g., PHF) or directly conjugating with a PBRM, is any one of the compounds in Tables D and E below.

TABLE D

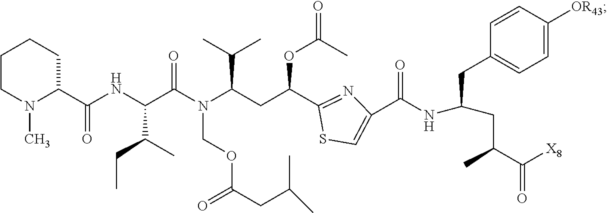

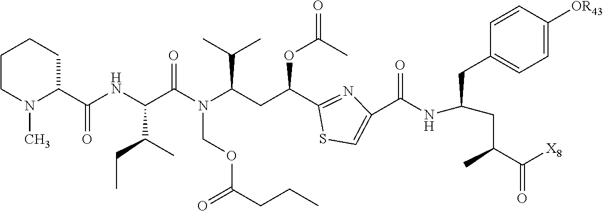

R₄₃

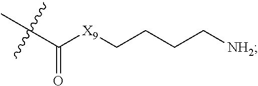

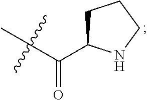

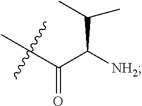

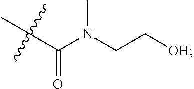

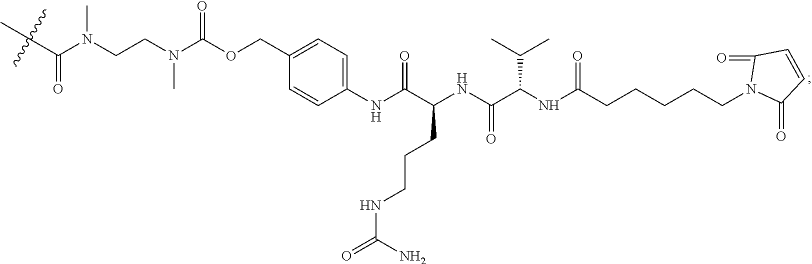

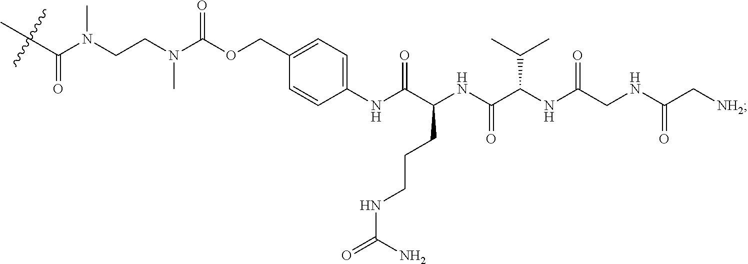

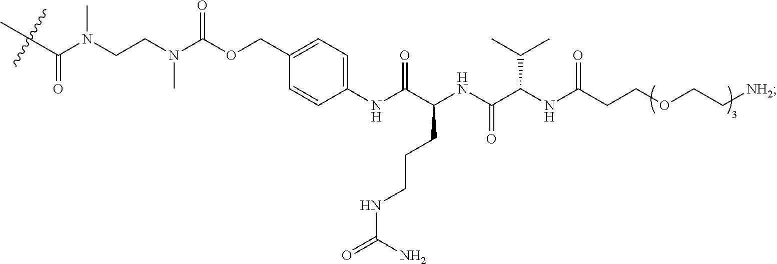

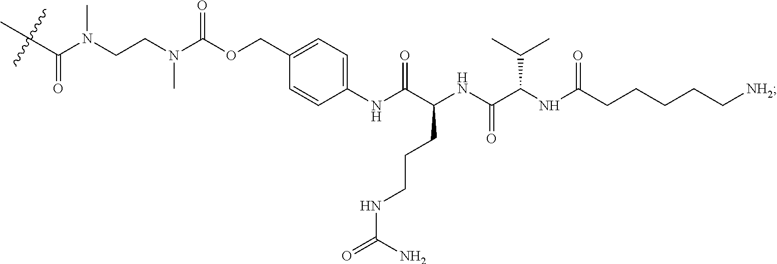

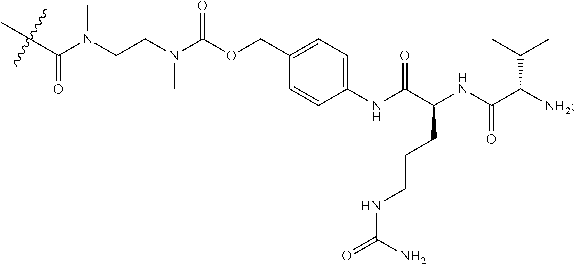

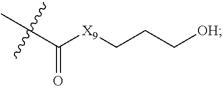

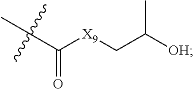

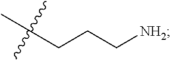

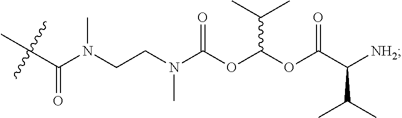

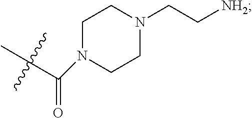

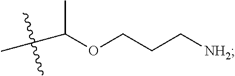

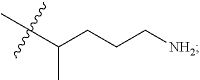

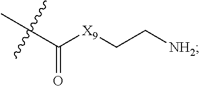

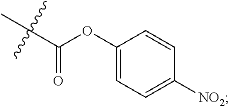

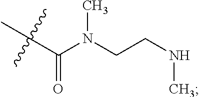

wherein:

X₈ is —OH, —NH₂ or mono- or di-alkylamino; and

X₉ is —O or —NH.

TABLE E

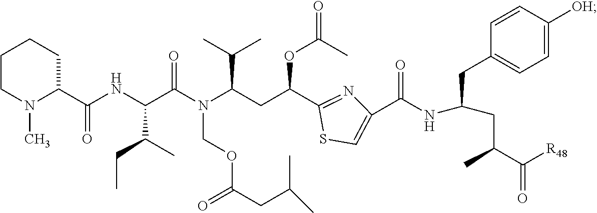

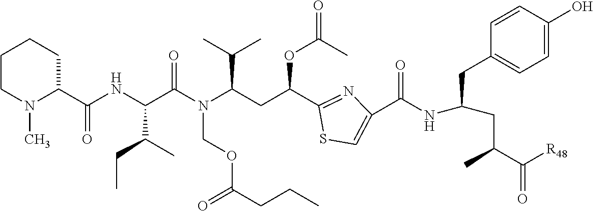

R₄₈

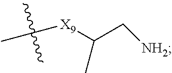

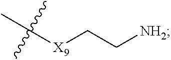

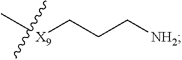

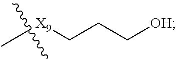

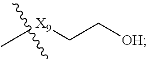

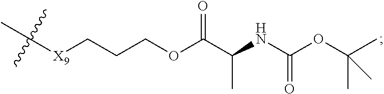

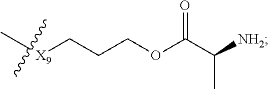

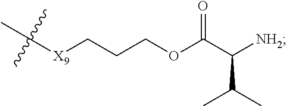

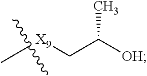

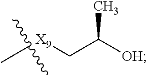

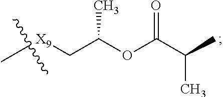

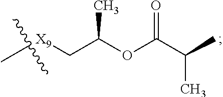

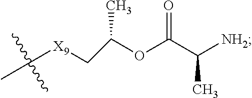

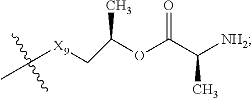

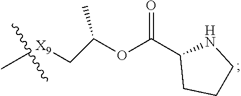

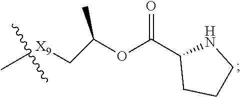

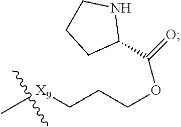

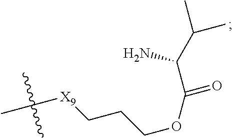

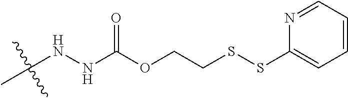

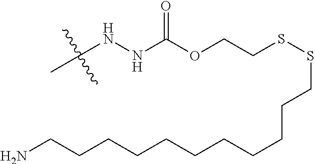

wherein X₉ is —O or —NH

For example, D is of the tubulysin compound represented by Formula (IIC) or a pharmaceutically acceptable salt thereof:

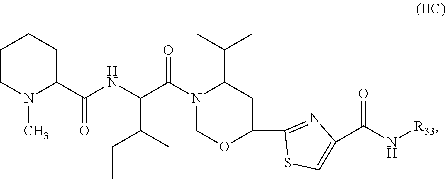

wherein

R₃₃ is

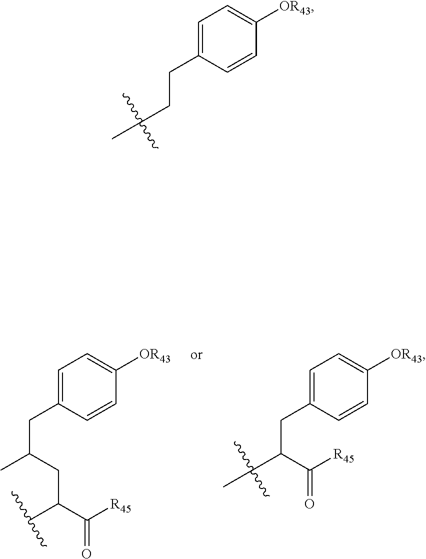

wherein R₄₅ is mono- or di-alkylamino —OR₄₂ or —NHR₄₀, and R₄₀, R₄₂ and R₄₃ are as defined herein for Formula (II); provided that at least one of R₄₃, R₄₂ and R₄₀ cannot be hydrogen.

One skilled in the art of therapeutic agents will readily understand that each of the tubulysin compounds described herein can be modified in such a manner that the resulting compound still retains the specificity and/or activity of the original compound. The skilled artisan will also understand that many of these compounds can be used in place of the therapeutic agents described herein. Thus, the therapeutic agents of the present invention include analogs and derivatives of the compounds described herein.

Additional examples of tubulysin compounds suitable for the present invention are describe in US 2011/0021568, US 2011/0294998, WO 2008/076333, WO 2008/106080, WO 2008/112873, WO 2009/002993, WO 2009/012958, WO 2009/026177, WO 2009/055562, WO 2009/134279, WO 2010/033733, WO 2010/034724, each of which is hereby incorporated by reference in its entirety.

### Protein-Based Recognition Molecules (PBRMs)

The protein-based recognition molecule directs the drug-polymer carrier conjugates to specific tissues, cells, or locations in a cell. The protein-based recognition molecule can direct the modified polymer in culture or in a whole organism, or both. In each case, the protein-based recognition molecule has a ligand that is present on the cell surface of the targeted cell(s) to which it binds with an effective specificity, affinity and avidity. In some embodiments, the protein-based recognition molecule targets the modified polymer to tissues other than the liver. In other embodiments the protein-based recognition molecule targets the modified polymer to a specific tissue such as the liver, kidney, lung or pancreas. The protein-based recognition molecule can target the modified polymer to a target cell such as a cancer cell, such as a receptor expressed on a cell such as a cancer cell, a matrix tissue, or a protein associated with cancer such as tumor antigen. Alternatively, cells comprising the tumor vasculature may be targeted. Protein-based recognition molecules can direct the polymer to specific types of cells such as specific targeting to hepatocytes in the liver as opposed to Kupffer cells. In other cases, protein-based recognition molecules can direct the polymer to cells of the reticular endothelial or lymphatic system, or to professional phagocytic cells such as macrophages or eosinophils. (In such cases the polymer itself might also be an effective delivery system, without the need for specific targeting).

In still other embodiments, the protein based recognition molecule can target the modified polymer to a location within the cell, such as the nucleus, the cytoplasm, or the endosome, for example. In specific embodiments, the protein based recognition molecule can enhance cellular binding to receptors, or cytoplasmic transport to the nucleus and nuclear entry or release from endosomes or other intracellular vesicles.

In specific embodiments the protein based recognition molecules include antibodies, proteins and peptides or peptide mimics.

Exemplary antibodies or antibodies derived from Fab, Fab2, scFv or camel antibody heavy-chain fragments specific to the cell surface markers, include, but are not limited to, 5T4, AOC3, ALK, AXL, C242, CA-125, CCL11, CCR 5, CD2, CD3, CD4, CD5, CD6, CD8, CD11, CD15, CA15-3, CD18, CD19, CA19-9, CD20, CD22, CD23, CD25, CD26, CD28, CD30, CD31, CD33, CD34, CD37, CD38, CD40, CD41, CD44, CD44 v6, CD46, CD51, CD52, CD54, CD56, CD62E, CD62P, CD62L, CD70, CD74, CD79, CD79-B, CD80, CD105, CD125, CD138, CD141, CD147, CD152, CD 154, CD326, CEA, clumping factor, CTLA-4, CXCR2, EGFR, EGFRvIII, ErbB2, ErbB3, EpCAM, EPHA2, EPHB2, EPHB4, FGFR (i.e. FGFR1, FGFR2, FGFR3, FGFR4), FLT3, folate receptor, FAP, GD2, GD3, GPNMB, HGF, HER2, HER3, HER4, ICAM, IGF-1 receptor, VEGFR1, EphA2, EphB, TRPV1, CFTR, gpNMB, CA9, Cripto, c-KIT, c-MET, ACE, APP, adrenergic receptor-beta2, Claudine 3, Mesothelin, MUC1, RON, ROR1, PD-L1, PD-L2, B7-H3, B7-B4, IL-2 receptor, IL-4 receptor, IL-13 receptor, integrins (including α₄, αᵥβ₃, αᵥβ₅, αᵥβ₆, α₁β₄, α₄β₁, α₄β₇, α₅β₁, α₆β₄, α_{IIb}β₃ integrins), IFN-α, IFN-γ, IgE, IgE, IGF-1 receptor, IL-1, IL-8, IL-12, IL-23, IL-13, IL-22, IL-4, IL-5, IL-6, interferon receptor, ITGB2 (CD18), LFA-1 (CD11a), L-selectin (CD62L), flk2/flt3, FLT3, PD-1, PD-L1, PD-L2, p150.95, Mac1, mucin, MUC1, myostatin, NCA-90, NGF, PDGFRα, phosphatidylserine, prostatic carcinoma cell, Pseudomonas aeruginosa, rabies, RANKL, respiratory syncytial virus, Rhesus factor, transferrin, SLAMF7, sphingosine-1-phosphate, TAG-72, T-cell receptor, tenascin C, TGF-1, TGF-β2, TGF-β, TNF-α, TRAIL-R1, TRAIL-R2, tumor antigen CTAA16.88, VEGF, VEGF-A, VEGFR2, VLA-4, VCAM, vimentin, and the like.

In one embodiment the antibodies or antibody derived from Fab, Fab2, scFv or camel antibody heavy-chain fragments specific to the cell surface markers include 5T4, CA-125, C242, CD3, CD8, CD19, CD22, CD25, CD30, CD31, CD33, CD34, CD37, CD40, CD44, CD46, CD51, CD54, CD56, CD62E, CD62P, CD62L, CD70, CD138, CD141, CD326, CEA, CTLA-4, EGFR, ErbB2, ErbB3, FAP, folate receptor, IGF-1 receptor, GD3, GPNMB, HGF, HER2, HER3, HER4, VEGF-A, VEGFR2, VEGFR1, EphA2, EpCAM, 5T4, TAG-72, tenascin C, TRPV1, CFTR, gpNMB, CA9, Cripto, ACE, APP, PDGFR α, phosphatidylserine, prostatic carcinoma cells, adrenergic receptor-beta2, Claudine 3, mesothelin, FLT3, PD-1, PD-L1, PD-L2, mucin, MUC1, Mesothelin, IL-2 receptor, IL-4 receptor, IL-13 receptor and integrins (including αᵥβ₃, αᵥβ₅, αᵥβ₆, α₁β₄, α₄β₁, α₅β₁, α₆β₄ integrins), tenascin C, TRAIL-R₂ and vimentin.

Exemplary antibodies include 3F8, abagovomab, abciximab (REOPRO), adalimumab (HUMIRA), adecatumumab, afelimomab, afutuzumab, alacizumab, ALD518, alemtuzumab (CAMPATH), altumomab, amatuximab, anatumomab, anrukinzumab, apolizumab, arcitumomab (CEA-SCAN), aselizumab, atlizumab (tocilizumab, Actemra, RoActemra), atorolimumab, bapineuzumab, basiliximab (Simulect), bavituximab, bectumomab (LYMPHOSCAN), belimumab (BENLYSTA), benralizumab, bertilimumab, besilesomab (SCINITIMUN), bevacizumab (AVASTIN), biciromab (FIBRISCINT), bivatuzumab, blinatumomab, brentuximab, briakinumab, canakinumab (ILARIS), cantuzumab, capromab, catumaxomab (REMOVAB), CC49, cedelizumab, certolizumab, cetuximab (ERBITUX), citatuzumab, cixutumumab, clenoliximab, clivatuzumab, conatumumab, CR6261, dacetuzumab, daclizumab (ZENAPAX), daratumumab, denosumab (PROLIA), detumomab, dorlimomab, dorlixizumab, ecromeximab, eculizumab (SOLIRIS), edobacomab, edrecolomab (PANOREX), efalizumab (RAPTIVA), efungumab (MYCOGRAB), elotuzumab, elsilimomab, enlimomab, epitumomab, epratuzumab, erlizumab, ertumaxomab (REXOMUN), etaracizumab (ABEGRIN), exbivirumab, fanolesomab (NEUTROSPEC), faralimomab, farletuzumab, felvizumab, fezakinumab, figitumumab, fontolizumab (HuZAF), foravirumab, fresolimumab, galiximab, gantenerumab, gavilimomab, gemtuzumab girentuximab, glembatumumab, golimumab (SIMPONI), gomiliximab, ibalizumab, ibritumomab, igovomab (INDIMACIS-125), imciromab (MYOSCINT), infliximab (REMICADE), intetumumab, inolimomab, inotuzumab, ipilimumab, iratumumab, keliximab, labetuzumab (CEA-CIDE), lebrikizumab, lemalesomab, lerdelimumab, lexatumumab, libivirumab, lintuzumab, lucatumumab, lumiliximab, mapatumumab, maslimomab, matuzumab, mepolizumab (BOSATRIA), metelimumab, milatuzumab, minretumomab, mitumomab, morolimumab, motavizumab (NUMAX), muromonab-CD3 (ORTHOCLONE OKT3), nacolomab, naptumomab, natalizumab (TYSABRI), nebacumab, necitumumab, nerelimomab, nimotuzumab (THERACIM), nofetumomab, ocrelizumab, odulimomab, ofatumumab (ARZERRA), olaratumab, omalizumab (XOLAIR), ontecizumab, oportuzumab, oregovomab (OVAREX), otelixizumab, pagibaximab, palivizumab (SYNAGIS), panitumumab (VECTIBIX), panobacumab, pascolizumab, pemtumomab (THERAGYN), pertuzumab (OMNITARG), pexelizumab, pintumomab, priliximab, pritumumab, PRO 140, rafivirumab, ramucirumab, ranibizumab (LUCENTIS), raxibacumab, regavirumab, reslizumab, rilotumumab, rituximab (RITUXAN), robatumumab, rontalizumab, rovelizumab (LEUKARREST), ruplizumab (ANTOVA), satumomab pendetide, sevirumab, sibrotuzumab, sifalimumab, siltuximab, siplizumab, solanezumab, sonepcizumab, sontuzumab, stamulumab, sulesomab (LEUKOSCAN), tacatuzumab (AFP-CIDE), tetraxetan, tadocizumab, talizumab, tanezumab, taplitumomab paptox, tefibazumab (AUREXIS), telimomab, tenatumomab, teneliximab, teplizumab, TGN1412, ticilimumab (tremelimumab), tigatuzumab, TNX-650, tocilizumab (atlizumab, ACTEMRA), toralizumab, tositumomab (BEXXAR), trastuzumab (HERCEPTIN), tremelimumab, tucotuzumab, tuvirumab, urtoxazumab, ustekinumab (STELERA), vapaliximab, vedolizumab, veltuzumab, vepalimomab, visilizumab (NUVION), volociximab (HUMASPECT), votumumab, zalutumumab (HuMEX-EGFr), zanolimumab (HuMAX-CD4), ziralimumab and zolimomab.

In some embodiments the antibodies are directed to cell surface markers for 5T4, CA-125, CEA, CD2, CD3, CD4, CD5, CD6, CD11, CD19, CD20, CD22, CD26, CD30, CD33, CD34, CD37, CD38, CD40, CD44, CD46, CD51, CD56, CD79, Cd105, CD138, CTLA-4, EphA, EphB, EpCAM, HER2, HER3, HER4, EGFR, FAP, folate receptor, HGF, integrin αᵥβ₃, integrin α₅β₃, IGF-1 receptor, GD3, GPNMB, CA9, FLT3, PD-1, PD-L1, PD-L2, mucin, mesothelin, MUC1, phosphatidylserine, prostatic carcinoma cells, PDGFR α, TAG-72, tenascin C, TRAIL-R2, VEGF-A and VEGFR2. In this embodiment the antibodies are abagovomab, adecatumumab, alacizumab, altumomab, anatumomab, arcitumomab, bavituximab, bevacizumab (AVASTIN), bivatuzumab, blinatumomab, brentuximab, cantuzumab, catumaxomab, capromab, cetuximab, citatuzumab, clivatuzumab, conatumumab, dacetuzumab, edrecolomab, epratuzumab, ertumaxomab, etaracizumab, farletuzumab, figitumumab, gemtuzumab, glembatumumab, ibritumomab, igovomab, intetumumab, inotuzumab, labetuzumab, lexatumumab, lintuzumab, lucatumumab, matuzumab, mitumomab, naptumomab estafenatox, necitumumab, oportuzumab, oregovomab, panitumumab, pemtumomab, pertuzumab, pritumumab, rituximab (RITUXAN), rilotumumab, robatumumab, satumomab, sibrotuzumab, taplitumomab, tenatumomab, tenatumomab, ticilimumab (tremelimumab), tigatuzumab, trastuzumab (HERCEPTIN), tositumomab, tremelimumab, tucotuzumab celmoleukin, volociximab and zalutumumab.

In specific embodiments the antibodies directed to cell surface markers for HER2 are pertuzumab or trastuzumab and for EGFR the antibody is cetuximab and for CD20 the antibody is rituximab and for VEGF-A the antibody is bevacizumab and for CD-22 the antibody is epratuzumab or veltuzumab and for CEA the antibody is labetuzumab and for CD44 the antibody is bivatuzumab and for FAP the antibody is sibrotuzumab.

Exemplary peptides or peptide mimics include integrin targeting peptides (RGD peptides), LHRH receptor targeting peptides, ErbB2 (HER2) receptor targeting peptides, prostate specific membrane bound antigen (PSMA) targeting peptides, lipoprotein receptor LRP1 targeting, ApoE protein derived peptides, ApoA protein peptides, somatostatin receptor targeting peptides, chlorotoxin derived peptides, and bombesin.

In specific embodiments the peptides or peptide mimics are LHRH receptor targeting peptides and ErbB2 (HER2) receptor targeting peptides.

Exemplary proteins and polypeptides comprise interferons such as α, β, γ; lymphokines such as IL-2, IL-3, IL-4 and IL-6; hormones such as insulin, TRH (thyrotropin releasing hormones) MSH (melanocyte-stimulating hormones), steroid hormones such as androgens and estrogens; transferrin, fibrinogen-gamma fragment, thrombospondin, claudin, apolipoprotein E, Affibody molecules such as, for example, ABY-025; Ankyrin repeat proteins, ankyrin-like repeats proteins and synthetic peptides.

In some embodiments of the invention the protein drug polymer conjugates comprise broad spectrum cytotoxins in combination with cell surface markers for HER2 such as pertuzumab or trastuzumab; for EGFR such as cetuximab; for CEA such as labetuzumab; for CD20 such as rituximab; for VEGF-A such as bevacizumab; or for CD-22 such as epratuzumab or veltuzumab.

In other embodiments of the invention the protein-polymer-drug conjugates or protein-polymer conjugates used in the invention comprise combinations of two or more protein based recognition molecules, such as, for example, combination of bispecific antibodies directed to the EGF receptor (EGFR) on tumor cells and to CD3 and CD28 on T cells; combination of bispecific antibodies directed to CD33 and FLT3; combination of antibodies or antibody derived from Fab, Fab2, scFv or camel antibody heavy-chain fragments and peptides or peptide mimetics; combination of antibodies or antibody derived from Fab, Fab2, scFv or camel antibody heavy-chain fragments and proteins; combination of two bispecific antibodies such as CD3×CD19 plus CD28×CD22 bispecific antibodies.

In other embodiments of the invention the protein-polymer-drug conjugates or protein-polymer conjugates used in the invention comprise protein based recognition molecules which are antibodies against antigens, such as, for example B7-H4, B7-H3, CA125, CD33, CXCR2, EGFR, FGFR1, FGFR2, FGFR3, FGFR4, HER2, PD-L1 and 5T4.

Table F below provides more examples of the PBRM described hereof, which are suitable for conjugation to form the polymer-drug-protein conjugates or polymer-PBRM scaffolds of the invention.

TABLE F

| Ref # | PBRM |
|---|---|
| Ex 3 | 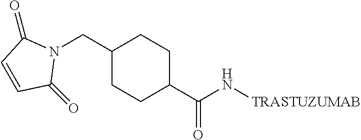 |
| Ex 4 | 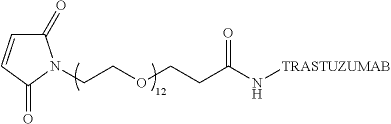 |
| Ex 5 | 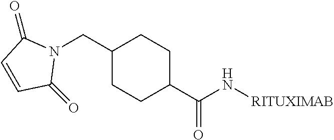 |
| | TRASTUZUMAB-Fab′-SH |
| Ex 9 | TRASTUZUMAB-F(ab′)₂ |
| Ex 10 | TRASTUZUMAB-(Fab) |
| | 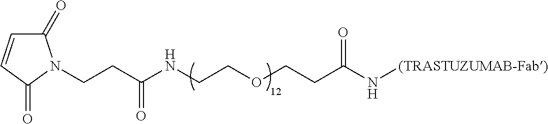 |
| Ex 13 | 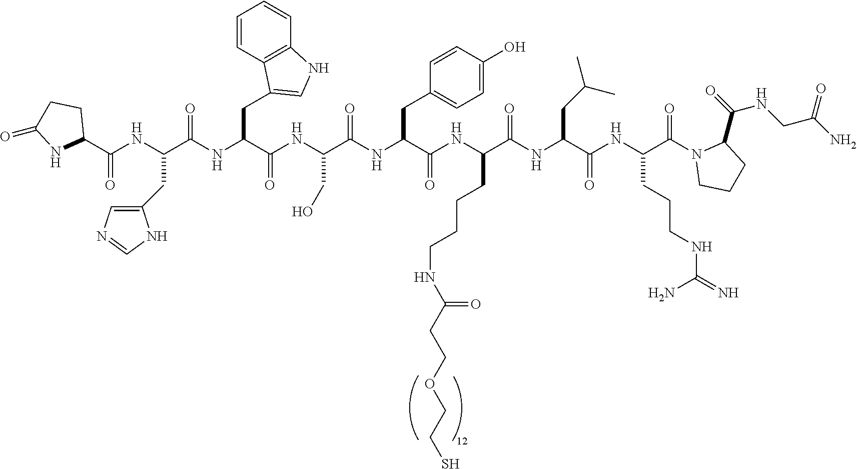 |
| Ex 15 | 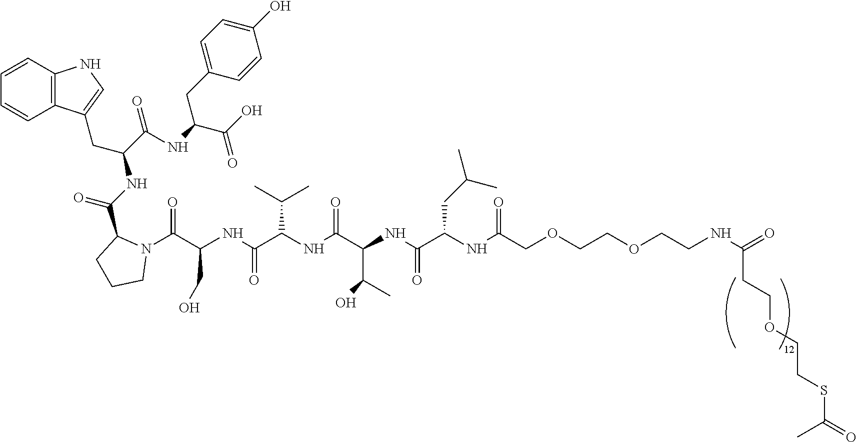 |
| Ex 14 | 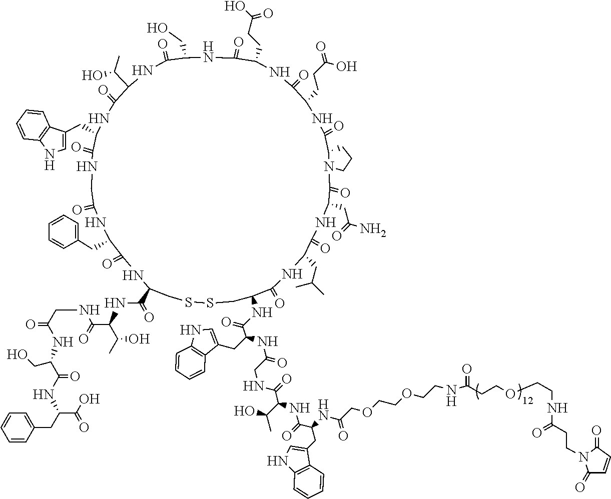 |

Linkers (L^{D} and L^{P})

As described above, the drug or PBRM is connected to the polymeric carrier via a linker L^{D} or L^{P}. In some embodiments, the linker is biocleavable/biodegradable under intracellular conditions, such that the cleavage of the linker releases the drug (i.e., tubulysin compound) or PBRM from the polymer unit in the intracellular environment.

A linker is any chemical moiety that is capable of linking a drug or a PBRM to a polymer backbone through chemical bonds such that the drug or PBRM and the polymer are chemically coupled (e.g., covalently bonded) to each other. In some embodiments, the linker comprises a biodegradable linker moiety (e.g., a biodegradable bond such as an ester or amide bond).

In other embodiments, the linker L^{D} or L^{P} is biodegradable under mild conditions, i.e., conditions within a cell under which the activity of the drug is not affected. Examples of suitable biodegradable linker moiety include disulfide linkers, acid labile linkers, photolabile linkers, peptidase labile linkers, and esterase labile linkers.

In some embodiments, the linker L^{D} or L^{P} is biocleavable under reducing conditions (e.g., a disulfide linker). In this embodiment the drug or PBRM moiety is linked to the polymer through a disulfide bond. The linker molecule comprises a reactive chemical group that can react with the drug. Preferred reactive chemical groups for reaction with the drug or PBRM moiety are N-succinimidyl esters and N-sulfosuccinimidyl esters. Additionally the linker molecule comprises a reactive chemical group, preferably a dithiopyridyl group that can react with the drug to form a disulfide bond. In some embodiments the linker molecules include, for example, N-succinimidyl 3-(2-pyridyldithio)propionate (SPDP), N-succinimidyl 4-(2-pyridyldithio)butanoate (SPDB), N-succinimidyl 4-(2-pyridyldithio)pentanoate (SPP), N-succinimidyl-S-acetylthioacetate (SATA) and N-succinimidyl-oxycarbonyl-alpha-methyl-alpha-(2-pyridyl-dithio)toluene or 2,5-dioxopyrrolidin-1-yl 4-(1-(pyridin-2-yldisulfanyl)ethyl)benzoate (SMPT).

In other embodiments, the biocleavable linker L^{D} or L^{P} is pH-sensitive, i.e., sensitive to hydrolysis at certain pH values. Typically, the pH-sensitive linker is hydrolysable under acidic conditions. For example, an acid-labile linker that is hydrolysable in the lysosome or endosome (e.g., a hydrazone, semicarbazone, thiosemicarbazone, cis-aconitic amide, orthoester, acetal, ketal, or the like) can be used. Such linkers are relatively stable under neutral pH conditions, such as those in the blood, but are unstable at below pH 5.5 or 5.0, the approximate pH of the lysosome. In certain embodiments, the hydrolysable linker is a thioether linker (such as, e.g., a thioether attached to the tubulysin compound via an acylhydrazone bond.

In other embodiments the linker L^{D} or L^{P} is photo-labile and is useful at the body surface and in many body cavities that are accessible to light. Furthermore, L^{D} or L^{P} is biocleavable by infrared light which can penetrate tissue. Accordingly, L^{D} or L^{P} is useful for both applications on the body surface and in the tissue.

In some embodiments, the linker L^{D} or L^{P} is biocleavable by a cleaving agent that is present in the intracellular environment (e.g., within a lysosome or endosome or caveolea). The linker can be, for example, a peptidyl linker that is cleaved by an intracellular peptidase or protease enzyme, including, but not limited to, a lysosomal or endosomal protease.

In some embodiments the linker L^{D} or L^{P} is cleaved by esterases. Only certain esters can be cleaved by esterases present inside or outside cells. Esters are formed by the condensation of a carboxylic acid and an alcohol. Simple esters are esters produced with simple alcohols, such as aliphatic alcohols, and small cyclic and small aromatic alcohols.

In yet other embodiments, the linker L^{D} or L^{P} is not biocleavable and the drug is released by antibody degradation. See, for example, U.S. Pat. No. 7,498,298, which is incorporated by reference herein in its entirety and for all purposes.

Typically, the linker L^{D} or L^{P} is not substantially sensitive to the extracellular environment. As used herein, “not substantially sensitive to the extracellular environment,” in the context of a linker, means that no more than about 20%, typically no more than about 15%, more typically no more than about 10%, and even more typically no more than about 5%, no more than about 3%, or no more than about 1% of the linkers, in a sample of Polymer Drug Conjugate, are cleaved when the Polymer Drug Conjugate presents in an extracellular environment (e.g., in plasma) for 24 hours. Whether a linker is not substantially sensitive to the extracellular environment can be determined, for example, by incubating the Polymer Drug Conjugate with plasma for a predetermined time period (e.g., 2, 4, 8, 16, or 24 hours) and then quantitating the amount of free drug present in the plasma.

In embodiments, the linker L^{D} has the structure: —R^{L1}—C(═O)—X^{D}-M^{D1}-Y^{D}-M^{D2}-Z^{D}-M^{D3}-Q^{D}-M^{D4}-, with R^{L1} connected to an oxygen atom of the polymeric carrier and M^{D4} connected to the drug molecule to be delivered.

In embodiments, the linker L^{P} has the structure: —R^{L2}—C(═O)—X^{P}-M^{P1}-Y^{P}-M^{P2}-Z^{P}-M^{P3}-Q^{P}-M^{P4}-, with R^{L2} connected to an oxygen atom of the polymeric carrier and M^{P4} connected to the PBRM.

For example, each of R^{L1} and R^{L2} independently is absent, alkyl, alkenyl, alkynyl, cycloalkyl, heteroalkyl, heteroalkenyl, heteroalkynyl, heterocycloalkyl, aryl, or heteroaryl.

For example, each of R^{L1} and R^{L2} independently is absent, alkyl, cycloalkyl, heteroalkyl, or heterocycloalkyl.

For example, R^{L1} is absent.

For example, R^{L2} is absent.

For example, each of X^{D} and X^{P}, independently is —O—, —S—, —N(R¹)—, or absent, in which R¹ is hydrogen, an aliphatic, heteroaliphatic, carbocyclic, or heterocycloalkyl moiety, —C(═O)R^{1B}, —C(═O)OR^{1B}, —SO₂R^{1B} or —N(R¹)— is a heterocycloalkyl moiety, wherein R^{1B} is hydrogen, an aliphatic, heteroaliphatic, carbocyclic, or heterocycloalkyl moiety.

For example, each of Y^{D}, Y^{P}, Z^{D}, Z^{P}, Q^{D}, and Q^{P}, independently, is absent or a biodegradable linker moiety selected from the group consisting of —S—S—, —C(═O)O—, —C(═O)NR²—, —OC(═O)—, —NR²C(═O)—, —OC(═O)O—, —OC(═O)NR²—, —NR²C(═O)O—, —NR²C(═O)NR³—, —C(OR²)O—, —C(OR²)S—, —C(OR²)NR³—, —C(SR²)O—, —C(SR²)S—, —C(SR²)NR³—, —C(NR²R³)O—, —C(NR²R³)S—, —C(NR²R³)NR⁴—, —C(═O)S—, —SC(═O)—, —SC(═O)S—, —OC(═O)S—, —SC(═O)—, —C(═S)S—, —SC(═S)—, —OC(═S)—, —C(═S)O—, —SC(═S)O—, —OC(═S)S—, —OC(═S)O—, —SC(═S)S—, —C(═NR²)O—, —C(═NR²)S—, —C(═NR²)NR³—, —OC(═NR²)—, —SC(═NR²)—, —NR³C(═NR²)—, —NR²SO₂—, —NR²NR³—, —C(═O)NR²NR³—, —NR²NR³C(═O)—, —OC(═O)NR²NR³—, —NR²NR³C(═O)O—, —C(═S)NR²NR³—, —NR²NR³C(═S)—, —C(═NR⁴)NR²NR³—, —NR²NR³C(═NR⁴)—, —O(N═CR³)—, —(CR³═N)O—, —C(═O)NR²—(N═CR³)—, —(CR³═N)—NR²C(═O)—, —SO₃—, —NR²SO₂NR³—, —SO₂NR²—, and polyamide, wherein each occurrence of R², R³, and R⁴ independently is hydrogen or an aliphatic, heteroaliphatic, carbocyclic, or heterocyclic moiety, or each occurrence of —NR²— or —NR²NR³— is a heterocycloalkyl moiety.

For example, each of M^{D1}, M^{D2}, M^{D3}, M^{D4}, M^{P1}, M^{P2}, M^{P3} and M^{P4}, independently, is absent or a non-biodegradable linker moiety selected from the group consisting of alkyl, alkenyl, alkynyl, cycloalkyl, heteroalkyl, heteroalkenyl, heteroalkynyl, heterocycloalkyl, aryl, heteroaryl, and a combination thereof and each of M^{D1}, M^{D2}, M^{D3}, M^{P1}, M^{P2}, and M^{P3} optionally contains one or more —(C═O)— but does not contain any of the biodegradable linker moieties mentioned above.

For example, each of M^{D1}, M^{D2}, M^{D3}, M^{D4}, M^{P1}, M^{P2}, M^{P3} and M^{P4}, independently is C₁₋₆ alkyl, C₁₋₆ alkyl-C(O)—C₀₋₆ alkyl, C₁₋₆ alkyl-NH—C₀₋₆ alkyl, C₁₋₆ alkyl-O—C₀₋₆ alkyl, C₁₋₆ alkyl-S—C₀₋₆ alkyl, C₁₋₆ alkyl-C(O)—C₁₋₆ alkyl-NH, C₁₋₆ alkyl-C(O)—C₁₋₆ alkyl-O, C₁₋₆ alkyl-C(O)—C₁₋₆ alkyl-S, C₃₋₁₀ cycloalkyl-C(O)—C₀₋₆ alkyl, 3-19 membered heterocycloalkyl-C(O)—C₀₋₆ alkyl, aryl-C(O)—C₀₋₆ alkyl, (CH₂CH₂O)₁₋₁₂, and the like.

For example, for each L^{D}, M^{D1} is not absent when X^{D} is absent.

For example, for each L^{P}, M^{P1} is not absent when X^{P} is absent.

For example, for each L^{D}, at least one of X^{D}, Y^{D}, Z^{D}, and Q^{D} is not absent.

For example, for each L^{P}, at least one of X^{P}, Y^{P}, Z^{P}, and Q^{P} is not absent.

For example, each of M^{D1} and M^{P1} independently is C₁₋₆ alkyl or C₁₋₆ heteroalkyl.

For example, each of M^{D2}, M^{D3}, M^{D4}, M^{P2}, M^{P3}, and M^{P4}, independently is absent, C₁₋₆ alkyl, cycloalkyl, heteroalkyl, heterocycloalkyl, or a combination thereof.

For example, for each L^{D}, at most two of M^{D2}, M^{D3}, and M^{D4} are absent.

For example, for each L^{P}, at most two of M^{P2}, M^{P3}, and M^{P4} are absent.

For example, for each L^{D}, one of M^{D2} and M^{D3} has one of the following structures:

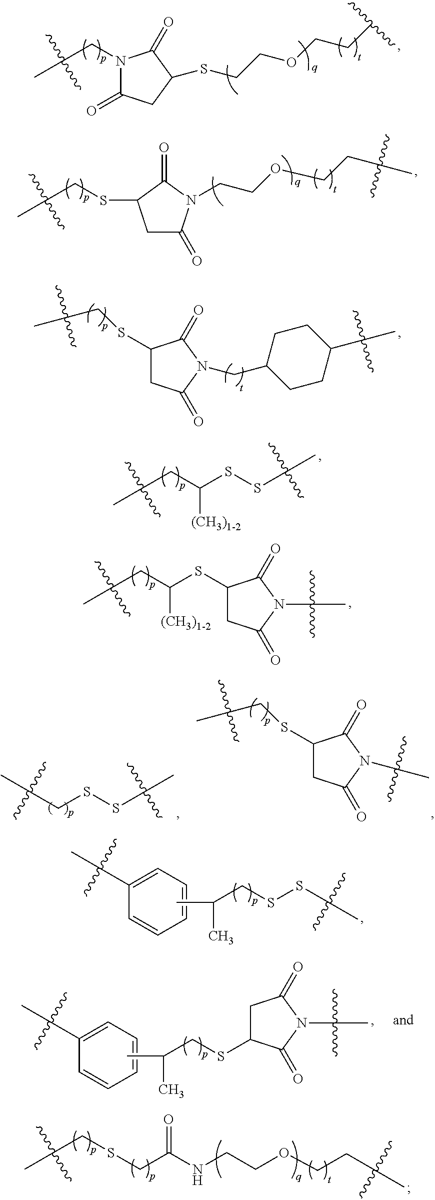

in which q is an integer from 0 to 12 and each of p and t independently is an integer from 0 to 3, and the other of M^{D2} or M^{D3} is either absent or a moiety different from the above, such as C₁₋₆ alkyl.

For example, for each L^{P}, one of M^{P2} and M^{P3} has one of the following structures:

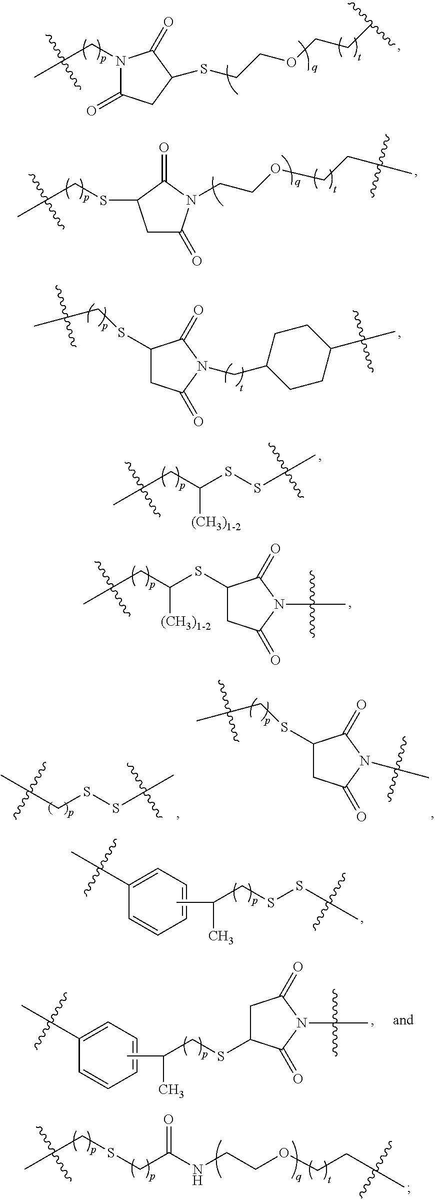

in which q is an integer from 0 to 12 and each of p and t independently is an integer from 0 to 3, and the other of M^{P2} or M^{P3} is either absent or a moiety different from the above, such as C₁₋₆ alkyl.

For example, p is 2.

For example, q is 0 or 12.

For example, t is 0 or 1.

For example, each of -M^{D2}-Z^{D}—, —Z^{D}-M^{D3}-, —Z^{D}-M^{D2}-, or -M^{D3}-Z^{D}—, independently has one of the following structures:

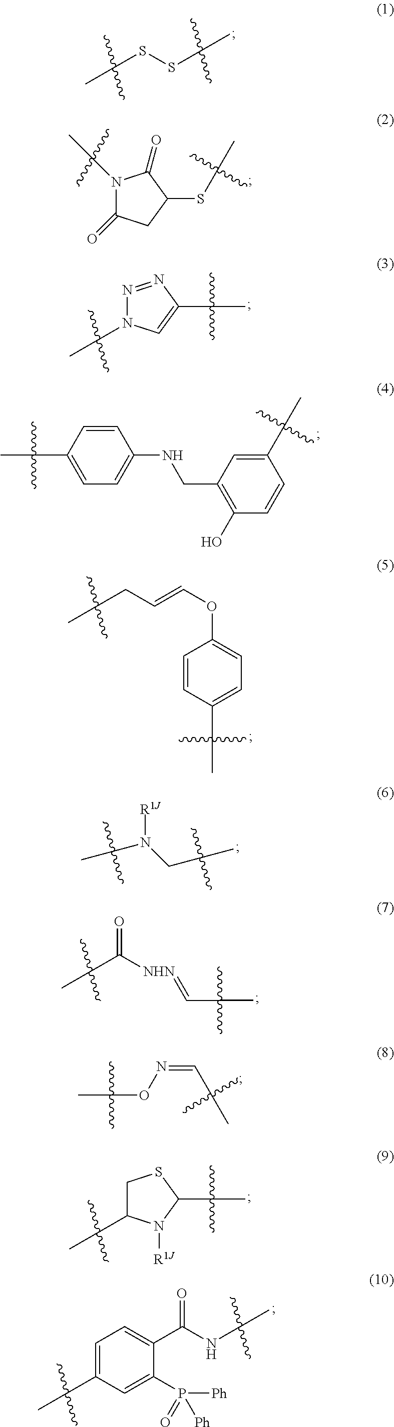

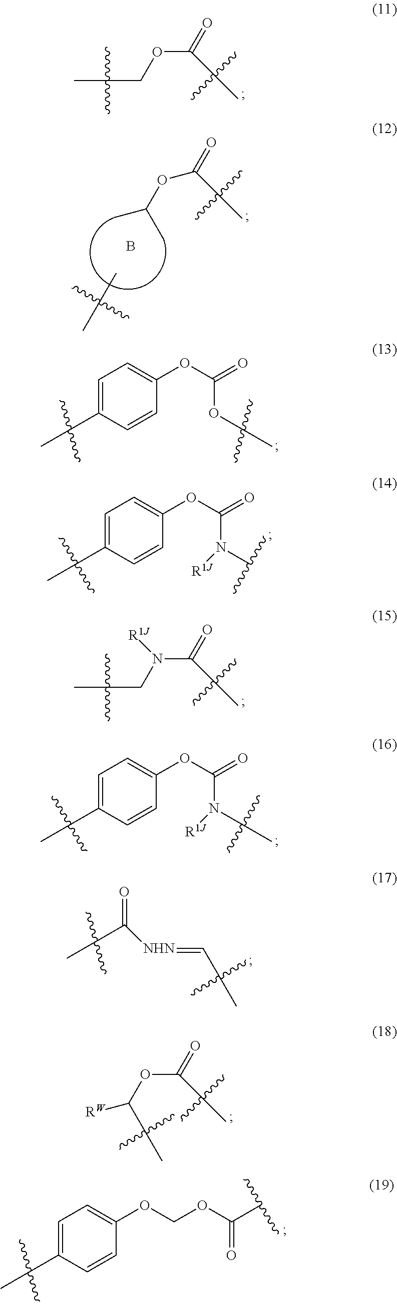

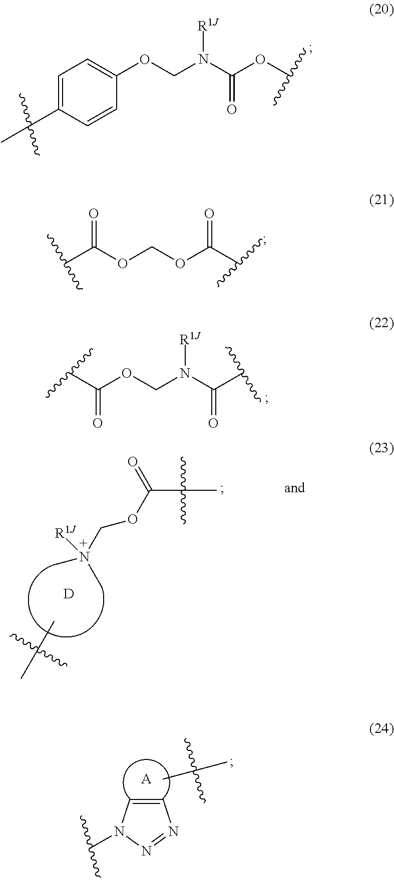

in which ring A or B independently is cycloalkyl or heterocycloalkyl; R^{W} is an aliphatic, heteroaliphatic, carbocyclic, or heterocycloalkyl moiety; R^{1J} is hydrogen, an aliphatic, heteroaliphatic, carbocyclic, or heterocycloalkyl moiety; and ring D is heterocycloalkyl.

For example, each of -M^{P2}-Z^{P}—, —Z^{P}-M^{P3}-, —Z^{P}-M^{P2}-, and -M^{P3}-Z^{P}— independently, has one of the following structures:

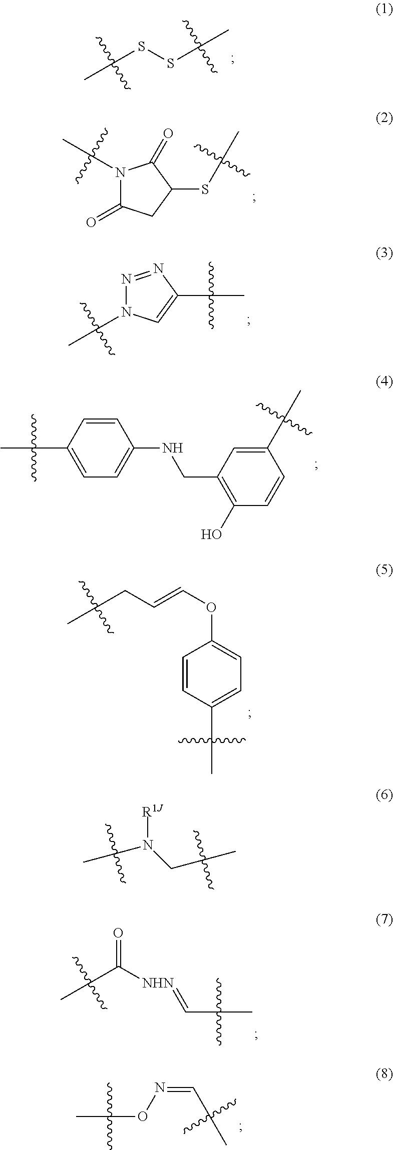

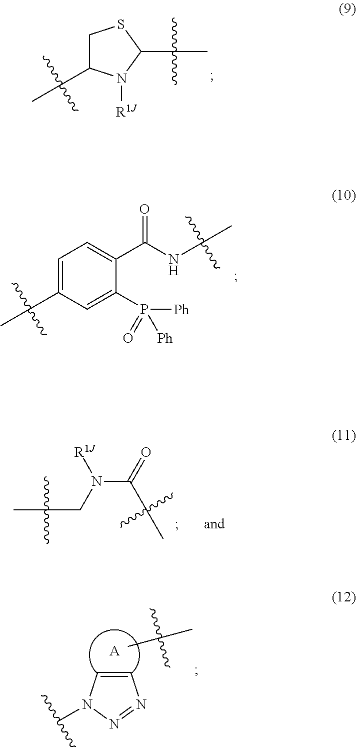

in which ring A is cycloalkyl or heterocycloalkyl and R^{1J} is hydrogen, an aliphatic, heteroaliphatic, carbocyclic, or heterocycloalkyl moiety.

For example, ring A is 5-19 membered heterocycloalkyl, e.g.,

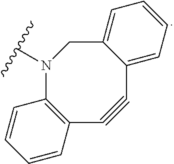 [SMILES: *N1Cc2ccccc2C#Cc2ccccc21]

For example, ring A is C₃₋₈ cycloalkyl.

For example, ring D is piperazinyl or piperidinyl.

For example, R^{W} is C₁₋₆ alkyl.

For example, R^{1J} is hydrogen or C₁₋₆ alkyl.

For example, Z^{D} is

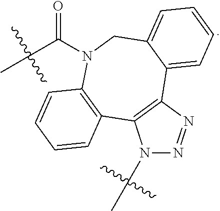 [SMILES: *C(=O)N1Cc2ccccc2-c2nnn(*)c2-c2ccccc21]

For example, Z^{P} is

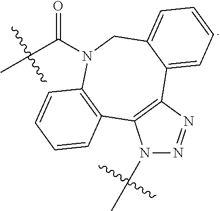 [SMILES: *C(=O)N1Cc2ccccc2-c2nnn(*)c2-c2ccccc21]

For example, X^{D} is absent, O or NH.

For example, X^{P} is absent, O or NH.

For example, each of X^{D} and X^{P}, independently is

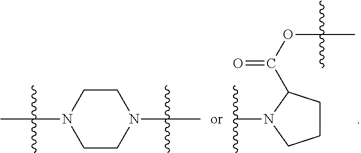 [SMILES: *N1CCN(*)CC1 ; *OC(=O)C1CCCN1*]

For example, each of Y^{D} and Y^{P} independently is —S—S—, —OCO—, —COO—, —CONH— or —NHCO—.

For example, each of Q^{D} and Q^{P} independently is absent, —S—S—, —OCO—, —COO—, —CONH—, —NHCO—, —OCONHNH—, or —NHNHCOO—.

For example, -L^{D}-D can have one of the following structures below, in which the wavy bond indicates that D (i.e., Drug) is either connected to the functional linker directly or via another moiety:

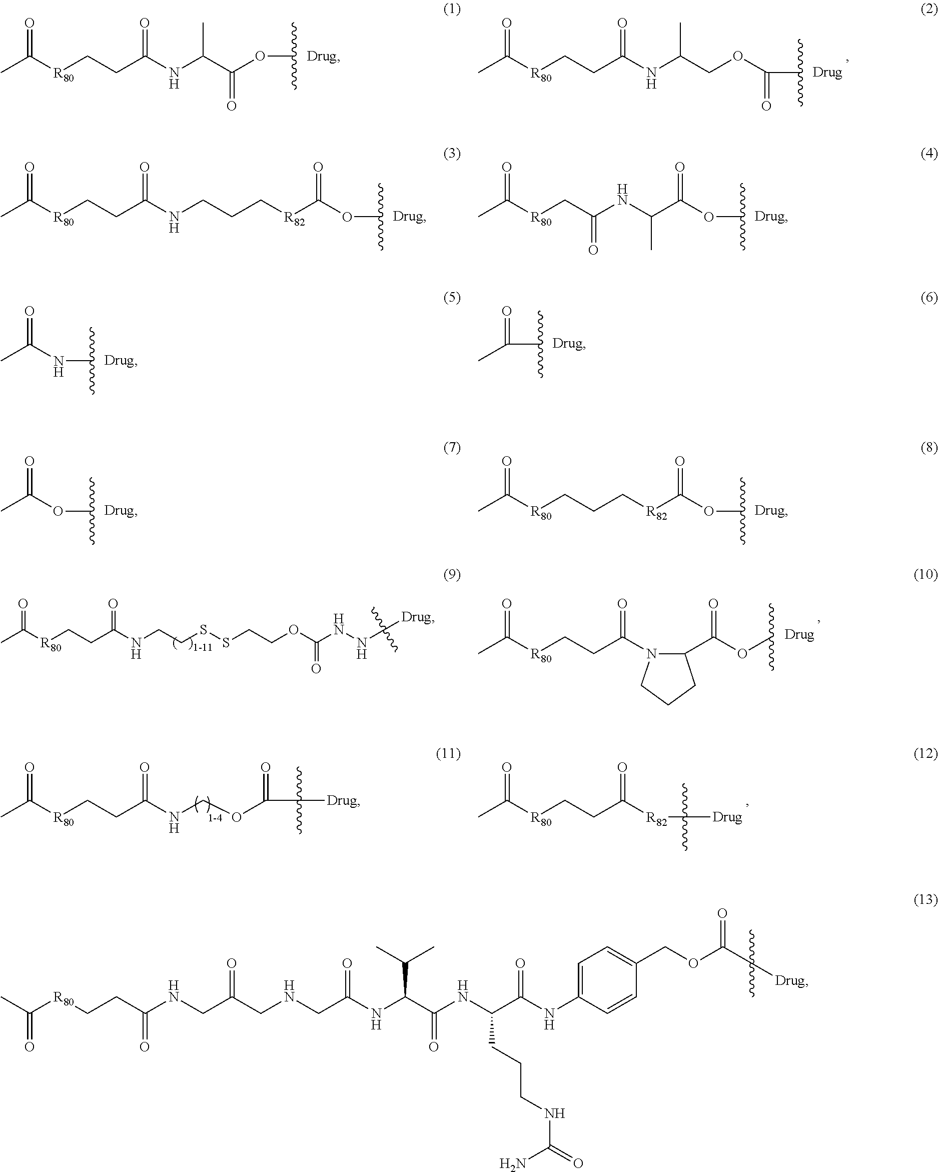

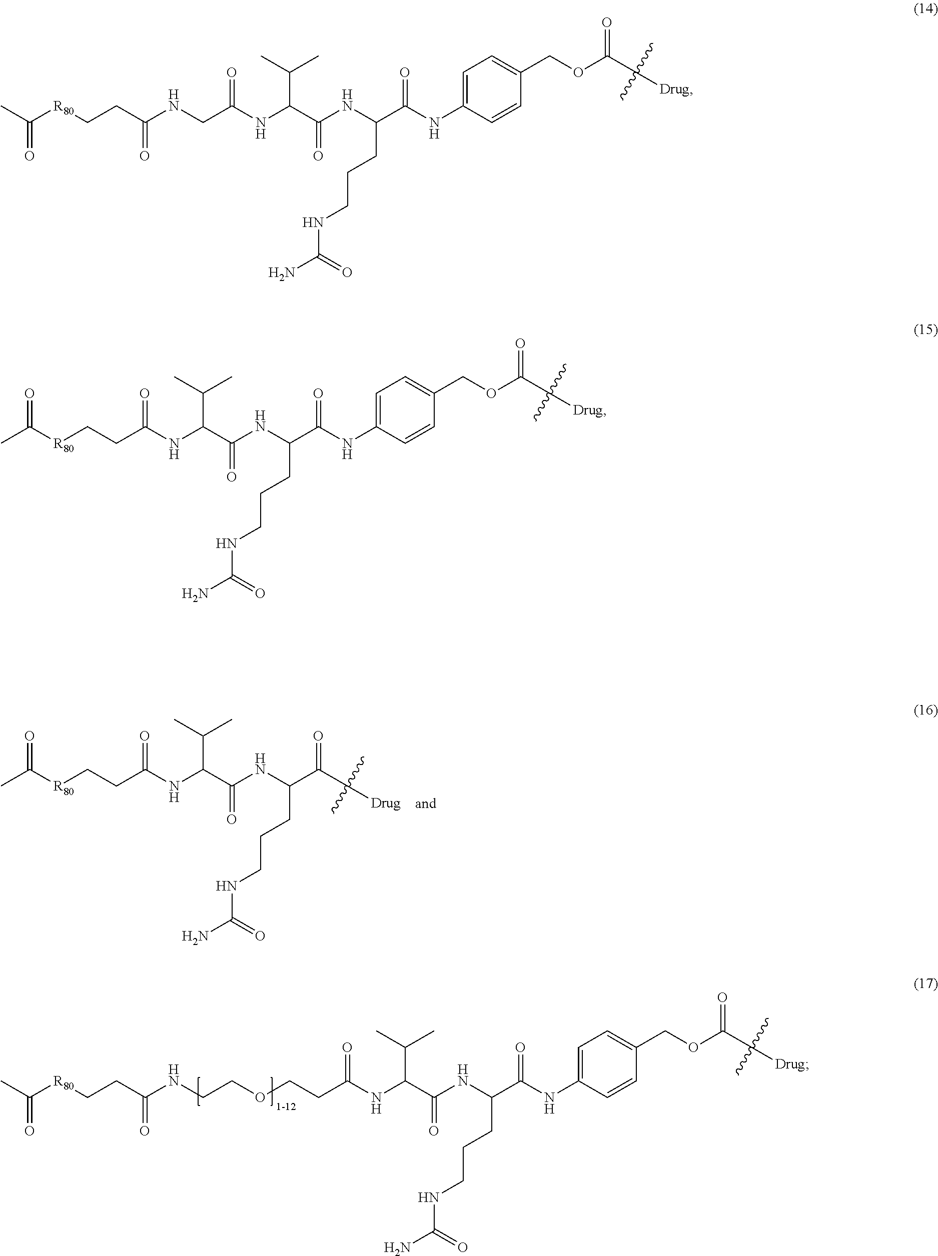

wherein R₅₀ is CH₂, —NH, or oxygen; and
R₈₂ is —NH or oxygen.

For example, polymeric carrier-L^{P}-PBRM can have one of the following structures below:

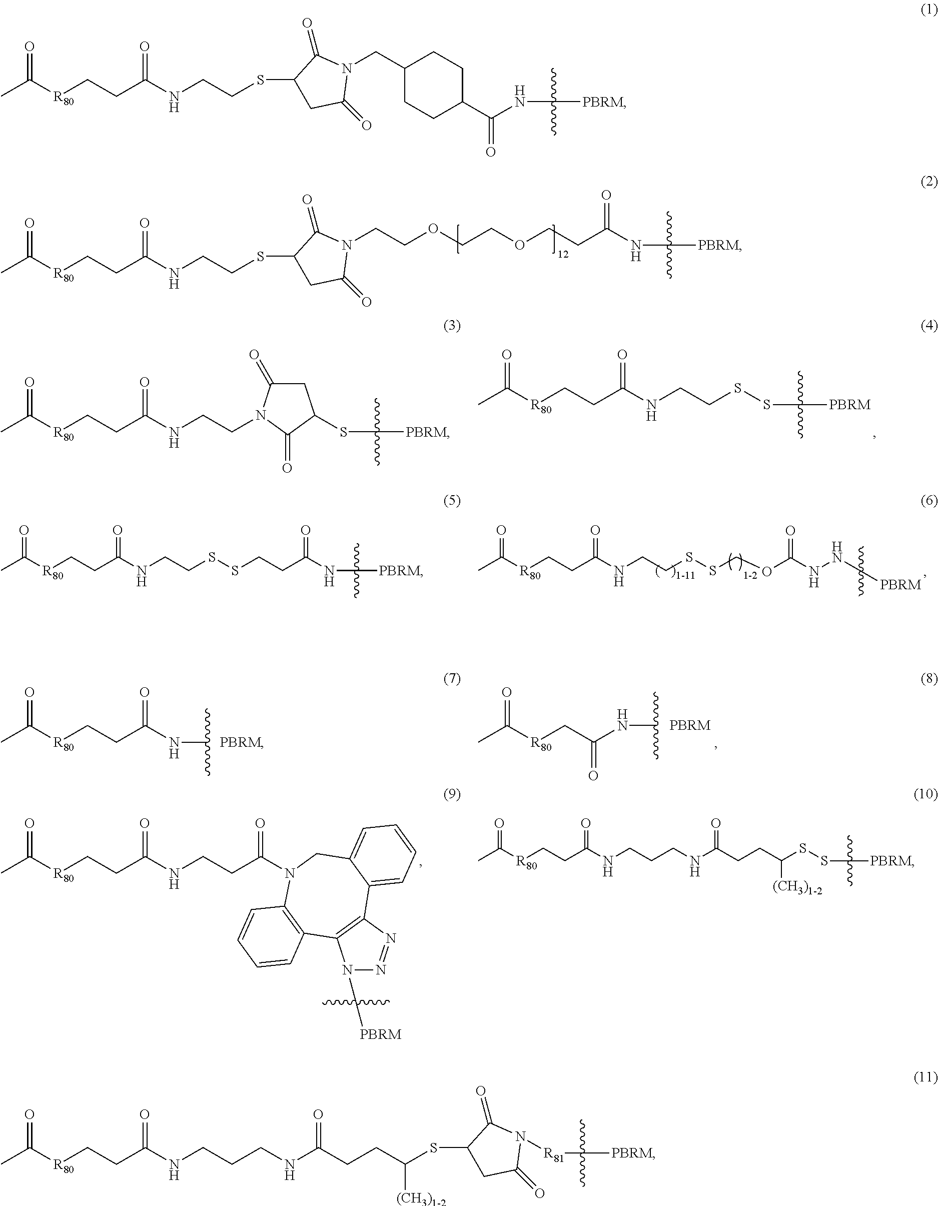

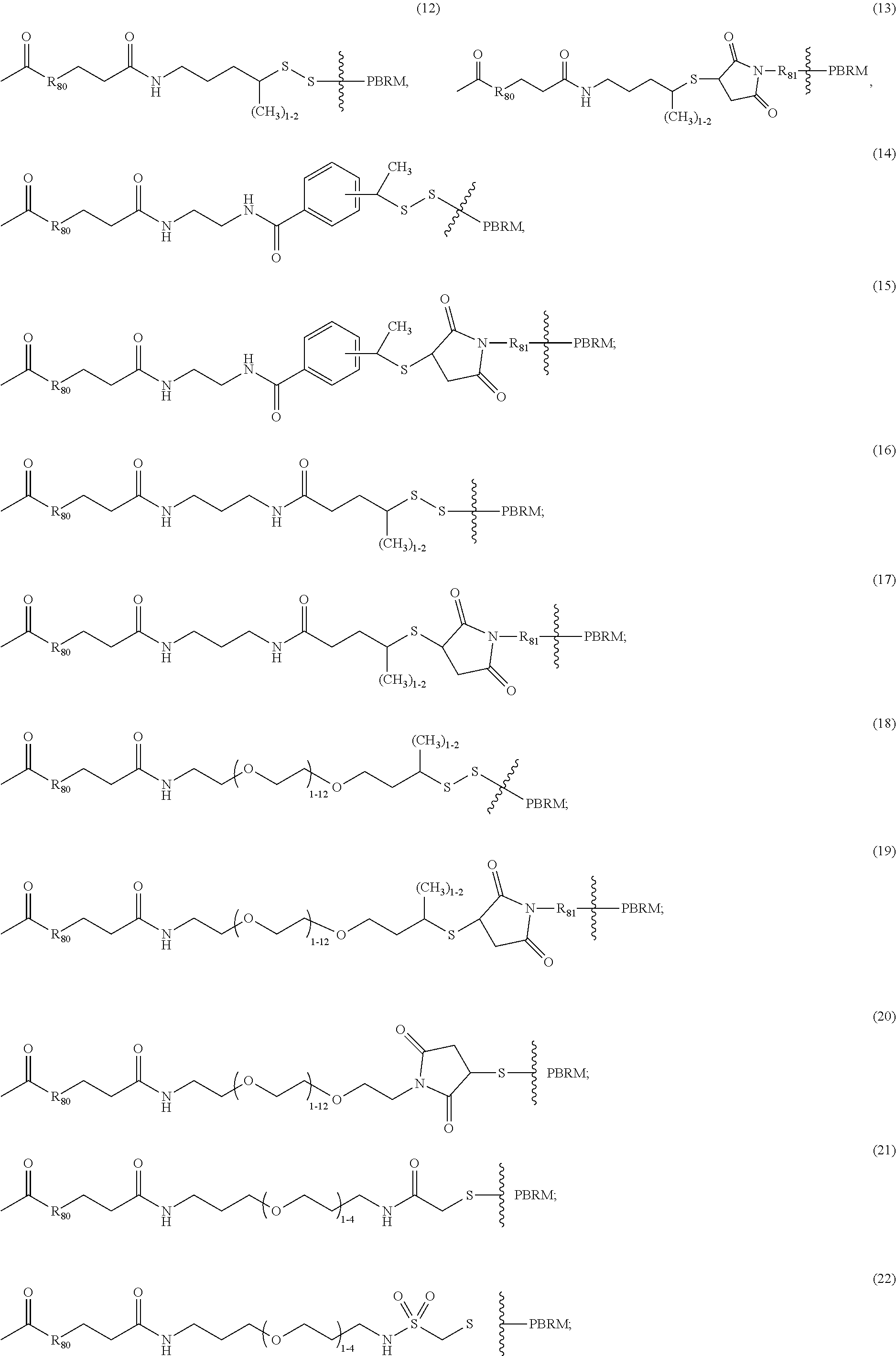

wherein:

R₈₀ is CH₂, NH or oxygen; and

R₈₁ is

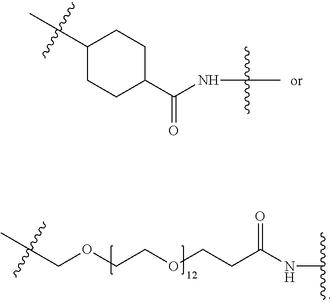

Additional examples of linker L^{D} and L^{P} which are suitable for use in the present invention are described in US 2012/0321583 and US 2013/0101546, each of which is hereby incorporated by reference in its entirety.

While biocleavable linkers preferably are used in the invention, a non-biocleavable linker also can be used to generate the above-described conjugate. A non-biocleavable linker is any chemical moiety that is capable of linking a drug or PBRM, to a polymer in a stable, covalent manner. Thus, non-biocleavable linkers are substantially resistant to acid-induced cleavage, light-induced cleavage, peptidase-induced cleavage, esterase-induced cleavage, and/or disulfide bond cleavage, at conditions under which the drug or polymer remains active.

In one embodiment, a substantial amount of the drug moiety is not cleaved from the conjugate until the protein-polymer-drug conjugate enters a cell with a cell-surface receptor specific for the PBRM of the protein-polymer-drug conjugate, and the drug moiety is cleaved from the protein-polymer-drug conjugate when the protein-polymer-drug conjugate does enter the cell.

In another embodiment, the bioavailability of the protein-polymer-drug conjugate or an intracellular metabolite of the protein-polymer-drug conjugate in a subject is improved when compared to a drug compound or conjugate comprising the drug moiety of the protein-polymer-drug conjugate, or when compared to an analog of the compound not having the drug moiety.

In another embodiment, the drug moiety is intracellularly cleaved in a subject from the protein-polymer-drug conjugate, or an intracellular metabolite of the protein-polymer-drug conjugate.

### Conjugates or Polymeric Scaffolds

Conjugates of the invention comprise one or more occurrences of D, where D is a tubulysin compound, wherein the one or more occurrences of D may be the same or different.

In certain other embodiments, one or more occurrences of PBRM is attached to the polymeric carrier, wherein the one or more occurrences of PBRM may be the same or different. In certain other embodiments, one or more polymer carriers that contains one or more occurrences of D are connected to a PBRM (e.g., an antibody).

As discussed more generally above, in certain embodiments, each polymeric carrier independently, has about 0.1 to about 25% monomers comprising a D, more preferably about 0.5 to about 20%, more preferably about 1 to about 15%, and even more preferably about 2 to about 10%.

In certain embodiments, the conjugate of this invention is of Formula (I):

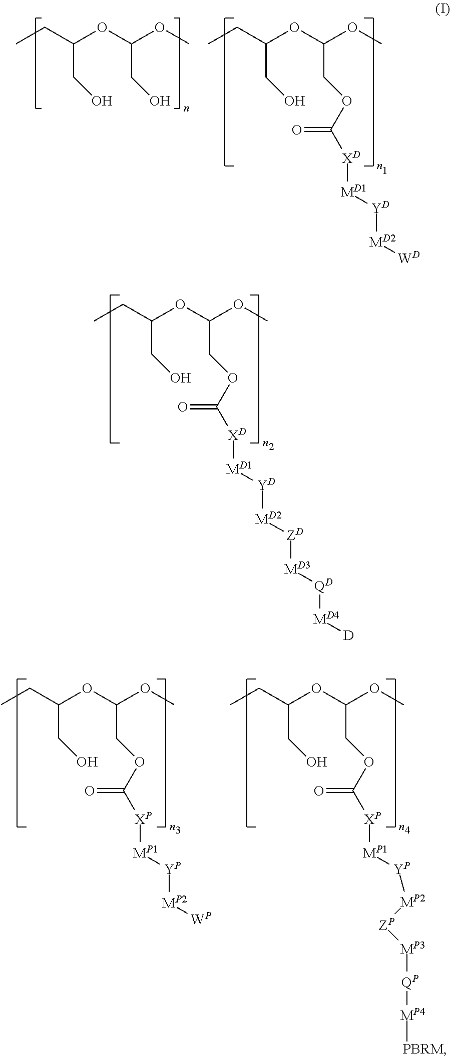

wherein:

each of n, n₁, n₂, n₃, and n₄, is the molar fraction of the corresponding polymer unit ranging between 0 and 1; n+n₁+n₂+n₃+n₄=1; provided that none of n, n₂, and n₄ is 0.

For example, the ratio between n₂ and n₄ is greater than 1:1 and ≤200:1.

For example, the ratio between n₂ and n₄ is between 10:1 and 50:1.

For example, the ratio between n₂ and n₄ is between 30:1 and 50:1.

For example, the ratio between n₂ and n₄ is about 50:1, 25:1, 10:1, 5:1 or 2:1.

In certain embodiments, the conjugates are formed in several steps. These steps include (1) modifying a polymer so that it contains a functional group that can react with a functional group of the drug or its derivative; (2) reacting the modified polymer with the drug or its derivative so that the drug is linked to the polymer; (3) modifying the polymer-drug conjugate so that the polymer contains a functional group that can react with a functional group of the PBRM or its derivative; and (4) reacting the modified polymer-drug conjugate with the PBRM or its derivative to form the conjugate of this invention. Step (3) may be omitted if the modified polymer produced by step (1) contains a functional group that can react with a functional group of the PBRM or its derivative.

In another embodiment the conjugates are formed in several steps: (1) modifying a polymer so that it contains a functional group that can react with a functional group of a first drug or its derivative; (2) reacting the modified polymer with the first drug or its derivative so that the first drug is linked to the polymer; (3) modifying the polymer-drug conjugate so that it contains a different functional group that can react with a functional group of a second drug or its derivative (4) reacting the modified polymer-drug conjugate with the second drug or its derivative so that the second drug is linked to the polymer-drug conjugate; (5) modifying the polymer-drug conjugate containing two different drugs so that the polymer contains a functional group that can react with a functional group of the PBRM or its derivative; and (6) reacting the modified polymer-drug conjugate of step (5) with the PBRM or its derivative to form the conjugate of this invention. Steps (5) and (6) may be repeated if two different PBRM or their derivatives are to be conjugated to form a polymer-drug conjugate comprising two different drugs and two different PBRMs.

In yet another embodiment, the conjugates are formed in several steps. These steps include (1) modifying a polymer so that it contains a functional group that can react with a functional group of the drug or its derivative; (2) further modifying the polymer so that it also contains a functional group that can react with a functional group of the PBRM or its derivative; (3) reacting the modified polymer with the drug or its derivative so that the drug is linked to the polymer; and (4) reacting the modified polymer-drug conjugate with the PBRM or its derivative to form the conjugate of this invention. The sequence of steps (1) and (2) or that of steps (3) and (4) can be reversed. Further either step (1) or (2) may be omitted if the modified polymer contains a functional group that can react with both a functional group of the drug or its derivatives and a functional group of the PBRM or its derivative.

In another embodiment the conjugates are formed in several steps: (1) modifying a polymer so that it contains a functional group that can react with a functional group of a first drug or its derivative; (2) further modifying a polymer so that it contains a functional group that can react with a functional group of the PBRM or its derivative; (3) reacting the modified polymer with the first drug or its derivative so that the first drug is linked to the polymer; (4) modifying the polymer-drug conjugate so that it contains a different functional group that can react with a functional group of a second drug or its derivative (5) reacting the modified polymer-drug conjugate with the second drug or its derivative so that the second drug is linked to the polymer-drug conjugate; (6) reacting the modified polymer-drug conjugate containing two different drugs so that the polymer with the PBRM or its derivative to form the conjugate of this invention. Step (6) may be repeated if two different PBRM or their derivatives are to be conjugated to form a polymer-drug conjugate comprising two different drugs and two different PBRMs. Step (4) may be carried out after step (1) so that the modified polymer contains two different functional groups that can react with two different drugs or their derivatives. In this embodiment, the modified polymer containing two different functional group that can react with two different drugs or their derivatives can be further modified so that it contains a functional group that can react with a functional group of the PBRM or its derivative; prior to the reaction of the modified polymer with either the two different drugs (step (3) and step (5) or PBRM (step (6).

The biodegradable biocompatible conjugates of the invention can be prepared to meet desired requirements of biodegradability and hydrophilicity. For example, under physiological conditions, a balance between biodegradability and stability can be reached. For instance, it is known that molecules with molecular weights beyond a certain threshold (generally, above 40-100 kDa, depending on the physical shape of the molecule) are not excreted through kidneys, as small molecules are, and can be cleared from the body only through uptake by cells and degradation in intracellular compartments, most notably lysosomes. This observation exemplifies how functionally stable yet biodegradable materials may be designed by modulating their stability under general physiological conditions (pH=7.5±0.5) and at lysosomal pH (pH near 5). For example, hydrolysis of acetal and ketal groups is known to be catalyzed by acids, therefore polyals will be in general less stable in acidic lysosomal environment than, for example, in blood plasma. One can design a test to compare polymer degradation profile at, for example, pH=5 and pH=7.5 at 37° C. in aqueous media, and thus to determine the expected balance of polymer stability in normal physiological environment and in the “digestive” lysosomal compartment after uptake by cells. Polymer integrity in such tests can be measured, for example, by size exclusion HPLC. One skilled on the art can select other suitable methods for studying various fragments of the degraded conjugates of this invention.

In many cases, it will be preferable that at pH=7.5 the effective size of the polymer will not detectably change over 1 to 7 days, and remain within 50% from the original for at least several weeks. At pH=5, on the other hand, the polymer should preferably detectably degrade over 1 to 5 days, and be completely transformed into low molecular weight fragments within a two-week to several-month time frame. Although faster degradation may be in some cases preferable, in general it may be more desirable that the polymer degrades in cells with the rate that does not exceed the rate of metabolization or excretion of polymer fragments by the cells. Accordingly, in certain embodiments, the conjugates of the present invention are expected to be biodegradable, in particular upon uptake by cells, and relatively “inert” in relation to biological systems. The products of carrier degradation are preferably uncharged and do not significantly shift the pH of the environment. It is proposed that the abundance of alcohol groups may provide low rate of polymer recognition by cell receptors, particularly of phagocytes. The polymer backbones of the present invention generally contain few, if any, antigenic determinants (characteristic, for example, for some polysaccharides and polypeptides) and generally do not comprise rigid structures capable of engaging in “key-and-lock” type interactions in vivo unless the latter are desirable. Thus, the soluble, crosslinked and solid conjugates of this invention are predicted to have low toxicity and bioadhesivity, which makes them suitable for several biomedical applications.

In certain embodiments of the present invention, the biodegradable biocompatible conjugates can form linear or branched structures. For example, the biodegradable biocompatible polyal conjugates of the present invention can be chiral (optically active). Optionally, the biodegradable biocompatible polyal conjugates of the present invention can be scalemic.

In certain embodiments, the conjugates of the invention are water-soluble. In certain embodiments, the conjugates of the invention are water-insoluble. In certain embodiments, the inventive conjugate is in a solid form. In certain embodiments, the conjugates of the invention are colloids. In certain embodiments, the conjugates of the invention are in particle form. In certain embodiments, the conjugates of the invention are in gel form.

This invention also features a polymeric scaffold useful for conjugating with a PBRM to form a polymer-drug-PBRM conjugate described herein. The scaffold comprises a polymeric carrier, one or more L^{D}-D connected to the polymeric carrier, and one or more L^{P} connected to the polymeric carrier which is suitable for connecting a PBRM to the polymeric carrier, wherein:

each occurrence of D is independently a tubulysin compound (e.g., a naturally occurring tubulysin or an analog or derivative thereof) having a molecular weight of ≤5 kDa;

the polymeric carrier is a polyacetal or a polyketal,

L^{D} is a first linker having the structure:

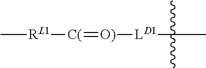

with R^{L1} connected to an oxygen atom of the polymeric carrier and L^{D1} connected to D, and

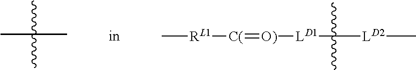

denotes direct or indirect attachment of D to L^{D1}, and L^{D} contains a biodegradable bond so that when the bond is broken, D is released in an active form for its intended therapeutic effect;

L^{D1} is a carbonyl-containing moiety;

L^{P} is a second linker having the structure: —R^{L2}—C(═O)-L^{P1} with R^{L2} connected to an oxygen atom of the polymeric carrier and L^{P1} suitable for connecting and not yet connected directly or indirectly to a PBRM, and each occurrence of the second linker is distinct from each occurrence of the first linker;

each of R^{L1} and R^{L2} independently is absent, alkyl, heteroalkyl, cycloalkyl, or heterocycloalkyl; and

L^{P1} is a moiety containing a functional group that is capable of forming a covalent bond and not yet formed with a functional group of a PBRM.

For example, L^{P} is a linker having the structure:

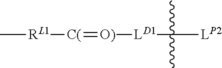

in which L^{P2} is a moiety containing a functional group that is capable of forming and not yet formed a covalent bond with a functional group of a PBRM, an

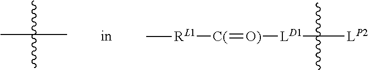

denotes direct or indirect attachment of L^{P2} to L^{D1}.

For example, the functional group of L^{P1} or L^{P2} is selected from —SR^{p}, —S—S-LG, maleimido, and halo, in which LG is a leaving group and R^{p} is H or a sulfur protecting group.

For example, L^{D1} comprises —X—(CH₂)ᵥ—C(═O)— with X directly connected to the carbonyl group of R^{L1}—C(═O), in which X is CH₂, O, or NH, and v is an integer from 1 to 6.

For example, L^{P1} or L^{P2} contains a biodegradable bond.

For example, each of R^{L1} and R^{L2} is absent.

For example, the polymeric carrier of the scaffold of the invention is a polyacetal, e.g., a PHF having a molecular weight (i.e., MW of the unmodified PHF) ranging from about 2 kDa to about 300 kDa. The selection of a polymeric carrier with a specific MW range may depend on the size of the PBRM to be conjugated.

For example, for conjugating a PBRM having a molecular weight of 40 kDa or greater (e.g., 60 kDa or greater, 80 kDa or greater, 100 kDa or greater, 120 kDa or greater, 140 kDa or greater, 160 kDa or greater, or 180 kDa or greater, or 200 kDa or greater, or about 40-200 kDa, 40-180 kDa, 40-140 kDa, 60-200 kDa, 60-180 kDa, 60-140 kDa, 80-200 kDa, 80-180 kDa, 80-140 kDa, 100-200 kDa, 100-180 kDa, or 100-140 kDa), the polymeric carrier of the scaffold of the invention is a polyacetal, e.g., a PHF having a molecular weight (i.e., MW of the unmodified PHF) ranging from about 2 kDa to about 40 kDa (e.g., about 6-20 kDa or about 8-15 kDa).

For example, for conjugating a PBRM having a molecular weight of 40 kDa to 200 kDa, the polymeric carrier of the scaffold of the invention is a polyacetal, e.g., a PHF having a molecular weight (i.e., MW of the unmodified PHF) ranging from about 2 kDa to about 40 kDa (e.g., about 6-20 kDa or about 8-15 kDa).

For example, for conjugating a PBRM having a molecular weight of 60 kDa to 120 kDa, the polymeric carrier of the scaffold of the invention is a polyacetal, e.g., a PHF having a molecular weight (i.e., MW of the unmodified PHF) ranging from about 8 kDa to about 40 kDa (e.g., about 8-30 kDa, about 8-20 kDa or about 8-15 kDa). For example the PHF has a molecular weight of about 10 kDa, 20 kDa, 30 kDa or 40 kDa.

PBRMs in this molecular weight range, include but are not limited to, for example, camelids, scFvFc, Fab2, and the like.

For example, for conjugating a PBRM having a molecular weight of 140 kDa to 180 kDa, the polymeric carrier of the scaffold of the invention is a polyacetal, e.g., a PHF having a molecular weight (i.e., MW of the unmodified PHF) ranging from about 2 kDa to about 40 kDa (e.g., about 6-20 kDa or about 8-15 kDa). For example the PHF has a molecular weight of about 8 kDa, 10 kDa or 15 kDa.

PBRMs in this molecular weight range, include but are not limited to, for example, full length antibodies, such as, IgG and IgM.

For example, for conjugating a PBRM having a molecular weight of 200 kDa or less (e.g., 120 kDa or less, 80 kDa or less, 60 kDa or less, 40 kDa or less, 20 kDa or less or 10 kDa or less), the polymeric carrier of the scaffold of the invention is a polyacetal, e.g., a PHF having a molecular weight (i.e., MW of the unmodified PHF) ranging from about 20 kDa to about 300 kDa (e.g., about 20-150 kDa, about 30-150 kDa, about 50-150 kDa, about 30-100 kDa, or about 50-100 kDa).

For example, for conjugating a PBRM having a molecular weight of 4 kDa to 80 kDa (e.g., 4-20 kDa, 20-30 kDa, or 30-70 kDa), the polymeric carrier of the scaffold of the invention is a polyacetal, e.g., a PHF having a molecular weight (i.e., MW of the unmodified PHF) ranging from about 20 kDa to about 300 kDa (e.g., about 20-150 kDa, about 30-150 kDa, about 50-150 kDa, about 30-100 kDa, or about 50-100 kDa).

For example, for conjugating a PBRM having a molecular weight of 80 kDa or less (e.g., 70 kDa or less, 60 kDa or less, 50 kDa or less or 40 kDa or less), the polymeric carrier of the scaffold of the invention is a polyacetal, e.g., a PHF having a molecular weight (i.e., MW of the unmodified PHF) ranging from about 20 kDa to about 300 kDa (e.g., about 20-150 kDa, about 30-150 kDa, about 50-150 kDa, about 30-100 kDa, or about 50-100 kDa). For example the PHF has a molecular weight of about 50 kDa, 70 kDa or 100 kDa.

PBRMs in this molecular weight range, include but are not limited to, for example, antibody fragments such as, for example Fabs.

For example, for conjugating a PBRM having a molecular weight of 30 kDa or less (e.g., about 20 kDa or less), the polymeric carrier of the scaffold of the invention is a polyacetal, e.g., a PHF having a molecular weight (i.e., MW of the unmodified PHF) ranging from about 20 kDa to about 300 kDa (e.g., about 20-150 kDa, about 30-150 kDa, about 50-150 kDa, about 30-100 kDa, or about 50-100 kDa). For example the PHF has a molecular weight of about 30 kDa, 40 kDa, 50 kDa, 70 kDa, 100 kDa, 120 kDa or 150 kDa.

PBRMs in this molecular weight range, include but are not limited to, for example, antibody fragments, such as, scFv.

For example, for conjugating a PBRM having a molecular weight of 20 kDa or less (e.g., 10 kDa or less), the polymeric carrier of the scaffold of the invention is a polyacetal, e.g., a PHF having a molecular weight (i.e., MW of the unmodified PHF) ranging from about 20 kDa to about 300 kDa (e.g., about 20-150 kDa, about 30-150 kDa, about 50-150 kDa, about 30-100 kDa, or about 50-100 kDa). For example the PHF has a molecular weight of about 30 kDa, 40 kDa, 50 kDa, 70 kDa, 100 kDa, 120 kDa or 150 kDa.

PBRMs in this molecular weight range, include but are not limited to, for example, small proteins and peptides.

For example, the scaffold is of Formula (Ia):

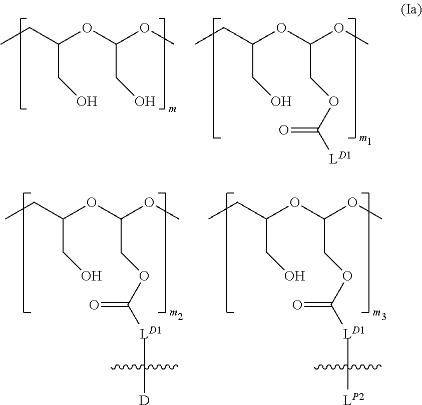

wherein:

m is an integer from 1 to about 2200,

m₁ is an integer from 1 to about 660,

m₂ is an integer from 1 to about 300,

m₃ is an integer from 1 to about 110, and

the sum of m, m₁, m₂ and m₃ ranges from about 15 to about 2200.

For example, when the PHF in Formula (Ia) has a molecular weight ranging from about 2 kDa to about 40 kDa (i.e., the sum of m, m₁, m₂, and m₃ ranging from about 15 to about 300), m₂ is an integer from 1 to about 40, m₃ is an integer from 1 to about 18, and/or m₁ is an integer from 1 to about 140 (e.g., m₁ being about 1-90).

For example, when the PHF in Formula (Ia) has a molecular weight ranging from about 6 kDa to about 20 kDa (i.e., the sum of m, m₁, m₂, and m₃ ranging from about 45 to about 150), m₂ is an integer from 2 to about 20, m₃ is an integer from 1 to about 9, and/or m₁ is an integer from 1 to about 75 (e.g., m₁ being about 4-45).

For example, when the PHF in Formula (Ia) has a molecular weight ranging from about 8 kDa to about 15 kDa (i.e., the sum of m, m₁, m₂, and m₃ ranging from about 60 to about 110), m₂ is an integer from 2 to about 15, m₃ is an integer from 1 to about 7, and/or m₁ is an integer from 1 to about 55 (e.g., m₁ being about 4-30).

For example, when the PHF in Formula (Ia) has a molecular weight ranging from 20 kDa to 300 kDa (i.e., the sum of m, m₁, m₂, and m₃ ranging from about 150 to about 2200), m₂ is an integer from 3 to about 300, m₃ is an integer from 1 to about 110, and/or m₁ is an integer from 1 to about 660 (e.g., m₁ being about 10-250).

For example, when the PHF in Formula (Ia) has a molecular weight ranging from 20 kDa to 150 kDa (i.e., the sum of m, m₁, m₂, m₃, and m₄ ranging from about 150 to about 1100), m₂ is an integer from 3 to about 150, m₃ is an integer from 1 to about 55, and/or m₁ is an integer from 1 to about 330 (e.g., m₁ being about 10-330 or about 15-100). This scaffold can be used, for example, for conjugating a PBRM having a molecular weight of about 4 kDa to about 80 kDa.

For example, when the PHF in Formula (Ia) has a molecular weight ranging from 40 kDa to 150 kDa (i.e., the sum of m, m₁, m₂, and m₃ ranging from about 300 to about 1100), m₂ is an integer from 4 to about 150, m₃ is an integer from 1 to about 75 (e.g., from 1 to about 55), and/or m₁ is an integer from 1 to about 330 (e.g., m₁ being about 15-100).

For example, when the PHF in Formula (Ia) has a molecular weight ranging from 30 kDa to 100 kDa (i.e., the sum of m, m₁, m₂, and m₃ ranging from about 220 to about 740), m₂ is an integer from 3 to 100 (e.g., 5-100), m₃ is an integer from 1 to about 40, and/or m₁ is an integer from 1 to about 220 (e.g., m₁ being about 15-80).

### Drug-Linker-Polymer Conjugates

For example, the scaffold further comprises a PBRM connected to the polymeric carrier via L^{P}.

For example, when the PHF has a molecular weight ranging from 20 kDa to 300 kDa, (e.g., about 20-150 kDa, about 30-150 kDa, about 50-150 kDa, about 30-100 kDa, or about 50-100 kDa), the number of drugs per PHF (e.g., m₂) is an integer from about 3 to about 300, (e.g., about 3 to about 150 or about 3 to about 100). This scaffold can be used, for example, for conjugating a PBRM having a molecular weight of 200 kDa or less (e.g., 80 kDa or less, 60 kDa or less, 40 kDa or less, 20 kDa or less or 10 kDa or less). In this embodiment the ratio of PBRM per PHF is between about 1:1 and about 60:1, for example, between about 1:1 and about 30:1; between about 1:1 and about 20:1, between about 1:1 and about 10:1, between about 1:1 and about 9:1, between about 1:1 and about 8:1, between about 1:1 and about 7:1, between about 1:1 and about 6:1, between about 1:1 and about 5:1, between about 1:1 and about 4:1, between about 1:1 and about 3:1, or between about 1:1 and about 2:1. See, for example, Formula (Ib).

For example, the scaffold further comprises a PBRM connected to the polymeric carrier via L^{P}. For example, one or more PBRMs are connected to one drug-carrying polymeric carrier.

For example, the scaffold (e.g., a PBRM-polymer-drug conjugate) is of Formula (Ib):

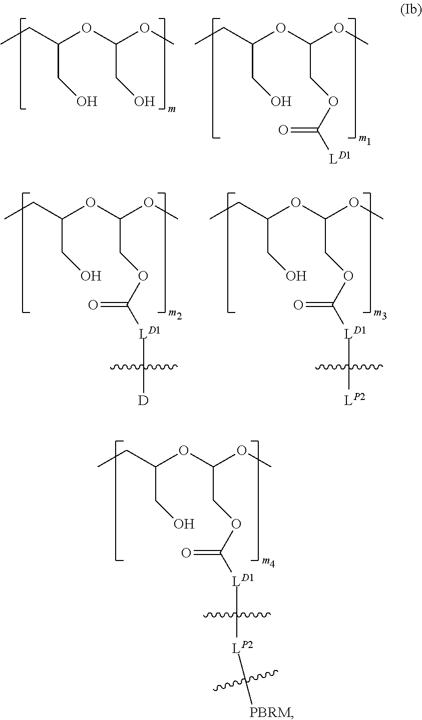

wherein:

the

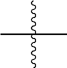

between L^{P2} and PBRM in

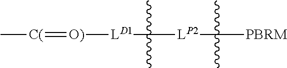

denotes direct or indirect attachment of PBRM to L^{P2}, such that the D-carrying polymeric carrier is connected to the PBRM,

each occurrence of PBRM independently has a molecular weight of less than 200 kDa (e.g., less than 80 kDa),

m is an integer from 1 to about 2200,

m₁ is an integer from 1 to about 660,

m₂ is an integer from 3 to about 300,

m₃ is an integer from 0 to about 110,

m₄ is an integer from 1 to about 60; and

the sum of m, m₁, m₂, m₃ and m₄ ranges from about 150 to about 2200.

For example, in Formula (Ib), m₁ is an integer from about 10 to about 660 (e.g., about 10-250).

For example, when the PHF in Formula (Ib) has a molecular weight ranging from 20 kDa to 150 kDa (i.e., the sum of m, m₁, m₂, m₃, and m₄ ranging from about 150 to about 1100), m₂ is an integer from 3 to about 150, m₃ is an integer from 1 to about 55, m₄ is an integer from 1 to about 30, and/or m₁ is an integer from 1 to about 330 (e.g., m₁ being about 10-330 or about 15-100). The PBRM in Formula (Ib), can have, for example, a molecular weight of about 4 kDa to about 70 kDa.

For example, when the PHF in Formula (Ib) has a molecular weight ranging from about 30 kDa to about 100 kDa (i.e., the sum of m, m₁, m₂, m₃, and m₄ ranging from about 225 to about 740), m₂ is an integer from 3 to about 100, m₃ is an integer from 1 to about 40, m₄ is an integer from 1 to about 20, and/or m₁ is an integer from 1 to about 220 (e.g., m₁ being about 15-80). In this embodiment the ratio of PBRM per PHF is between about 1:1 to 10:1, between about 1:1 and about 9:1, between about 1:1 and about 8:1, between about 1:1 and about 7:1, between about 1:1 and about 6:1, between about 1:1 and about 5:1, between about 1:1 and about 4:1, between about 1:1 and about 3:1, or between about 1:1 and about 2:1.

PBRMs in this molecular weight range, include but are not limited to, for example, small proteins and peptides.

For example, when the PHF has a molecular weight ranging from 20 kDa to 150 kDa, (e.g., 50-100 kDa), the number of drugs per PHF (e.g., m₂) is an integer from about 3 to about 150 (e.g., about 3 to about 100). This scaffold can be used, for example, for conjugating a PBRM having a molecular weight of about 30 kDa to about 70 kDa In this embodiment the ratio of PBRM per PHF is between about 1:1 and about 30:1, between about 1:1 and about 10:1, between about 1:1 and about 9:1, between about 1:1 and about 8:1, between about 1:1 and about 7:1, between about 1:1 and about 6:1, between about 1:1 and about 5:1, between about 1:1 and about 4:1, between about 1:1 and about 3:1, or between about 1:1 and about 2:1.

PBRMs in this molecular weight range, include but are not limited to, for example, antibody fragments such as, for example Fab.

Alternatively or additionally, one or more drug-carrying polymeric carriers are connected to one PBRM. For example, the scaffold (e.g., a PBRM-polymer-drug conjugate) comprises a PBRM with a molecular weight of greater than 40 kDa and one or more D-carrying polymeric carriers connected to the PBRM, in which each of the D-carrying polymeric carrier independently is of Formula (Ic):

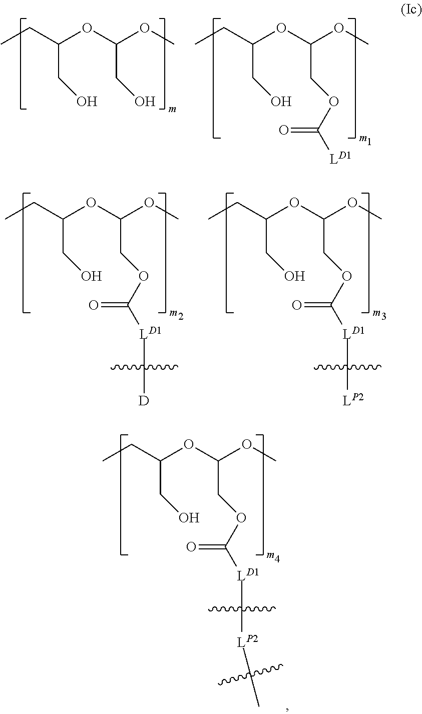

wherein:

terminal

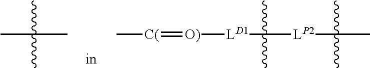

denotes direct or indirect attachment of L^{P2} to PBRM such that the D carrying polymeric carrier is connected to the PBRM,

m is an integer from 1 to 300,

m₁ is an integer from 1 to 140,

m₂ is an integer from 1 to 40,

m₃ is an integer from 0 to 18,

m₄ is an integer from 1 to 10; and

the sum of m, m₁, m₂, m₃, and m₄ ranges from 15 to 300; provided that the total number of L^{P2} attached to the PBRM is 10 or less.

For example, in Formula (Ic), m₁ is an integer from 1 to about 120 (e.g., about 1-90) and/or m₃ is an integer from 1 to about 10 (e.g., about 1-8).

For example, when the PHF in Formula (Ic) has a molecular weight ranging from about 6 kDa to about 20 kDa (i.e., the sum of m, m₁, m₂, m₃, and m₄ ranging from about 45 to about 150), m₂ is an integer from 2 to about 20, m₃ is an integer from 1 to about 9, and/or m₁ is an integer from 1 to about 75 (e.g., m₁ being about 4-45).

For example, when the PHF in Formula (Ic) has a molecular weight ranging from about 8 kDa to about 15 kDa (i.e., the sum of m, m₁, m₂, m₃, and m₄ ranging from about 60 to about 110), m₂ is an integer from 2 to about 15, m₃ is an integer from 1 to about 7, and/or m₁ is an integer from 1 to about 55 (e.g., m₁ being about 4-30).

For example, when the PHF has a molecular weight ranging from 2 kDa to 40 kDa, (e.g., about 6-20 kDa or about 8-15 kDa), the number of drugs per PHF (e.g., m₂) is an integer from 1 to about 40, (e.g., about 2-20 or about 2-15). This scaffold can be used, for example, for conjugating a PBRM having a molecular weight of 40 kDa or greater (e.g., 60 kDa or greater; 80 kDa or greater; or 100 kDa or greater; 120 kDa or greater; 140 kDa or greater; 160 kDa or greater or 180 kDa or greater). In this embodiment the ratio of PBRM per PHF is between about 1:1 and about 1:10, between about 1:1 and about 1:9, between about 1:1 and about 1:8, between about 1:1 and about 1:7, between about 1:1 and about 1:6, between about 1:1 and about 1:5, between about 1:1 and about 1:4, between about 1:1 and about 1:3, or between about 1:1 and about 1:2.

For example, when the PHF has a molecular weight ranging from 2 kDa to 40 kDa, (e.g., about 6-20 kDa or about 8-15 kDa), the number of drugs per PHF (e.g., m₂) is an integer from 1 to about 40, (e.g., about 1:10 or about 1-15). This scaffold can be used, for example, for conjugating a PBRM having a molecular weight of 140 kDa to 180 kDa. In this embodiment the ratio of PBRM per PHF is between about 1:1 and about 1:10, between about 1:1 and about 1:9, between about 1:1 and about 1:8, between about 1:1 and about 1:7, between about 1:1 and about 1:6, between about 1:1 and about 1:5, between about 1:1 and about 1:4, between about 1:1 and about 1:3, or between about 1:1 and about 1:2.

PBRMs in this molecular weight range, include but are not limited to, for example, full length antibodies, such as, IgG and IgM.

For example, when the PHF has a molecular weight ranging from 2 kDa to 40 kDa, the number of drugs per PHF (e.g., m₂) is an integer from 1 to about 40, (e.g., about 1:20 or about 1:15). This scaffold can be used, for example, for conjugating a PBRM having a molecular weight of 60 kDa to 120 kDa. In this embodiment the ratio of PBRM per PHF is between about 1:1 and about 1:10, between about 1:1 and about 1:9, between about 1:1 and about 1:8, between about 1:1 and about 1:7, between about 1:1 and about 1:6, between about 1:1 and about 1:5, between about 1:1 and about 1:4, between about 1:1 and about 1:3, or between about 1:1 and about 1:2.

PBRMs in this molecular weight range, include but are not limited to, for example, antibody fragments such as, for example Fab2 and camelids.

In one embodiment the protein-polymer tubulysin compound conjugate comprises a PBRM having a molecular weight of about 140 kDa to about 180 kDa (e.g., an antibody), the PHF has a molecular weight of about 8 to 15 kDa, and a load range of about 1 to about 15 of a tubulysin compound.

In one embodiment the protein-polymer tubulysin compound conjugate comprises a PBRM having a molecular weight of about 60 kDa to about 120 kDa (e.g., Fab2, camelids), the PHF has a molecular weight of about 8 to 40 kDa, and a load range of about 1 to about 20 of a tubulysin compound.

In one embodiment the protein-polymer tubulysin compound conjugate comprises a PBRM having a molecular weight of about 30 kDa to about 70 kDa (e.g., Fab), the PHF has a molecular weight of about 50 to 100 kDa, and a load range of about 5 to about 100 of a tubulysin compound.

In one embodiment the protein-polymer tubulysin compound conjugate comprises a PBRM having a molecular weight of about 20 kDa to about 30 kDa (e.g., scFv), the PHF has a molecular weight of about 50 to 150 kDa, and a load range of about 5 to about 150 of a tubulysin compound.

In one embodiment the protein-polymer tubulysin compound conjugate comprises a PBRM having a molecular weight of about 4 kDa to about 20 kDa (e.g., a small protein), the PHF has a molecular weight of about 50 to 150 kDa, and a load range of about 5 to about 150 of a tubulysin compound.

In some embodiments, the protein-polymer tubulysin compound conjugate includes PHF having a MW of up to 60 kDa (e.g., up to 50 kDa) and a drug to PHF ratio of up to 50:1 (e.g., about 45:1, 40:1, or 35:1).

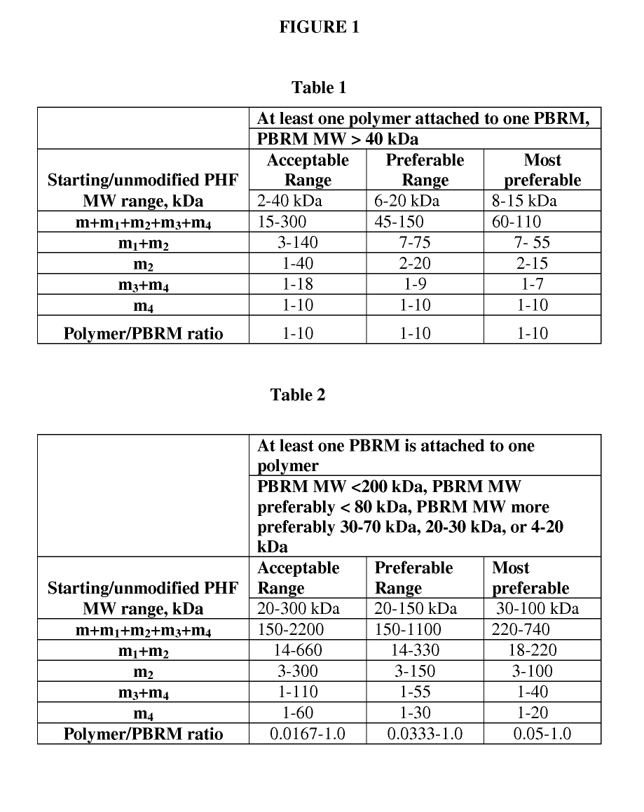

In some embodiments, the protein-polymer tubulysin compound conjugate is one of those characterized by Table 1 of FIG. 1.

In some embodiment, the protein-polymer tubulysin compound conjugate is one of those characterized by Table 2 of FIG. 1.

In some embodiments, the polymeric scaffold (e.g., a polyacetal polymer such as PHF) is conjugated with PBRMs by utilizing random lysine modification. In other embodiments, the polymeric scaffold (e.g., a polyacetal polymer such as PHF) is conjugated with PBRMs by utilizing cysteine-based bioconjugation strategy. See, e.g., WO2010100430 and U.S. Pat. No. 7,595,292, the contents of which are hereby incorporated by reference in their entireties. In one embodiment, the polymeric scaffold (e.g., a polyacetal polymer such as PHF) conjugates with a PBRM (e.g., an antibody) via cysteine residues in the antibody hinge region. Without wishing to be bound by the theory, the resulting conjugate is stabilized through the formation of inter-chain bridge structures.

Accordingly, the invention also relates to a polymeric scaffold comprising at least two -G^{X} moieties connected to the polymeric scaffold, in which each -G^{X} is capable of conjugation to a thiol group from an amino acid (e.g., cysteine) in a PBRM so as to form a protein-polymer conjugate. In embodiments, -G^{X} is a maleimide group, a disulfide group, a thiol group, a triflate group, a tosylate group, an aziridine group, a 5-pyridyl functional group, a vinylsulfone group, a vinyl pyridine group, an alkyl halide group, an acrylate group or a methacrylate group.

In embodiments, one or more free thiol groups of a PBRM are produced by reducing a protein. The one or more free thiol groups of the PBRM then react with the at least two -G^{X} moieties contained in the polymer scaffold so as to conjugate the PBRM with the polymer scaffold.

In embodiments, the free thiol groups of the PBRM that are used for the conjugation are derived from a disulfide bridge of a native protein or a disulfide bridge of a protein complex consisting of two or more protein chains connected by the disulfide bridge. A disulfide bridge may be intrachain or interchain bridge. Alternatively, the free thiol groups of the PBRM are from cysteine residues or the unpaired thiol groups of the native protein that are not involved in inter or intra disulfide bridge formation.

Disulfide bonds can be reduced, for example, with dithiothreitol, mercaptoethanol, tris-carboxyethylphosphine, dehydroascorbic acid, copper sulfate, using conventional methods. A protein can contain one or more disulfide bridges. Reduction to give free thiol groups can be controlled to reduce one or more specific disulfide bridges in a protein. Depending on the extent of disulfide reduction and the stoichiometry of the -G^{X} moieties on the polymeric scaffold polymeric, it is possible to conjugate one or more polymer scaffolds to the protein. Immobilized reducing agents may be used if it is desired to reduce less than the total number of disulfides, as can partial reduction using different reaction conditions or the addition of denaturants.

Advantages of conjugating a polymer to a protein via a thiol include, but are not limited to optimized efficacy, improved dose to dose consistency and homogeneity (as the number of conjugated polymer molecules per protein is the substantially the same for each protein molecule), specific conjugation directed to a specific residue or residues on each protein, and easier purification. Also, the protein-polymer conjugates via the thiol conjugation exhibits substantially improved half-life, mean residence time, and/or clearance rate in circulation as compared to the unconjugated protein.

In one embodiment, the scaffold for conjugating to thiol groups in a PBRM is of Formula (IIIaa):

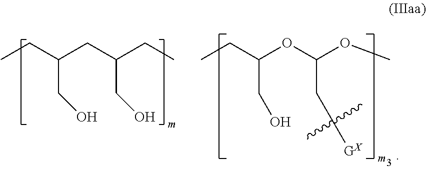

The wavy line in Formula (IIIaa) above denotes direct or indirect attachment of -G^{X} to the backbone of PHF. m and m₃ are as defined herein. For example, -G^{X} is connected to the polymeric scaffold by a linker -L^{S} having the structure:

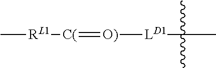

with R^{L1} and L^{D1} defined as herein and

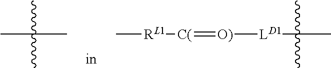

denoting direct or indirect attachment of L^{D1} to G^{X}.

For example, m is an integer from 1 to 2200.

For example, m₃ is an integer from 2 to 20 (e.g., an integer from 2 to 10, or an integer from 2 to 6).

In another embodiment, the scaffold for conjugating to thiol groups in a PBRM is of Formula (IIIbb):

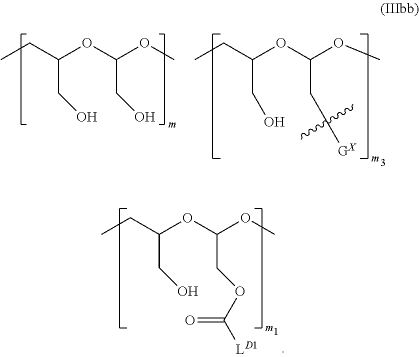

The wavy line

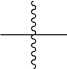

in Formula (IIIbb) above denotes direct or indirect attachment of -G^{X} to the backbone of PHF. For example, -G^{X} is connected to the polymeric scaffold by a linker -L^{S} having the structure:

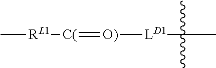

with R^{L1} and L^{D1} defined as herein and

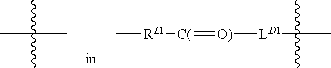

denoting direct or indirect attachment of L^{D1} to G^{X}, and m, m₁, and m₃ are as defined herein.

For example, m is an integer from 1 to 2200.

For example, m₃ is an integer from 2 to 20 (e.g., an integer from 2 to 10, or an integer from 2 to 6).

For example, m₁ is an integer from 1 to 660.

In yet another embodiment, the scaffold for conjugating to thiol groups in a PBRM is of Formula (IIIcc):

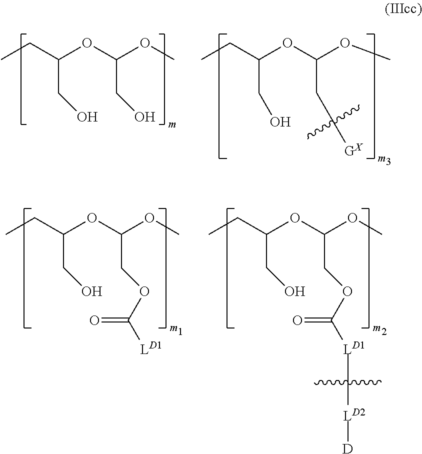

The wavy line

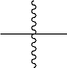

in Formula (IIIcc) above denotes direct or indirect attachment of -G^{X} to the backbone of PHF. For example, -G^{X} is connected to the polymeric scaffold by a linker -L^{S} having the structure:

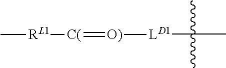

with R^{L1} and L^{D1} defined as herein and

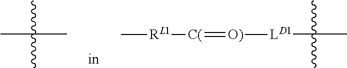

denoting direct or indirect attachment of L^{D1} to G^{X}, and L^{D2}, D, m, m₁, m₂, and m₃ are as defined herein.

For example, m is an integer from 1 to 2200.

For example, m₃ is an integer from 2 to 20 (e.g., an integer from 2 to 10, or an integer from 2 to 6).

For example, m₁ is an integer from 1 to 660.

For example, m₂ is an integer from 1 to 300.

In some embodiments, the tubulysin compound-polymer-PBRM conjugates, tubulysin compound-polymer conjugates, tubulysin compound carrying-polymeric scaffolds, or PBRM-carrying polymer scaffolds described herein each have a polydispersity index (PDI) of less than 2 (e.g., less than 1.5).

PBRM-tubulysin compound-polymer conjugates, tubulysin compound carrying-polymeric scaffolds, or PBRM-carrying polymer scaffolds can be purified (i.e., removal of residual unreacted tubulysin compound, PBRM, or polymeric starting materials) by extensive diafiltration. If necessary, additional purification by size exclusion chromatography can be conducted to remove any aggregated PBRM-tubulysin compound polymer conjugates. In general, the PBRM-drug polymer conjugates as purified typically contain <5% aggregated PBRM-tubulysin compound polymer conjugates as determined by SEC or SDS-PAGE; <1% polymer-drug conjugate as determined by SEC and <2% unconjugated PBRM as determined by HPLC.

Tables G and H below provide examples of the drug-carrying polymeric scaffolds and the polymer-drug-protein conjugates of the invention respectively.

TABLE G

| Ref # | Structure |
|---|---|
| | 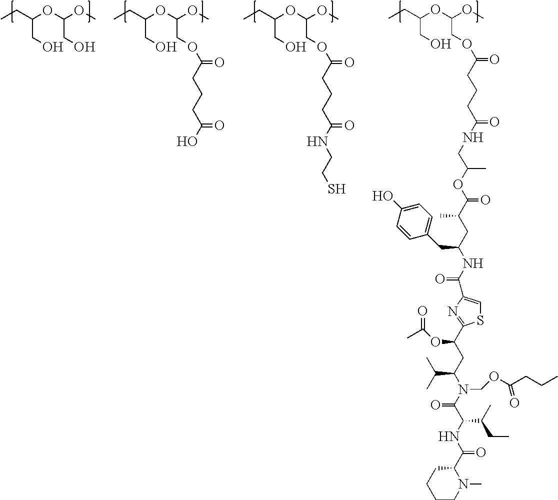 |
| Ex 18 | 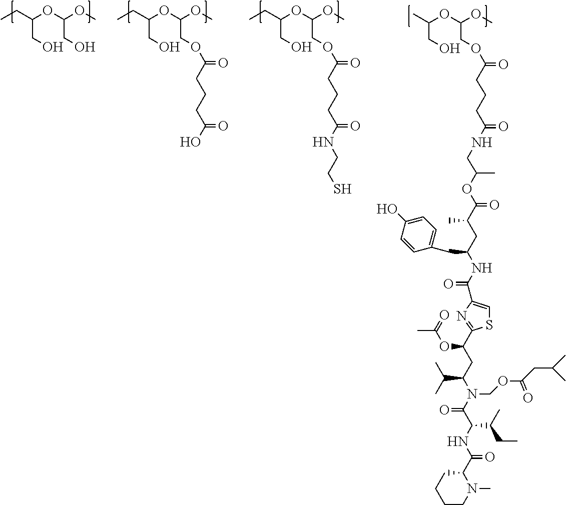 |
| Ex 21 | 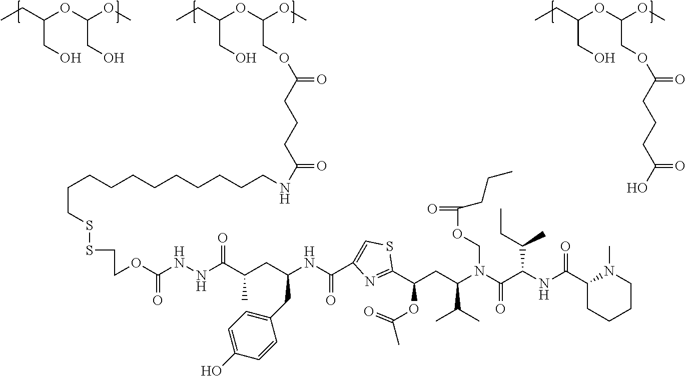 |
| | 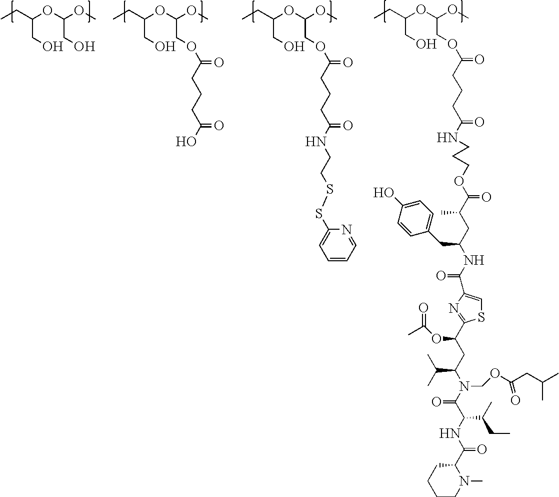 |
| | 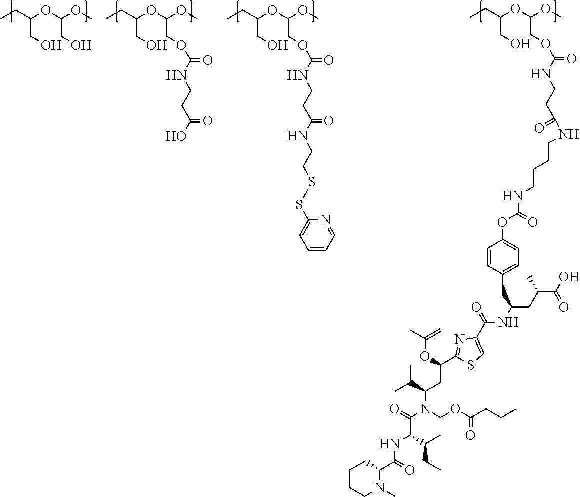 |
| Ex 27 | 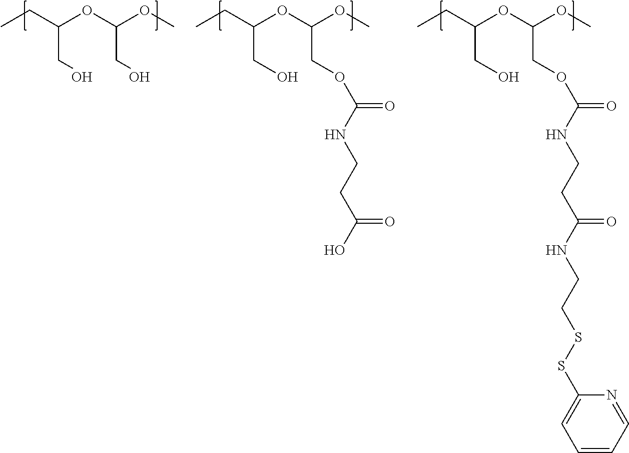 |
| | 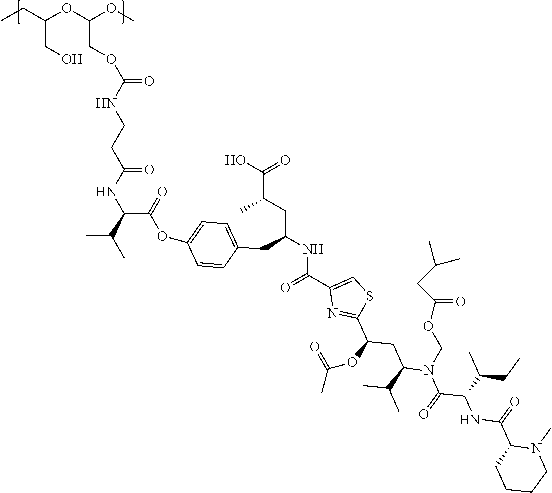 |
| Ex 30 | 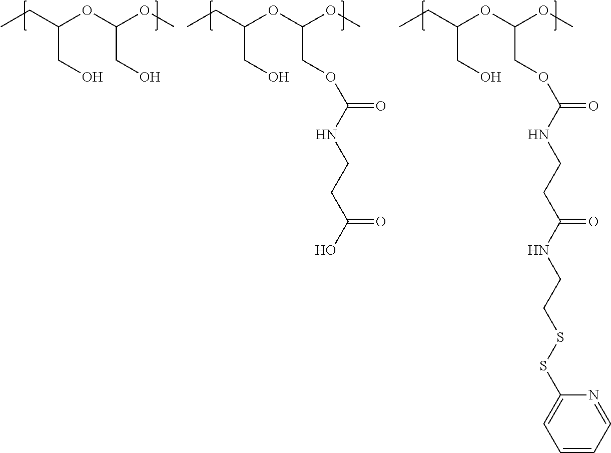 |
| | 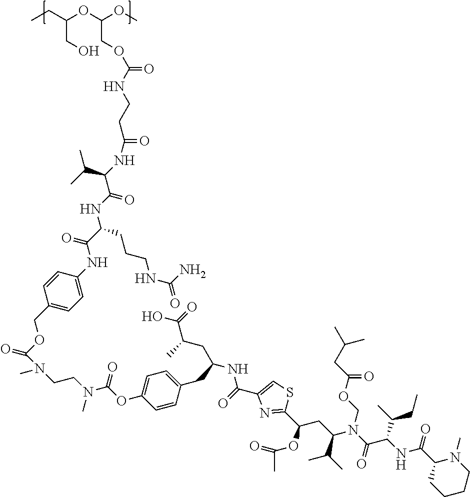 |
| | 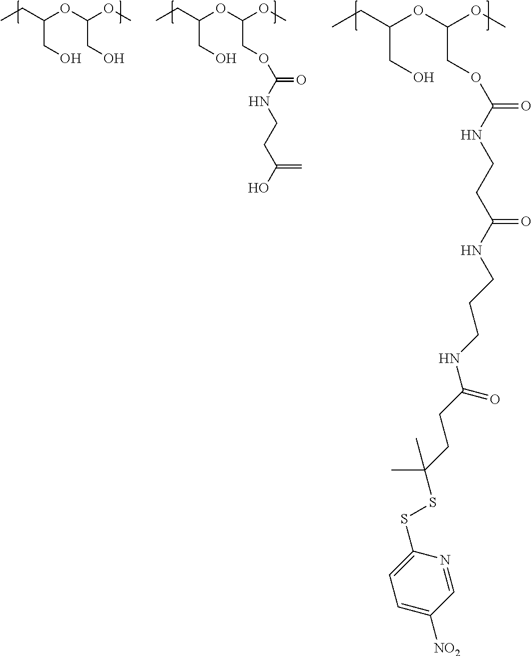 |
| | 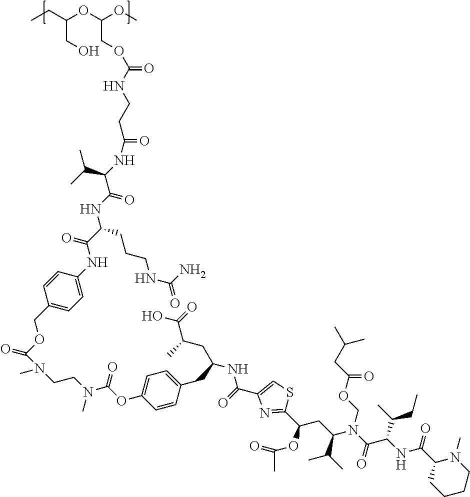 |
| | 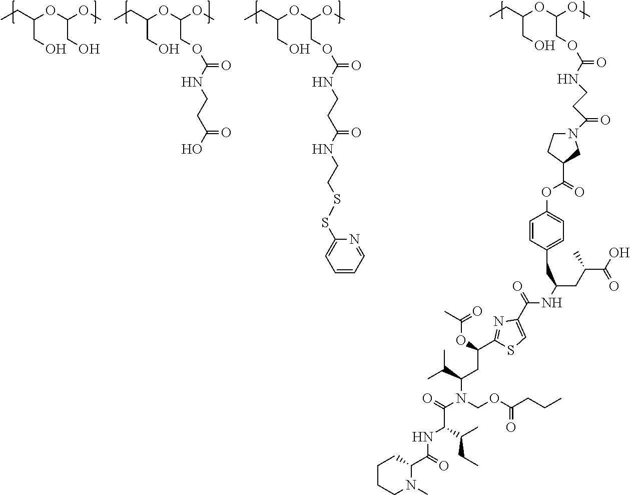 |
| | 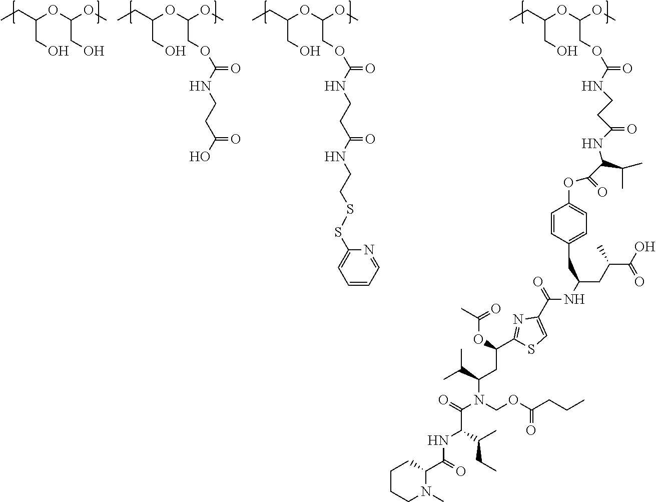 |
| Ex 24 | 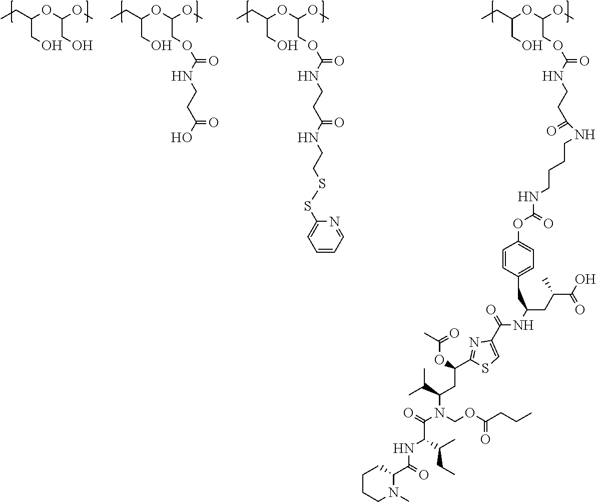 |
| Ex 34 | 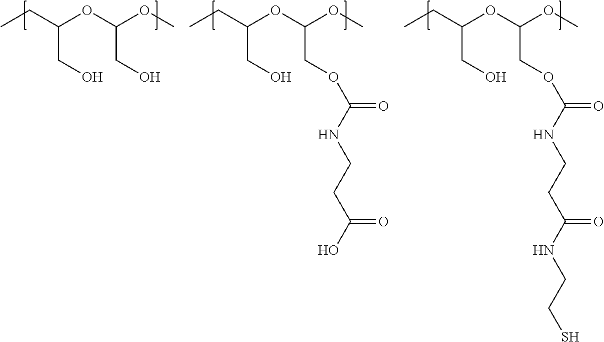 |
| | 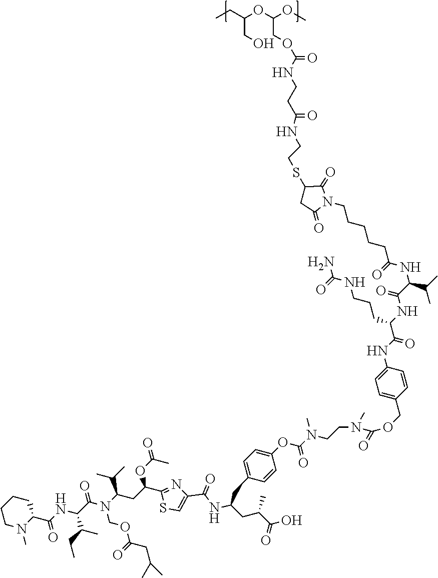 |

TABLE H

| Ref | Structure |
|---|---|
| | 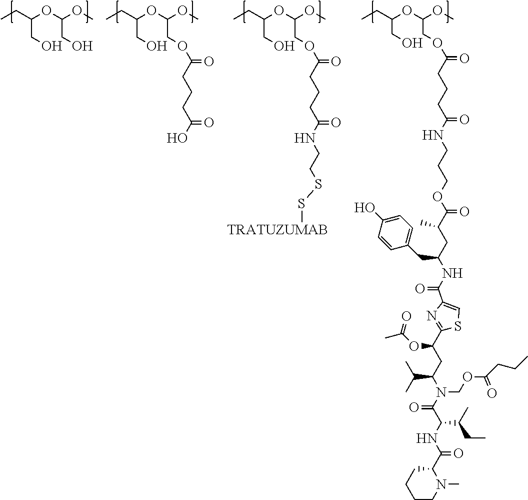 |
| | 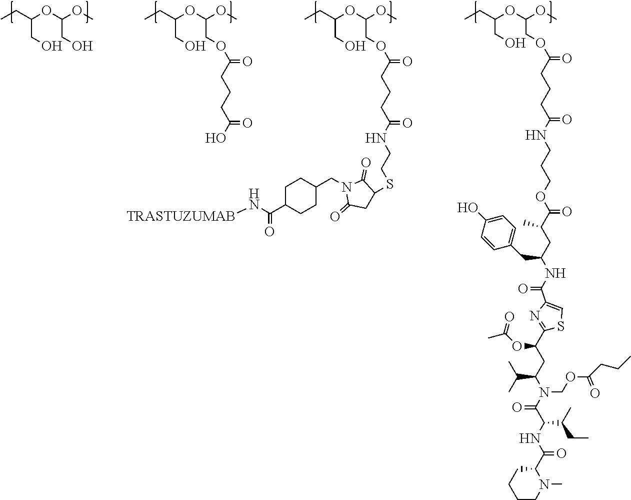 |
| | 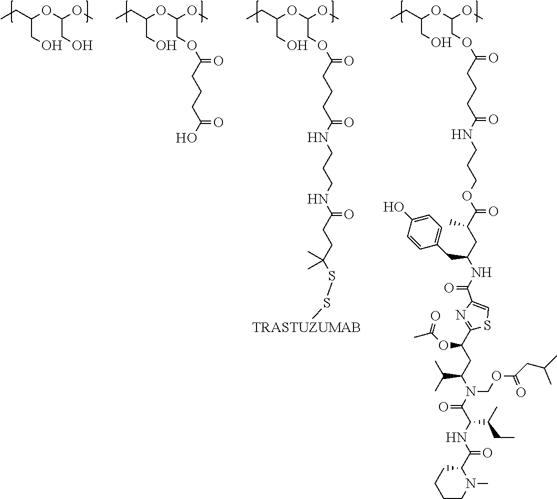 |
| | 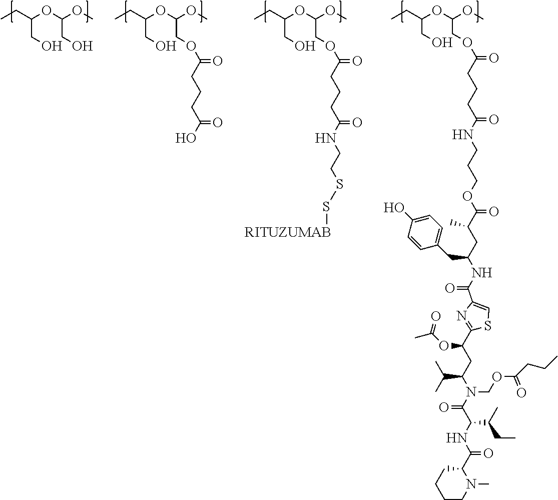 |
| | 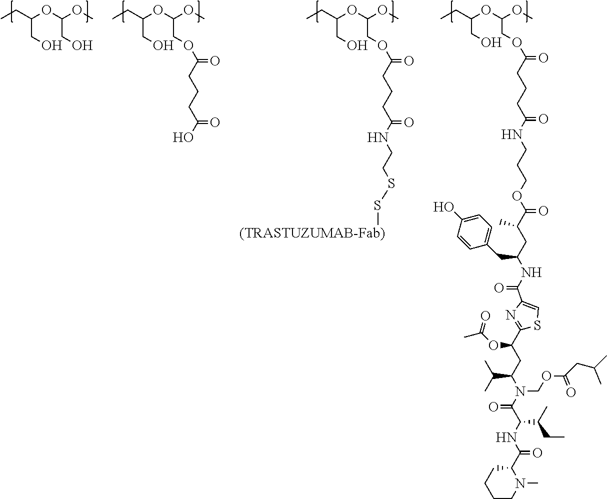 |
| | 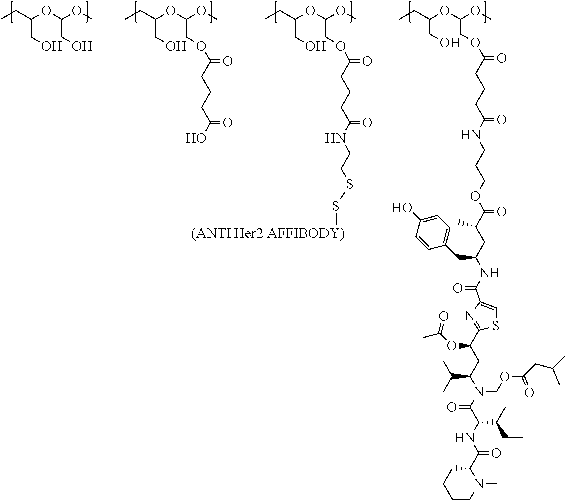 |
| Ex 25 | 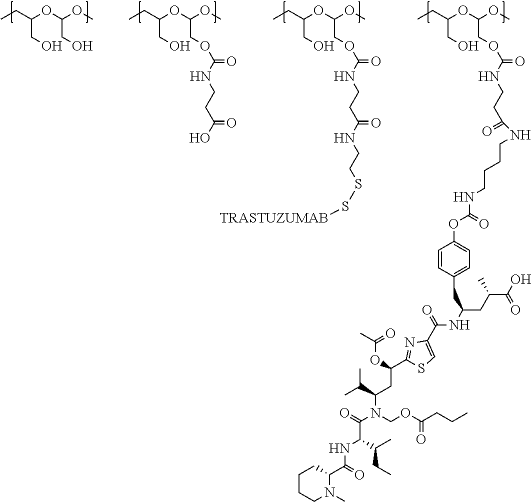 |
| Ex 28 | 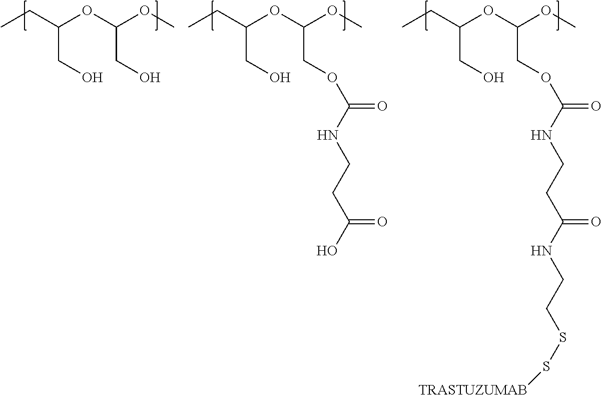 |
| | 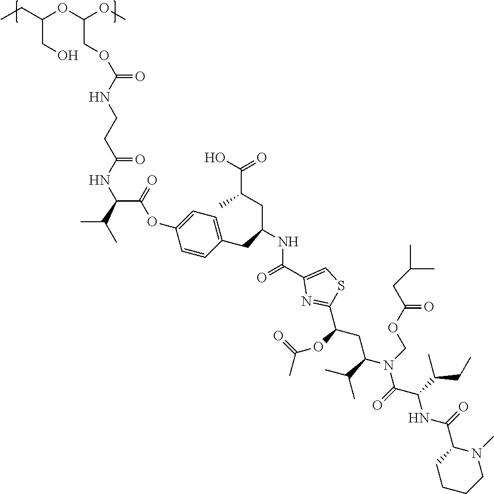 |
| Ex 31 | 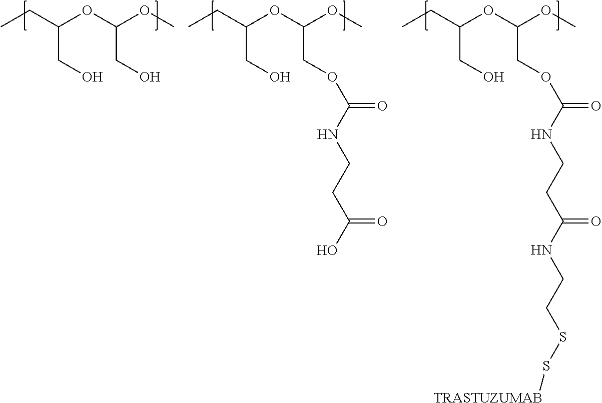 |
| | 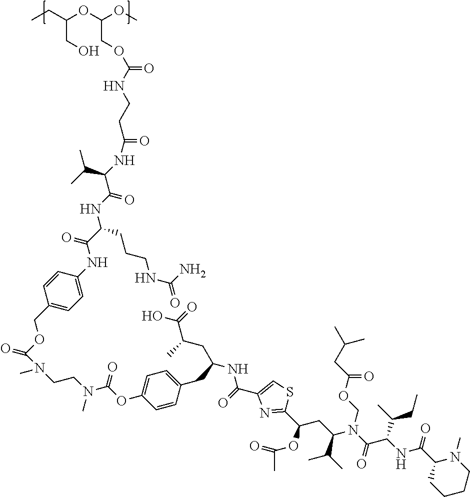 |
| Ex 35 | 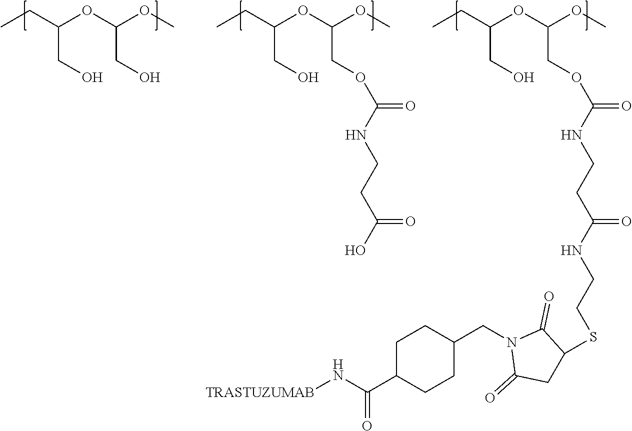 |
| | 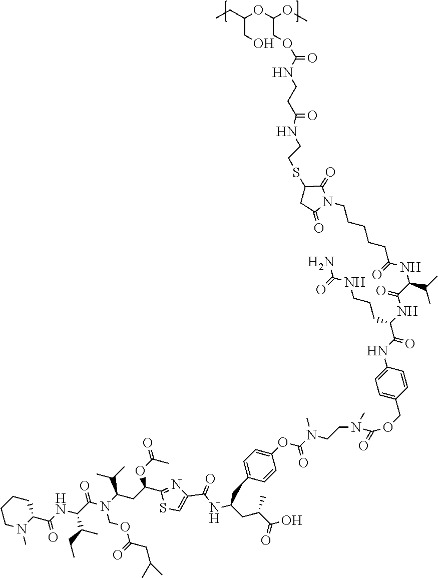 |
| Ex 36 | 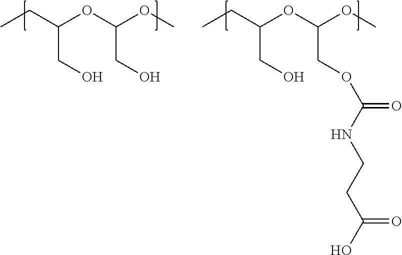 |
| | 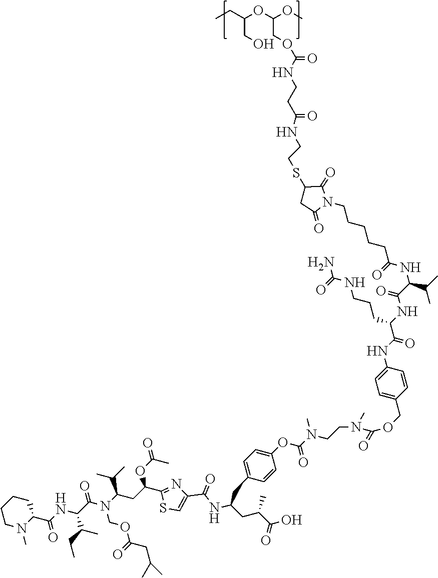 |
| | 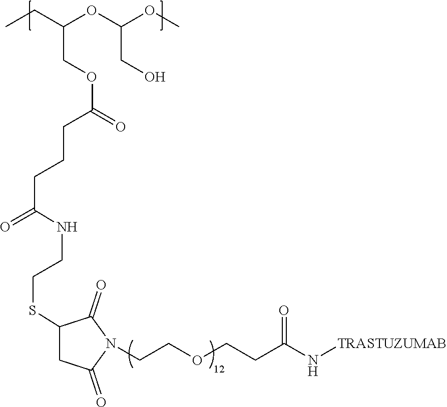 |

### Synthetic Methods

According to the present invention, any available techniques can be used to make the inventive conjugates or compositions including them, and intermediates and components (e.g., carriers and modifiers) useful for making them. For example, semi-synthetic and fully synthetic methods such as those discussed in detail below may be used.

### Carriers

Methods for preparing polymer carriers (e.g., biocompatible, biodegradable polymer carriers) suitable for conjugation to modifiers are known in the art. For example, synthetic guidance can be found in U.S. Pat. Nos. 5,811,510; 5,863,990; 5,958,398; 7,838,619; and

7,790,150; and U.S. Publication No. 2012/0321583 and 2013/0101546. The skilled practitioner will know how to adapt these methods to make polymer carriers for use in the practice of the invention.

For example, semi-synthetic polyals may be prepared from polyaldoses and polyketoses via complete lateral cleavage of carbohydrate rings with periodate in aqueous solutions, with subsequent conversion into hydrophilic moieties (e.g., via borohydride reduction) for conjugation of hydroxyl groups with one or more drug molecules or PBRMs, via a dicarboxylic acid linker (e.g., glutaric acid or β-alanine linker). In an exemplary embodiment, the carbohydrate rings of a suitable polysaccharide can be oxidized by glycol-specific reagents, resulting in the cleavage of carbon-carbon bonds between carbon atoms that are each connected to a hydroxyl group. An example of application of this methodology to dextran B-512 is illustrated below:

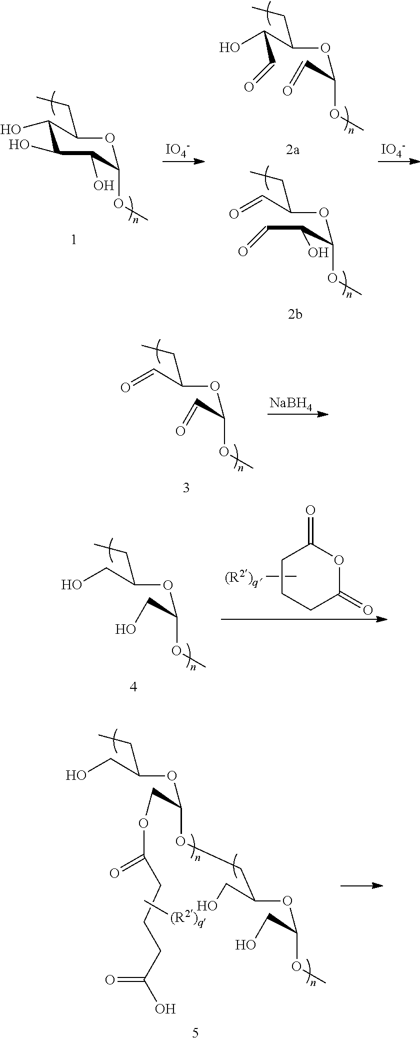

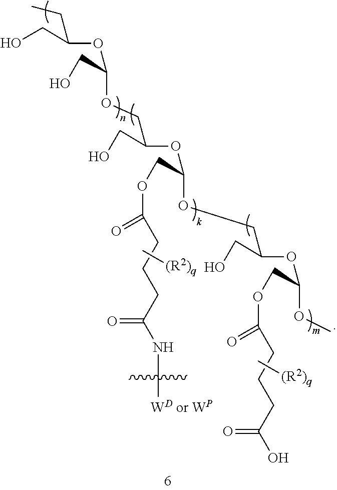

A similar approach may be used with Levan:

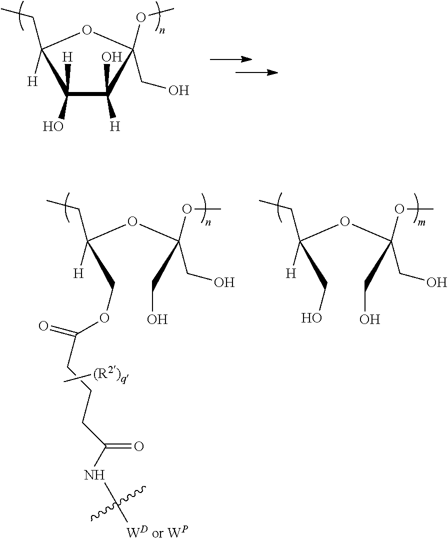

### and Inulin:

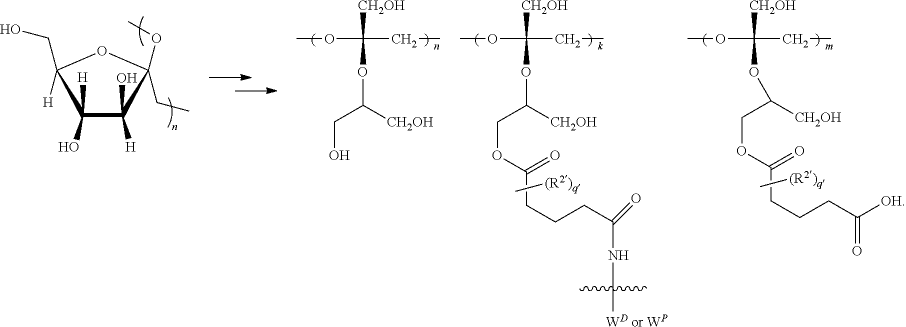

In the above schemes, the wavy bond indicates that W^{D} or W^{P} are connected directly as shown or via another moiety such as M^{D2} or M^{P2} respectively.

In the above schemes, q′ is an integer from 0 to 4; and each occurrence of R^{2′} is independently hydrogen, halogen, —CN, NO₂, an aliphatic, heteroaliphatic, carbocyclic, or heterocycloalkyl moiety, or -GR^{G1} wherein G is —O—, —S—, —NR^{G2}—, —C(═O)—, —S(═O)—, —SO₂—, —C(═O)O—, —C(═O)NR^{G2}—, —OC(═O)—, —NR^{G2}C(═O)—, —OC(═O)O—, —OC(═O)NR^{G2}—, —NR^{G2}C(═O)O—, —NR^{G2}C(═O)NR^{G2}—, —C(═S)—, —C(═S)S—, —SC(═S)—, —SC(═S)S—, —C(═NR^{G2})—, —C(═NR^{G2})O—, —C(═NR^{G2})NR^{G3}—, —OC(═NR^{G2})—, —NR^{G2}C(═NR^{G3})—, —NR^{G2}SO₂—, —NR^{G2}SO₂NR^{G3}—, or —SO₂NR^{G2}—, wherein each occurrence of R^{G1}, R^{G2} and R^{G3} is independently hydrogen, halogen, or an aliphatic, heteroaliphatic, carbocyclic, or heterocycloalkyl moiety, each of which is optionally substituted.

In certain embodiments, each occurrence of R^{2′} is independently hydrogen, alkyl, alkenyl, alkynyl, cycloalkyl, heteroalkyl, heteroalkenyl, heteroalkynyl, heterocycloalkyl, aryl, heteroaryl, —C(═O)R^{2A} or —ZR^{2A}, wherein Z is O, S, NR^{2B}, wherein each occurrence of R^{2A} and R^{2B} is independently hydrogen, or an alkyl, alkenyl, alkynyl, cycloalkyl, heteroalkyl, heteroalkenyl, heteroalkynyl, heterocycloalkyl, aryl or heteroaryl moiety. In certain embodiments, each occurrence of R² is hydrogen. In certain embodiments, one or more occurrences of R^{2′} is a C₁₋₁₀ alkyl moiety. In certain embodiments, one or more occurrences of R^{2′} is lower alkyl. In certain embodiments, one or more occurrences of R^{2′} is a hydrophobic group. In certain embodiments, one or more occurrences of R^{2′} is a hydrophilic group. In certain embodiments, one or more occurrences of R² is an anionic group. In certain embodiments, one or more occurrences of R^{2′} is a cationic group. In certain embodiments, one or more occurrences of R^{2′} is a receptor ligand.

In one embodiment, a method for forming the biodegradable biocompatible polyal conjugates of the present invention comprises a process by which a suitable polysaccharide is combined with an efficient amount of a glycol-specific oxidizing agent to form an aldehyde intermediate. The aldehyde intermediate, which is a polyal itself, may then be reduced to the corresponding polyol, succinylated, and coupled with one or more suitable modifiers to form a biodegradable biocompatible polyal conjugate comprising succinamide-containing linkages.

In another preferred embodiment, fully synthetic biodegradable biocompatible polyals for used in the present invention can be prepared by reacting a suitable initiator with a suitable precursor compound.

For example, fully synthetic polyals may be prepared by condensation of vinyl ethers with protected substituted diols. Other methods, such as cycle opening polymerization, may be used, in which the method efficacy may depend on the degree of substitution and bulkiness of the protective groups.

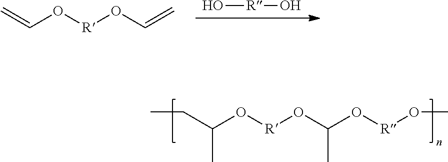

One of ordinary skill in the art will appreciate that solvent systems, catalysts and other factors may be optimized to obtain high molecular weight products.

In certain embodiments, the carrier is PHF.

In embodiments, the polymer carrier is PHF having a polydispersity index (PDI) of less than 2 (e.g., less than 1.5).

### Tubulysin Compounds

This invention also relates to a tubulysin compound so modified that it can be directly conjugated to a PBRM absent a polymeric carrier, and the drug-PBRM conjugates thereof. For example, the drug derivative is a compound of Formula (X), wherein R₄₇ comprises a terminal maleimido group, i.e.,

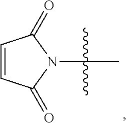 [SMILES: *N1C(=O)C=CC1=O]

and m₅ is an integer between 1 and 20.

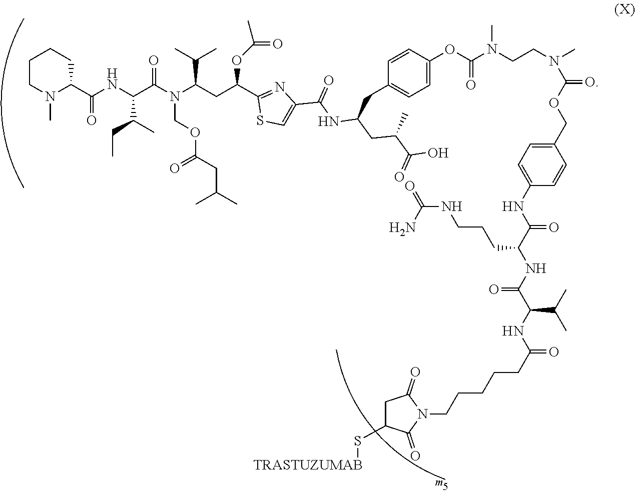

### Conjugates or Polymeric Scaffolds

The general methods of producing the conjugates or polymeric scaffolds of this invention have been described above. Schemes 1-7 below exemplify how the conjugates or polymeric scaffolds are synthesized. The variables (e.g., X, X^{D}, X^{P}, L^{D1}, and L^{P2} etc) in these schemes have the same definitions as described herein unless otherwise specified. Each W^{D1} is a function moiety that is capable of reacting with W^{D} to form Z^{D}-M^{D3} and each W^{P1} is a function moiety that is capable of reacting with W^{P} to form Z^{P}-M^{P3}. —X^{D}-M^{D1}-Y^{D}-M^{D2}-W^{D} and —X^{P}-M^{P1}-Y^{P}-M^{P2}-W^{P} may be different (such as in Schemes 5 and 5A) or the same (such as in Scheme 6). In some embodiments —X^{P}-M^{P1}-Y^{P}-M^{P2}-W^{P} is formed by further modification of —X^{D}-M^{D1}-Y^{D}-M^{D2}-W^{D}

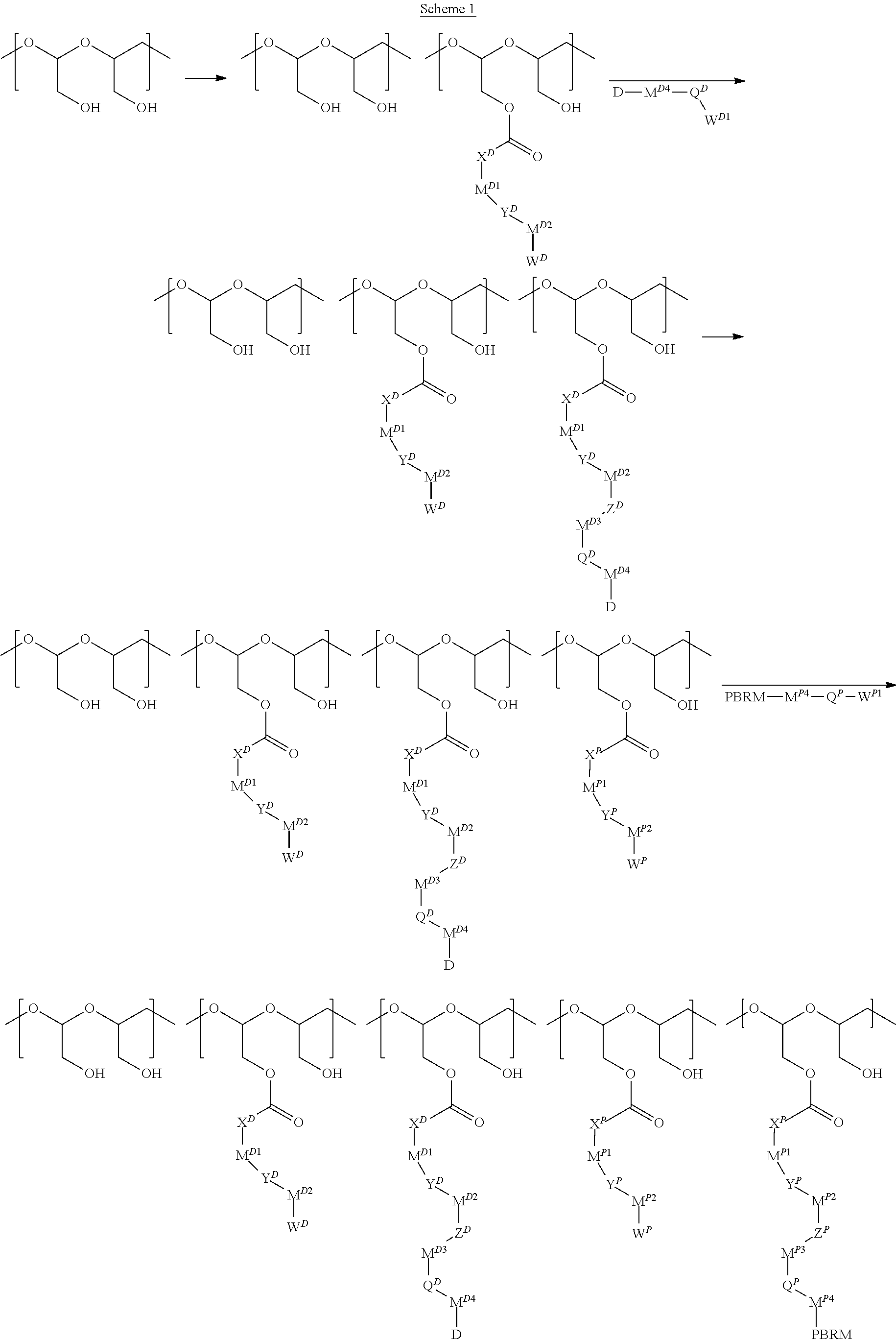

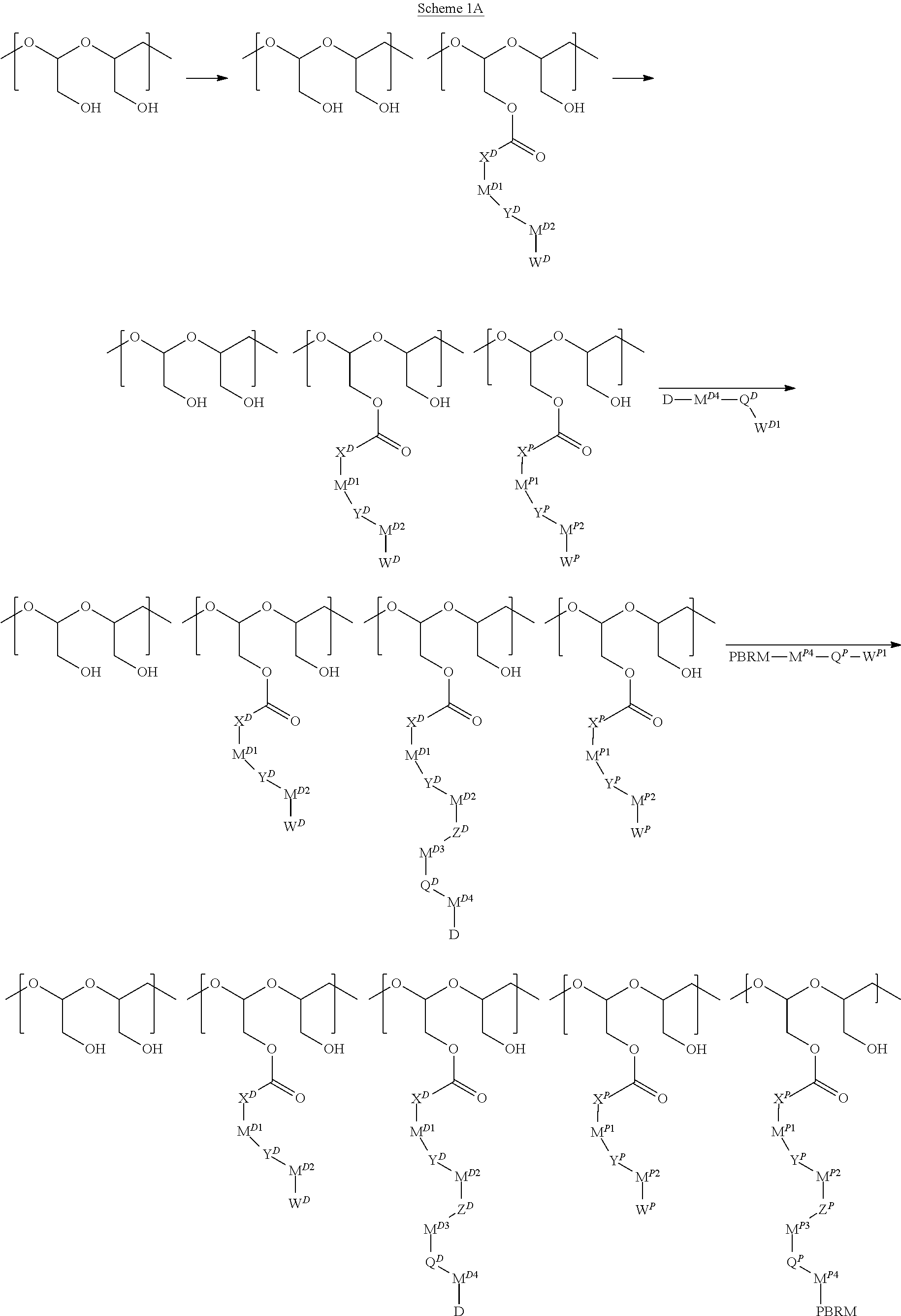

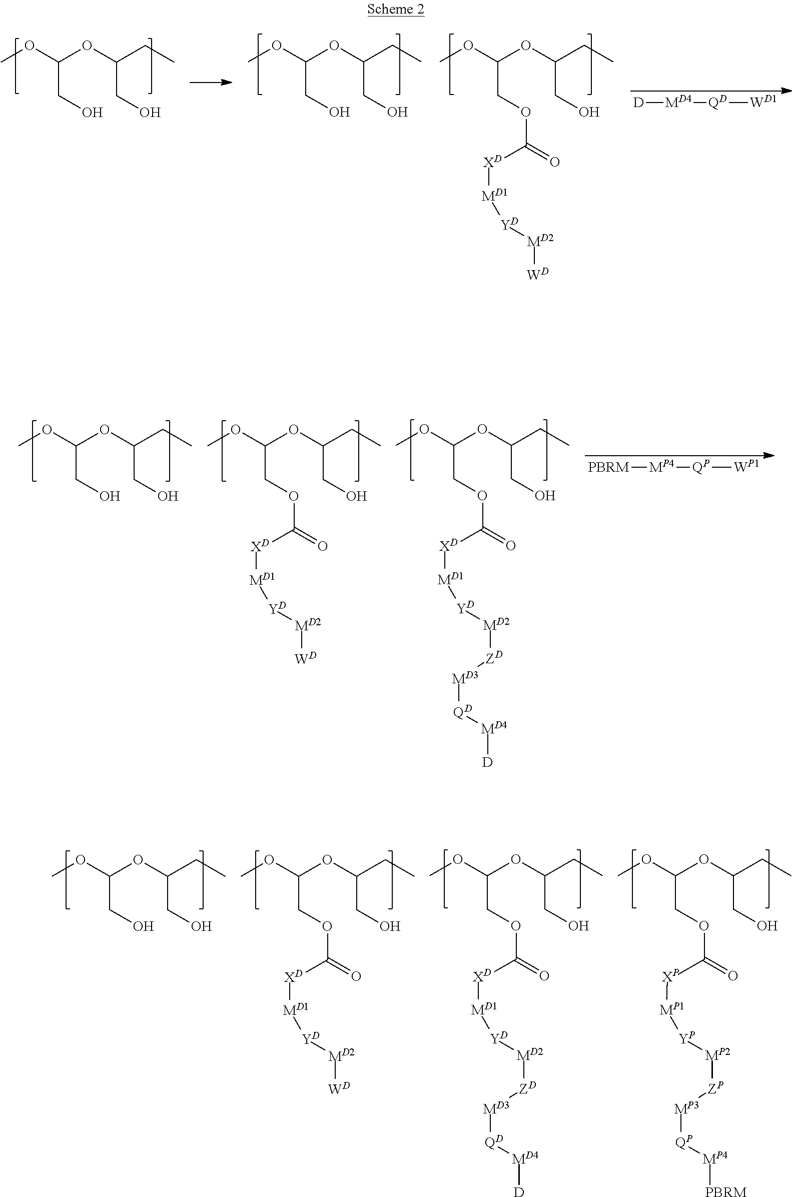

The PBRM can be linked to the drug-polymer conjugate to form the protein-polymer-drug conjugate using standard synthetic methods for protein conjugation, including, but not limited to, reactions based on reductive amination, Staudinger ligation, oxime formation, thiazolidine formation and the methods and reactions described herein.

Scheme 3 below shows the synthesis of a PBRM-drug-polymer conjugate in which the PBRM is linked to the drug polymer conjugate using click chemistry.

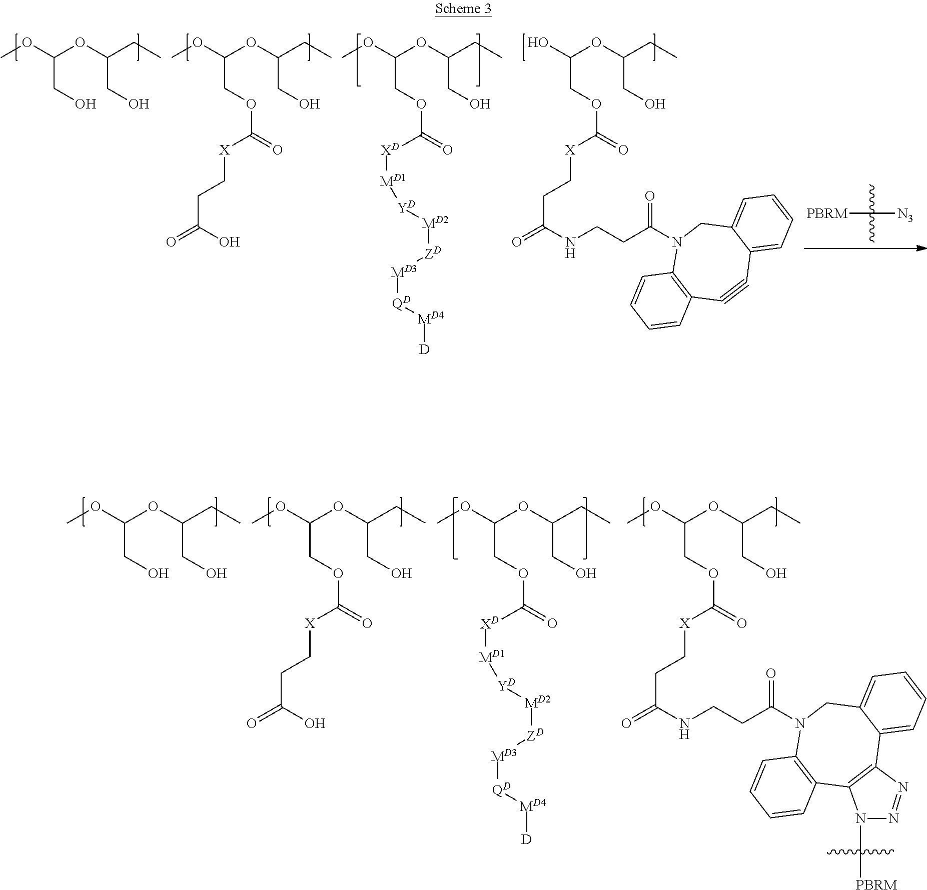

Scheme 4 below shows the synthesis of a PBRM-drug-polymer conjugate is which the PBRM is linked to the drug polymer conjugate by a Mannich reaction.

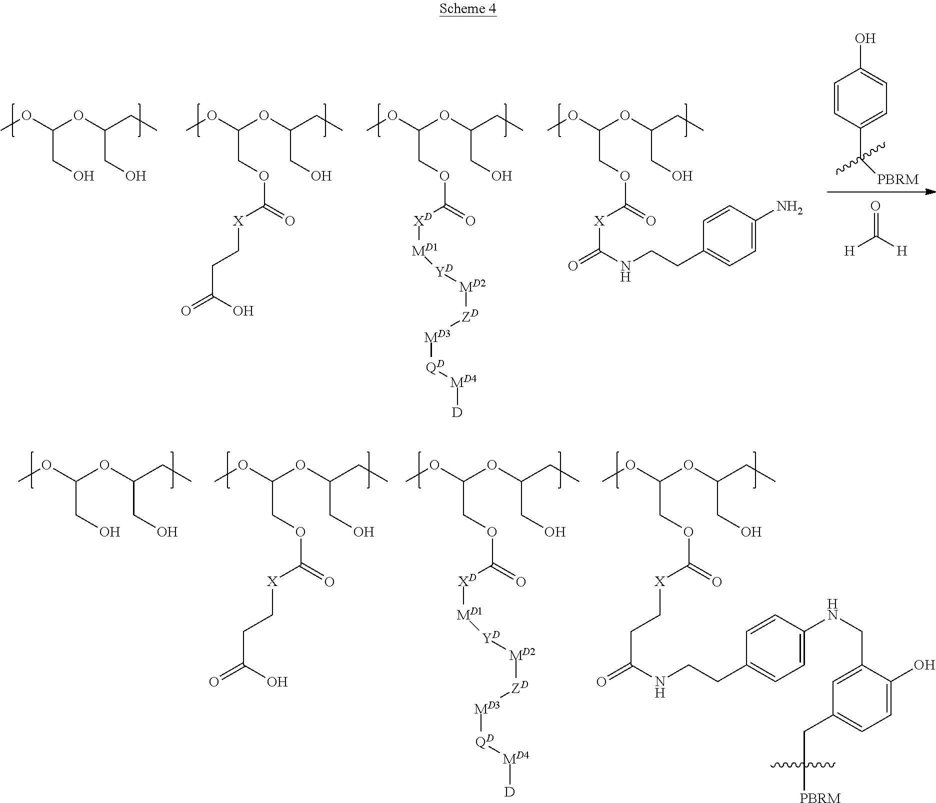

Scheme 5 below shows the synthesis of a PBRM-drug-polymer conjugate is which the PBRM is linked to the drug polymer conjugate by palladium catalyzed cross coupling.

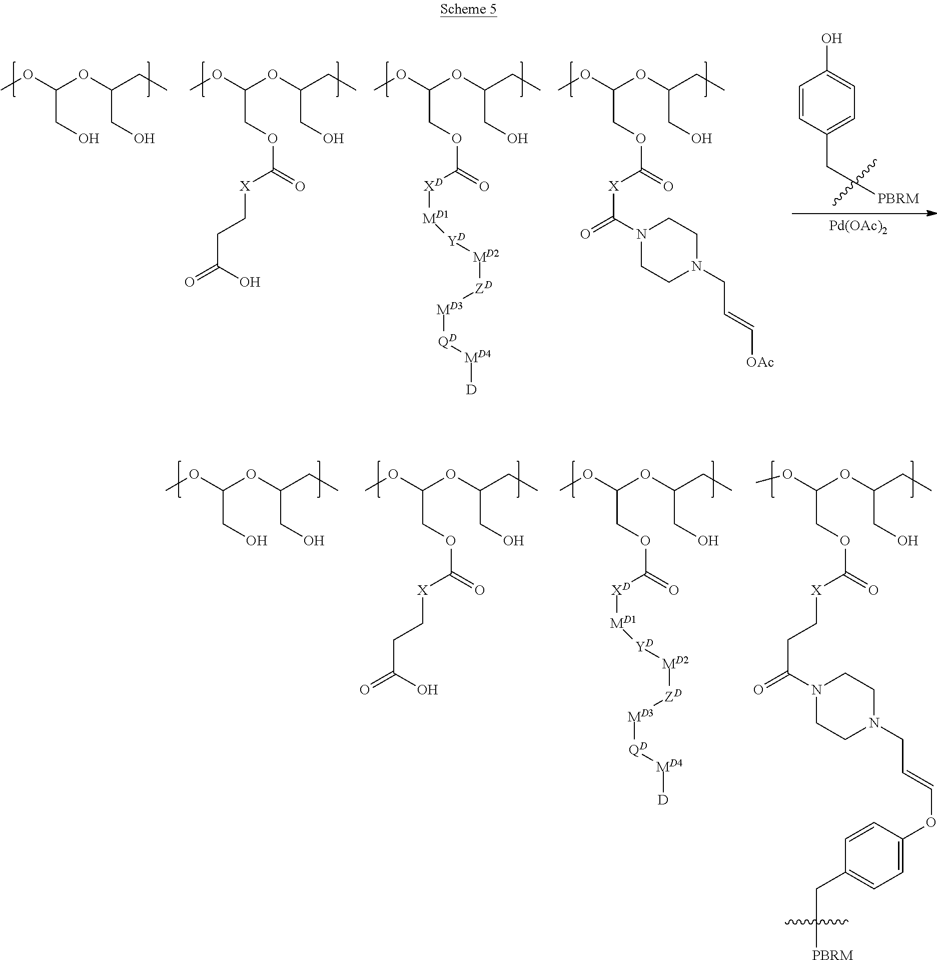

In Schemes 3-5 above, the wavy bond

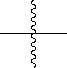

indicates that PBRM is either connected to the functional modifier directly or via another moiety such as alkyl, cycloalkyl, aryl, etc.

Scheme 6 below shows a general synthetic scheme of making the polymeric scaffolds of the invention. The wavy bond indicates direct or indirect connection between L^{D1} and D or L^{P2}. The conjugates are formed in several steps: (1) the polymer, PHF is modified to contain a —O—CO-L^{D1} moiety; (2) the polymer is then further modified so that it contains a L^{P2} moiety that is capable of forming a covalent bond with a functional group of a PBRM; (3) the modified polymer, containing two different functional groups, is reacted with a functional group of a drug or its derivative (e.g., D) to form a polymer-drug conjugate; (4) the PBRM is then reacted with the polymer-drug conjugate to form the protein-polymer-drug conjugate as depicted in the right side route in Scheme 6 below. In another embodiment the order of steps (2) and (3) can be reversed as depicted in the left side route in Scheme 6 below

The PBRM can be linked to the drug-polymer conjugate to form the protein-polymer-drug conjugate using standard synthetic methods for protein conjugation, including, but not limited to, reactions based on reductive amination, Staudinger ligation, oxime formation, thiazolidine formation and the methods and reactions described herein.

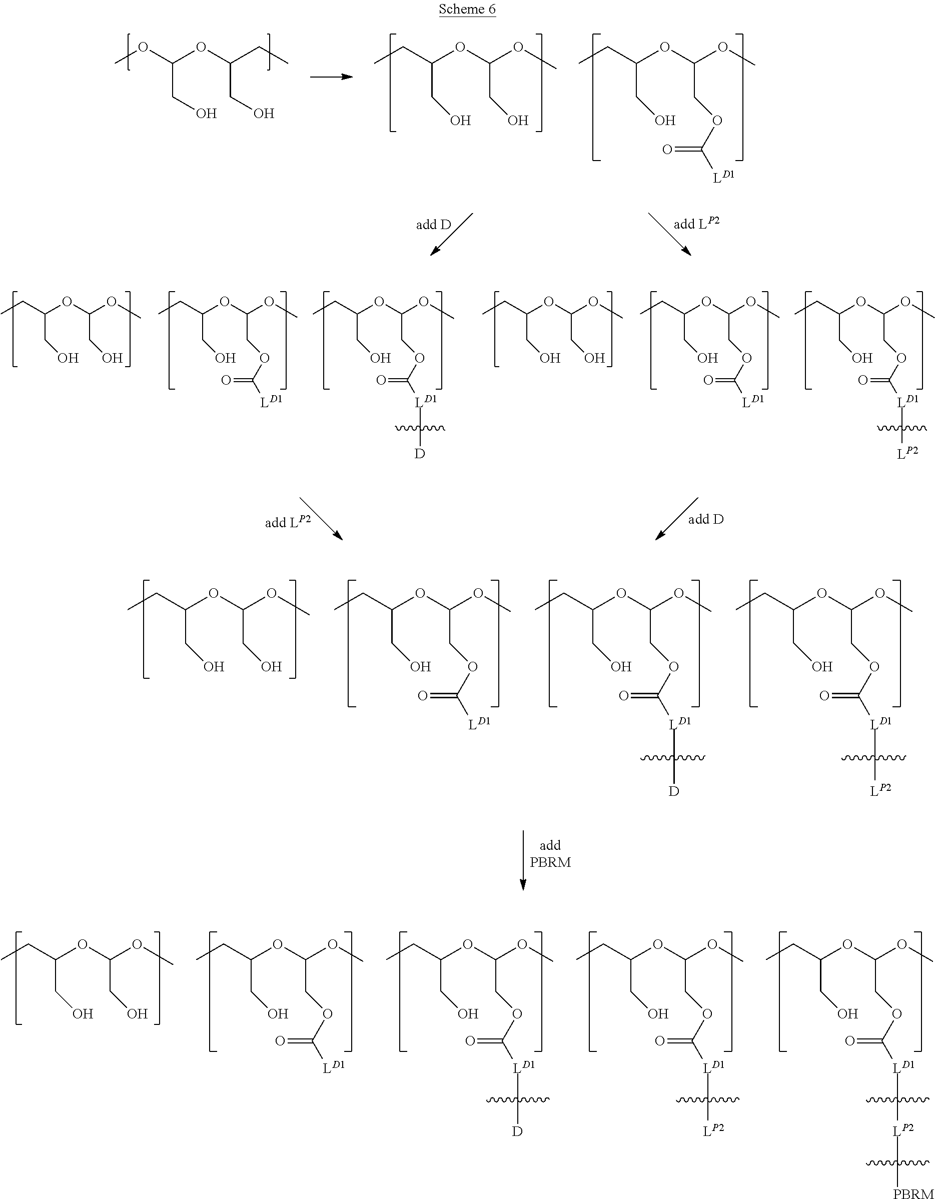

In yet another embodiment, steps (2) and (3) above are carried out simultaneously as depicted in Scheme 7 below.

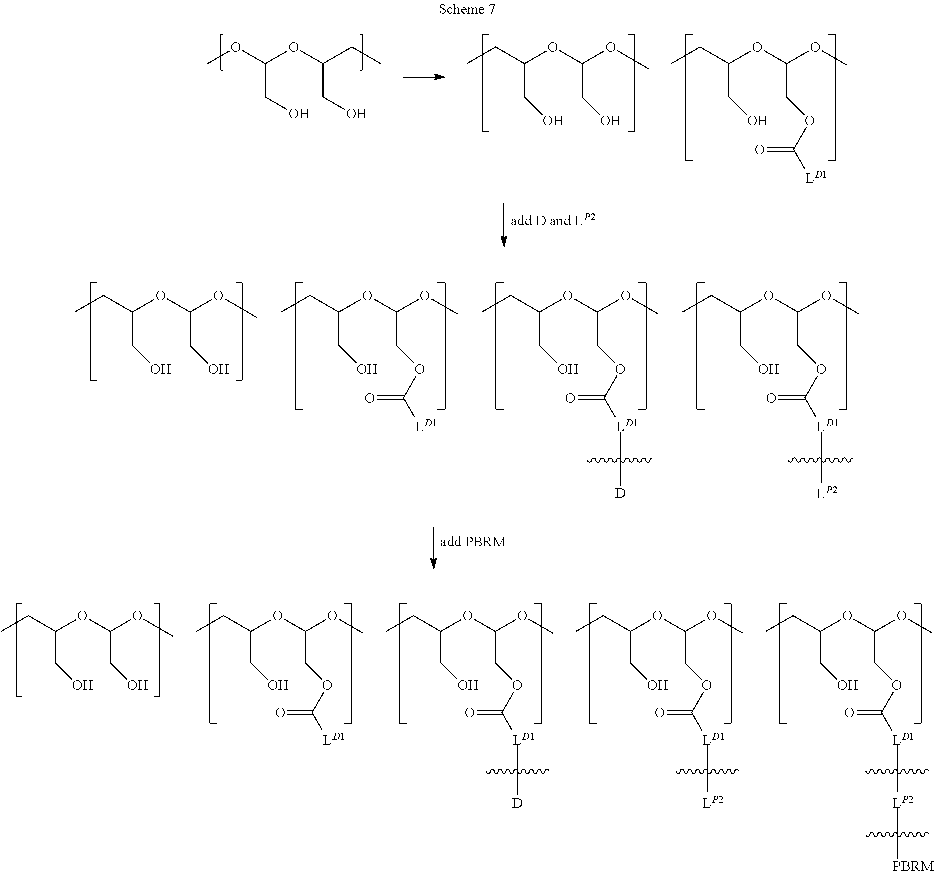

### Pharmaceutical Compositions

Also included are pharmaceutical compositions comprising one or more protein-polymer-drug conjugates as disclosed herein in an acceptable carrier, such as a stabilizer, buffer, and the like. The conjugates can be administered and introduced into a subject by standard means, with or without stabilizers, buffers, and the like, to form a pharmaceutical composition. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal, oral or parenteral administration including intravenous, intraarterial, subcutaneous, intraperitoneal or intramuscular injection or infusion or intracranial, e.g., intrathecal or intraventricular, administration. The conjugates can be formulated and used as sterile solutions and/or suspensions for injectable administration; lyophilized powders for reconstitution prior to injection/infusion; topical compositions; as tablets, capsules, or elixirs for oral administration; or suppositories for rectal administration, and the other compositions known in the art.

A pharmacological composition or formulation refers to a composition or formulation in a form suitable for administration, e.g., systemic administration, into a cell or subject, including for example a human. Suitable forms, in part, depend upon the use or the route of entry, for example oral, inhaled, transdermal, or by injection/infusion. Such forms should not prevent the composition or formulation from reaching a target cell (i.e., a cell to which the drug is desirable for delivery). For example, pharmacological compositions injected into the blood stream should be soluble. Other factors are known in the art, and include considerations such as toxicity and forms that prevent the composition or formulation from exerting its effect.

By “systemic administration” is meant in vivo systemic absorption or accumulation of the modified polymer in the blood stream followed by distribution throughout the entire body. Administration routes that lead to systemic absorption include, without limitation: intravenous, subcutaneous, intraperitoneal, inhalation, oral, intrapulmonary, and intramuscular. Each of these administration routes exposes the modified polymers to an accessible diseased tissue. The rate of entry of an active agent into the circulation has been shown to be a function of molecular weight or size. The use of a conjugate of this invention can localize the drug delivery in certain cells, such as cancer cells via the specificity of PBRMs.

A “pharmaceutically acceptable formulation” means a composition or formulation that allows for the effective distribution of the conjugates in the physical location most suitable for their desired activity. In one embodiment, effective delivery occurs before clearance by the reticuloendothelial system or the production of off-target binding which can result in reduced efficacy or toxicity. Non-limiting examples of agents suitable for formulation with the conjugates include: P-glycoprotein inhibitors (such as Pluronic P85), which can enhance entry of active agents into the CNS; biodegradable polymers, such as poly (DL-lactide-coglycolide) microspheres for sustained release delivery after intracerebral implantation; and loaded nanoparticles, such as those made of polybutylcyanoacrylate, which can deliver active agents across the blood brain barrier and can alter neuronal uptake mechanisms.

Also included herein are pharmaceutical compositions prepared for storage or administration, which include a pharmaceutically effective amount of the desired conjugates in a pharmaceutically acceptable carrier or diluent. Acceptable carriers, diluents, and/or excipients for therapeutic use are well known in the pharmaceutical art. For example, buffers, preservatives, bulking agents, dispersants, stabilizers, dyes, can be provided. In addition, antioxidants and suspending agents can be used Examples of suitable carriers, diluents and/or excipients include, but are not limited to: (1) Dulbecco's phosphate buffered saline, pH about 6.5, which would contain about 1 mg/ml to 25 mg/ml human serum albumin, (2) 0.9% saline (0.9% w/v NaCl), and (3) 5% (w/v) dextrose.

The term “pharmaceutically effective amount”, as used herein, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Pharmaceutically effective amounts for a given situation can be determined by routine experimentation that is within the skill and judgment of the clinician. In a preferred aspect, the disease or condition to can be treated via gene silencing.

For any conjugate, the pharmaceutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

For example, a drug or its derivatives, drug-polymer conjugates or PBRM-drug-polymer conjugates can be evaluated for their ability to inhibit tumor growth in several cell lines using Cell titer Glo. Dose response curves can be generated using SoftMax Pro software and IC₅₀ values can be determined from four-parameter curve fitting. Cell lines employed can include those which are the targets of the PBRM and a control cell line that is not the target of the PBRM contained in the test conjugates.

In one embodiment, the conjugates are formulated for parenteral administration by injection including using conventional catheterization techniques or infusion. Formulations for injection may be presented in unit dosage form, e.g., in ampules or in multi-dose containers, with an added preservative. The conjugates can be administered parenterally in a sterile medium. The conjugate, depending on the vehicle and concentration used, can either be suspended or dissolved in the vehicle. Advantageously, adjuvants such as local anesthetics, preservatives, and buffering agents can be dissolved in the vehicle. The term “parenteral” as used herein includes percutaneous, subcutaneous, intravascular (e.g., intravenous), intramuscular, or intrathecal injection or infusion techniques and the like. In addition, there is provided a pharmaceutical formulation comprising conjugates and a pharmaceutically acceptable carrier. One or more of the conjugates can be present in association with one or more non-toxic pharmaceutically acceptable carriers and/or diluents and/or adjuvants, and if desired other active ingredients.

The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution, and isotonic sodium chloride solution. In addition, sterile, fixed oils are conventionally employed as a solvent or suspending medium. For this purpose, a bland fixed oil can be employed including synthetic mono- or diglycerides. In addition, fatty acids such as oleic acid find use in the preparation of injectables.

The conjugates and compositions described herein may be administered in appropriate form, preferably parenterally, more preferably intravenously. For parenteral administration, the conjugates or compositions can be aqueous or nonaqueous sterile solutions, suspensions or emulsions. Propylene glycol, vegetable oils and injectable organic esters, such as ethyl oleate, can be used as the solvent or vehicle. The compositions can also contain adjuvants, emulsifiers or dispersants.

Dosage levels of the order of from between about 0.01 mg and about 140 mg per kilogram of body weight per day are useful in the treatment of the above-indicated conditions (between about 0.05 mg and about 7 g per subject per day). In some embodiments, the dosage administered to a patient is between about 0.01 mg/kg to about 100 mg/kg of the subject's body weight. In some embodiments, the dosage administered to a patient is between about 0.01 mg/kg to about 15 mg/kg of the subject's body weight. In some embodiments, the dosage administered to a patient is between about 0.1 mg/kg and about 15 mg/kg of the subject's body weight. In some embodiments, the dosage administered to a patient is between about 0.1 mg/kg and about 20 mg/kg of the subject's body weight. In some embodiments, the dosage administered is between about 0.1 mg/kg to about 5 mg/kg or about 0.1 mg/kg to about 10 mg/kg of the subject's body weight. In some embodiments, the dosage administered is between about 1 mg/kg to about 15 mg/kg of the subject's body weight. In some embodiments, the dosage administered is between about 1 mg/kg to about 10 mg/kg of the subject's body weight. The amount of conjugate that can be combined with the carrier materials to produce a single dosage form varies depending upon the host treated and the particular mode of administration. Dosage unit forms can generally contain from between about 0.01 mg and about 100 mg; between about 0.01 mg and about 75 mg; or between about 0.01 mg and about 50 mg; or between about 0.01 mg and about 25 mg; of a conjugate.

For intravenous administration, the dosage levels can comprise from about 0.01 to about 200 mg of a conjugate per kg of the animal's body weight. In one aspect, the composition can include from about 1 to about 100 mg of a conjugate per kg of the animal's body weight. In another aspect, the amount administered will be in the range from about 0.1 to about 25 mg/kg of body weight of a compound.

In some embodiments, the conjugates can be administered are as follows. The conjugates can be given daily for about 5 days either as an i.v., bolus each day for about 5 days, or as a continuous infusion for about 5 days.

Alternatively, the conjugates can be administered once a week for six weeks or longer. As another alternative, the conjugates can be administered once every two or three weeks. Bolus doses are given in about 50 to about 400 ml of normal saline to which about 5 to about 10 ml of human serum albumin can be added. Continuous infusions are given in about 250 to about 500 ml of normal saline, to which about 25 to about 50 ml of human serum albumin can be added, per 24 hour period.

In some embodiments about one to about four weeks after treatment, the patient can receive a second course of treatment. Specific clinical protocols with regard to route of administration, excipients, diluents, dosages, and times can be determined by the skilled artisan as the clinical situation warrants.

It is understood that the specific dose level for a particular subject depends upon a variety of factors including the activity of the specific conjugate, the age, body weight, general health, sex, diet, time of administration, route of administration, and rate of excretion, combination with other active agents, and the severity of the particular disease undergoing therapy.

For administration to non-human animals, the conjugates can also be added to the animal feed or drinking water. It can be convenient to formulate the animal feed and drinking water so that the animal takes in a therapeutically appropriate quantity of the conjugates along with its diet. It can also be convenient to present the conjugates as a premix for addition to the feed or drinking water.

The conjugates can also be administered to a subject in combination with other therapeutic compounds to increase the overall therapeutic effect. The use of multiple compounds to treat an indication can increase the beneficial effects while reducing the presence of side effects. In some embodiment the conjugates are used in combination with chemotherapeutic agents, such as those disclosed in U.S. Pat. No. 7,303,749. In other embodiments the chemotherapeutic agents, include, but are not limited to letrozole, oxaliplatin, docetaxel, 5-FU, lapatinib, capecitabine, leucovorin, erlotinib, pertuzumab, bevacizumab, and gemcitabine.

The present invention also provides pharmaceutical kits comprising one or more containers filled with one or more of the conjugates and/or compositions of the present invention, including, one or more chemotherapeutic agents. Such kits can also include, for example, other compounds and/or compositions, a device(s) for administering the compounds and/or compositions, and written instructions in a form prescribed by a governmental agency regulating the manufacture, use or sale of pharmaceuticals or biological products.

### Methods of Use

### Methods of Treating

In certain preferred embodiments of the invention, the protein-polymer-drug conjugate of the invention are used in methods of treating animals (preferably mammals, most preferably humans and includes males, females, infants, children and adults). In one embodiment, the conjugates of the present invention may be used in a method of treating animals which comprises administering to the animal a biodegradable biocompatible conjugate of the invention. For example, conjugates in accordance with the invention can be administered in the form of soluble linear polymers, copolymers, conjugates, colloids, particles, gels, solid items, fibers, films, etc. Biodegradable biocompatible conjugates of this invention can be used as drug carriers and drug carrier components, in systems of controlled drug release, preparations for low-invasive surgical procedures, etc. Pharmaceutical formulations can be injectable, implantable, etc.

In yet another aspect, the invention provides a method of treating a disease or disorder in a subject in need thereof, comprising administering to the subject an efficient amount of at least one conjugate of the invention; wherein said conjugate releases one or more tubulysin compounds upon biodegradation.

In another embodiment the conjugates can be administered in vitro, in vivo and/or ex vivo to treat patients and/or to modulate the growth of selected cell populations including, for example, cancer. In some embodiments, the particular types of cancers that can be treated with the conjugates include, but are not limited to: (1) solid tumors, including but not limited to fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon cancer, colorectal cancer, kidney cancer, pancreatic cancer, bone cancer, breast cancer, ovarian cancer, prostate cancer, esophogeal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma bile duct carcinoma, choriocarcinoma, seminoma, embryonal carcinoma, Wilms' tumor, cervical cancer, uterine cancer, testicular cancer, small cell lung carcinoma, bladder carcinoma, lung cancer, epithelial carcinoma, glioma, glioblastoma, multiforme astrocytoma, medulloblastoma, craniopharyngioma, ependymoma, pinealoma, hemangioblastoma, acoustic neuroma, oligodendroglioma, meningioma, skin cancer, melanoma, neuroblastoma, and retinoblastoma; (2) blood-borne cancers, including but not limited to acute lymphoblastic leukemia “ALL”, acute lymphoblastic B-cell leukemia, acute lymphoblastic T-cell leukemia, acute myeloblastic leukemia “AML”, acute promyelocytic leukemia “APL”, acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia “CML”, chronic lymphocytic leukemia “CLL”, hairy cell leukemia, multiple myeloma, acute and chronic leukemias, e.g., lymphoblastic myelogenous and lymphocytic myelocytic leukemias; and (3) lymphomas such as Hodgkin's disease, non-Hodgkin's Lymphoma, Multiple myeloma, Waldenstrom's macroglobulinemia, Heavy chain disease, and Polycythemia vera.

In another embodiment the conjugates can be administered in vitro, in vivo and/or ex vivo to treat autoimmune diseases, such as systemic lupus, rheumatoid arthritis, psoriasis, and multiple sclerosis; graft rejections, such as renal transplant rejection, liver transplant rejection, lung transplant rejection, cardiac transplant rejection, and bone marrow transplant rejection; graft versus host disease; viral infections, such as CMV infection, HIV infection, and AIDS; and parasite infections, such as giardiasis, amoebiasis, schistosomiasis, and the like.

In certain embodiments the conjugates can also be used for the manufacture of a medicament useful for treating or lessening the severity of disorders, such as, characterized by abnormal growth of cells (e.g., cancer).

In certain embodiments, the tubulysin compound is locally delivered to a specific target cell, tissue, or organ.

In certain embodiments, in practicing the method of the invention, the conjugate further comprises or is associated with a diagnostic label. In certain exemplary embodiments, the diagnostic label is selected from the group consisting of: radiopharmaceutical or radioactive isotopes for gamma scintigraphy and PET, contrast agent for Magnetic Resonance Imaging (MRI), contrast agent for computed tomography, contrast agent for X-ray imaging method, agent for ultrasound diagnostic method, agent for neutron activation, moiety which can reflect, scatter or affect X-rays, ultrasounds, radiowaves and microwaves and fluorophores. In certain exemplary embodiments, the conjugate is further monitored in vivo.

Examples of diagnostic labels include, but are not limited to, diagnostic radiopharmaceutical or radioactive isotopes for gamma scintigraphy and PET, contrast agent for Magnetic Resonance Imaging (MRI) (for example paramagnetic atoms and superparamagnetic nanocrystals), contrast agent for computed tomography, contrast agent for X-ray imaging method, agent for ultrasound diagnostic method, agent for neutron activation, and moiety which can reflect, scatter or affect X-rays, ultrasounds, radiowaves and microwaves, fluorophores in various optical procedures, etc. Diagnostic radiopharmaceuticals include γ-emitting radionuclides, e.g., indium-111, technetium-99m and iodine-131, etc. Contrast agents for MRI (Magnetic Resonance Imaging) include magnetic compounds, e.g., paramagnetic ions, iron, manganese, gadolinium, lanthanides, organic paramagnetic moieties and superparamagnetic, ferromagnetic and antiferromagnetic compounds, e.g., iron oxide colloids, ferrite colloids, etc. Contrast agents for computed tomography and other X-ray based imaging methods include compounds absorbing X-rays, e.g., iodine, barium, etc. Contrast agents for ultrasound based methods include compounds which can absorb, reflect and scatter ultrasound waves, e.g., emulsions, crystals, gas bubbles, etc. Still other examples include substances useful for neutron activation, such as boron and gadolinium. Further, labels can be employed which can reflect, refract, scatter, or otherwise affect X-rays, ultrasound, radiowaves, microwaves and other rays useful in diagnostic procedures. Fluorescent labels can be used for photo imaging. In certain embodiments a modifier comprises a paramagnetic ion or group.

In another aspect, the invention provides a method of treating a disease or disorder in a subject, comprising preparing an aqueous formulation of at least one conjugate of the invention and parenterally injecting said formulation in the subject.

In another aspect, the invention provides a method of treating a disease or disorder in a subject, comprising preparing an implant comprising at least one conjugate of the invention, and implanting said implant into the subject. In certain exemplary embodiments, the implant is a biodegradable gel matrix.

In another aspect, the invention provides a method for treating of an animal in need thereof, comprising administering a conjugate according to the methods described above.

In another aspect, the invention provides a method for eliciting an immune response in an animal, comprising administering a conjugate as in the methods described above.

In another aspect, the invention provides a method of diagnosing a disease in an animal, comprising steps of:

administering a conjugate as in the methods described above, wherein said conjugate comprises a detectable molecule; and
detecting the detectable molecule.

In certain exemplary embodiments, the step of detecting the detectable molecule is performed non-invasively. In certain exemplary embodiments, the step of detecting the detectable molecule is performed using suitable imaging equipment.

In one embodiment, a method for treating an animal comprises administering to the animal a biodegradable biocompatible conjugate of the invention as a packing for a surgical wound from which a tumor or growth has been removed. The biodegradable biocompatible conjugate packing will replace the tumor site during recovery and degrade and dissipate as the wound heals.

In certain embodiments, the conjugate is associated with a diagnostic label for in vivo monitoring.

The conjugates described above can be used for therapeutic, preventative, and analytical (diagnostic) treatment of animals. The conjugates are intended, generally, for parenteral administration, but in some cases may be administered by other routes.

In one embodiment, soluble or colloidal conjugates are administered intravenously. In another embodiment, soluble or colloidal conjugates are administered via local (e.g., subcutaneous, intramuscular) injection. In another embodiment, solid conjugates (e.g., particles, implants, drug delivery systems) are administered via implantation or injection.

In another embodiment, conjugates comprising a detectable label are administered to study the patterns and dynamics of label distribution in animal body.

In certain embodiments, any one or more of the conjugates disclosed herein may be used in practicing any of the methods described above. In certain exemplary embodiments, the conjugate is a Trastuzumab-PHF-, Rituximab-PHF-, Lintuzumab-PHF, anti-5T4-PHF, anti-mesothelin-PHF, or LHRH-PHF-drug conjugate.

Throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the invention remains operable. Moreover, two or more steps or actions can be conducted simultaneously.

The synthetic processes of the invention can tolerate a wide variety of functional groups; therefore various substituted starting materials can be used. The processes generally provide the desired final compound at or near the end of the overall process, although it may be desirable in certain instances to further convert the compound to a pharmaceutically acceptable salt, ester or prodrug thereof.

Drug compounds used for the conjugates of the present invention can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5^{th} edition, John Wiley & Sons: New York, 2001; and Greene, T. W., Wuts, P. G. M., Protective Groups in Organic Synthesis, 3^{rd} edition, John Wiley & Sons: New York, 1999, incorporated by reference herein, are useful and recognized reference textbooks of organic synthesis known to those in the art. The following descriptions of synthetic methods are designed to illustrate, but not to limit, general procedures for the preparation of compounds of the present invention.

Conjugates of the present invention and the drug compounds included therein can be conveniently prepared by a variety of methods familiar to those skilled in the art. The conjugates or compounds of this invention with each of the formulae described herein may be prepared according to the following procedures from commercially available starting materials or starting materials which can be prepared using literature procedures. These procedures show the preparation of representative conjugates of this invention.

Conjugates designed, selected and/or optimized by methods described above, once produced, can be characterized using a variety of assays known to those skilled in the art to determine whether the conjugates have biological activity. For example, the conjugates can be characterized by conventional assays, including but not limited to those assays described below, to determine whether they have a predicted activity, binding activity and/or binding specificity.

Furthermore, high-throughput screening can be used to speed up analysis using such assays. As a result, it can be possible to rapidly screen the conjugate molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

### EXAMPLES

Conjugates described herein can be prepared by the schemes generally outlined above and by methods described in the Examples below. The term “content” as used in certain examples below, unless otherwise specified, means the molar fraction of the polymer units that are substituted with the intended moiety, such as the linker, the drug molecule (i.e., tubulysin compound), or PBRM.

### Abbreviations

The following abbreviations are used in the reaction schemes and synthetic examples, which follow. This list is not meant to be an all-inclusive list of abbreviations used in the application as additional standard abbreviations, which are readily understood by those skilled in the art of organic synthesis, can also be used in the synthetic schemes and examples.
BA β-Alanine
BOC tert-Butyloxycarbonyl
DIC N,N′-Diisopropylcarbodiimide
DMA Dimethylacetamide
DMF Dimethylformamide
DMAP 4-Dimethylaminopyridine
DMSO Dimethylsulfoxide
DTT (2S,3S)-1,4-dimercaptobutane-2,3-diol
EDC 1-Ethyl-3-[3-dimethylaminopropyl]carbodiimide hydrochloride
GA Glutaric acid
HOBt Hydroxybenzotriazole
HPLC High pressure liquid chromatography
MCC (N-maleimidomethyl) 1,4-cyclohexyl carbamate
MI Maleimido
M-(PEG)₁₂ N-maleimido-PEG₁₂-propionamide
MWCO Molecular Weight Cut-Off
NHS 1-Hydroxypyrrolidine-2,5-dione
NMP N-methyl-2-pyrrolidone
PABA p-Amino benzoic acid
PBS Phosphate buffered saline, 0.9% NaCl
PHF poly(1-hydroxymethylethylene hydroxylmethylformal), or FLEXIMER®
PNP p-Nitrophenoxide
SATA N-Succinimidyl-S-acetylthioacetate
SEC Size exclusion chromatography
SMCC Succinimidyl-4-(N-maleimidomethyl)cyclohexane-1-carboxylate
—SS— Indicates a covalently bound disulfide group
SSPy 2-(pyridine-2-yldisulfanyl)
TCEP Tris[2-carboxyethyl] phosphine
TEA Triethylamine
TFA Trifluoroacetic acid

### General Information

Fmoc-Val-Cit-PABA-PNP was purchased from Concortis Biosystems.

N-Boc-D-valine was purchased from Alfa Aesar.

Anti-Her2 affibody, 14 K, was purchased from Affibody AB.

HPLC purification was performed on a Phenomenex Gemini 5 μm 110 Å, 250×10 mm, 5 micron, semi-preparation column using the following solvent system: Solvent A: water (0.1% TFA); Solvent B: CH₃CN (0.1% TFA).

Whenever possible the drug content of the conjugates was determined. spectrophotometrically otherwise LC/MS was performed for quantitative determination of the drug content.

Protein content of the conjugates was determined spectrophotometrically at 280 nm.

Disulfide content in -SSPy conjugates was determined spectrophotometrically at 340 nm after pyridinethione release (10 mM DTT, 10 min, ambient temperature).

The molecular weights of the polymer conjugates were determined by SEC with either polysaccharide or protein molecular weight standards. More specifically, for the polymer or polymer drug conjugates, polysaccharide molecular weights standard are used, and for protein-polymer-drug conjugates, protein standards are used. Unless specifically indicated the reported polymer carrier molecular weight is the weight average molecular weight of PHF. The polymer and polymer conjugates synthesized/measured all had a polydispersity <2.

PBRM-drug polymer conjugates were isolated from residual unreacted drug polymer conjugates by extensive diafiltration. If necessary, additional purification by size exclusion chromatography was conducted to remove any aggregated PBRM-drug polymer conjugates. In general, the PBRM-drug polymer conjugates typically contained <5% aggregated PBRM-drug polymer conjugates as determined by SEC or SDS-PAGE; <1% free (unconjugated) drug as determined by SEC and <2% unconjugated PBRM as determined by HPLC.

Reduced or partially reduced antibodies were prepared using procedures described in the literature, see, for example, Francisco et al., Blood 102 (4): 1458-1465 (2003).

### Example 1. Synthesis of PHF-β-Alanine

### A. Synthesis of 30 kDa PHF-β-Alanine

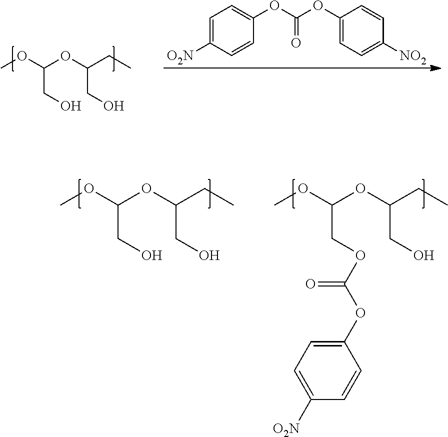

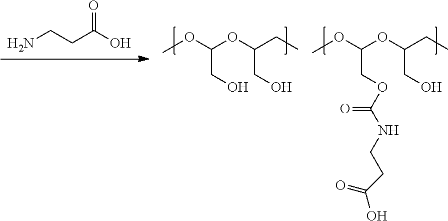

PHF (30 kDa, 4.54 g, 33.6 mmol PHF monomer) was dissolved in 150 mL anhydrous DMF, followed by the addition of bis(nitrophenol) carbonate (3.07 g, 10.1 mmol). The solution was stirred at 40° C. for 4 h. β-Alanine (1.50 g, 16.8 mmol) dissolved in water (10 mL) was added to the PHF mixture. The pH was adjusted to 7.5-8 with TEA and the reaction mixture stirred at 23° C. for 18 h, diluted to 400 mL with water and the pH adjusted to 11 with 5N NaOH. The resulting mixture was stirred for 1 h at ambient temperature, the pH was adjusted to 6.5 and then the mixture was diluted to 10% organics with water. The product (30 kDa PHF-(3-Alanine) was purified using ultrafiltration cartridge equipped with 5K Biomax membrane filter. The purified product was lyophilized to give the title compound as a white solid (2.07 g, 36% yield). The molar fraction of the PHF monomer units substituted with β-alanine was 13%, as determined by ¹H NMR.

### B. Synthesis of 13 kDa PHF-β-Alanine

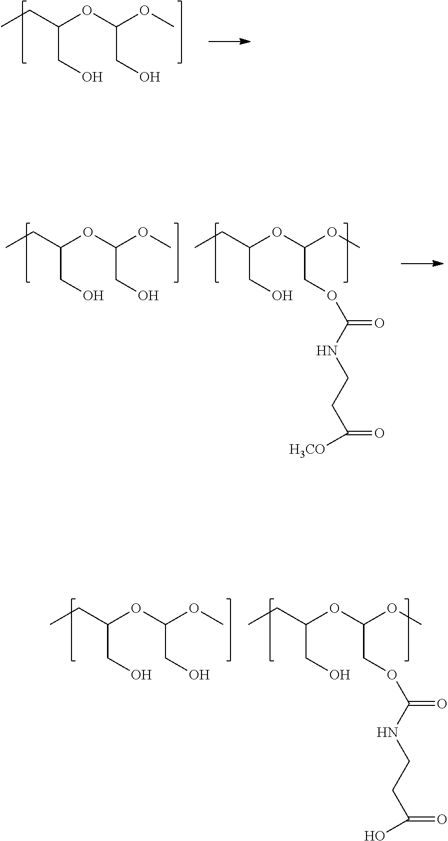

PHF (12 g), DMA (100 g) and pyridine (7.4 g) were stirred at 40° C. for ˜3 hours. To the clear solution was added methyl-3-isocyanatopropanoate (3.8 g, 0.33 mole % to PHF) over a period of 5 minutes and the stirring continued for an additional 24 hours at 45° C. The reaction mixture was then diluted with water (320 g) and 5N NaOH (32 g) at 25° C. was added over 2 minutes, final pH 13. The mixture was stirred at 25° C. for 18 h, the pH of the reaction mixture was adjusted to 7 with 1N HCl, followed by dilution to ˜3.5 L with water, concentrated by diafiltration using a membrane filter, 3 kDa MWCO, followed by purification on a Sephadex G-25 column. The resulting PHF BA was characterized to have a molecular weight of ˜13 kDa. (BA -31%, 14 g, yield 90%).

### Example 2. Synthesis of 30 kDa PHF-GA

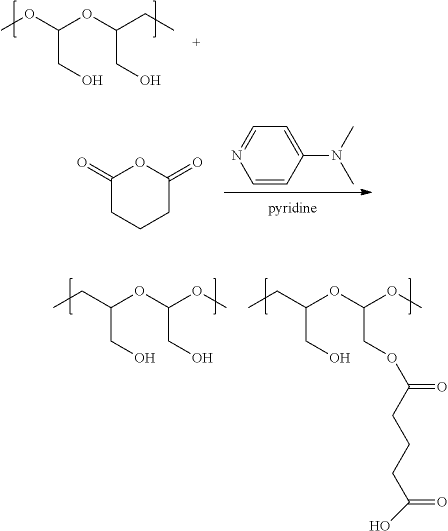

N,N-Dimethylpyridin-4-amine (0.268 g, 2.91 mmol) and glutaric anhydride (1.375 g, 12.06 mmol) was added to a solution of PHF (30 kDa, 1.48 g, 10.96 mmol PHF monomer) in DMA (300 mL) and anhydrous pyridine (33.3 mL). The reaction mixture was stirred at 60° C. for 18 h. The solvents were removed under reduced pressure and the resulting thick oil was taken up in water (100 mL). The pH was adjusted to pH 6.0-6.5 with 5N NaOH. The resulting clear solution was diluted to 200 mL with water, filtered through a 0.2 micron filter, and purified by diafiltration using a membrane filter, 5000 molecular weight cut-off. The water was removed by lyophilization to give 30 kDa PHF-GA as a white solid (1.28 g, 48% yield). The fraction of the total PHF monomer units substituted with glutaric acid as determined by ¹H NMR was 96%.

### Example 3. Synthesis of Trastuzumab-MCC Derivative

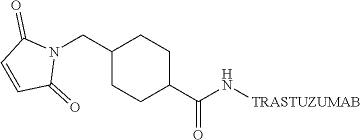

Trastuzumab (10 mg) was dissolved in PBS buffer (1 ml, pH 7.0), then a solution of SMCC in DMSO (5 μL, 30 mg/ml) was added. The resulting solution was stirred at room temperature for 2 h. The trastuzumab-MCC was purified by gel filtration using a PBS equilibrated PD-10 column (90% yield). Analysis showed that on average 5 to 6 MCC groups were linked to one trastuzumab.

Other PBRM-MCC derivatives, such as, MCC derivatives of cetuximab, rituximab, bevacizumab, nimotuzumab, gemtuzumab, alemtuzumab lintuzumab, anti-5T4 or anti-mesothelin antibodies, are synthesized with methods similar to the procedure described above.

### Example 4. Synthesis of Trastuzumab-M-(PEG)₁₂ Derivative

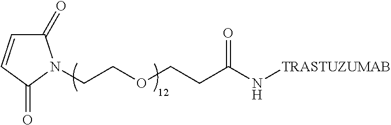

Trastuzumab (10 mg) was dissolved in PBS buffer (1 ml, pH 7.0), then a solution of SM-(PEG)₁₂ in DMSO (4 μL, 100 mg/ml) was added. The resulting solution was stirred at room temperature for 2 h. Trastuzumab-M-(PEG)₁₂ was purified by gel filtration using a PBS equilibrated PD-10 column (˜90% yield). Analysis showed that on average 5 to 6 polyethylene groups were linked to one trastuzumab.

Other PBRM-M-(PEG)₁₂ derivatives, such as, M-(PEG)₁₂ derivatives of cetuximab, rituximab, bevacizumab, nimotuzumab, gemtuzumab, alemtuzumab, lintuzumab, anti-5T4 or anti-mesothelin antibodies are synthesized with methods similar to the procedure described above.

### Example 5. Synthesis of Rituximab-MCC Derivative

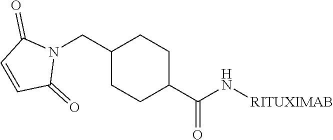

The title compound was prepared as described in Example 3 except Rituximab was used instead of trastuzumab. Analysis showed that on average 5 to 6 MCC groups were linked to one Rituximab.

Other PBRM-MCC derivatives, such as, MCC derivatives of cetuximab, bevacizumab, nimotuzumab, gemtuzumab, alemtuzumab, lintuzumab, anti-5T4 or anti-mesothelin antibodies are synthesized with methods similar to the procedure described above

### Example 6. Synthesis of 10 kDa PHF-GA-SSpy

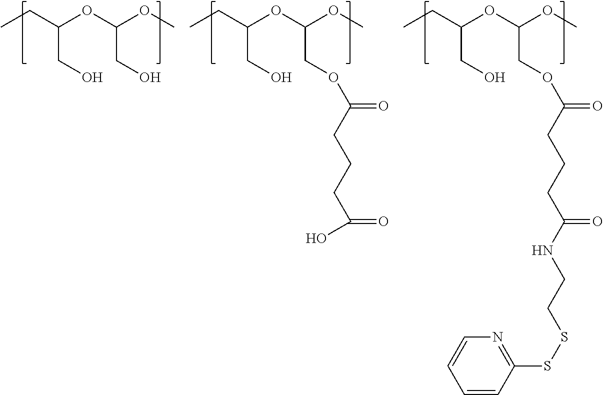

10 kDa PHF-GA (1.63 g 11.12 mmol, prepared using the procedure described in Example 2 with PHF 10,000 Da, 25% GA) was dissolved in water (10 mL) and NHS (0.154 g, 1.33 mmol) was added. The mixture was cooled to 0° C. and then an aqueous solution of EDC (0.256 g, 1.33 mmol) was added followed by 2-(pyridine-2-yldisulfanyl)ethaneamine hydrochloride (0.297 g, 1.33 mmol). The pH of the resulting mixture was adjusted to 5.5-6.0 then stirred at 23° C. for 18 h, followed by purification by dialysis through a Regenerated Cellulose membrane, and lyophilization to give the title compound (1.66 g, 86%) as a white solid. The SSPy content was 3%.

### Example 7. Synthesis of 13 kDa PHF-BA-SSpy

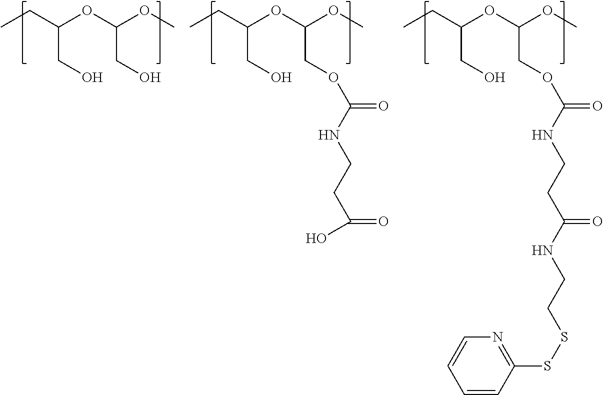

PHF-BA (2.5 g, prepared using the procedure described in Example 1, MW ˜13 kDa), NHS (0.103 g) and 2-(pyridine-2-yldisulfanyl)ethaneamine hydrochloride (0.2 g) were dissolved in water (50 mL). The mixture was cooled to 5-10° C. and then an aqueous solution of EDC (0.344 g) was added. The pH of the resulting mixture was adjusted to 5.5-6.0 then stirred at 23° C. for 18 h, followed by purification using a membrane filter, 3 kDa MWCO, and lyophilization to give the title compound (1.9 g, 75% yield) as a white solid. The SSPy content was 5.9%.

### Example 8. Synthesis of 30 kDa PHF-GA-Maleimide

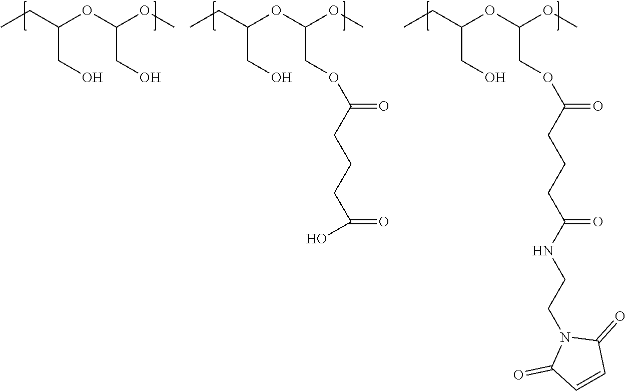

30 kDa PHF-GA (7.98 g, 50.2 mmol, prepared as described in Example 2, GA 15%) was taken up in water (502 mL) and cooled to 0° C. NHS (0.087 g, 0.752 mmol) was added followed by an aqueous solution of EDC (0.144 g, 0.752 mmol). The pH was adjusted to pH 7 to 8 with 1N NaOH and the reaction mixture stirred for 1 h at room temperature. N-aminoethyl-maleimide (0.080 g, 0.451 mmol) was added at 0° C. and the reaction mixture was warmed to room temperature and then left stirring overnight. The mixture was filtered through a 2 micron filter, concentrated to 200 mL, purified by dialysis through a Biomax (polyethersulfone) cartridge (5K) by washing with 1 liter of water, followed by lyophilization to give the title compound (2.19 g, 28% yield) as a white solid. Maleimide content as determined by CHN elemental analysis was 2.6%: (CHN average): C: 44.81, H: 6.91, N: 0.49.

### Example 9. Synthesis of Trastuzumab-F(ab′)₂

Trastuzumab-F(ab′)₂ was prepared from immobilized pepsin (15 mL settled gel) and trastuzumab (440 mg, 2.4 μmol) according to the manufacturer's (Pierce) instructions to give the title compound (265.2 mg, 92% yield).

By substituting trastuzumab with other PBRMs, such as, for example, cetuximab, rituximab, bevacizumab, nimotuzumab, gemtuzumab, alemtuzumab, lintuzumab, anti-5T4 or anti-mesothelin antibodies, in the procedure described above it is possible to synthesize other PBRM F(ab)′2 fragments.

### Example 10. Synthesis of Trastuzumab-Fab

Trastuzumab-Fab was prepared from immobilized papain (6.5 mL resin) and trastuzumab (192 mg) to give two Fab fragments and an Fc fragments. Purification resulted in Trastuzumab Fab (51.4 mg). MW (SDS PAGE), ˜45 kDa.

Other PBRM-Fab fragments, such as, Fab fragments of cetuximab, rituximab, bevacizumab, nimotuzumab, gemtuzumab, alemtuzumab, lintuzumab, anti-5T4 or anti-mesothelin antibodies, are synthesized with methods similar to the procedure described above.

### Example 11. Synthesis of N-3(aminopropyl)4-methyl-4-((5-nitropyridin-2-yl)disulfanyl)pentanamide

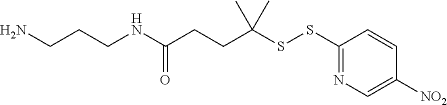 [SMILES: CC(C)(CCC(=O)NCCCN)SSc1ccc([N+](=O)[O-])cn1]

To tert-butyl 3-aminopropylcarbamate (0.437 mL, 2.50 mmol) in DMF (1 mL) was added N-ethyl-N-isopropylpropan-2-amine (0.437 mL, 2.50 mmol) and 1H-benzo[d][1,2,3]triazol-1-ol (846 mg, 6.26 mmol). The reaction mixture was stirred for 10 minutes at 25° C. and 2,5-dioxopyrrolidin-1-yl-4-methyl-4-((5-nitropyridin-2-yl)disulfanyl)pentanoate (500 mg, 1.25 mmol) in DMF (1 mL) was added. The reaction mixture was stirred at 25° C. for 18 hours. Purification by HPLC afforded the title compound as its tert butyl carbamate (476.7 mg, 1.04 mmol, 83%) as a beige solid: m/z 459 [M+H]⁺.

To the title compound as its tert butyl carbamate (699.7 mg, 1.53 mmol) in DMF (5.00 mL) was added 2,2,2-trifluoroacetic acid (2.35 mL, 30.5 mmol). The mixture was stirred at 25° C. for 1 hour. After removal of the solvent the resulting title compound was used without further purification: m/z 359 [M+H]⁺.

### Example 12. 10K PHF-GA (25%)-SS-Dimethyl-NO₂ (5%)

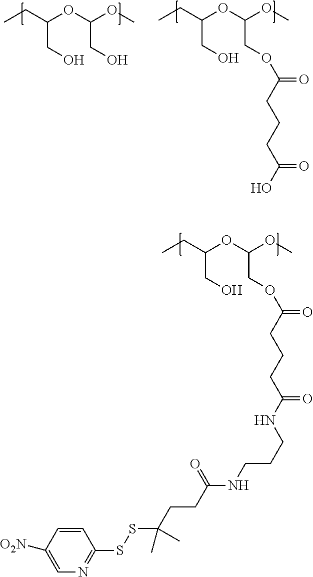

10 kDa PHF-GA (2.37 g, 14.5 mmol, prepared using the procedure described in Example 2 with PHF 10,000 Da, 25% GA) was diluted to 100 mL with water and NHS (0.133 g, 1.16 mmol) was added. The mixture was cooled to 0° C., pH adjusted to 5.5-6.0 and then N-3(aminopropyl)-4-methyl-4-((5-nitropyridin-2-yl)disulfanyl)pentanamide (547.0 mg, 1.16 mmol, Example 11) in CH₃CN (4 mL) and DMF (0.5 mL) were added followed by EDC (0.222 g, 1.16 mmol). The pH of the reaction mixture was again adjusted to 5.5-6.0 and stirred at room temperature for 18 hours. Additional EDC (0.150 mg, 0.782 mmol) was added and the mixture stirred for an additional 1.5 hours. The sample was purified via dialysis through a Regenerated Cellulose membrane to give the title compound (2.05 g).

### Example 13. Synthesis of LHRH-PEG₁₂-SH

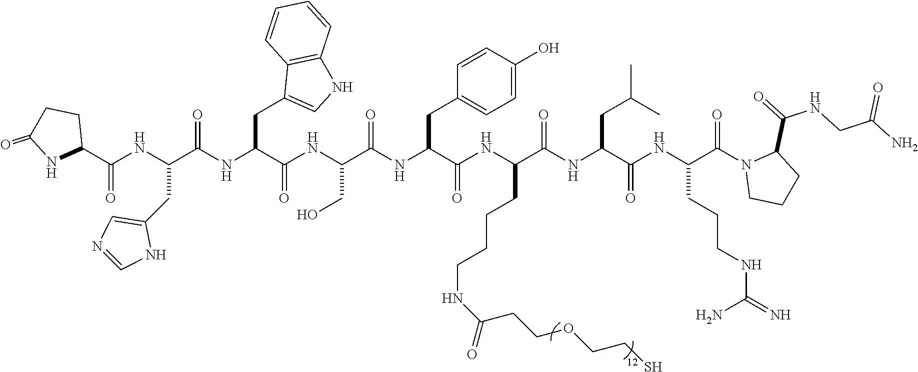

LHRH (10 mg) was dissolved in a mixture of acetonitrile: water (1:1, 500 μL) and to it was added PEG₁₂-SATA stock solution (9.2 μL, 0.0025 mmol, 1.9 mg). The resulting mixture was stirred for 3 h at ambient temperature. The product was purified by RP-HPLC followed by lyophilization (60% yield).

Purified LHRH-PEG₁₂-SH (2 mg) was dissolved in water (400 μL), pH was adjusted to 11.8 with TEA, and the mixture was stir for 40 min under argon and used in the next step.

### Example 14. Synthesis of EC-1-Adoa-M-(PEG)₁₂

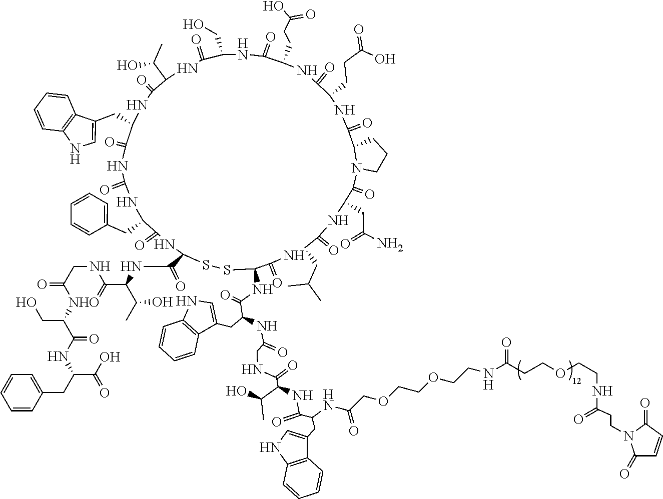

To a mixture of EC-1-Adoa-NH₂ (10 mg, 4 15 μmol) in CH₃CN/H₂O/DMSO (750 μL, 7:7:1) was added M-(PEG)₁₂-NHS (63 μL, 4.1 mg, 4.7 μmol) stock solution (0.064 mg/mL) in CH₃CN. The pH was adjusted to 7.4 and then DMSO (50 μL) and NMP (50 μL) were added to make the mixture more homogenous. The mixture was stirred under argon overnight, protected from light. An additional aliquot (13 μL, 1 mg) of freshly prepared M-(PEG)₁₂-NHS stock (0.077 mg/mL) was added and the resulting mixture was stirred for 30 min. The crude product was purified by HPLC (Gradient: 10% solvent B to 90% solvent B over 25 min). The title compound eluted at 16 min. and was concentrated to give 2 mg of a colorless solid. ESI-MS calc for C₁₄₆H₂₀₉N₂₇O₅₀S₂ 801.1 (M+4H⁺), found 802.1.

### Example 15. Synthesis of LTVSPNY-Adoa-PEG₁₂-Thioester

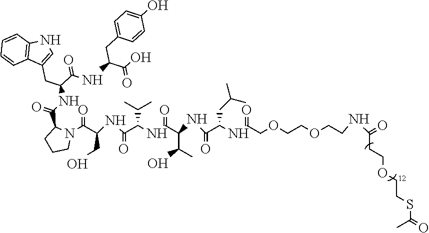

To a solution of LTVSPNY-Adoa-NH₂ (10 mg, 10.7 μmol) in a mixture of CH₃CN/H₂O (500 μL, 1:1) was added (46 μL, 20.8 μmol, 16.1 mg) of a freshly prepared stock solution of S-Acetyl-PEG₁₂-NHS (350 mg/mL) in DMSO. The pH was adjusted to 6.5-7.0 and the reaction mixture stirred overnight. The pH was then adjusted to 7.5-8.0 and the reaction mixture was stirred for ˜2 h. The crude product was purified by HPLC (Gradient: 10% solvent B to 70% solvent B over 25 min) to afford, after concentration, 9 mg of the title compound as a colorless solid (51% yield). ESI-MS calc for C₇₈H₁₂₆N₉NaO₂₈S 845.9, found 845.9 (M+H⁺+Na⁺).

### Example 16. Synthesis of Tubulysin-A BOC-amine

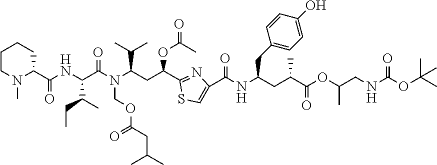 [SMILES: CC[C@H](C)[C@H](NC(=O)[C@H]1CCCCN1C)C(=O)N(COC(=O)CC(C)C)[C@H](C[C@@H](OC(C)=O)c1nc(C(=O)N[C@@H](Cc2ccc(O)cc2)C[C@H](C)C(=O)OC(C)CNC(=O)OC(C)(C)C)cs1)C(C)C]

Tubulysin-A (0.055 mmol) is taken up in CH₂Cl₂ (1 mL) and the solution is cooled to 0° C. EDC (0.82 mmol) and N,N-dimethylpyridin-4-amine (0.273 mmol) is added. The reaction mixture is stirred at 0° C. for 20 min and then t-butyl 2-hydroxypropylcarbamate (0.546 mmol) is added. The reaction mixture is allowed to warm to room temperature and stirred for 24 h. The sample can be purified by preparative HPLC, eluting with 0.1% TFA/CH₃CN and 0.1% TFA/water, followed by lyophilization to give the title compound.

### Example 17. Synthesis of Tubulysin-A Amine

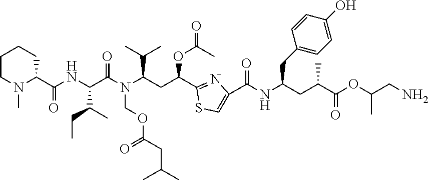 [SMILES: CC[C@H](C)[C@H](NC(=O)[C@H]1CCCCN1C)C(=O)N(COC(=O)CC(C)C)[C@H](C[C@@H](OC(C)=O)c1nc(C(=O)N[C@@H](Cc2ccc(O)cc2)C[C@H](C)C(=O)OC(C)CN)cs1)C(C)C]

Tubulysin-A BOC-Amine (0.022 mmol, prepared as described in Example 16) is taken up in CH₂Cl₂ (0.500 mL) and cooled to 0° C. 2,2,2-Trifluoroacetic acid (2.61 mmol) is added drop wise, then stirred at room temperature for 30 min. The solvent is removed under reduced pressure. The resulting oil can be taken up in CH₂Cl₂ followed by the addition of ether to give the title compound.

### Example 18. Synthesis of PHF-GA-Tubulysin-A Amine-SH

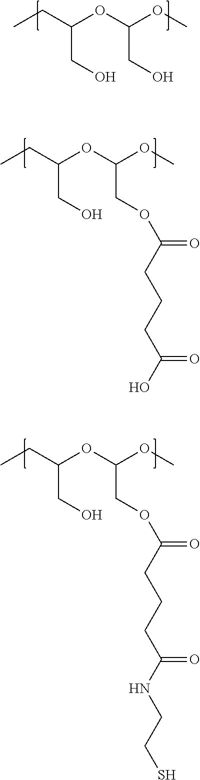

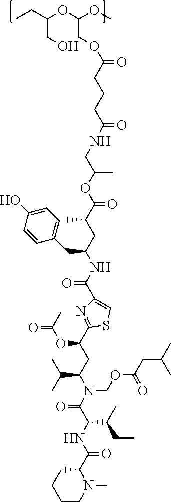

PHF-GA-SSpy (3.19 μmol, PHF-GA-SSpy prepared as described in Example 6) is taken up in a mixture of water (2 mL) and CH₃CN (2 mL) and cooled to 0° C. NHS (0.032 mmol) is added followed by an aqueous solution of EDC (0.032 mmol) and Tubulysin-A amine (0.019 mmol, prepared as described in Example 17) in water (1 mL). The pH of the resulting mixture is adjusted to 6.0 to 6.5, and then stirred at room temperature overnight. The pH is adjusted to 7.5 with 1M NaHCO₃ and DTT (0.065 mmol) is added. The reaction mixture is stirred at room temperature for 30 min, diluted to 15 mL with water, filtered through a 2 micron filter and can be purified by dialysis using a Regenerated cellulose membrane (3 K MW cut-off) by washing with 1% NaCl/water (3×10 mL) followed by water (2×10 mL). The title product is obtained and its SH content is determined.

By substituting tubulysin-A amine with other drug moieties or drug derivatives in the procedures described above it is possible to synthesize other drug-polymer conjugates.

### Example 19. Synthesis of Tubulysin-B2

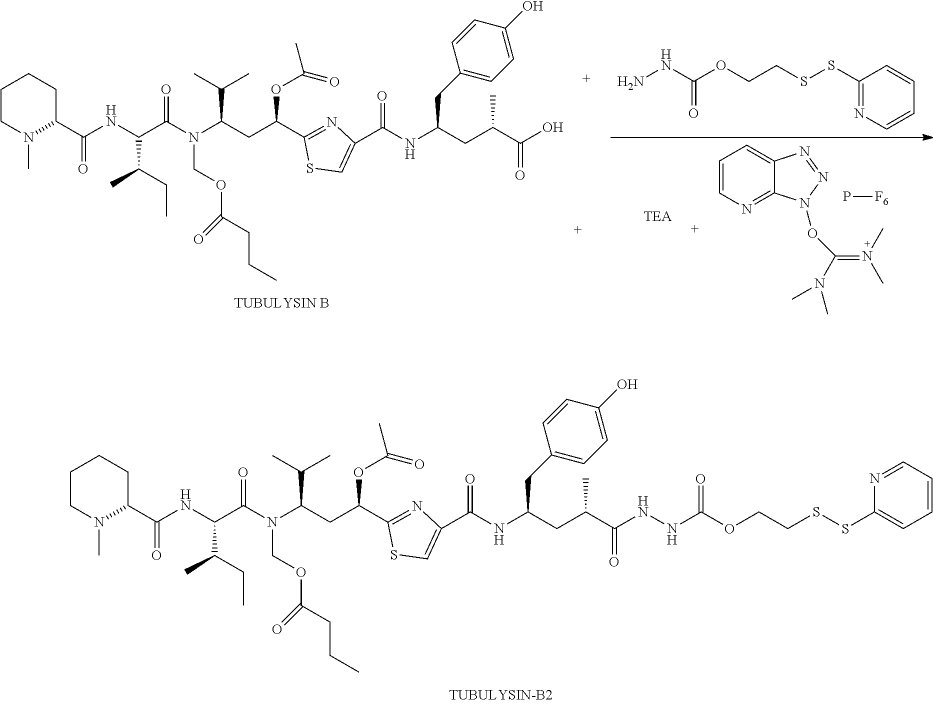

To a solution of Tubulysin-B (6.74 μmol) in DMF (33 μL) at 0° C. under argon is added TEA (1.88 μL, 0.013 mmol). The mixture is stirred for 5 min and then (2-(pyridine-2-yldisulfanyl)ethyl hydrazinecarboxylate (10.1 μmol) in DMF (20 μL) and HATU (10.1 μmol) are added. The reaction mixture is allowed to warm to room temperature, stirred for 2.5 h, diluted with a mixture of water (750 μL) and CH₃CN (1 mL) and then can be purified by preparative HPLC eluting with 0.1% TFA/CH₃CN and 0.1% TFA/water, followed by lyophilization to give the title compound.

### Example 20. Synthesis of Tubulysin-B3

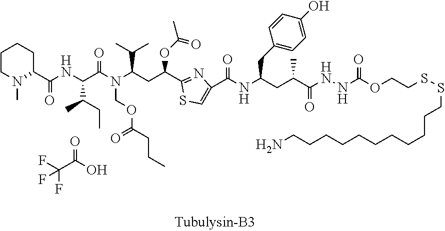

Tublysin-B2 (0.012 mmol, prepared as described in Example 19) is dissolved in DMF (0.3 mL) and 11-aminoundecane-1-thiol hydrochloride (0.123 mmol) in DMF (0.3 mL) is added at 0° C. The reaction mixture is allowed to warm to room temperature and is stirred for 2 days, diluted with water (2 mL) and can be purified by preparative HPLC, followed by lyophilization to give the title compound.

### Example 21. Synthesis of 70 kDa PHF-GA-(Tubulysin-B3)

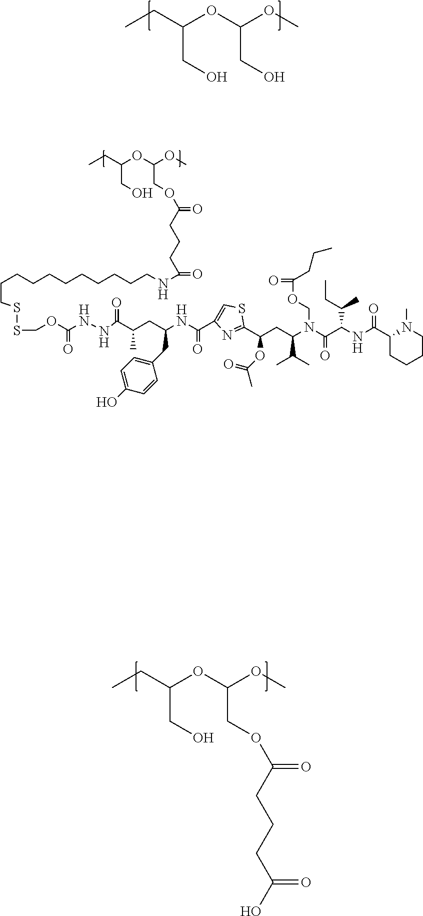

70 KDa PHF-GA (0.217 mmol, prepared using the procedure described in Example 2 with 70 KDa PHF, 9% GA) is dissolved in a mixture of water (2.17 mL) and DMF (0.05 mL). Tubulysin-B3 (10.9 μmol, prepared as described in Example 20) in DMF (0.05 mL) is added and the pH is adjusted to 5 to 6. The resulting solution is cooled to 0° C. and EDC (0.022 mmol) is added portion-wise over 4 h. The reaction mixture is stirred for 6 h at pH 5.0 to 6.0. Purification by size exclusion chromatography eluting with water can give the title compound

### Example 22. Synthesis of Tubulysin-B Proline Ester

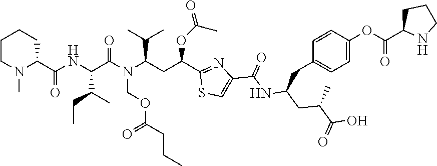 [SMILES: CCCC(=O)OCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(OC(=O)[C@H]3CCCN3)cc2)C[C@H](C)C(=O)O)cs1)C(C)C]

To an ice cold solution of (S)-1-(tert-butoxycarbonyl)pyrrolidine-2-carboxylic acid (0.013 mmol) in DMF (250 μL) is added DIC (2.018 μL, 0.013 mmol) and the resulting mixture is stirred for 15 mins and then added to a solution of Tubulysin-B (6.48 μmol) and DMAP (0.019 mmol) in DMF (250 μL). The reaction mixture is stirred cold and then at room temperature. After 4 h another aliquot of (S)-1-(tert-butoxycarbonyl) pyrrolidine-2-carboxylic acid (0.013 mmol), DIC (0.013 mmol) in 100 μL of DMF is added and the stirring is continued overnight at room temperature. The crude product can be purified by HPLC followed by lyophilized to give the Boc-protected Tubulysin-B.

To an ice cold solution of the Boc-protected Tubulysin-B compound with 2,2,2-trifluoroacetic acid (1:1) (4.06 μmol) in DCM (300 μL) is added TFA (0.406 mmol) and the resulting mixture is stirred cold for 1 h followed by stirring at room temperature for 1 h. The reaction mixture is concentrated, dissolved in acetonitrile and lyophilized to a give the title compound.

### Example 23. Synthesis of Tubulysin-B Diaminobutyl Carbamate

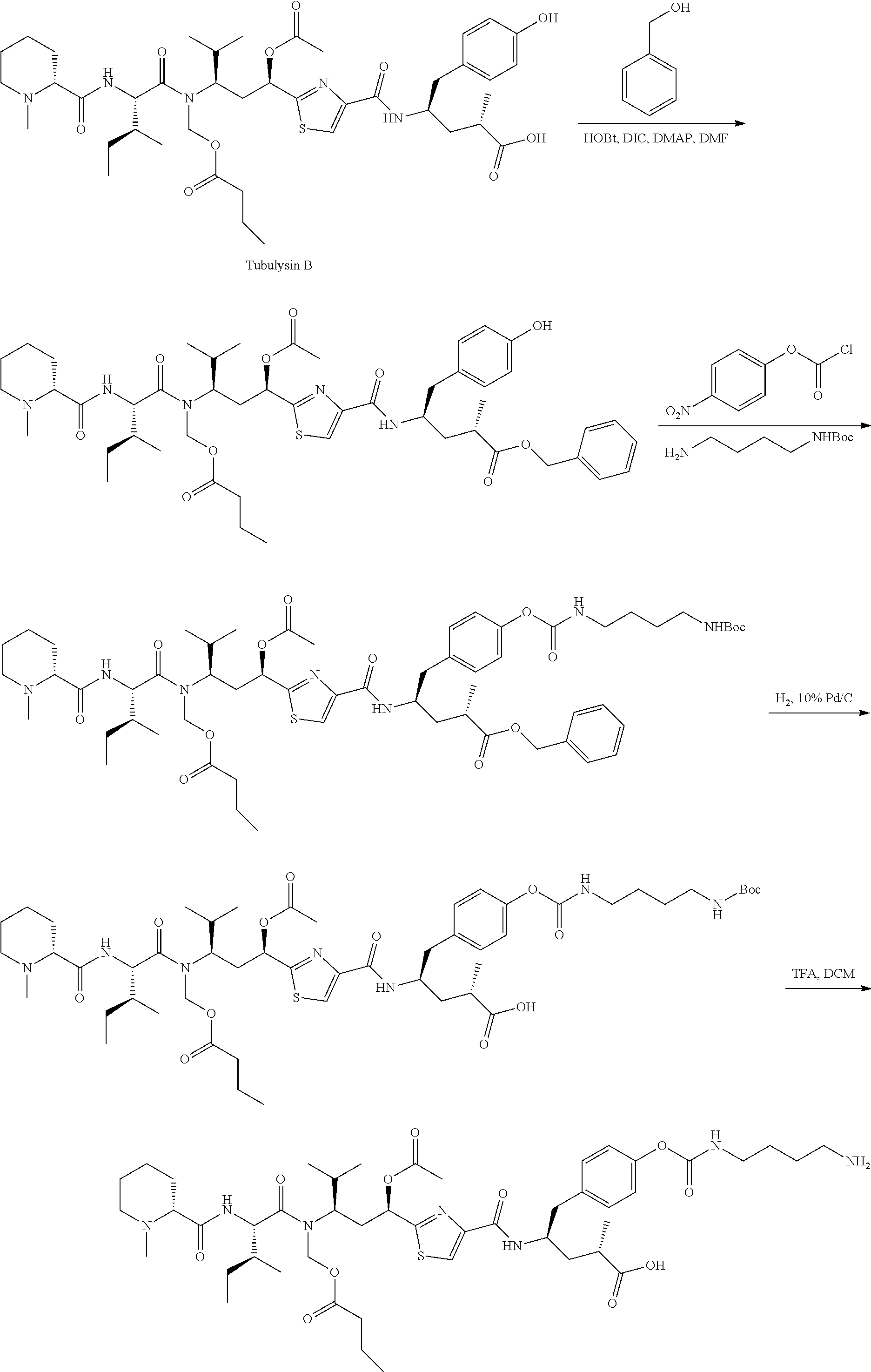

Tubulysin-B (0.129 mmol), HOBt (0.257 mmol) and DMF (5 ml) are combined at room temperature with stirring. After 15 min phenylmethanol (0.643 mmol) and DMAP (0.386 mmol) is added. After 10 min DIC (0.193 mmol) is added. After 16 h at room temperature the crude reaction mixture can be purified to give Tubulysin-B benzyl ester.

To a solution of Tubulysin-B benzyl ester (0.024 mmol) in THF (4 ml) at room temperature is added HOBt (0.047 mmol), 4-nitrophenyl carbonochloridate (0.047 mmol), and triethylamine (0.033 ml, 0.236 mmol). The reaction is monitored by LC/MS or HPLC for the appearance of the p-nitrophenyl carbonate Tubulysin-B intermediate. A second aliquot of HOBt (0.047 mmol), 4-nitrophenyl carbonochloridate (0.047 mmol) and triethylamine (0.236 mmol) is added. After 45 min, to the reaction mixture is added tert-butyl 4-aminobutylcarbamate (0.118 mmol) and triethylamine (0.033 ml, 0.236 mmol). After ˜30 minutes, the reaction mixture can be purified to give Tubulysin-B Boc-amino butylcarbamate benzyl ester.

To an argon bubbled solution of Tubulysin-B Boc-diamino butylcarbamate benzyl ester (0.015 mmol) in THF (2 mL) and ethanol (2.000 ml) is added palladium on carbon (0.015 mmol) followed by attachment of a balloon to the flask to deliver hydrogen (0.015 mmol). The reaction mixture is stirred vigorously until LC/MS or HPLC indicates that the reaction is complete. The crude reaction mixture can be purified to give Tubulysin-B Boc-amino butylcarbamate.

To an ice cold solution of tubulysin-B Boc-diamino butylcarbamate (5.91 μmol) in DCM (1 mL) is added TFA (5.91 μmol) and the reaction mixture is stirred cold for 1 h and then at room temperature for 1 h. After LC/MS or HPLC indicates that the reaction is complete the reaction mixture is concentrated and the residue taken up in acetonitrile and water with 0.1% TFA and then it can be lyophilized to give the title compound.

### Example 24. Synthesis of 22 kDa PHF-BA-SSPyr-(Tubulysin-B Diaminobutyl Carbamate)

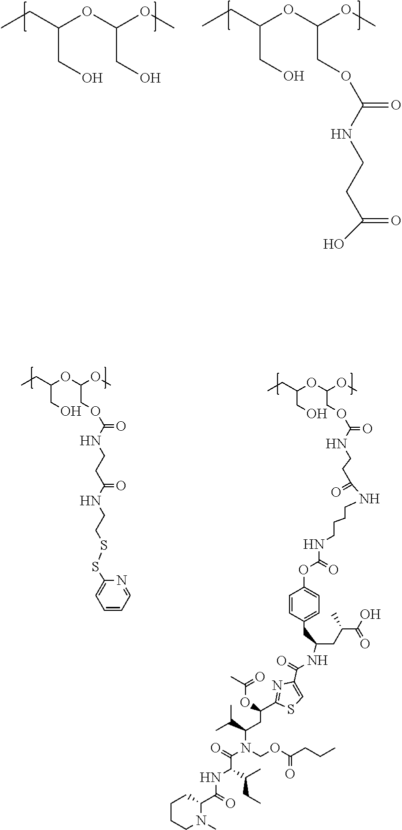

22 kDa PHF-BA (29%)-SSPy (5%) (0.492 μmol, prepared using the procedure described in Example 7 with PHF-BA (29%) (MW ˜22 kDa) which is prepared as described in Example 1) is dissolved in NMP (0.5 mL) with heating. The reaction mixture is cooled to room temperature and HOBt (0.012 mmol) in NMP (0.1 mL) and EDC (0.012 mmol) in NMP (0.2 mL) are added. The mixture is stirred for 10 minutes and a solution of DIPEA (7.86 μmol) and Tubulysin-B butylcarbamate (5.90 μmol, prepared as described in Example 23) in NMP (0.300 mL) are added. After stirring at room temperature for 18 h the mixture can be diluted to 5% organics with deionized water, concentrated via dialysis using a Regenerated cellulose membrane (3K) followed by purification by HPLC to give the title compound.

### Example 25. Synthesis of 22 kDa PHF-BA (29%) (Tubulysin-B Diaminobutyl Carbamate)-(S—S-Trastuzumab)

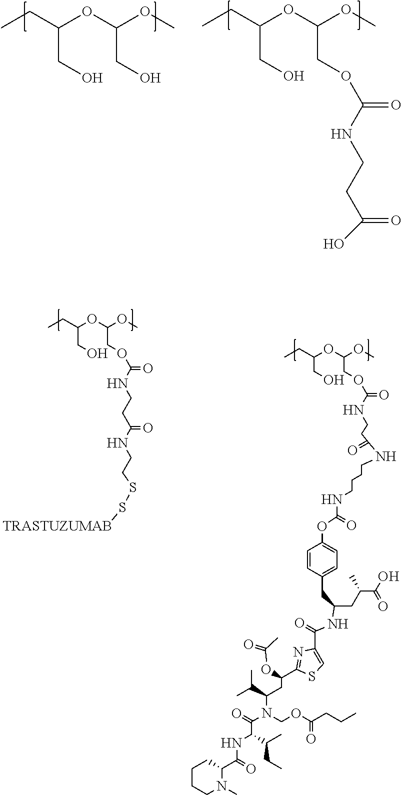

To trastuzumab-F(ab′)₂ (3.44 mL, 0.325 μmol of 10.4 mg/mL stock, prepared as described in Example 9) in PBS, pH 7.4 was added an aliquot (138 μL, 0.467 mg) of freshly prepared TCEP stock (3.38 mg/mL in Et₃NHOAc buffer). The mixture was incubated 1 h at 37° C. The reaction mixture was cooled to room temperature and then purified on a PD10 column which was preequilibrated with Et₃NHOAc buffer to give trastuzumab-Fab′, MW (SDS PAGE), about 50 to 55 kDa.

To reduced trastuzumab (5 mg, prepared as described above) in triethylamine acetate buffer (1 mL, 50 mM, containing 1 mM EDTA, pH=7.0) is added 22 kDa PHF-BA (29%)-SSPy (5%)-(tubulysin-B diaminobutyl carbamate) (3.5 mg, prepared as described in Example 24). After 18 h at room temperature the resulting conjugate can be isolated and purified by diafiltration. The Tubulysin-B to Trastuzumab ratio will be about 12:1 to about 15:1.

Other protein-polymer-drug conjugates are synthesized with methods similar to the procedure described above, involving other PBRM derivatives, such as, for example, reduced form of cetuximab, rituximab, bevacizumab, nimotuzumab, gemtuzumab, alemtuzumab, lintuzumab, anti-5T4 or anti-mesothelin antibodies. Also PBRM-drug polymer conjugates with varying ratios of drug to PBRM are obtained by varying the amount of PBRM and drug-polymer scaffold used in the Examples above.

### Example 26. Synthesis of Tubulysin-A Valine Ester

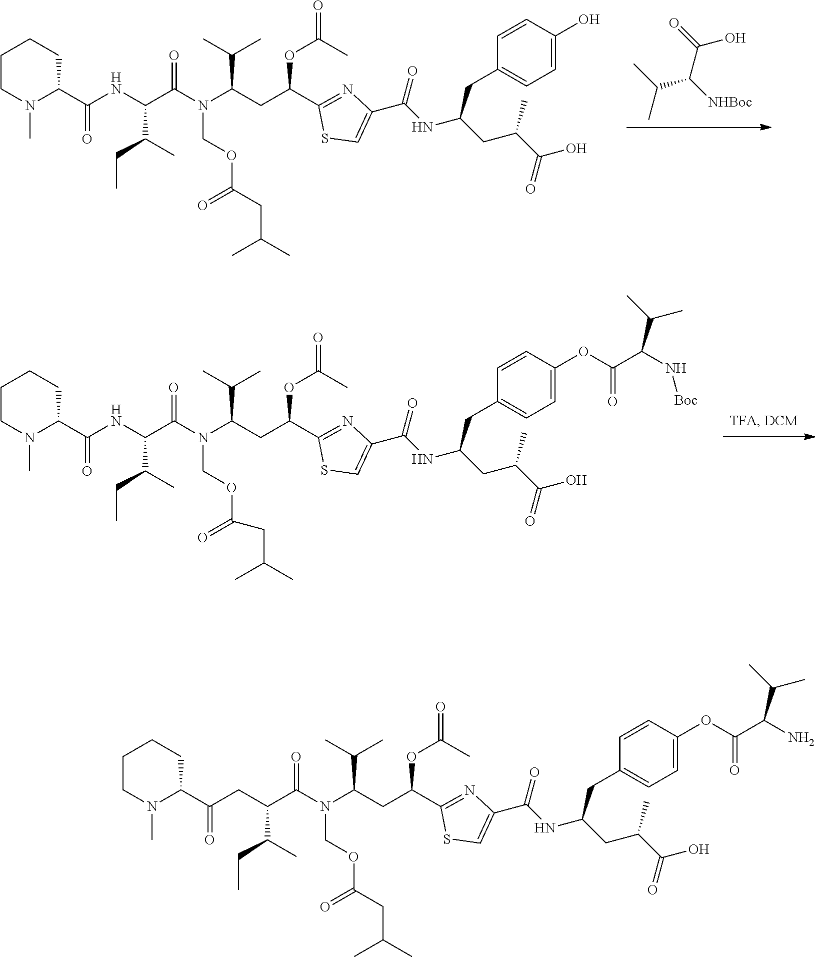 [SMILES: CC[C@H](C)[C@H](NC(=O)[C@H]1CCCCN1C)C(=O)N(COC(=O)CC(C)C)[C@H](C[C@@H](OC(C)=O)c1nc(C(=O)N[C@@H](Cc2ccc(O)cc2)C[C@H](C)C(=O)O)cs1)C(C)C ; CC(C)[C@@H](NC(=O)OC(C)(C)C)C(=O)O ; CC[C@H](C)[C@H](NC(=O)[C@H]1CCCCN1C)C(=O)N(COC(=O)CC(C)C)[C@H](C[C@@H](OC(C)=O)c1nc(C(=O)N[C@@H](Cc2ccc(OC(=O)[C@H](NC(=O)OC(C)(C)C)C(C)C)cc2)C[C@H](C)C(=O)O)cs1)C(C)C ; CC[C@H](C)[C@H](CC(=O)[C@H]1CCCCN1C)C(=O)N(COC(=O)CC(C)C)[C@H](C[C@@H](OC(C)=O)c1nc(C(=O)N[C@@H](Cc2ccc(OC(=O)[C@H](N)C(C)C)cc2)C[C@H](C)C(=O)O)cs1)C(C)C]

To an ice cold solution of N-Boc-D-valine (0.080 mmol) in DMF (500 μL) is added DIC (0.072 mmol) and the resulting solution is stirred cold for 15 min, then it is added to a solution of Tubulysin-A (0.034 mmol) and DMAP (0.109 mmol) in DMF (500 μL). The resulting mixture is stirred cold for 15 min and then overnight at room temperature. The crude reaction mixture can be purified to give tubulysin-A N-Boc-D-valine.

To an ice cold solution of tubulysin-A N-Boc-D-valine ester (0.018 mmol) and 2,2,2-trifluoroacetic acid (1:1) in DCM (3 mL) is added TFA (3.69 mmol). The resulting mixture is stirred cold for 1 h then at room temperature for 1 h, followed by purification gives the title compound.

### Example 27. Synthesis of 7 kDa PHF-BA-SSPyr-(Tubulysin-A Valine)

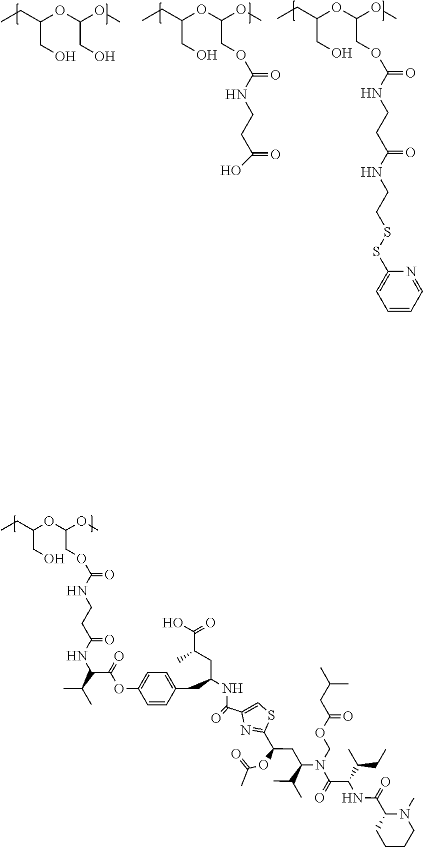

7K PHF-BA(29%)-SSPyr(6%) (258 mg, 0.378 mmol, prepared using the procedure described in Example 7 with PHF BA (29%) (MW ˜7 kDa) which was prepared as described in Example 1) is reacted with Tubulysin-A valine ester (10.2 mg, 0.011 mmol, prepared as described in Example 26) using the procedure of Example 24.

### Example 28. Synthesis of 7 kDa PHF-BA (29%) (Tubulysin-A Valine)-(S—S-Trastuzumab)

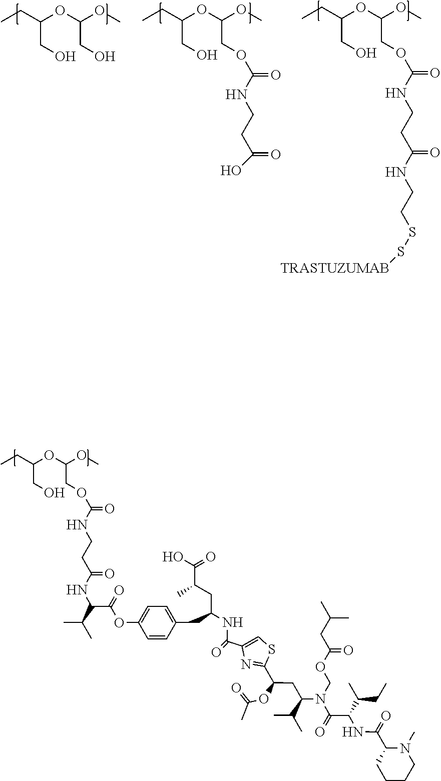

The title compound can be prepared using the procedure described in Example 25 except 7 kDa PHF-BA-SSPyr-(Tubulysin-A valine) (prepared as described in Example 27) can be used. The Tubulysin-A to Trastuzumab ratio will be about 6:1 to about 10:1.

Other protein-polymer-drug conjugates are synthesized with methods similar to the procedure described above, involving other PBRM derivatives, such as, for example, reduced form of cetuximab, rituximab, bevacizumab, nimotuzumab, gemtuzumab, alemtuzumab, lintuzumab, anti-5T4 or anti-mesothelin antibodies as described above. Also PBRM-drug polymer conjugates with varying ratios of drug to PBRM are obtained by varying the amount of PBRM and drug-polymer scaffold used in the Examples above.

### Example 29. Synthesis of Tubulysin-A Dimethyl-Diaminoethyl-PABA-Val Cit-NH₂

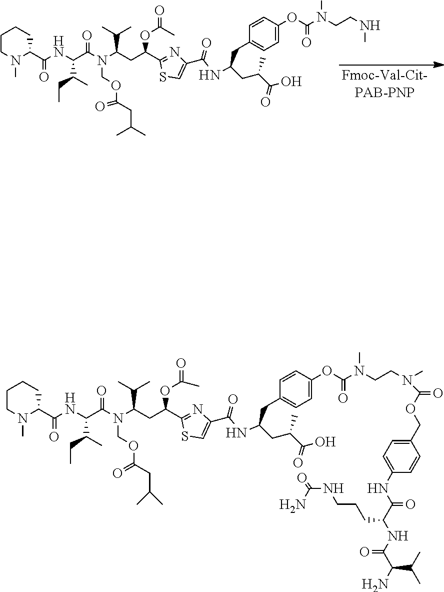 [SMILES: CC[C@H](C)[C@H](NC(=O)[C@H]1CCCCN1C)C(=O)N(COC(=O)CC(C)C)[C@H](C[C@@H](OC(C)=O)c1nc(C(=O)N[C@@H](Cc2ccc(OC(=O)N(C)CCNC)cc2)C[C@H](C)C(=O)O)cs1)C(C)C ; CC[C@H](C)[C@H](NC(=O)[C@H]1CCCCN1C)C(=O)N(COC(=O)CC(C)C)[C@H](C[C@@H](OC(C)=O)c1nc(C(=O)N[C@@H](Cc2ccc(OC(=O)N(C)CCN(C)C(=O)OCc3ccc(NC(=O)[C@@H](CCCNC(N)=O)NC(=O)[C@H](N)C(C)C)cc3)cc2)C[C@H](C)C(=O)O)cs1)C(C)C]

Fmoc-Val-Cit-PABA-PNP (0.027 mmol), tubulysin-A dimethyl-diamino ethyl carbamate (22.75 mg, 0.023 mmol, prepared using the procedure described in Example 23 except tert-butyl methyl(2-methylamino)ethylcarbamate can be used instead of tert-butyl 4-methylamino)butyl carbamate), 2,2,2-trifluoroacetic acid (1:1) and HOBt (0.025 mmol) in DMF (2 mL) are stirred at room temperature under argon. To the reaction mixture is added triethylamine (0.114 mmol). After 16 h the crude reaction mixture can be purified to give the title compound.

### Example 30. Synthesis of 13 kDa PHF-BA-SSPyr-(Tubulysin-A-Dimethyl-Diaminoethyl-PABA-Val Cit)

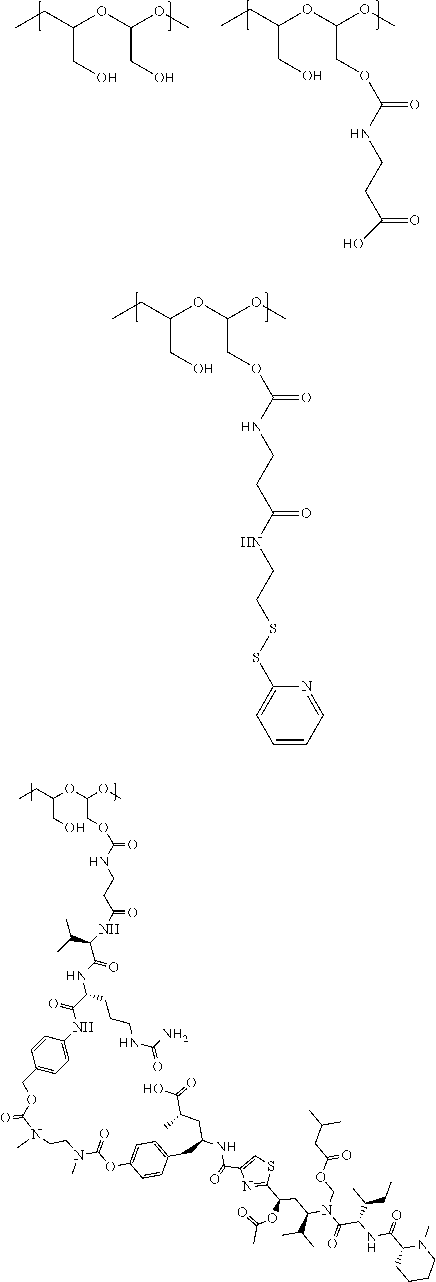

13K PHF-BA(31%)-SSPyr(6%) (1.38 μmol, prepared using the procedure described in Example 7 with PHF BA (31%) (MW ˜13 kDa), prepared as described in Example 1) is reacted with Tubulysin-A-Dimethyl-diaminoethyl-PABA-Val Cit NH₂ (6.90 μmol, prepared as described in Example 29) using the procedure of Example 24.

### Example 31. Synthesis of 13 kDa PHF-BA-(Tubulysin-A-Dimethyl-Diaminoethyl-PABA-Val Cit)-(S—S-Trastuzumab)

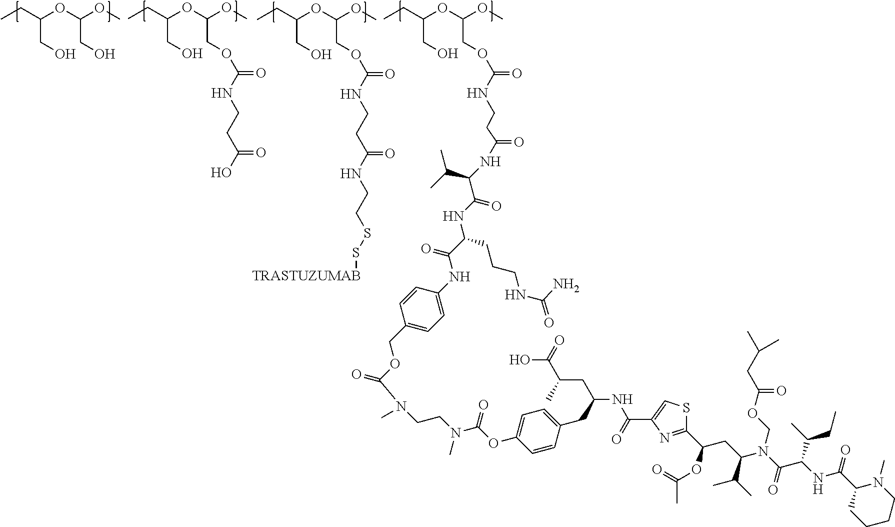

The title compound can be prepared using the procedure described in Example 25 except 13 kDa PHF-BA-SSPyr-(Tubulysin-A-Dimethyl-diaminoethyl-PABA-Val Cit) prepared as described in Example 30 will be used. The Tubulysin-A to Trastuzumab ratio will be about 6:1 to about 10:1.

Other protein-polymer-drug conjugates are synthesized with methods similar to the procedure described above, involving other PBRM derivatives, such as, for example, reduced form of cetuximab, rituximab, bevacizumab, nimotuzumab, gemtuzumab, alemtuzumab, lintuzumab, anti-5T4 or anti-mesothelin antibodies as described above. Also PBRM-polymer-drug conjugates with varying ratios of drug to PBRM are obtained by varying the amount of PBRM and drug-polymer scaffold used in the Examples above.

### Example 32. Synthesis of Tubulysin-A-Dimethyldiamino Ethyl-PABA-Val Cit-Maleimide

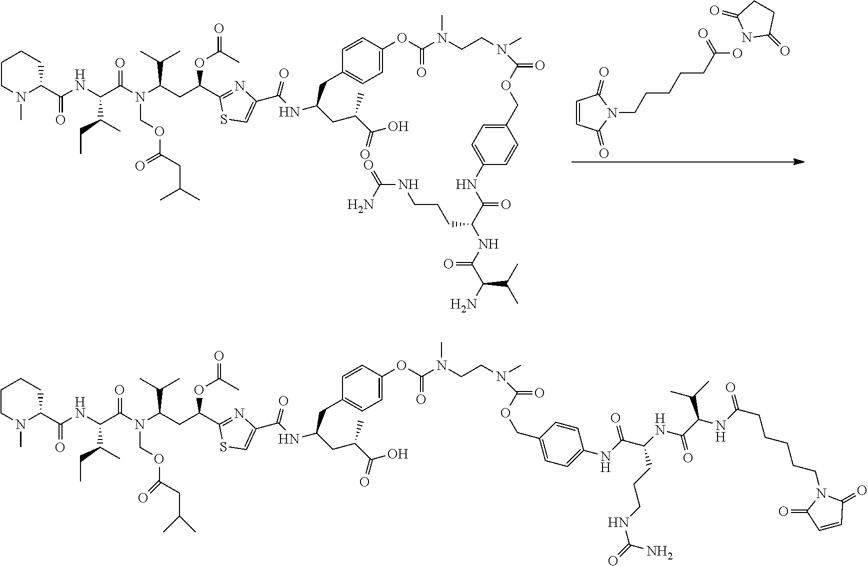

Tubulysin-A Dimethyl-diaminoethyl-PABA-Val Cit-NH₂ (9.05 μmol, prepared as described in Example 29), 2,5-dioxopyrrolidin-1-yl 6-(2,5-dioxo-2,5-dihydro-1H-pyrrol-1-yl)hexanoate (0.018 mmol) and HOBt (0.018 mmol) in DMF (1 mL) is stirred at room temperature, followed by the addition of triethylamine (0.013 ml, 0.091 mmol). After 4 h the reaction mixture can be purified to give the title compound.

### Example 33. Synthesis of Tubulysin-A-Dimethyldiamino Ethyl-PABA-Val Cit-Maleimido-S-Trastuzumab

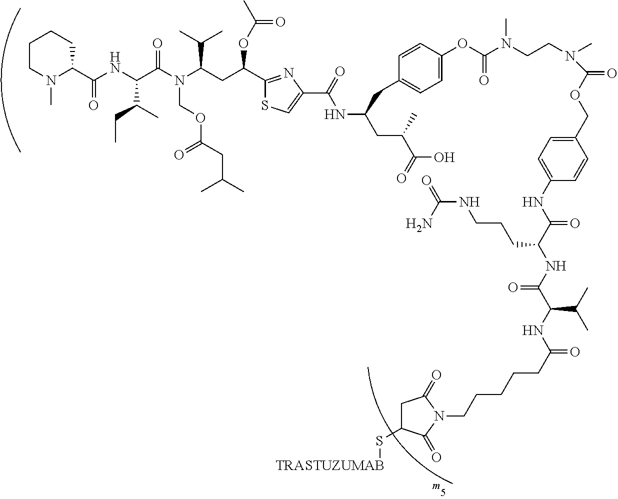

Reduced trastuzumab can be reacted with Tubulysin-A-dimethyldiamino ethyl-PABA-Val Cit-Maleimide using the procedure described in Example 32. The Tubulysin-A to Trastuzumab ratio obtained can be about 9:1 to about 13:1.

By substituting reduced rituximab in the procedure described above, tubulysin-A dimethyldiamino ethyl-PABA-Val Cit-Maleimido-S-rituximab can be synthesized. The tubulysin-A to Rituxiumab ratio can be about 6:1 to about 11:1.

Other protein-drug conjugates are synthesized with methods similar to the procedure described above, involving other PBRM derivatives, such as, for example, reduced form of cetuximab, rituximab, bevacizumab, nimotuzumab, gemtuzumab, alemtuzumab, lintuzumab, anti-5T4 or anti-mesothelin antibodies. Also PBRM-drug polymer conjugates with varying ratios of drug to PBRM are obtained by varying the amount of PBRM and drug-polymer scaffold used in the Examples above.

### Example 34. Synthesis of 13 kDa PHF BA-Tubulysin-A-MI-SH

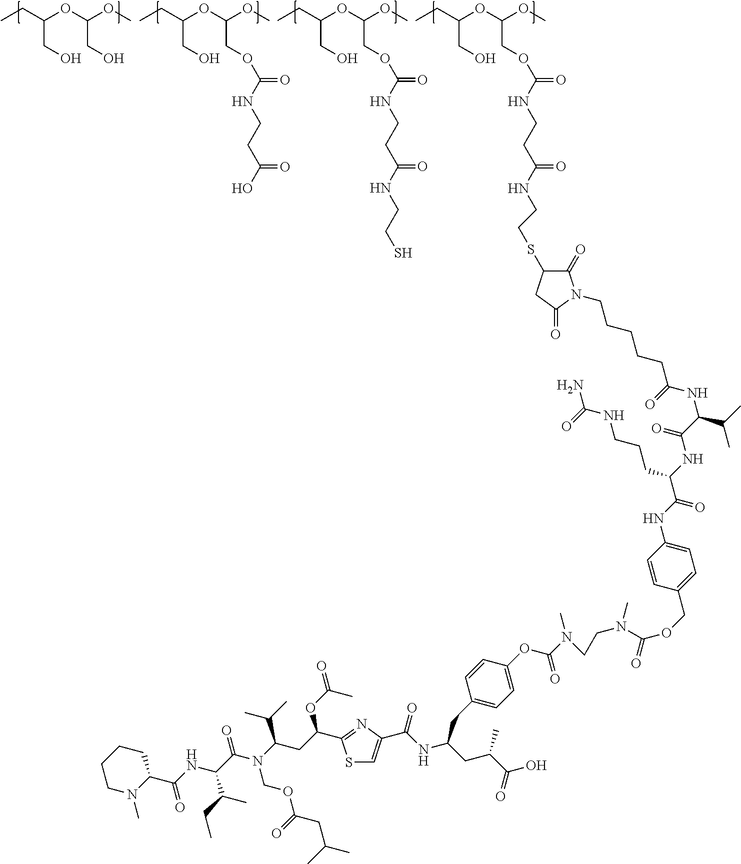

13K PHF-BA(31%)-S—S-pyr(5.9%) (prepared using the procedure described in Example 7 with PHF-BA (31%) (MW ˜13 kDa) is taken up in a mixture of water (8 mL) and acetonitrile (4 mL). The pH is adjusted to 7.5 with 1M NaHCO₃ and DTT (37.8 mg, 0.245 mmol) is added. The reaction mixture is stirred at 23° C. for 30 min then Tubulysin-A-MI prepared as described in Example 32) is added and the resulting mixture is stirred at room temperature for 4 h, pH 7.0. The conjugate can be purified by diafiltration to give the title compound.

### Example 35. Synthesis of 13 kDa PHF BA Tubulysin-A-MI-Trastuzumab-MCC

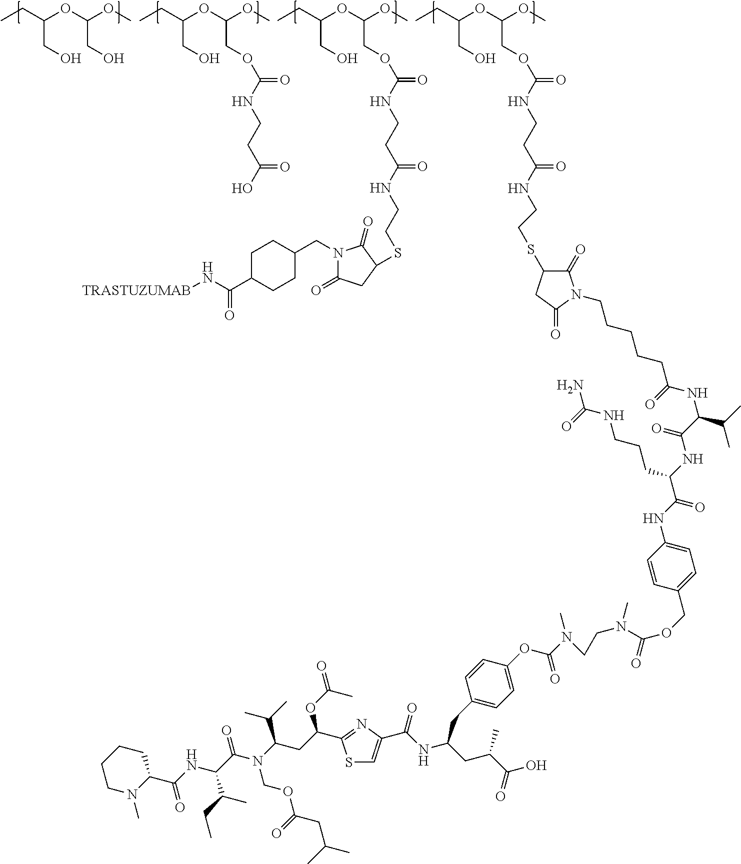

To Trastuzumab-MCC (20 mg, prepared as described in Example 3) in PBS (2 mL, pH 7.0) is added 13 kDa PHF BA-Tubulysin-A-MI-SH (11.2 mg, prepared as described in Example 34) in water (0.5 mL). The solution is stirred at room temperature for 4 h at pH 7.0. The resulting conjugate is purified to give the title compound.

Other protein-polymer-drug conjugates are synthesized with methods similar to the procedure described above, involving other PBRM derivatives, such as, for example, MCC derivatives of cetuximab, rituximab, bevacizumab, nimotuzumab, gemtuzumab, alemtuzumab, lintuzumab, anti-5T4 or anti-mesothelin antibodies as described in Example 3 above. Also PBRM-drug polymer conjugates with varying ratios of drug to PBRM are obtained by varying the amount of PBRM and drug-polymer scaffold used in the Examples above.

### Example 36. Synthesis of 13 kDa PHF BA Tubulysin-A-MI-(Trastuzumab-M-(PEG)₁₂)

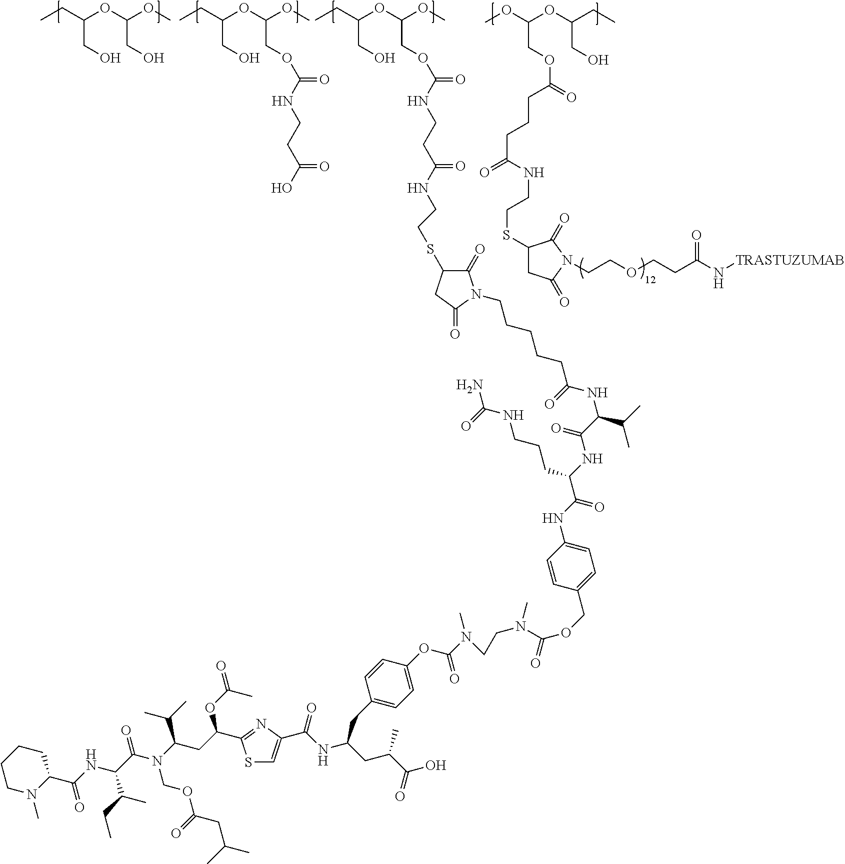

The title compound is prepared as described in Example 35 except Trastuzumab-MCC is replaced by Trastuzumab-M-(PEG)₁₂ (prepared as described in Example 4). The molecular weight of the resulting PHF BA Tubulysin-A-MI-(Trastuzumab-M-(PEG)₁₂) conjugate is determined by SEC.

Other protein-polymer-tubulysin compound conjugates are synthesized with methods similar to the procedure described above, involving other PBRM derivatives, such as, for example, M-(PEG)₁₂ derivatives cetuximab, rituximab, bevacizumab, nimotuzumab, gemtuzumab, alemtuzumab, lintuzumab, anti-5T4 or anti-mesothelin antibodies as described in Example 4 above. Also PBRM-drug polymer conjugates with varying ratios of drug to PBRM are obtained by varying the amount of PBRM and drug-polymer scaffold used in the Examples above.

### Example 37. Cell Viability Assay for PBRM-Drug Polymer Conjugates

PBRM-tubulysin compound polymer conjugates are evaluated for their tumor viability using Cell Titer-Glo (Promega Corp). Cells are plated in black walled 96-well plate and allowed to adhere overnight at 37° C. in a humidified atmosphere of 5% CO₂. HER2 expressing cells SKBR3, BT474, NCI-N87 and cells expressing low levels of HER2-MCF7 are plated at a density of 5,000 cells per well. The next day the medium is replaced with 50 μL fresh medium and 50 μL of 2× stocks of PBRM-drug polymer conjugate, tubulysin compound polymer conjugate or tubulysin compound is added to appropriate wells, mixed and incubated for 72 h. Cell Titer-Glo reagent is added to the wells at room temperature and the luminescent signal is measured after 10 min using a SpectraMax M5 plate reader (Molecular Devices). Dose response curves are generated using SoftMax Pro software. IC₅₀ values are determined from four-parameter curve fitting.

### Example 38. In Vivo Efficacy, Pharmacokinetic and Biodistribution Studies

In order to evaluate the efficacy and pharmacokinetics of the protein drug conjugate mouse and rat subcutaneous and orthotopic xenograft models are used.

Test articles, along with appropriate controls are administered intravenously (IV) via tail-vein injection or intraperitoneally. To assess circulating levels of test article blood sample is collected at designated times via terminal cardiac-puncture. Samples are kept at room temperature for 30 min to coagulate, then centrifuged for 10 min at 1,000×g at 4° C. and immediately frozen at −80° C. Total PBRM concentrations in serum samples are measured using ELISA. Circulating tubulysin compound concentration (conjugated and free) is determined by LC/MS methods.

To assess efficacy of the PBRM-tubulysin compound polymer conjugates the tumor size are measured using digital calipers. Tumor volume is calculated and used to determine the delay in tumor growth.

For the determination of drug biodistribution, tumor, and major organs such as, for example, liver, kidney, spleen, lung, heart, muscles, and brain are harvested, immediately frozen in liquid nitrogen, stored at −80° C. PBRM and/or tubulysin compound levels are determined in tissue homogenates by standard methods, such as, for example, ELISA or LC/MS/MS methods respectively.

### Example 39. Tumor Growth Response to Administration of PBRM-Drug Polymer Conjugates

Female CB-17 SCID mice are inoculated subcutaneously with NCI-N87 cells (n=10 for each group) or BT474 tumors (n=12 or n=10 for each group). Test compounds or vehicle are dosed IV as a single dose on day 1; once every week for 3 weeks on day 1, day 8 and day 15 respectively; or once every week for 3 weeks on day 17, day 24 and day respectively. The tubulysin compound polymer conjugate dose is determined such that it delivered the same amount of tubulysin compound as that present in the highest dose of the corresponding PBRM-tubulysin compound polymer conjugate is administered Tumor size is measured at several different time points using digital calipers. Tumor volume is calculated and is used to determine the delay in tumor growth. Mice are sacrificed when tumors reach a size of 1000 mm³, 800 mm³, or 700 mm³. Tumor volumes are reported as the mean±SEM for each group.

### Example 40. In Vitro Stability of PBRM-Tubulysin Compound Polymer Conjugates

The in vitro stability of PBRM-tubulysin compound polymer conjugates are evaluated by incubation of the PBRM-tubulysin compound polymer conjugate in physiological saline or animal plasma at 37° C., pH 7.4. The rate of PBRM-tubulysin compound polymer conjugate degradation is determined by monitoring the amount of drug released into the matrix by LC/MS analysis after isolation of released drug from the PBRM-tubulysin compound polymer conjugate by liquid-liquid extraction.

### Example 41. Ligand Binding Studies by BIAcore Surface Plasmon Resonance (SPR)

The kinetic binding of the PBRM-tubulysin compound polymer conjugate to an immobilized receptor is determined by BIAcore SPR. The binding constants for the PBRM in the PBRM-tubulysin compound-conjugate and PBRM alone are determined using standard BIAcore procedures.

### Example 42. Mouse Plasma PK and Tissue Distribution after Administration of PBRM-Tubulysin Compound Polymer Conjugates

The plasma PK stability and the tissue distribution of PBRM-tubulysin compound-conjugate is determined after administration of PBRM-tubulysin compound-conjugate in female CB-17 SCID mice with NCI-N87 tumors (n=3). The conjugated tubulysin compound concentration is determined by LC/MS analysis. The concentration of the tubulysin compound-PBRM-conjugate is estimated from the conjugated tubulysin compound data. Total PBRM concentration is determined by ELISA.

### INCORPORATION BY REFERENCE

The entire disclosure of each of the patent documents and scientific articles referred to herein is incorporated by reference for all purposes.

### EQUIVALENTS

The invention can be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

1. A polymeric scaffold of Formula (Ia) useful to conjugate with a protein based recognition-molecule (PBRM): 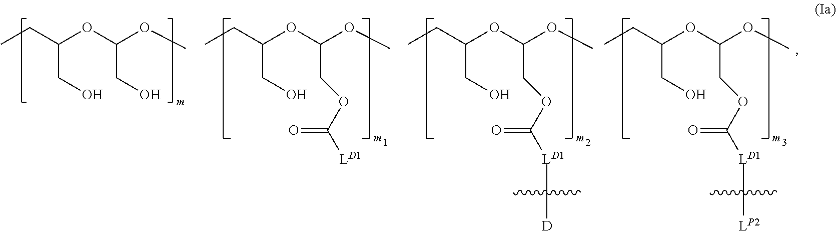
wherein:
the scaffold comprises poly(1-hydroxymethylethylene hydroxymethyl-formal) (PHF) having a molecular weight ranging from 2 kDa to 40 kDa when the PBRM to be conjugated has a molecular weight of greater than 40 kDa, or the scaffold comprises PHF having a molecular weight ranging from 20 kDa to 300 kDa when the PBRM to be conjugated has a molecular weight of less than 80 kDa;
L^{D1} is a carbonyl-containing moiety;
each occurrence of 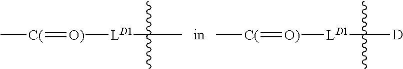
is independently a first linker that contains a biodegradable bond so that when the bond is broken, D is released in an active form for its intended therapeutic effect; the 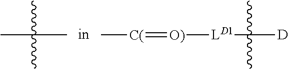
between L^{D1} and D denotes direct or indirect attachment of D to L^{D1};
each occurrence of 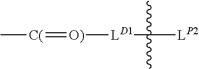
is independently a second linker not yet connected to the PBRM, in which L^{P2} is a moiety containing a functional group that is capable of forming a covalent bond and not yet formed with a functional group of a PBRM, and the 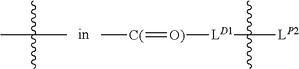
between L^{D1} and L^{P2} denotes direct or indirect attachment of L^{P2} to L^{D1}, and each occurrence of the second linker is distinct from each occurrence of the first linker;
m is an integer from 1 to 2200
m₁ is an integer from 1 to 660,
m₂ is an integer from 1 to 300,
m₃ is an integer from 1 to 110, the sum of m, m₁, m₂ and m₃ ranges from 15 to about 2200;
each occurrence of D contains a functional group that is capable of forming a covalent bond so as to attach D to L^{D1} and is independently a compound of Formula (II) or a pharmaceutically acceptable salt thereof: 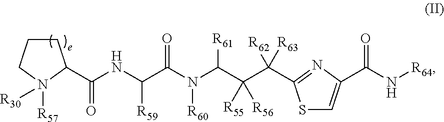
wherein:
e is 2;
R₅₅ is hydrogen;
R₅₆ is hydrogen or OH; or R₅₅ and R₅₆ together form an oxo group (═O);
R₅₇ is methyl or ethyl, or —C(O)R₅₈ and R₃₀ is absent or R₅₇ is methyl and R₃₀ is O;
R₅₈ is C₁₋₆ alkyl, CF₃ or C₆₋₁₀ aryl;
R₅₉ is C₁₋₆ alkyl;
R₆₀ is hydrogen, methyl, —CH₂OR₆₅, or —CH₂NHR₆₅;
R₆₁ is C₁₋₆ alkyl optionally substituted with C₃₋₁₀ cycloalkyl, or C₃₋₁₀ cycloalkyl optionally substituted with C₁₋₆ alkyl;
R₆₂ is hydrogen or alkyl;
R₆₃ is hydrogen, halo, OH, —O—C₁₋₄ alkyl or O—C(O)—R₃₄, in which R₃₄ is C₁₋₄ alkyl, C₂₋₇ alkenyl, or C₆₋₁₀ aryl; or R₆₂ and R₆₃ together form an oxo group (═O);
R₆₅ is hydrogen, C₁₋₆ alkyl optionally substituted with OH or SH, C₂₋₇ alkenyl, or C(O)R₆₇; and
R₆₇ is C₁₋₆ alkyl, C₂₋₇ alkenyl, C₆₋₁₀ aryl or heteroaryl;
R₆₄ is 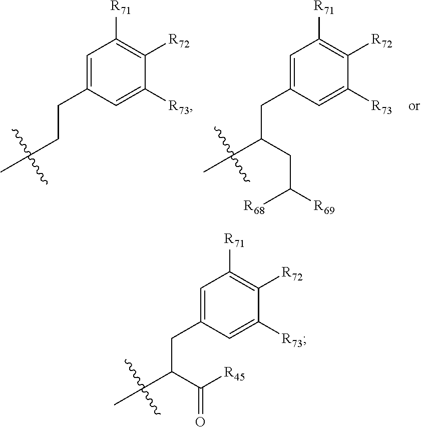
R₆₈ is hydrogen or C₁-C₆ alkyl;
R₆₉ is CO₂R₇₀, C(O)—R₄₅, CONHNH₂, OH, NH₂, SH, or an optionally substituted alkyl, an optionally substituted cycloalkyl, an optionally substituted heteroalkyl or an optionally substituted heterocycloalkyl group;
R₇₀ is an optionally substituted alkyl, an optionally substituted heteroalkyl or an optionally substituted heterocycloalkyl group;
each of R₇₁ and R₇₃ independently is hydrogen, OH, mono- or di-alkylamino, halo, —NO₂, —CN, —NHR₇₄, C₁₋₆ alkyl, haloalkyl, alkoxy or haloalkoxy;
R₇₂ is hydrogen, OR₄₃, alkoxy, halogen, —NHR₇₄, —O—C(O)—R₄₇, NO₂, —CN, C₆₋₁₀ aryl, C₁₋₆ alkyl, amino or dialkylamino;
R₇₄ is hydrogen, —CHO, —C(O)—C₁₋₄ alkyl, OH, amino group, alkyl amino or —[C(R₂₀R₂₁)]ₐ—R₂₂;
R₄₅ is mono- or di-alkylamino, —OR₄₂ or —NHR₄₀, and provided that at least one of R₄₃, R₄₂ and R₄₀ cannot be hydrogen;
R₄₀ is hydrogen, —OH, or —NH₂; R₄₂ is hydrogen; or each of R₄₀ and R₄₂, independently is selected from the following structures: 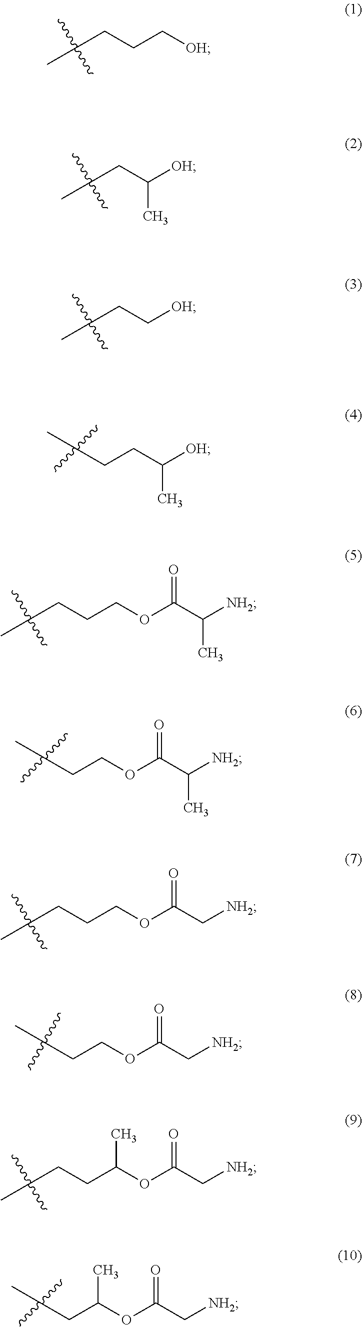 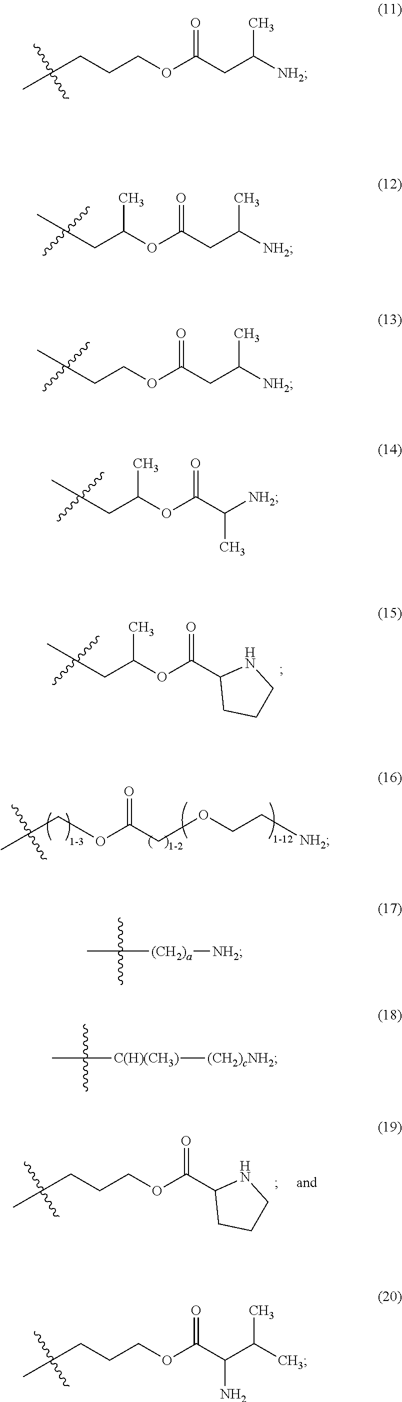
in which a is an integer from 1 to 6; and c is an integer from 0 to 3;
R₄₃ is H or —R₄₆—R₄₇;
R₄₆ is —C(O)—; —C(O)—O—, —C(O)—NH— or absent;
R₄₇ is an amino group, —R₉—[C(R₂₀R₂₁)]ₐ—R₁₀, —R₉—C₅₋₁₂ heterocycloalkyl-C₁₋₆ alkyl-R₁₀, 5 to 12-membered heterocycloalkyl, or —R₉—C₆₋₁₀ aryl;
R₉ is absent, N—(R₈₃) or oxygen;
R₁₀ is —OH, —NHR₈₃, —N—(R₈₃)R₁₁, —COOH, —R₈₂—C(O)(CH₂)_{c}—C(H)(R₂₃)—N(H)(R₂₃), —R₈₂—C(O)(CH₂)_{d}—(OCH₂—CH₂)_{f}—N(H)(R₂₃), —R₈₂—(C(O)—CH(X²)—NH)_{d}—R₇₇ or —R₈₂—C(O)—[C(R₂₀R₂₁)]ₐ—R₈₂—R₈₃ or 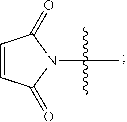 [SMILES: *N1C(=O)C=CC1=O]
X² is a side chain of a natural or unnatural amino acid;
R₇₇ is hydrogen or X² and NR₇₇ form a nitrogen containing cyclic compound;
R₈₂ is —NH or oxygen;
R₈₃ is hydrogen or CH₃;
each of R₂₀ and R₂₁ independently is hydrogen, C₁₋₆ alkyl, C₆₋₁₀ aryl, hydroxylated C₆₋₁₀ aryl, polyhydroxylated C₆₋₁₀ aryl, 5 to 12-membered heterocycle, C₃₋₈ cycloalkyl, hydroxylated C₃₋₈ cycloalkyl, polyhydroxylated C₃₋₈ cycloalkyl or a side chain of a natural or unnatural amino acid;
each R₂₃ independently is hydrogen, C₁₋₆ alkyl, C₆₋₁₀ aryl, C₃₋₈ cycloalkyl, —COOH, or —COO—C₁₋₆ alkyl;
a is an integer from 1 to 6;
c is an integer from 0 to 3;
d is an integer from 1 to 3;
f is an integer from 1 to 12;
R₁₁ is: 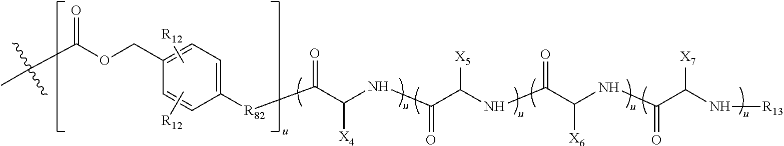
each R₁₂ independently is hydrogen, chloride, —CH₃ or —OCH₃;
R₁₃ is hydrogen or —C(O)—(CH₂)_{d}—(O—CH₂—CH₂)_{f}—NH₂;
R₈₂ is —NH or oxygen
X₄ is the side chain of lysine, arginine, citrulline, alanine or glycine;
X₅ is the side chain of phenylalanine, valine, leucine, isoleucine or tryptophan;
each of X₆ and X₇ is independently the side chain of glycine, alanine, serine, valine or proline;
each u independently is an integer 0 or 1;
or R₁₁ is —Yᵤ—W_{q}—R₈₈,
wherein:
Y is any one of the following structures: 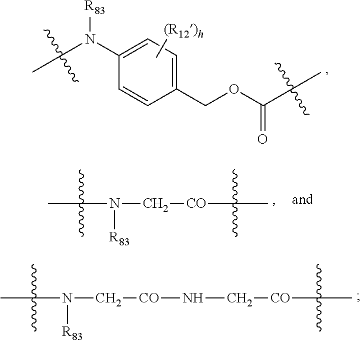
in each of which the terminal NR₈₃ group of Y is proximal to R₈₈;
R₈₃ is hydrogen or CH₃;
each W is an amino acid unit;
each R₁₂′ independently is halogen, —C₁₋₈ alkyl, —O—C₁₋₈ alkyl, nitro or cyano;
R₈₈ is hydrogen or —C(O)—(CH₂)_{ff}—(NH—C(O))ₐₐ-Eⱼ-(CH₂)_{bb}—R₈₅
R₈₅ is NH₂, OH or 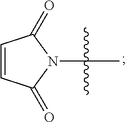 [SMILES: *N1C(=O)C=CC1=O]
E is —CH₂— or —CH₂CH₂O—;
q is an integer from 0 to 12;
aa is an integer 0 or 1;
bb is an integer 0 or 2;
ff is an integer from 0 to 10;
h is an integer from 0 to 4;
j is an integer from 0 to 12; and
when E is —CH₂—, bb is 0 and j is an integer from 0 to 10; and when E is —CH₂CH₂—O—, bb is 2 and j is an integer from 1 to 12;
or R₁₁ is 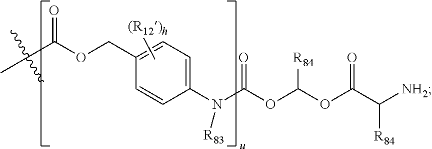
wherein:
R₈₃ is hydrogen or CH₃;
R₈₄ is C₁₋₆ alkyl or C₆₋₁₀ aryl;
each R₁₂′ independently is halogen, —C₁₋₈ alkyl, —O—C₁₋₈ alkyl, nitro or cyano; and
h is an integer from 0 to 4.

2. The scaffold of claim 1, wherein the PHF has a molecular weight ranging from 20 kDa to 150 kDa when the PBRM to be conjugated with has a molecular weight of less than 80 kDa, m₁ is an integer from 1 to 330, m₂ is an integer from 3 to 150, m₃ is an integer from 1 to 55 and the sum of m, m₁, m₂ and m₃, ranging from about 150 to about 1100.

3. The scaffold of claim 2, wherein the PHF has a molecular weight ranging from 30 kDa to 100 kDa, m₂ is an integer from 3 to about 100, m₃ is an integer from 1 to 40, m₁ is an integer from 1 to 220 and the sum of m, m₁, m₂, and m₃ ranging from about 220 to about 740.

4. The scaffold of claim 1, wherein the PHF has a molecular weight ranging from 6 kDa to 20 kDa when the PBRM to be conjugated with has a molecular weight of greater than 40 kDa, m₂ is an integer from 2 to 20, m₃ is an integer from 1 to 9, m₁ is an integer from 1 to 75 and the sum of m, m₁, m₂, and m₃ ranging from about 45 to about 150.

5. The scaffold of claim 4, wherein the PHF has a molecular weight ranging from 8 kDa to 15 kDa, m₂ is an integer from 2 to 15, m₃ is an integer from 1 to 7, m₁ is an integer from 1 to 55 and the sum of m, m₁, m₂, and m₃ ranging from about 60 to about 110.

6. The scaffold of claim 1, wherein the functional group of L^{P2} is selected from SR^{p}, —S—S-LG, maleimido, and halo, in which LG is a leaving group and R^{p} is H or a sulfur protecting group.

7. The scaffold of claim 1, wherein L^{D1} comprises —X—(CH₂)ᵥ—C(═O) with X directly connected to the carbonyl group of 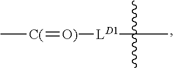
in which X is CH₂, O, or NH, and v is an integer from 1 to 6.

8. The scaffold of claim 1, wherein L^{P2} contains a biodegradable bond.

9. The scaffold of claim 1, further comprising a PBRM connected to the polymeric carrier via L^{P}.

10. The scaffold of claim 9, wherein the scaffold comprises one or more D-carrying polymeric carriers, each independently having Formula (Ic), connected to the PBRM: 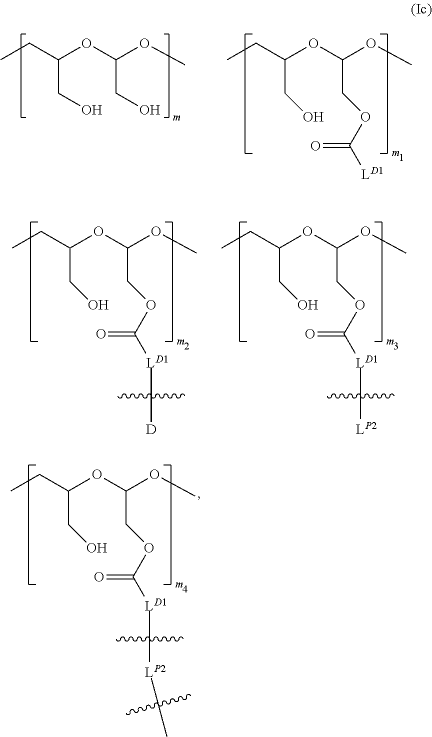
wherein:
the PBRM has a molecular weight of greater than 40 kDa,
the terminal 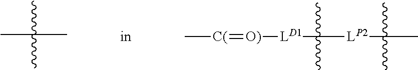
in denotes direct or indirect attachment of L^{P2} to PBRM such that the D-carrying polymeric carrier is connected to the PBRM,
m is an integer from 1 to 300,
m₁ is an integer from 1 to 140,
m₂ is an integer from 1 to 40,
m₃ is an integer from 0 to 18,
m₄ is an integer from 1 to 10; and
the sum of m, m₁, m₂, m₃, and m₄ ranges from 15 to 300;
provided that the total number of L^{P2} attached to the PBRM is 10 or less, and
the ratio of D to PBRM is between 5:1 and 40:1.

11. The scaffold of claim 10, wherein the sum of m, m₁, m₂, m₃ and m₄ ranges from 45 to 150, m₁ is an integer from 1 to 75, m₂ is an integer from 2 to 20, and m₃ is an integer from 1 to 9.

12. The scaffold of claim 10, wherein the sum of m, m₁, m₂, m₃ and m₄ ranges from 60 to 110, m₁ is an integer from 1 to 55, m₂ is an integer from 2 to 15, and m₃ is an integer from 1 to 7.

13. (canceled)

14. The scaffold of claim 9, wherein scaffold is of Formula (Ib): 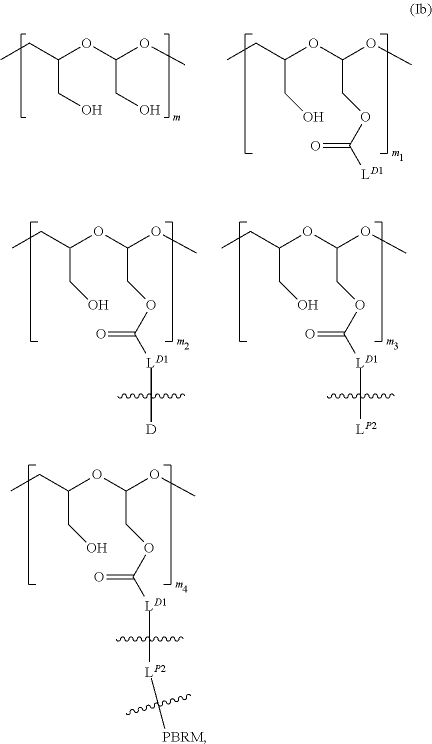
wherein:
the 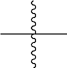
between L^{P2} and PBRM in 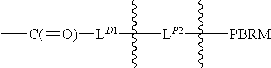
denotes direct or indirect attachment of PBRM to L^{P2}, such that the D-carrying polymeric carrier is connected to the PBRM,
each occurrence of PBRM independently has a molecular weight of less than 80 kDa,
m is an integer from 1 to 1100,
m₁ is an integer from 1 to 330,
m₂ is an integer from 3 to 150,
m₃ is an integer from 0 to 55,
m₄ is an integer from 1 to 30;
the sum of m, m₁, m₂, m₃ and m₄ ranges from 150 to 1100, and
the ratio of m₂ to m₄ is between 5:1 and 40:1.

15. The scaffold of claim 14, wherein the PHF has a molecular weight ranging from 30 kDa to 100 kDa, m₁ is an integer from 1 to 220, m₂ is an integer from 3 to 100, m₃ is an integer from 0 to 40, and m₄ is an integer from 1 to 20, and the sum of m₁ and m₂ is an integer from 18 to 220, and the sum of m₃ and m₄ is an integer from 1 to 40.

16. The scaffold of claim 14, wherein m₂ is an integer from 3 to about 150 and the sum of m₁ and m₂ is an integer from 14 to 330.

17. The scaffold of claim 14, wherein m₄ is an integer from 1 to about 10.

18. (canceled)

19. The scaffold of claim 1, wherein the second linker comprises a terminal group W^{P}, in which each W^{P} independently is: 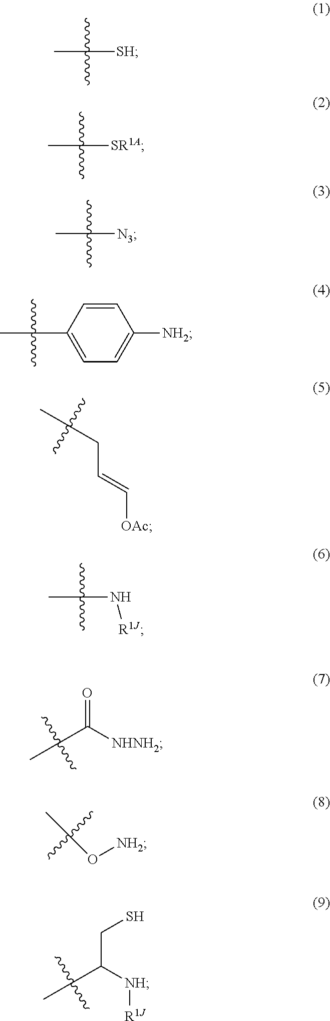 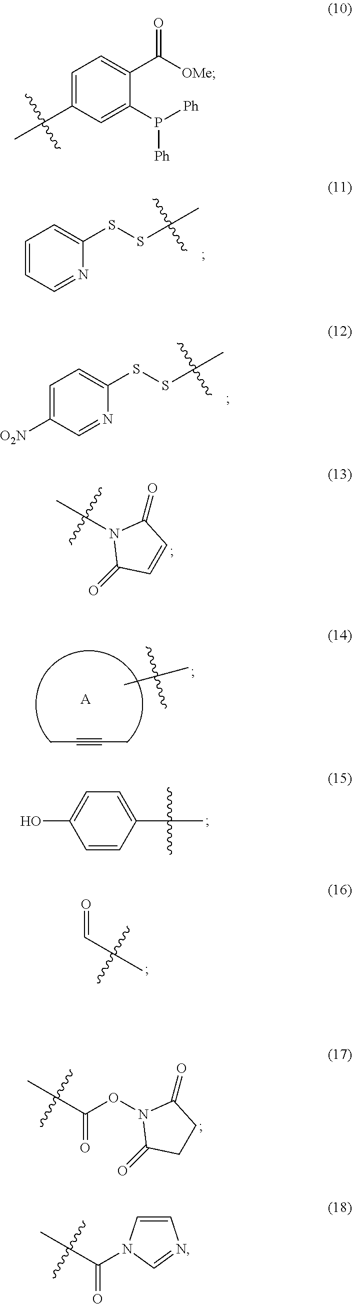 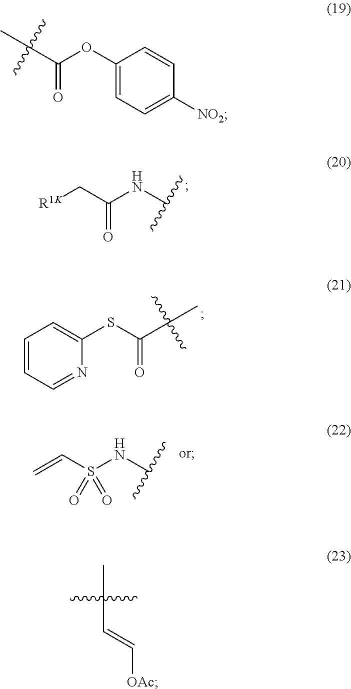
in which R^{1K} is a leaving group, R^{1A} is a sulfur protecting group, and ring A is cycloalkyl or heterocycloalkyl, and R^{1J} is hydrogen, an aliphatic, heteroaliphatic, carbocyclic, or heterocycloalkyl moiety.

20. The scaffold of claim 19, wherein R^{1A} is 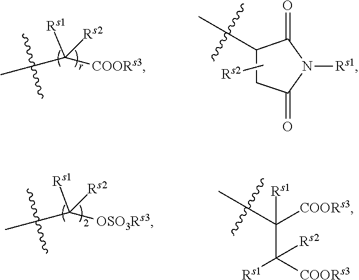
in which r is 1 or 2 and each of R^{s1}, R^{s2}, and R^{s3} is hydrogen, an aliphatic, heteroaliphatic, carbocyclic, or heterocycloalkyl moiety.

21-22. (canceled)

23. A pharmaceutical composition comprising a scaffold of claim 10 and a pharmaceutically acceptable carrier.

24. A method of treating a disorder in a subject in need thereof, comprising administering to the subject an effective amount of a scaffold of claim 10.

25-26. (canceled)

27. The scaffold of claim 1, wherein each occurrence of D is independently a compound of Formula (V) or a pharmaceutically acceptable salt thereof: 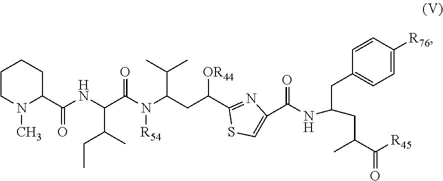
wherein R₄₄, R₅₄ and R₇₆ are as defined in the following table

| R₄₄ | R₅₄ | R₇₆ |
|---|---|---|
| —C(O)CH₃ | —CH₂OC(O)CH₂CH(CH₃)₂ | —OR₄₃ |
| —C(O)CH₃ | —CH₂OC(O)CH₂CH₂CH₃ | —OR₄₃ |
| —C(O)CH₃ | —CH₂OC(O)CH₂CH₃ | —OR₄₃ |
| —C(O)CH₃ | —CH₂OC(O)CH₂CH(CH₃)₂ | H |
| —C(O)CH₃ | —CH₂OC(O)CH₂CH₂CH₃ | H |
| —C(O)CH₃ | —CH₂OC(O)CH₂CH₃ | H |
| —C(O)CH₃ | —CH₂OC(O)CH═C(CH₃)₂ | —OR₄₃ |
| —C(O)CH₃ | —CH₂OC(O)CH₃ | H |
| —C(O)CH₃ | —CH₂OC(O)CH₃ | —OR₄₃ |
| —C(O)CH₃ | H | H |
| H | H | H |
| H | —CH₂OC(O)CH₂CH₂CH₃ | —OR₄₃ |
| —C(O)CH₃ | —CH₂OH | —OR₄₃ |
| —C(O)CH₃ | H | —OR₄₃ |
| H | H | —OR₄₃ |
| —C(O)CH₃ | H, CH₃, or CH₂CH₂CH₃ | Halogen |
| —C(O)CH₃ | CH₃ | —CH₃ |
| —C(O)CH₃ | CH₃ | —OCH₃ |
| —C(O)CH₃ | —CH₂OCH₃ | —OR₄₃ |
| —C(O)CH₃ | —CH₂O(CH₂)₂OH | —OR₄₃ |
| —C(O)CH₃ | —CH₂O(CH₂)₂CH(CH₃)₂ | —OR₄₃ |
| —C(O)CH₃ | —CH₂S (CH₂)₂SH | —OR₄₃ |
| —C(O)CH₃ | —(CH₂)₃—CH═CH₂ | —OR₄₃ |
| —C(O)CH₃ | —CH₂S(CH₂)₂OH | —OR₄₃ |
| —C(O)CH₃ | —CH₂OC(O)—CH═CH—CH₂Cl | —OR₄₃ |
| —C(O)CH₃ | —CH₂NHC(O)CH₂CH(CH₃)₂ | —OR₄₃ |
| —C(O)CH₃ | —CH₂O(CH₂)₂CH₃ | —OR₄₃ |
| —C(O)CH₃ | —CH₂S(CH₂)₂CH₃ | —OR₄₃ |

R₄₅ is mono- or di-alkylamino, —OR₄₂ or —NHR₄₀; and
at least one of R₄₃, R₄₂ and R₄₀ is not hydrogen.
